(19) United States
(12) Patent Application Publication
(10) Pub. No.: US 2024/0228524 A1
(43) Pub. Date: Jul. 11, 2024

(54) IRON(III) MACROCYCLIC COMPLEXES WITH MIXED HYROXYL PENDANTS AS MRI CONTRAST AGENTS

(71) Applicants: The Research Foundation for The State University of New York, Amherst, NY (US); Ferric Contrast Inc., Amherst, NY (US)

(72) Inventors: Janet R. MORROW, Williamsville, NY (US); Eric M. SNYDER, Tonawanda, NY (US); Patrick BURNS, Germantown, NY (US); Elizabeth A. KRAS, Amherst, NY (US); Jaclyn RAYMOND, Buffalo, NY (US)

(21) Appl. No.: 18/551,458

(22) PCT Filed: Mar. 21, 2022
(86) PCT No.: PCT/US2022/021216

§ 371 Date: Sep. 20, 2023

Related U.S. Application Data
(60) Provisional application No. 63/176,193, filed on Apr. 16, 2021.
(60) Provisional application No. 63/163,822, filed on Mar. 20, 2021.

(51) Int. Cl. C07F 15/02; A61K 49/10; A61K 49/14

(52) U.S. Cl. CPC C07F 15/025; A61K 49/106; A61K 49/143

(57) ABSTRACT

The present application describes novel Fe(III) macrocyclic complexes that have hydroxy pendants with a third anionic ancillary group for improved MR imaging in vivo. The complexes have the following general structure: (I) or (II) where high spin Fe(III) is chelated thereto. The present application also describes novel Fe(III) macrocyclic complexes that have hydroxypropyl pendants with a third anionic ancillary group for improved MR imaging in vivo. The complexes have the following general structure.

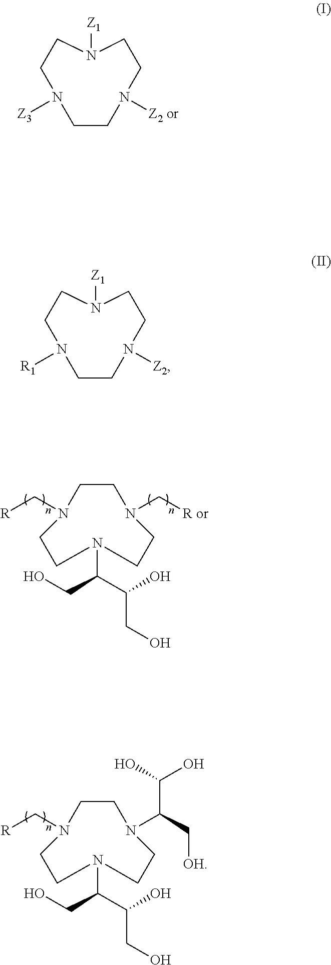

### CROSS-REFERENCE TO RELATED APPLICATION

This application claims priority to U.S. Provisional Patent Application No. 63/163,822, filed Mar. 20, 2021, and U.S. Provisional Patent Application No. 63/176,193, filed on Apr. 16, 2021, the disclosures of which are incorporated herein.

### STATEMENT REGARDING FEDERALLY SPONSORED RESEARCH

This invention was made with government support under grant STTR-1951127 awarded by the National Science Foundation. The government has certain rights in this invention.

### BACKGROUND OF THE DISCLOSURE

Nearly all clinically-used contrast agents contain gadolinium (Gd as trivalent Gd(III)), yet a substantial proportion of patients in the US population (ca 10%) are considered at risk for being given Gd(III) contrast agents due to toxicity arising from long-term exposure. In addition, there are new concerns that Gd(III) based MRI contrast agents are leading to the deposition of Gd(III) into brain, bone and skin of all patients. Alternatives to Gd(III) contrast agents include biologically relevant transition metal ions such as high-spin Fe(III) complexes.

An alternative approach in magnetic resonance imaging (MRI) is the development of contrast agents that utilize iron as an endogenous metal ion. Contrast agents containing Fe(III), as trivalent iron, would provide an alternative to Gd(III) contrast agents that are problematic for patients who cannot tolerate Gd(III). Most Fe(III) MRI contrast agents that have been reported to date contain simple linear chelates. There are three commonly used types of complexes. The most heavily studied are the class which contains an ethylene diamine backbone with a combination of phenol and carboxylate pendants such as EHBG (NN′-ethylenebis[(2-hydroxybenzyl)glycine). The second type contains polyaminocarboxylate ligands, such as Fe(III) complexes of EDTA. The third type contains the bacterial siderophore, desferrioxamine (DFO). All of these complexes have drawbacks including lack of exchangeable water ligands, reduction potentials that are amenable for ROS generation and/or difficulty of synthetic modification. Also, the aqueous solution chemistry of Fe(III) complexes is dominated by the formation of insoluble complexes with hydroxides and bridging oxide ligands. Improvements are needed to obtain Fe(III) complexes that are not effective catalysts for the production of ROS by tuning redox potential to stabilize Fe(III), are water soluble and are desirable T₁ relaxivity agents.

### SUMMARY OF THE DISCLOSURE

The present application describes novel Fe(III) macrocyclic complexes that have hydroxy pendants with a third anionic ancillary group for improved MR imaging in vivo. The complexes have the following general structure:

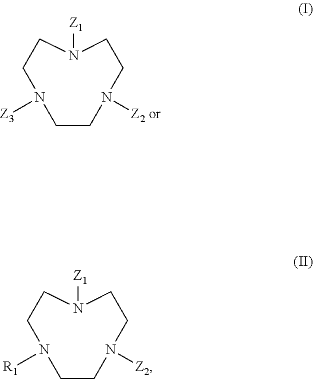

where high spin Fe(III) is chelated thereto. The present application also describes novel Fe(III) macrocyclic complexes that have hydroxypropyl pendants with a third anionic ancillary group for improved MR imaging in vivo. The complexes have the following general structure:

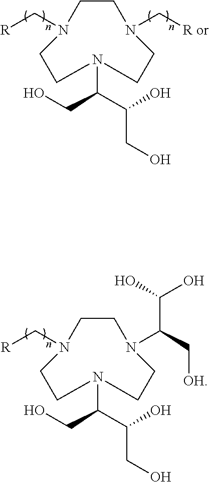

In an aspect, the present disclosure provides a macrocyclic compound having i) a macrocyclic core comprising at least one heteroatom as a ligand donor and ii) at least one pendant donor as a substituent of the macrocyclic core. A macrocyclic compound may be referred to as a ligand when the macrocyclic compound is coordinated to an iron(III) ion. The macrocyclic core has a ring structure comprising carbon atoms and at least one heteroatom (e.g. N atom). As used herein, “macrocycle donor” refers to a heteroatom with an available lone pair of electrons to donate to the Fe(III) center when present in the macrocyclic core of the macrocyclic compound. For example, the macrocycle donor can be a nitrogen atom (e.g. a tertiary amine, a secondary amine). As used herein, “pendant donor” refers to a heteroatom with an available lone pair of electrons to donate to the Fe(III) center when present in a substituent on the macrocyclic core of the macrocyclic compound. For example, the pendant donor can be a nitrogen-containing group (e.g., amino, benzimidazole, imidazole, aniline, pyrazoyl, triazole, benzotriazole, and the like), an oxygen-containing group (e.g., ketone, alcohol, alkoxide, amide, phosphonic acid, carboxylic acid, and the like). Some pendant donors, such as, for example, carboxylic acid, alcohol, imidazole or pyrazole may deprotonate when complexed with Fe(III) or at certain pHs. Such protonated and deprotonated forms are within the scope of the disclosure. For example, the pendant donor may be a phosphonate, phosphinate, phenolate or an oxide (e.g., an alkoxide or a phenoxide).

### BRIEF DESCRIPTION OF THE FIGURES

For a fuller understanding of the nature and objects of the disclosure, reference should be made to the following detailed description taken in conjunction with the accompanying figures.

FIG. 1 shows general synthesis of TACN (1,4,7-triazacyclononane) derivatives. a) N,N-Dimethylformamide Dimethylacetal, Toluene/Chloroform 4:1. b) R-X; R=Benzyl, methyl, propargyl, methylphenyl, methyl-benzoate, 2-(2-methoxy-ethoxy)ethane, 4-(methyl)-1,1′-biphenyl, Benzyl methyl ether; Dry THE and X=chloro, bromo or iodo c) Reflux; 12M HCl/MeOH 1:1 OR KOH solution, then extraction with chloroform. d) Addition of coordinating pendants by addition of chloro or bromomethyl derivatives of coordinating pendants such as bromomethyl-pyrazole, or bromoacetamide. Addition of pendants by reductive amination by addition of aldehyde with reducing agent such as imidazole-2-carboxaldehyde. Addition of pendants by addition of H₂O/ethanol mixture and (S)-(−) propylene oxide or (R)-(+)propylene oxide.

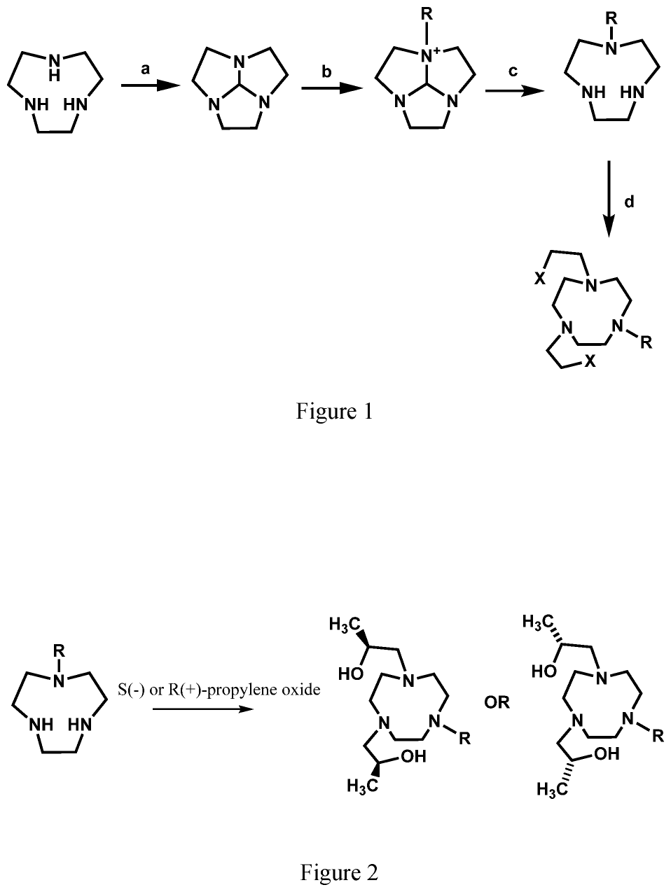

FIG. 2 shows general synthesis of TACN ligands with two chiral propyl alcohol pendants. Either R or S propylene oxide can be used to give pendants with opposite chirality. The non-coordinate group, R, is typically benzyl, methyl or biphenyl.

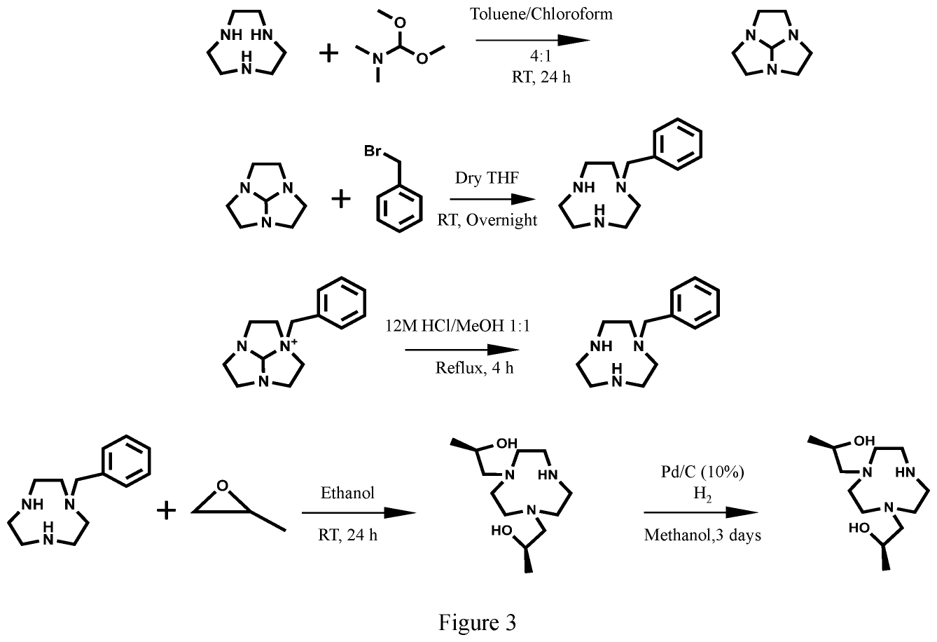

FIG. 3 shows synthesis of the TON ligand, a synthetic precursor from the TOB ligand. The benzyl group is removed by catalytic hydrogenation to produce TON.

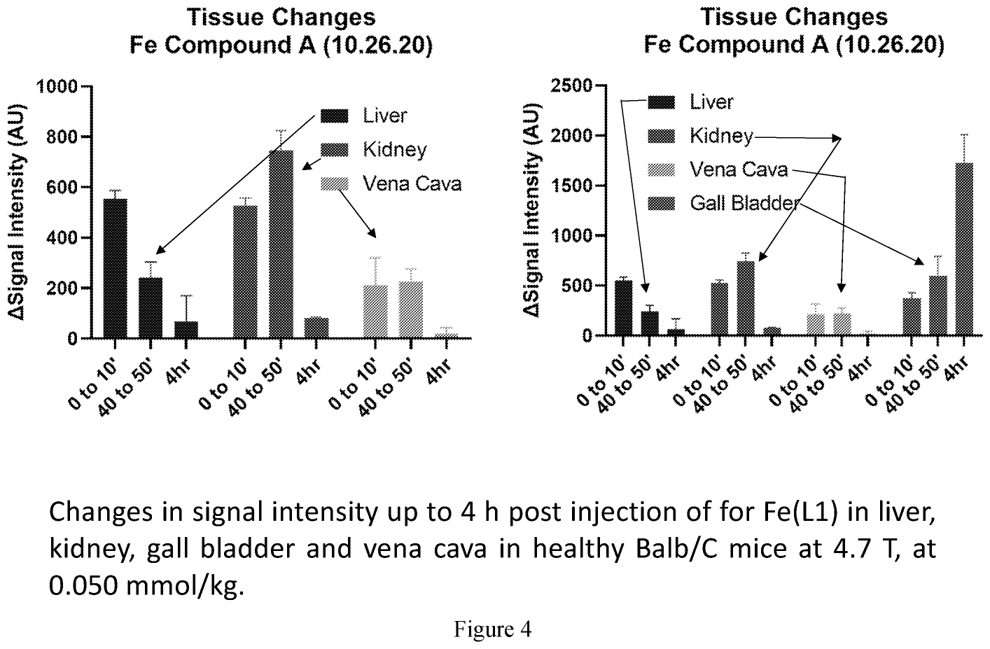

FIG. 4 shows changes in signal intensity up to 4 h post injection of for Fe(TOP) in liver, kidney, gall bladder and vena cava in healthy Balb/C mice at 4.7 T, at 0.050 mmol/kg.

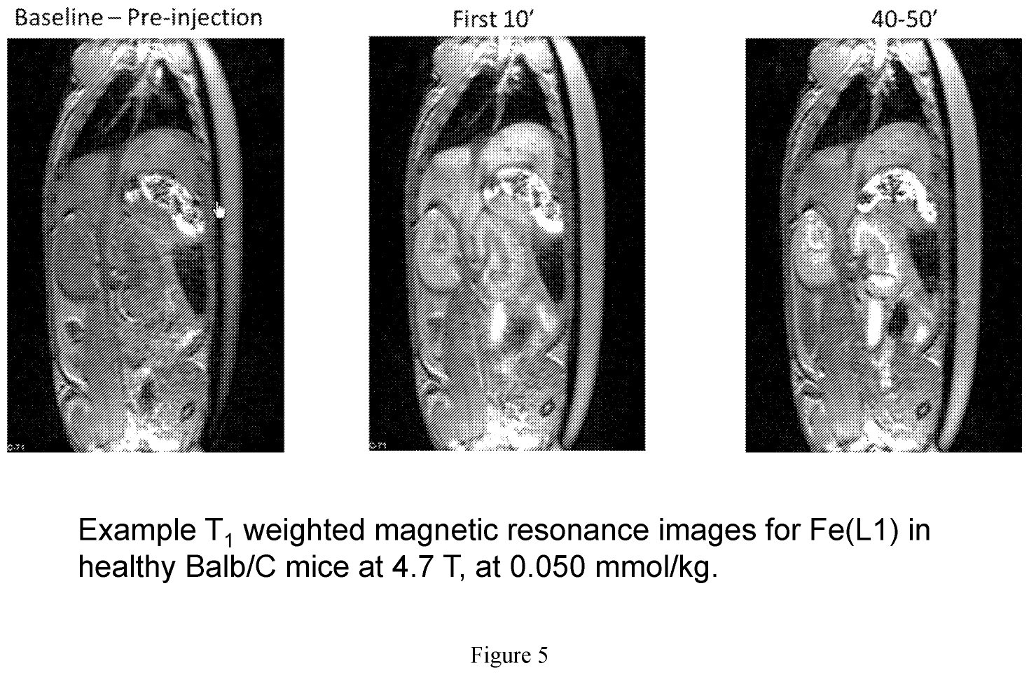

FIG. 5 shows changes in signal intensity up to 4 h post injection for Fe(TOP) in liver, kidney, gall bladder and vena cava in healthy Balb/C mice at 4.7 T, at 0.050 mmol/kg.

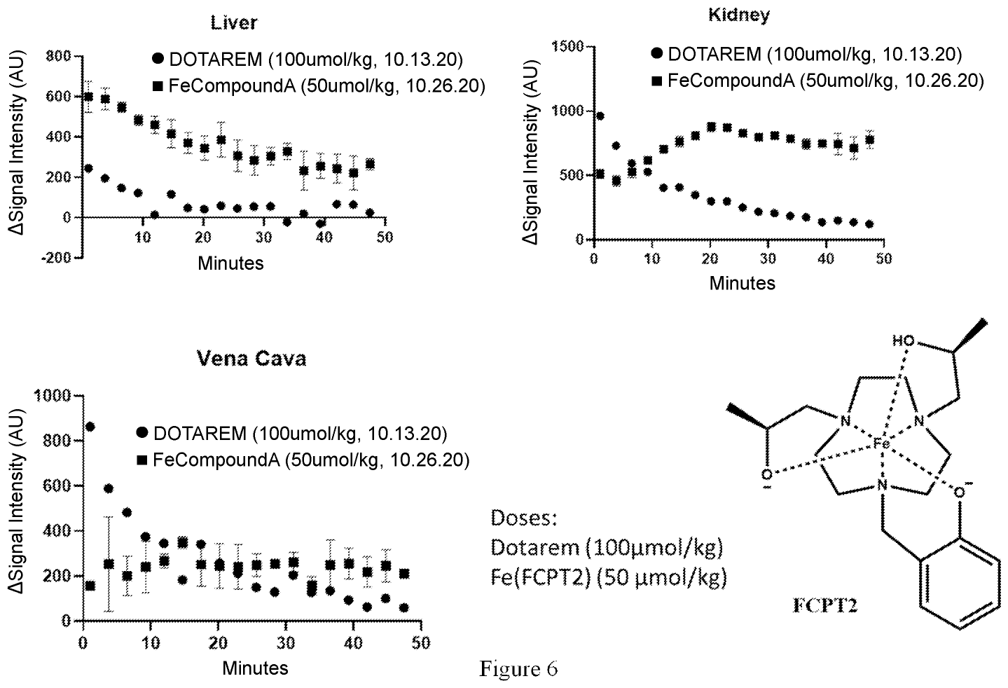

FIG. 6 show changes in signal intensity (T1 weighted imaging) post injection of Fe(TOP) over time in blood (Vena Cava, kidneys, liver in healthy Balb/C mice at 0.050 mmol/kg for iron or 0.10 mmol/kg for DOTAREM. Imaging was done on a 4.7 T MRI scanner in healthy Balb/C mice.

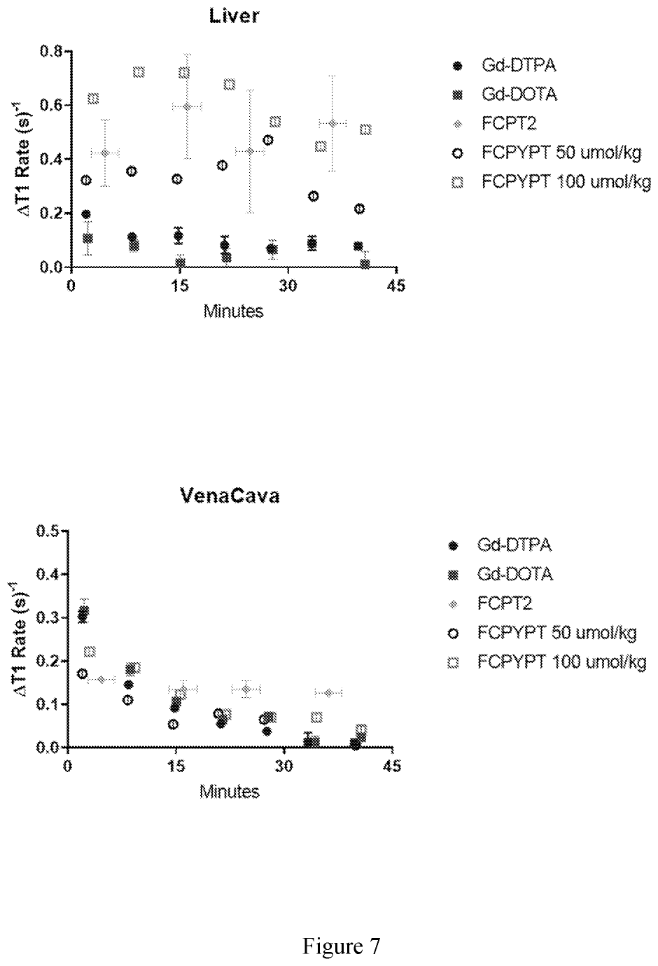

FIG. 7 shows changes in signal intensity post injection for FeFCPT2 and FCPYPT in liver, kidney, kidney vessel, liver and vena cava in healthy Balb/c mice at 4.7 T at 0.05 mmol/kg dose or 0.100 dose iron compared to Gd(III) agents Gd-DOTA or Gd(DTPA). Imaging was done on a 4.7 T MRI scanner in healthy BALB/c mice.

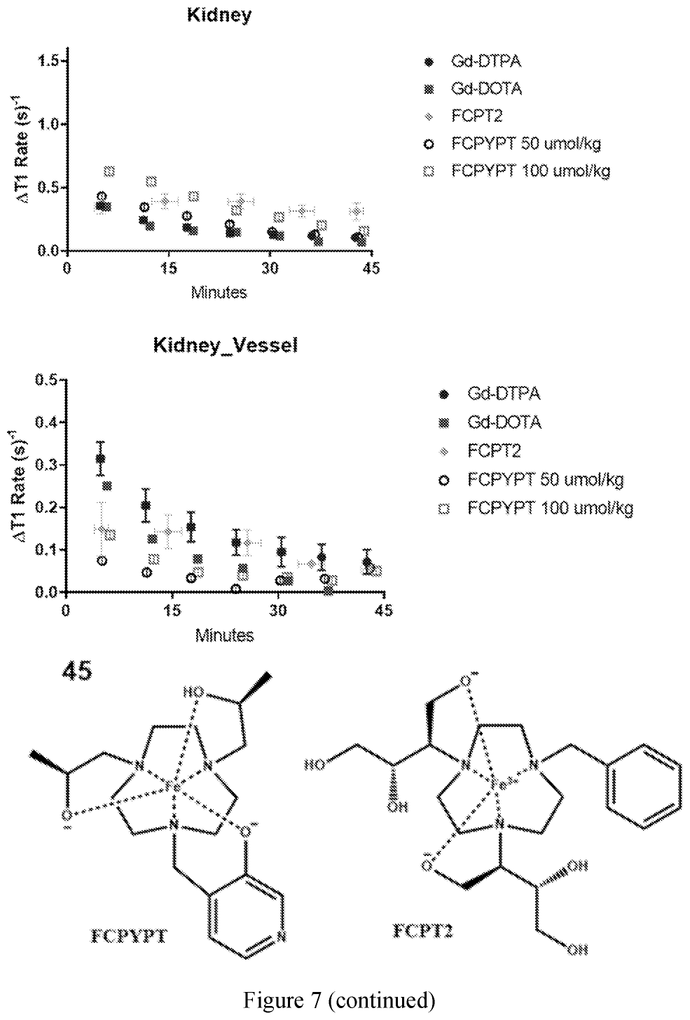

FIG. 8 shows an example of T1 weighted MRI for Fe(L1) in healthy Balb/C mice at 4.7 T. The figure shows changes in signal intensity post injection for Fe(TPP) in liver, kidney, kidney vessel, liver and vena cava in healthy BALB/c mice at 4.7 T at 0.100 mmol/kg dose compared to Gd(III) agents Gd-DOTA (Dotarem) or Gd(DTPA).

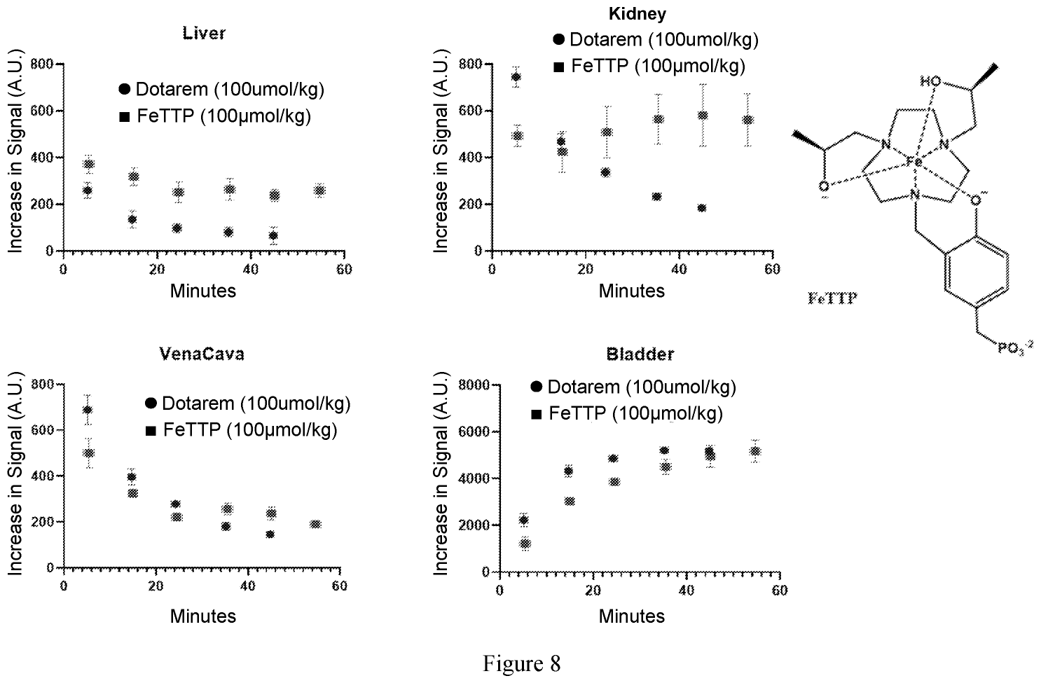

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

In this application, the use of the singular form encompasses the plural and vice versa.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

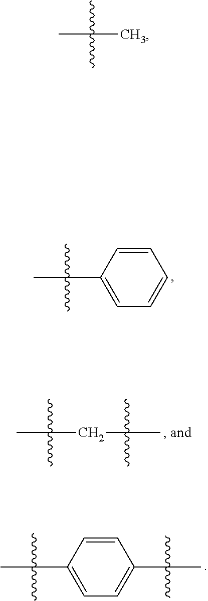

As used herein, unless otherwise indicated, the term “alkyl group” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₃₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). The alkyl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise indicated, the term “heteroaryl group” refers to a C₁ to C₁₄ monocyclic, polycyclic, or bicyclic ring groups (e.g., aryl groups) comprising one or two aromatic rings containing at least one heteroatom (e.g., nitrogen, oxygen, sulfur, and the like) in the aromatic ring(s), including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, and C₁₄). The heteroaryl groups may be substituted or unsubstituted. Examples of heteroaryl groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, and thiadiazolyl groups, and the like. Examples of substituents include, but are not limited to, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof.

The present application describes novel Fe(III) macrocyclic complexes that have hydroxy pendants with a third anionic ancillary group for improved MR imaging in vivo. The present application also describes novel Fe(III) macrocyclic complexes that have hydroxypropyl pendants with a third anionic ancillary group for improved MR imaging in vivo.

Contrast agents should be sufficiently hydrophilic to prevent strong protein binding and to accelerate pharmacokinetic clearance in vivo. One method of accomplishing this it to add tri(hydroxy)butyl groups as pendants on the macrocycles and Fe(III) macrocyclic complexes as shown here.

It is an object of the present disclosure to provide macrocyclic compounds, which may be Fe(III) macrocyclic complexes. Also provided are compositions and methods of making and using same. In various examples, macrocyclic complexes and compositions of the present disclosure are used as MRI contrast agents. The present application further describes novel Fe(III) macrocyclic complexes that have tri(hydroxy)butyl pendant groups. A tri(hydroxy)butyl pendant group has three alcohol groups for stronger interaction of the complex with water. The macrocyclic complex for improved MR imaging in vivo.

The macrocyclic compounds of the present disclosure as ligands have advantages towards accomplishing control over spin and oxidation state of the Fe(III) complexes and also interactions of the complex with innersphere and outersphere water and through proton exchange or the hydroxyalkyl groups. The cavity of these macrocyclic ligands can be suitable for stabilization of Fe(III) in high-spin form. Also, control of the aqueous solution chemistry of the Fe(III) complex can be accomplished with these macrocyclic compounds. The macrocyclic complexes described here nearly encapsulate the Fe(III), but in some cases, have a coordination site for water ligands that enhances their efficacy as T₁ MRI contrast agents. The compounds also have protons on the hydroxyalkyl pendants. Without intending to be bound by any particular theory, it is considered that the protons on the hydroxyalkyl pendant groups produce improved relaxivity through exchange with bulk water protons. Without intending to be bound to any particular theory, it is considered that the iron-based MRI contrast agents described herein (as high-spin, trivalent Fe(III)) produce contrast by paramagnetic mechanisms known for Gd(III) agents and are in small molecule form as coordination complexes, i.e., they are not iron-oxide based nanoparticles.

In the present disclosure, the macrocyclic compounds have a variety of macrocyclic core structures and a variety of substituents (also referred to as “pendant donor groups,” “pendant groups,” “pendant donors,” or “donor groups”) on the macrocyclic core. Most typically, donor groups contain amides, alcohols or phenols, but with at least two alcohol groups or other groups that can deprotonate to form anionic groups. The macrocyclic compounds are complexed to Fe(III) to provide a stabilized trivalent state.

In an aspect, the present disclosure provides a macrocyclic compound having i) a macrocyclic core comprising at least one heteroatom as a ligand donor and ii) at least one pendant donor as a substituent of the macrocyclic core. A macrocyclic compound may be referred to as a ligand when the macrocyclic compound is coordinated to an iron(III) ion. The macrocyclic core has a ring structure comprising carbon atoms and at least one heteroatom (e.g. N atom). As used herein, “macrocycle donor” refers to a heteroatom with an available lone pair of electrons to donate to the Fe(III) center when present in the macrocyclic core of the macrocyclic compound. For example, the macrocycle donor can be a nitrogen atom (e.g. a tertiary amine, a secondary amine). As used herein, “pendant donor” refers to a heteroatom with an available lone pair of electrons to donate to the Fe(III) center when present in a substituent on the macrocyclic core of the macrocyclic compound. For example, the pendant donor can be a nitrogen-containing group (e.g., amino, benzimidazole, imidazole, aniline, pyrazoyl, triazole, benzotriazole, and the like), an oxygen-containing group (e.g., ketone, alcohol, alkoxide, amide, phosphonic acid, carboxylic acid, and the like). Some pendant donors, such as, for example, carboxylic acid, alcohol, imidazole or pyrazole may deprotonate when complexed with Fe(III) or at certain pHs. Such protonated and deprotonated forms are within the scope of the disclosure. For example, the pendant donor may be a phosphonate, phosphinate, phenolate or an oxide (e.g., an alkoxide or a phenoxide).

In certain embodiments, the macrocyclic compounds have the following structure:

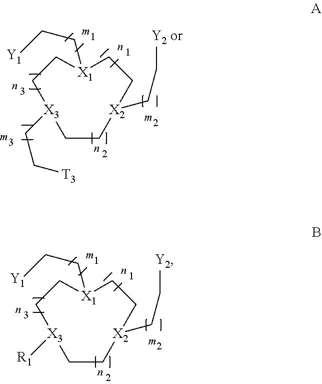

where X₁, X₂, and X₃, are N; Y₁, Y₂, or Y₃ are each independently pendant donors comprising O, wherein O has at least one lone pair of electrons but preferably two or three lone pairs (e.g., ketone, alcohol, alkoxide, carboxylic acid, phosphinic acid, phosphonic acid, amide, phenol or phenoxide, or a deprotonated form of the foregoing, such as, for example, a carboxylate ion, phosphinate, phosphonate, or an oxide, including an alkoxide or a phenoxide; m₁, m₂, or m₃ are each independently 0, 1, or 2; n₁, n₂, or n₃ are each independently 1 or 2; and R₁ is a substituted or unsubstituted aryl, substituted or unsubstituted heteroaryl or substituted or unsubstituted alkyl group and R₁ is not substituted by a pendant donor, wherein the alkyl segment of the alkyl-Y chain (alkyl-Y₁, alkyl-Y₂, and/or alkyl-Y₃) may each independently be substituted (e.g., Structure a or Structure b) or unsubstituted. For Structures a or b, the pendant may have either R or S configuration at the chiral carbon:

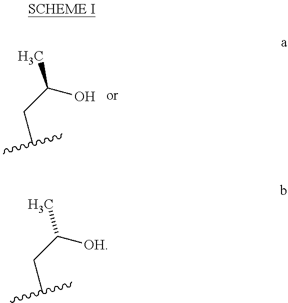

In an embodiment, the disclosure provides macrocyclic compounds having the structures and definitions set forth herein.

Examples of suitable macrocyclic compounds include:

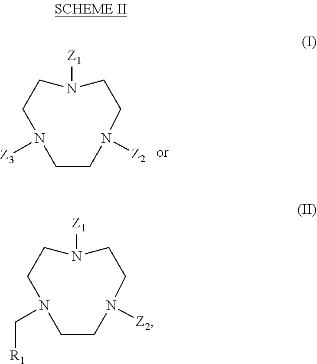

where R₁ is a substituted or unsubstituted phenyl group, a substituted or unsubstituted heteroaryl group (e.g., pyridinyl, pyrazolyl, or imidazolyl), a substituted or unsubstituted alkyl group wherein the substituted or unsubstituted alkyl group is not a methyl group and, optionally, R₁ is not a substituted pendant donor. For example, when the macrocyclic core has Structure I, Z₁ is H or one of the pendant groups in Scheme III and Z₂ and Z₃ each independently is a pendant group (e.g., one of the pendant groups in Scheme III); when the macrocyclic compound has Structure II, Z₁ and Z₂ each independently is a pendant group (e.g., one of the pendant groups in Scheme III).

The macrocyclic compound has at least two pendant donors on the macrocyclic core. For example, the pendant donor can have any one of the structures from Scheme III:

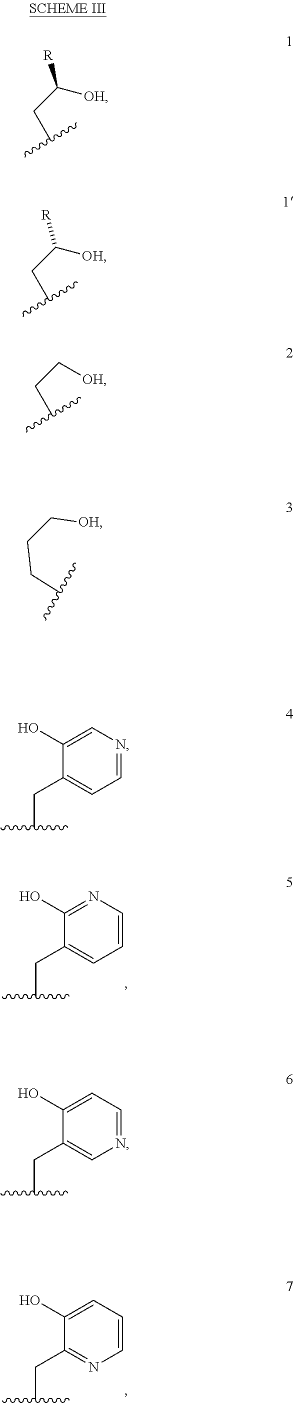

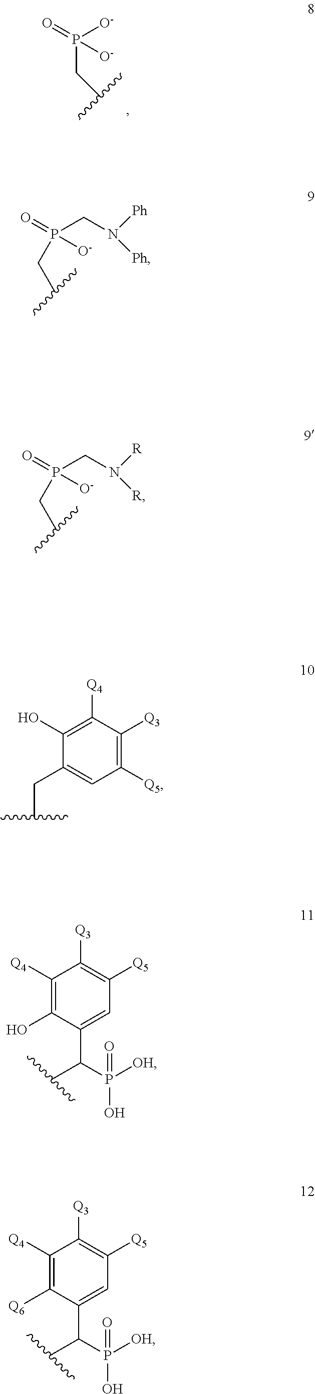

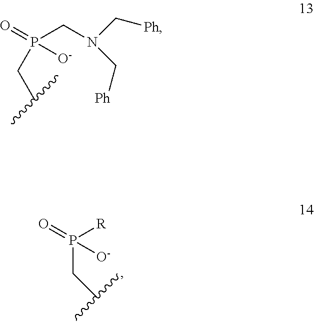

and protonated, partially deprotonated, or completely deprotonated species thereof (where applicable), where Q₃, Q₄ and Q₅ are each independently anionic groups or chosen from —H, —NR₂, —NO₂, —CN, —(CH₂)ₘNR₂, OH, OR, —CH₂P(O)(OH)₂, —(CH₂)ₘP(O)(OH)₂, —SO₃H, and deprotonated species thereof, where m is 1 or 2, where R is H, an alkyl group (e.g., methyl, trifluoromethyl, or the like), an aryl group (e.g., a phenyl group or a phenyl group substituted with a sulfonate), an alkyl carboxylate group, alkyl carboxylic acid group, or the like. The compounds have two of any of 1, 1′, 2, 3, 4, 8, 10, or a combination thereof. In various examples the pendant donors are different (e.g., a macrocycle has at least two different pendant donors). Some pendant donors, such as, for example, alcohols, phenolic pendants, and the like, may deprotonate when complexed with Fe(III) or at certain pH values. Such protonated and deprotonated forms are within the scope of the disclosure. For example, the pendant donor is an oxide (e.g., an alkoxide, a phenoxide, or the like).

In various non-limiting embodiments, when the macrocyclic core has Structure I, Z₁ and Z₂ are both 1, 1′, 2, 3, 4, 8, 10 or any combination thereof, R₁ is chosen from any one of SCHEME III pendant groups 5, 6, 7, 9, 11, 12, 13, or 14.

The macrocyclic compound may comprise one or more ancillary pendant groups. The ancillary pendant group (s) may be one or more coordinating ancillary pendant groups and/or one or more non-coordinating ancillary pendant groups.

A non-coordinating ancillary pendant group does not have a heteroatom that can bind to the Fe(III) metal ion to form a five-membered or six-membered chelate. Non-limiting examples of non-coordinating ancillary pendant groups include benzyl groups, phenyl groups, and other aromatic (e.g., aryl) groups that have one or more methylene group attached to aromatic group or no methylene groups), alkyl groups (both branched and linear groups), and the like. Other non-limiting examples of non-coordinating ancillary pendant groups include biphenyl, napthyl, anthracenyl, pyridyl, quinolyl, methyl, ethyl, isopropyl, n-propyl, ethyl methoxyether, PEG derivatives (polyethylene glycol), and the like.

In various other examples, the macrocyclic compounds have the following structure:

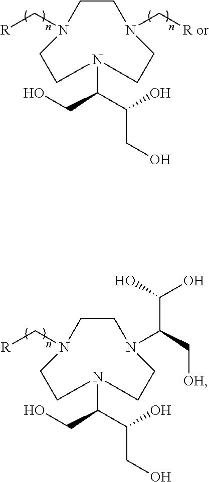

where the tri(hydroxy)butyl group(s) and —(CH₂)ₙR groups are pendant groups and each R is independently selected from alkyl groups; aryl groups; heteroaryl groups; alkyl groups comprising one or more —OH groups, one or more sulfonic acid groups, one or more carboxylic acid groups, one or more phosphonic acid groups, one or more alkyl groups, or the like, or combinations thereof, aryl groups comprising one or more —OH groups, one or more sulfonic acid groups, one or more carboxylic acid groups, one or more phosphonic acid groups, one or more alkyl groups, or the like, or combinations thereof, heteroaryl groups comprising one or more —OH groups, one or more sulfonic acid groups, one or more carboxylic acid groups, one or more phosphonic acid groups, one or more alkyl groups, or the like, or combinations thereof, and H. The R groups may substituted or unsubstituted R groups. Each n is independently selected from 1, 2, or 3. The pendant groups may have one or more chiral carbons.

Non-limiting examples of pendant groups are:

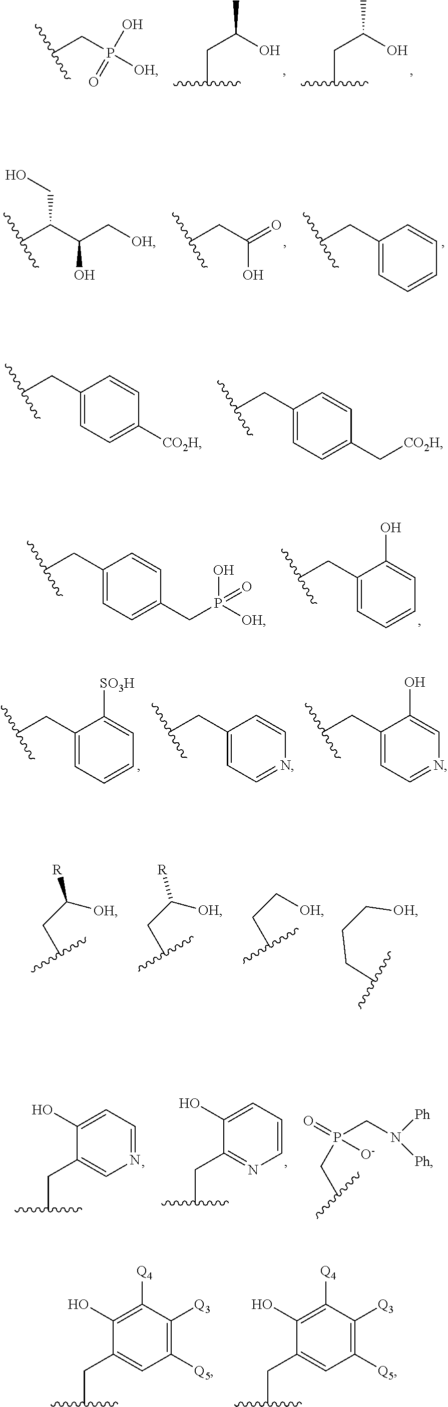

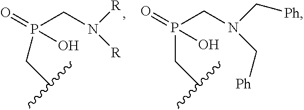

and protonated, partially deprotonated, and deprotonated species thereof (where applicable), Q₃, Q₄ and Q₅ are each independently anionic groups or chosen from —H, —NR₂, —NO₂, —CN, —(CH₂)ₘNR₂, OH, OR, —CH₂PO(OH)₂, —(CH₂)ₘP(O)(OH)₂, —SO₃H, and deprotonated, partially deprotonated, and protonated species thereof (where applicable), where m is 1 or 2, or a partially or completely deprotonated analog thereof, where R is H, an alkyl group (e.g., methyl, trifluoromethyl, or the like), an aryl group (e.g., a phenyl group or a phenyl group substituted with a sulfonate), an alkyl carboxylate group, alkyl carboxylic acid group, or the like.

The Fe(III) complex may have a bound water, hydroxide, or no bound water or hydroxide ligands.

In certain embodiments, an Fe(III) cation, which may be a high spin Fe(III) cation, is complexed to the macrocyclic compound. In certain other embodiments, Fe(III) cation is not complexed to the macrocyclic compound. The Fe(III), which may be a high spin Fe(III) cation, may be complexed to the macrocycle as shown herein.

As previously noted, some pendant donors, such as, for example, alcohol, phenolic pendant groups, may deprotonate when complexed with Fe(III). Their corresponding phenolate ions or oxides (e.g., alkoxide or phenoxide) are within the scope of the disclosure.

Non-limiting examples of coordinating ancillary pendant groups (e.g., which is an additional pendant group when two are already hydroxyl propyl) include oxygen or nitrogen donors that form five or six-membered chelates such as, for example, amides, carboxylates, phosphinates, phosphonates, alcohols, phenols, or derivatives of aminophenol, and the like. Some of these groups may deprotonate when bound to Fe(III).

A macrocyclic complex comprising one or more non-coordinating ancillary pendant group may have an open coordination site (have open coordination). A macrocyclic complex comprising one or more coordinating ancillary pendant group may not have an open coordination site (have closed coordination).

In various embodiments, Z₁, Z₂, and Z₃ are independently chosen from:

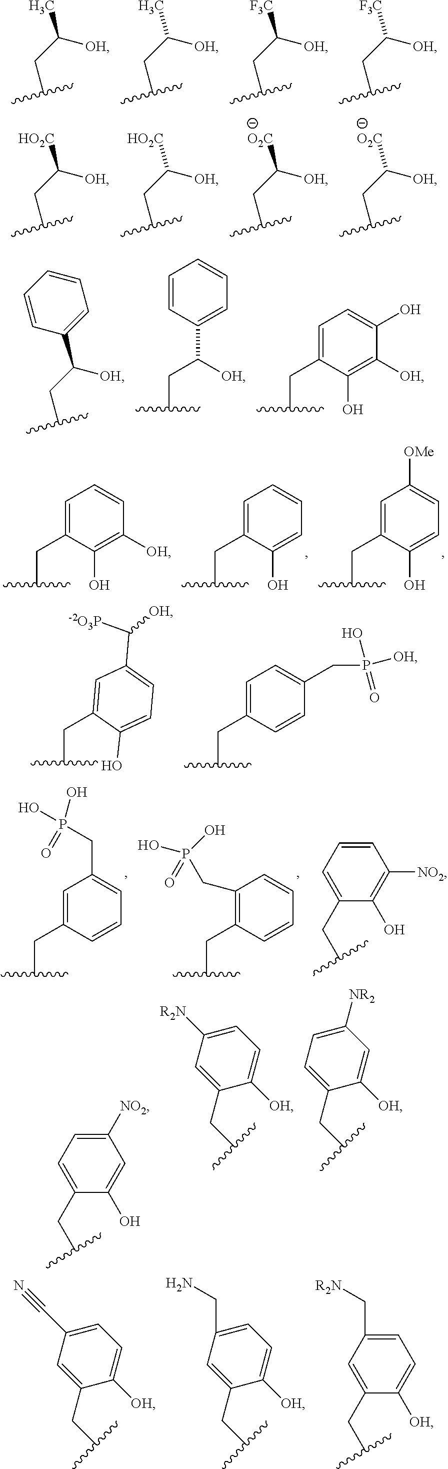

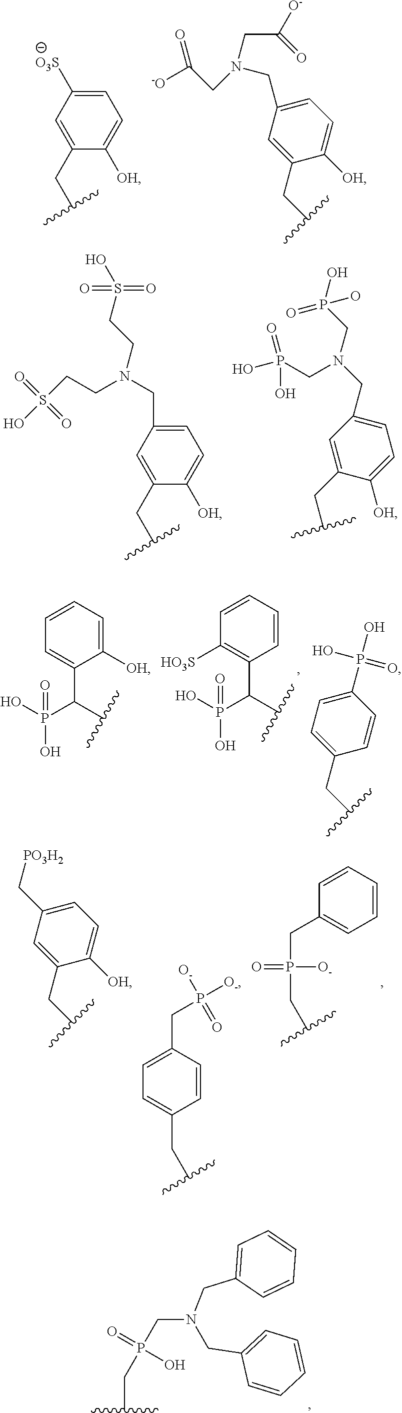

and protonated, deprotonated, and partially deprotonated species thereof (where applicable).

In one embodiment, the subject disclosure provides macrocyclic compounds having the structures and definitions set forth in Schemes II-III, where any or all of the following provisos apply when the macrocycle has Structure I: when Z₁=Z₂=Structure 1, Z₃≠Structure 1; when Z₁=Z₂=Structure 1′, Z₃≠Structure 1′; when Z₁=Z₂=Structure 2, Z₃≠Structure 2; when Q₁=Q₂=H; when Z₁=H, Z₂≠Structure 2.

In certain embodiments, an Fe(III) cation, which may be a high spin Fe(III) cation, is complexed to the macrocyclic compound. In certain other embodiments, Fe(III) cation is not complexed to the macrocyclic compound. The Fe(III), which may be a high spin Fe(III) cation, may be complexed to the macrocycle as shown herein.

As previously noted, some pendant donors, such as, for example, alcohol, phenolic pendant, may deprotonate when complexed with Fe(III). Their corresponding phenolate ions or oxides (e.g., alkoxide or phenoxide) are within the scope of the disclosure.

In an embodiment, the disclosure provides Fe(III) complex comprising Fe(III) complexed with a macromolecule having a structure set forth in Schemes II-III, as defined in Schemes II-III.

In another embodiment, the subject disclosure provides Fe(III) complexes comprising Fe(III) complexed with a macromolecule having a structure set forth in Schemes II-III, as defined in Schemes II-III, where any or all of the following provisos apply when the macromolecule has Structure I: when Z₁=Z₂=Structure 1, Z₃≠Structure 1; when Z₁=Z₂=Structure 1′, Z₃≠Structure 1′; when Z₁=Z₂=Structure 2, Z₃≠Structure 2.

Certain pendants may have more than one O donor atom (e.g., substituted alkylphenol pendants and the like) although generally only one is coordinated to metal ion. The polyols can be alkyl polyols, aryl polyols, or a combination thereof.

A macrocyclic compound can have various pendant groups and combinations of pendant groups. When more than one pendant donor is present, they may be the same or different.

In various examples, the macrocyclic core has 3 nitrogen atoms. In various examples, there are 2 carbon atoms separating the nitrogen atoms in the macrocyclic core. The one or more carbons in the macrocyclic core can be unsubstituted (e.g., —CH₂—) or substituted (e.g., —CHR—, or —CRR′-, where R and R′ are, for example, alkyl groups or aryl groups (e.g., benzyl groups) as described herein).

The pendant groups can be covalently attached to a macrocyclic core (e.g. at a nitrogen). For example, the pendant groups are covalently attached to a TACN (I) macrocyclic core.

Examples of macrocycles of the present disclosure include, but are not limited to,

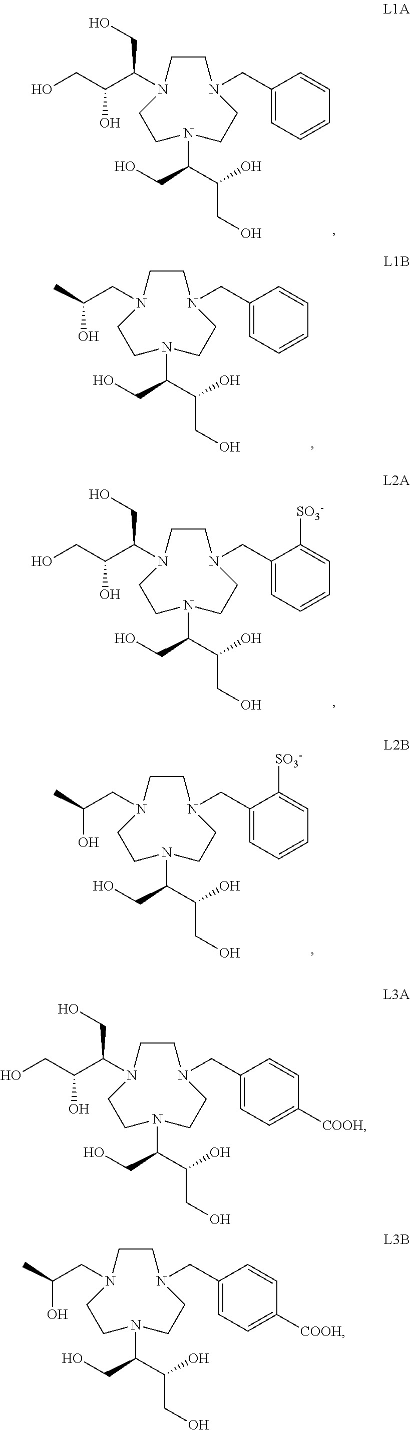

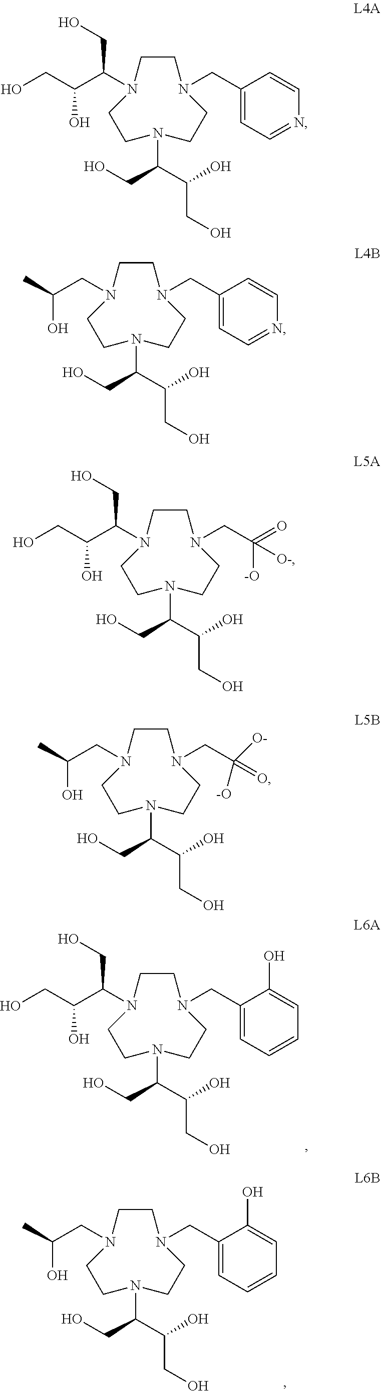

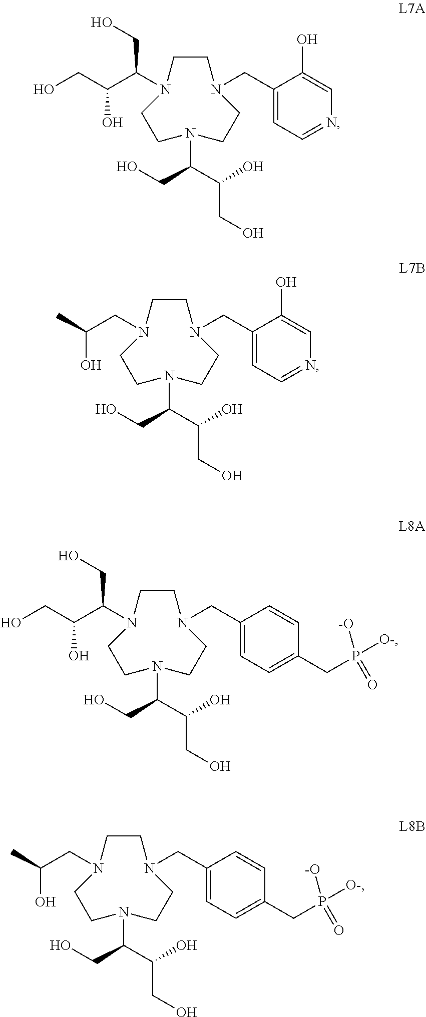

and protonated and deprotonated analogs thereof.

Examples of chelated macrocycles include, but are not, limited to:

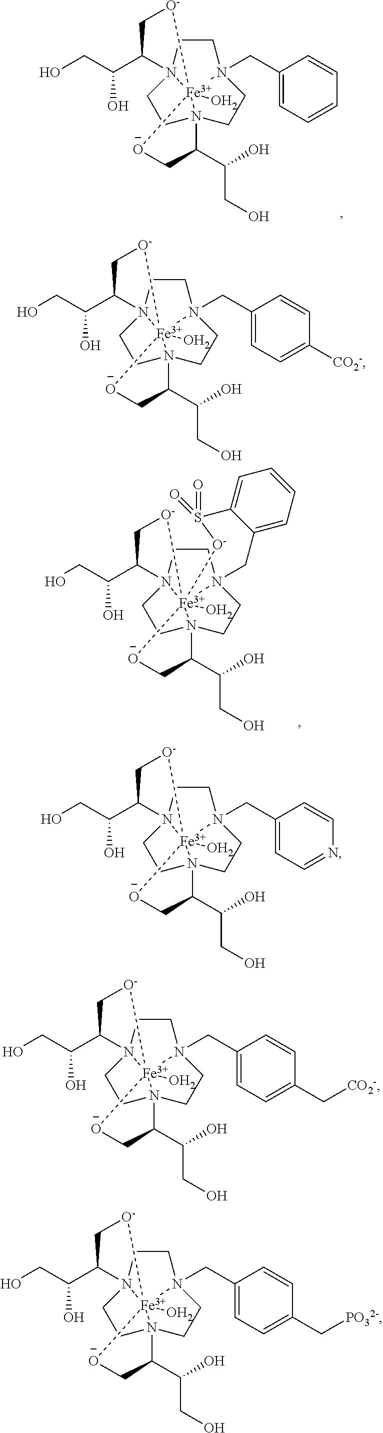

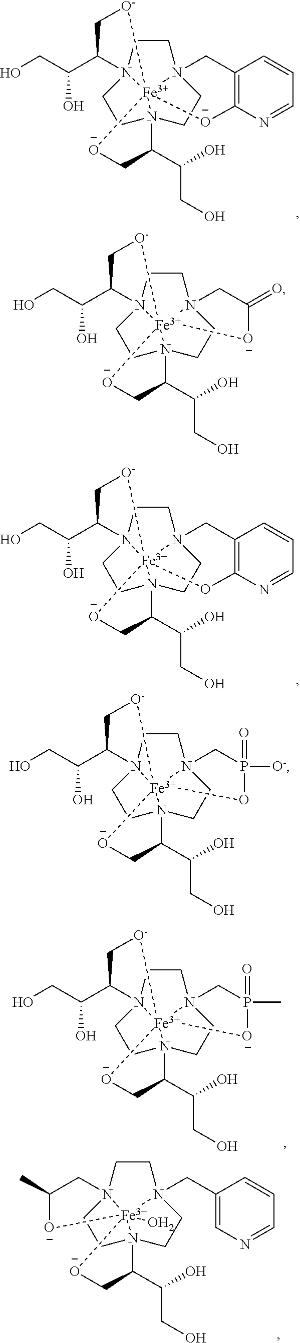

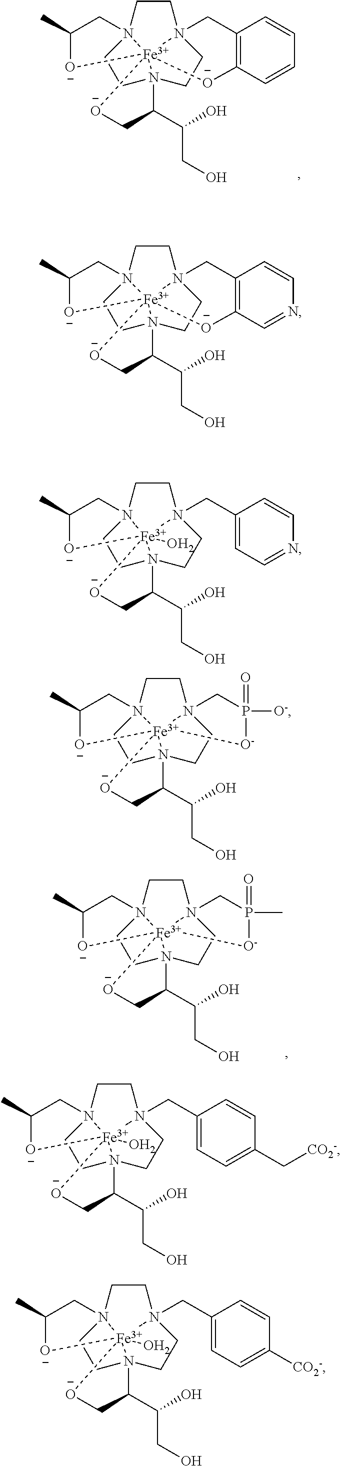

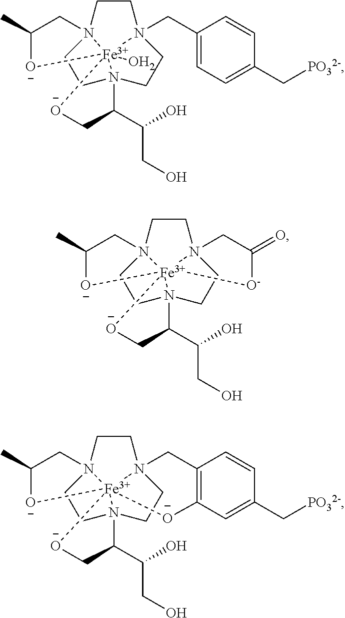

and protonated, partially deprotonated, and deprotonated species thereof (where applicable).

Examples of Fe(III) complexes with no inner-sphere water Fe(L6B) or with inner-sphere water, Fe(L1B):

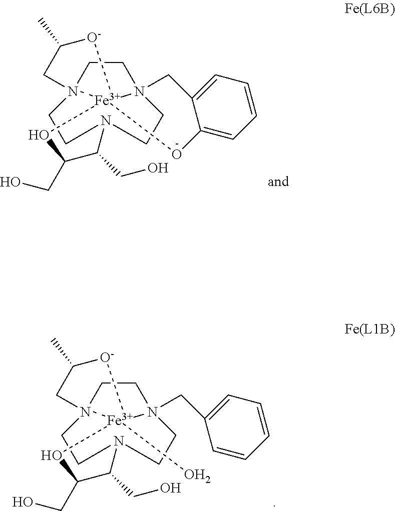

In various examples, other hydroxyl groups of the

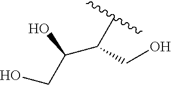 [SMILES: *[C@H](CO)[C@H](O)CO]

group may coordinate to the high-spin Fe(III).

Macrocyclic compounds can be macrocyclic ligands. The macrocyclic ligands described herein stabilize the trivalent iron (Fe(III)) state. The coordination geometry is designed for desirable binding of Fe(III) in comparison to Fe(II) to maintain the Fe(III) oxidation state, for example, under biologically relevant conditions. Stabilization of the Fe(III) state also serves to inhibit the production of reactive oxygen species that occur through reduction to the Fe(II) state of the complex.

It is desirable that the Fe(III) center is stabilized relative to Fe(II) so that there is no reaction with biological reductants to produce reactive oxygen species (ROS). Such redox-inactive (under biological conditions) Fe(III) centers have low redox potentials versus NHE. Examples of macrocyclic complexes of the present with macrocyclic core and pendant groups that produce stabilized Fe(III) include, but are not limited to, 1,4,9-triazacyclononane macrocyclic core and alcohol pendant groups that become deprotonated upon binding of Fe(III).

In various examples, a macrocyclic compound or compound of the present disclosure exhibits a reduction potential (Eₒ) of less than 0 mV vs. normal hydrogen electrode (NHE) in aqueous solution at a biologically relevant pH (e.g., a pH of 6.5-7.5 or 7.2-7.4, including all 0.1 pH values and ranges therebetween). In various other examples, a macrocyclic compound or compound of the present disclosure exhibits a reduction potential (Eₒ) of at least 300 mV, at least 250 mV, at least 200 mV, at least 150 mV, at least 100 mV or at least 50 mV or at least 0 mV, or at least −100, at least −150, at least −200, at least −300, at least −400, at least −500, or at least −600 mV vs. normal hydrogen electrode (NHE) in aqueous solution at a biologically relevant pH (e.g., a pH of 6.5-7.5 or 7.2-7.4, including all 0.1 pH values and ranges therebetween). In various other examples, a macrocyclic compound or compound of the present disclosure exhibits a reduction potential (Eₒ) of less than 0 to −600 mV vs. normal hydrogen electrode (NHE) in aqueous solution at a biologically relevant pH (e.g., a pH of 6.5-7.5 or 7.2-7.4, including all 0.1 pH values and ranges therebetween).

The shortening of the T₁ relaxation times of the protons of water by the Fe(III) complexes, T₁ relaxivity, is promoted by both innersphere water and outersphere water interactions and by proton exchange through water ligands or hydroxyalkyl pendants. Accordingly, in various examples, macrocyclic complexes and compounds of the present disclosure comprise one or more pendant donor groups that can hydrogen bond to water through heteroatoms such as, for example, oxygen or nitrogen. Non-limiting examples of such pendant donor groups are pendant alcohol groups that deprotonate to alkoxide groups. In addition, in various examples, macrocyclic compounds and compounds of the present disclosure comprise an open coordination site, which may bind water. These water ligands may ionize to form hydroxide ligands at neutral pH, for example, as shown by, pH-potentiometric titrations.

Coordination chemistry of Fe(III) is dependent on the coordination number. The macrocyclic compounds of the present disclosure have donor groups which can be part of the macrocyclic core, also referred to as macrocycle donors, and donor groups can be part of the substituents (e.g., pendant groups) on the macrocyclic core, also referred to as pendant donors. When Fe(III) is complexed to a macrocyclic compound of the present disclosure, 4 to 6 donors are complexed to the metal ion center. In an embodiment, the macrocyclic core can have from 2 to 3 donors and from 2 to 3 pendant donors. In various embodiments, there are 2 macrocycle donors and 3 pendant donors, 2 macrocycle donors and 4 pendant donors, 3 macrocycle donors and 2 pendant donors, 3 macrocycle donors and 3 pendant donors, 3 macrocycle donors and 4 pendant donors.

Examples of suitable macrocyclic cores with pedant donors include:

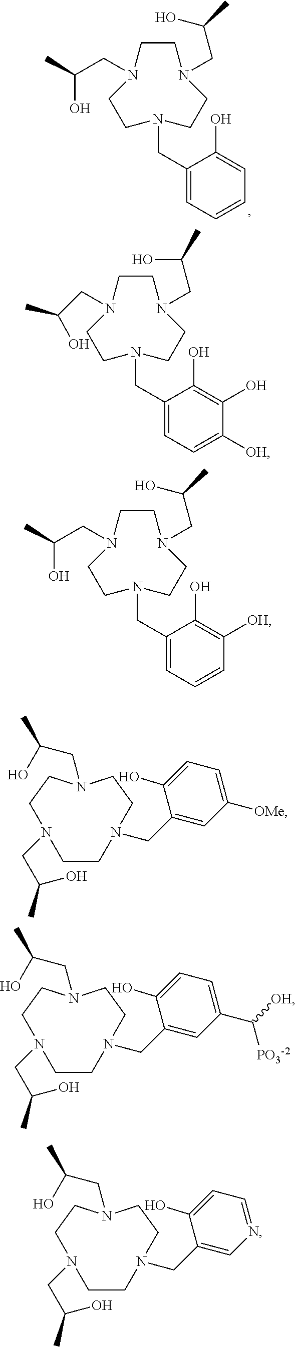

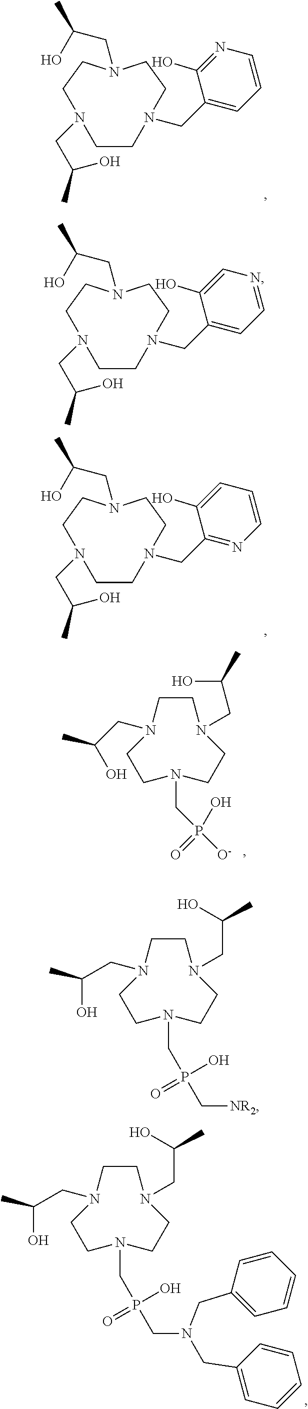

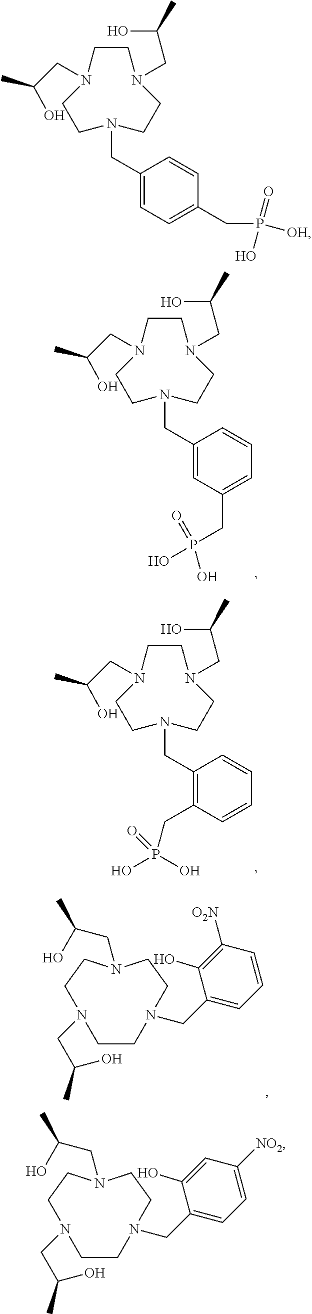

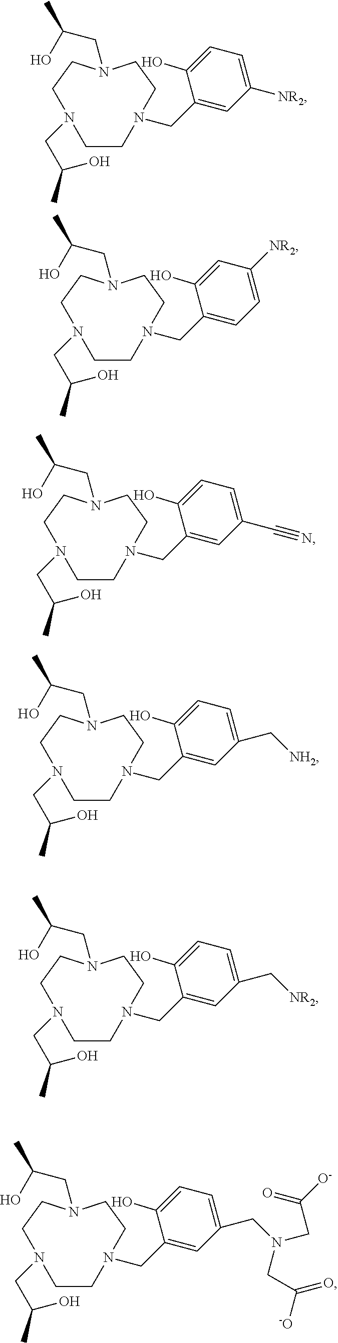

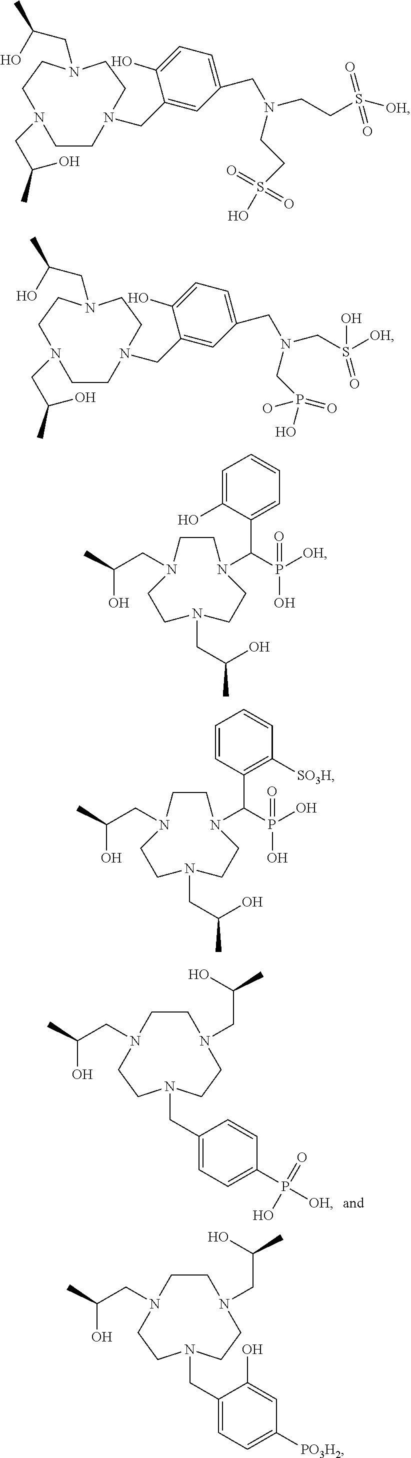

where Fe(III) may be complexed to the core.

Examples of macrocyclic compounds (e.g., macrocyclic complexes) include, but are not limited to,

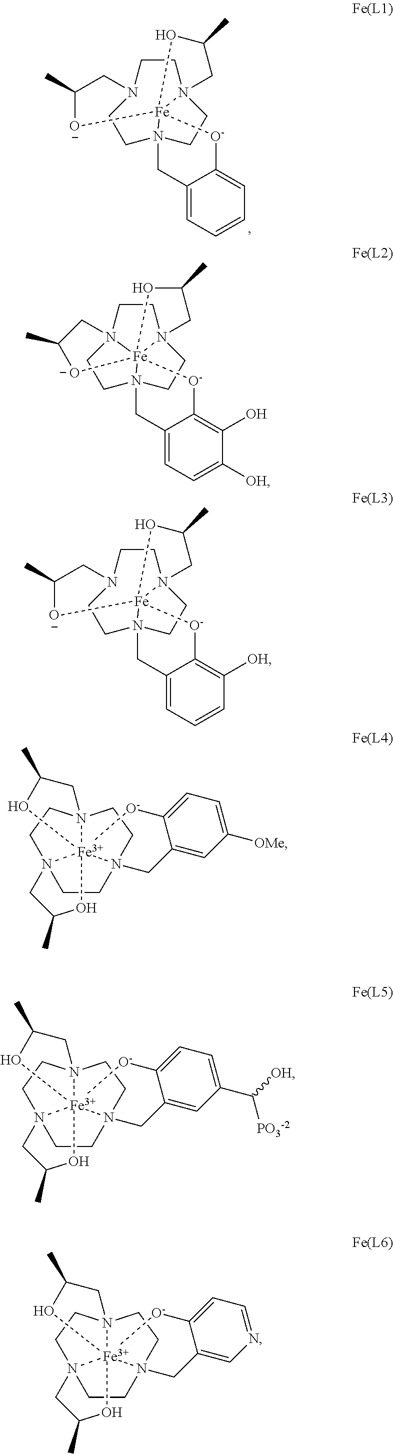

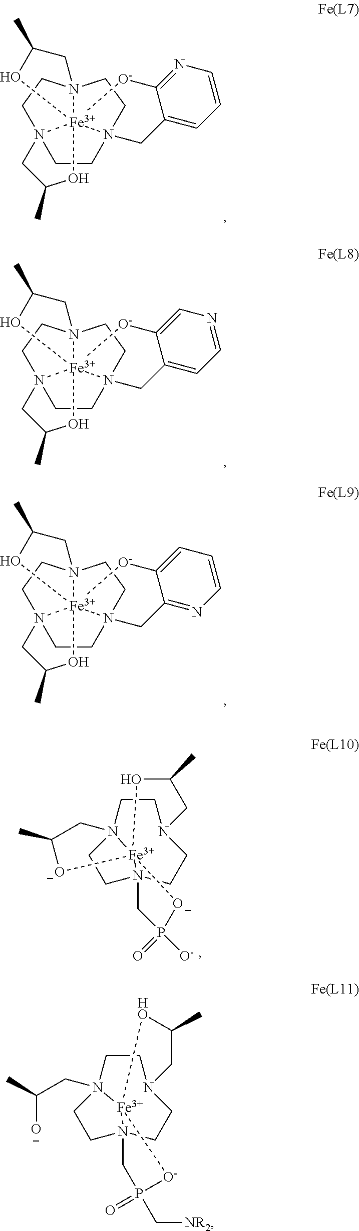

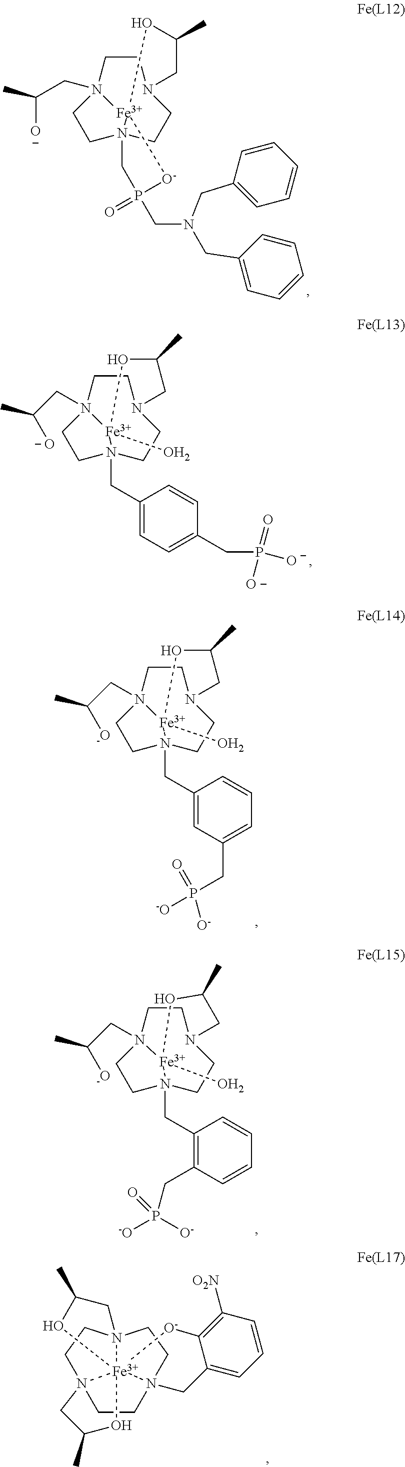

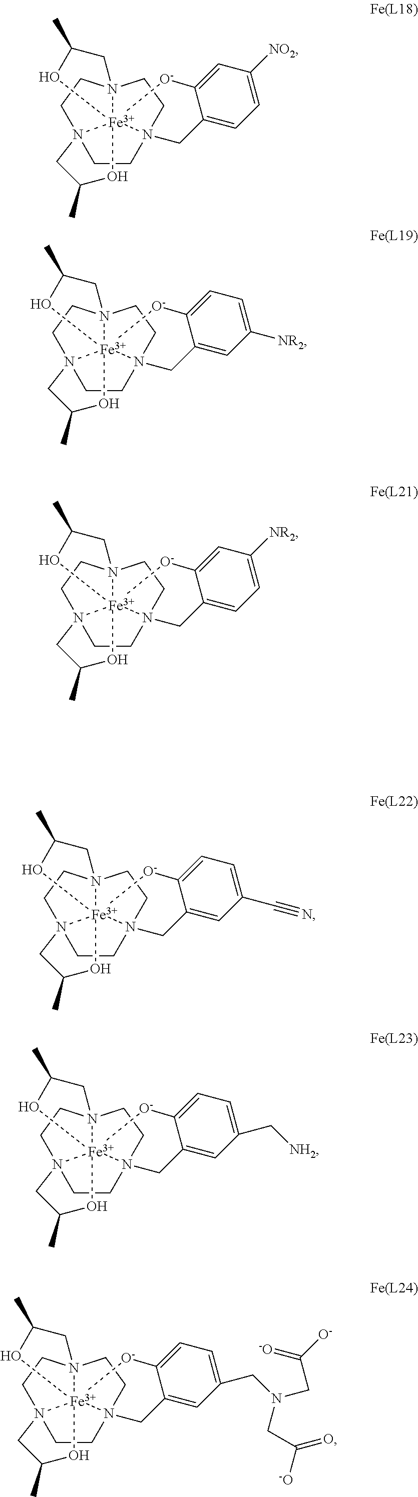

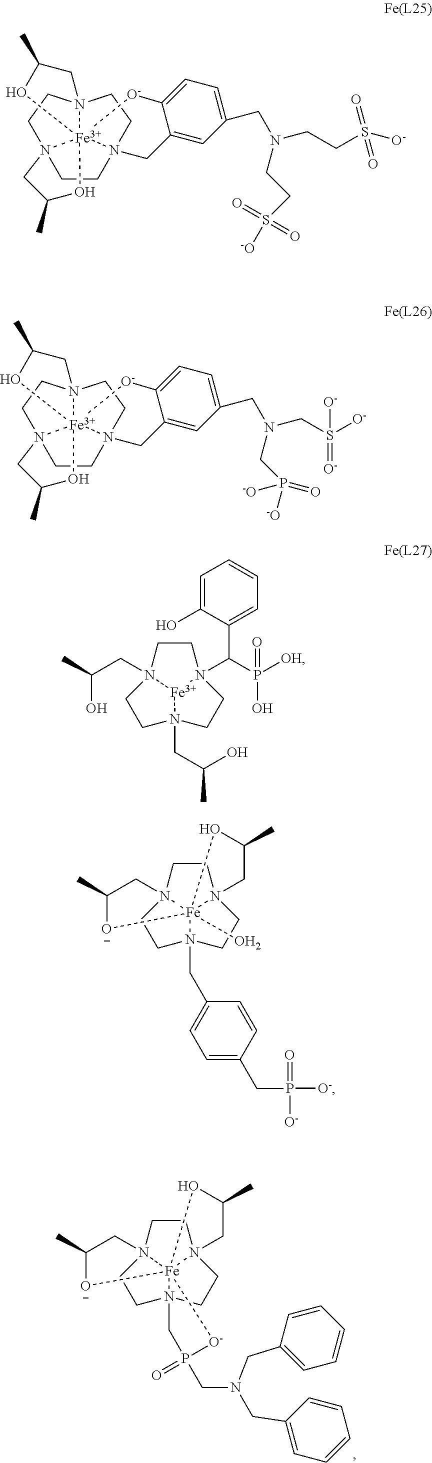

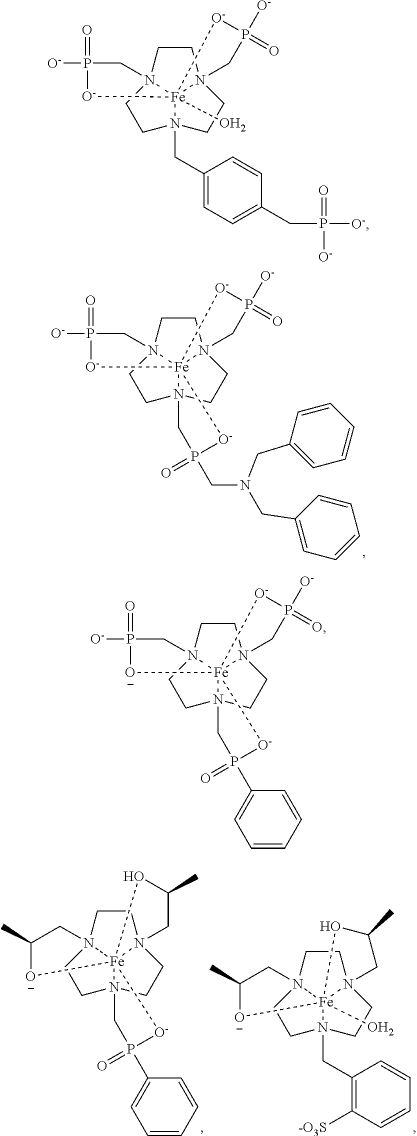

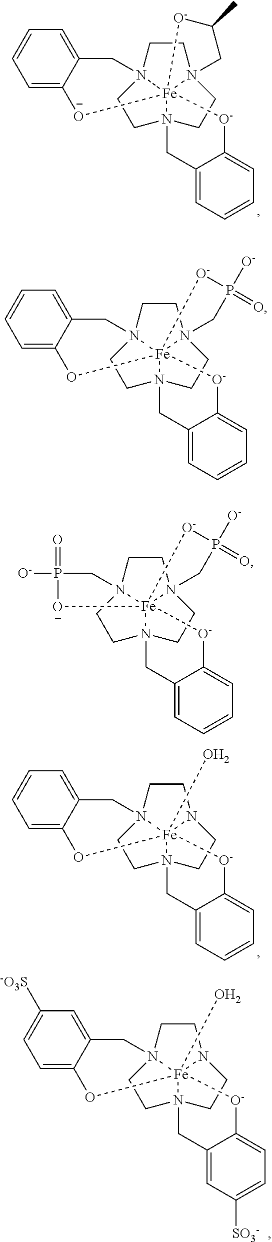

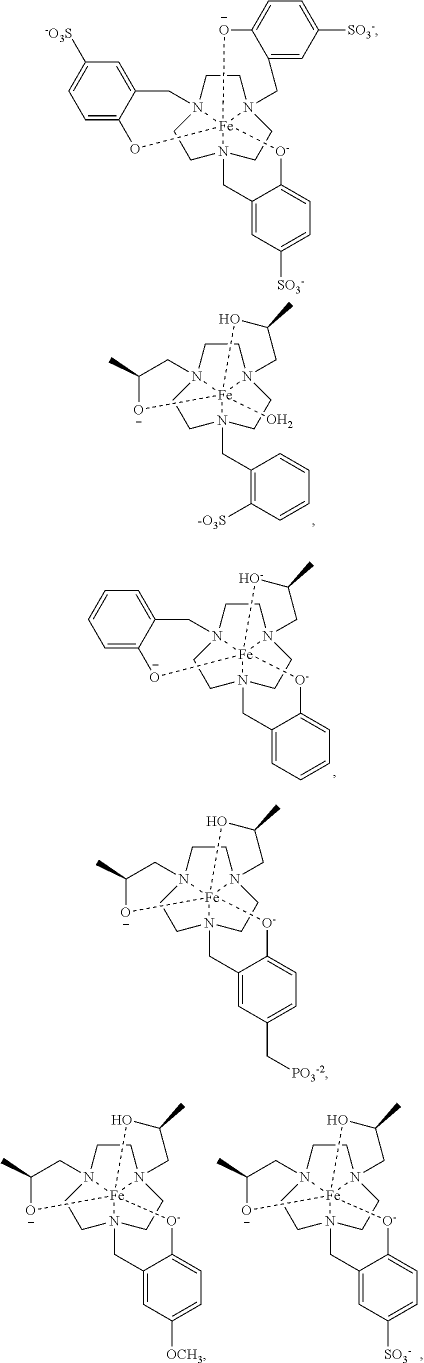

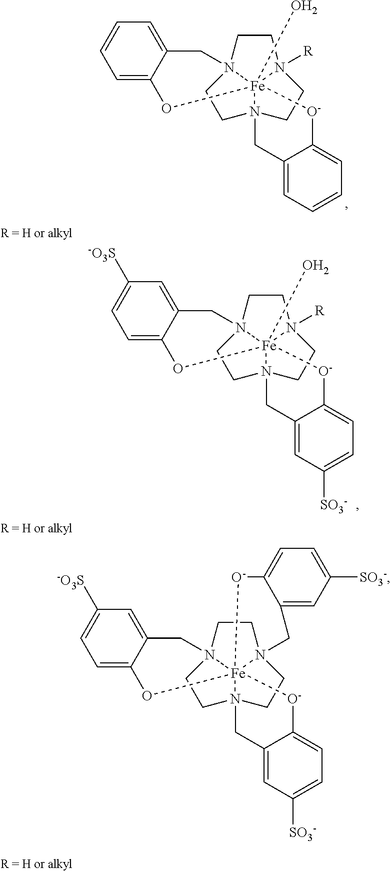

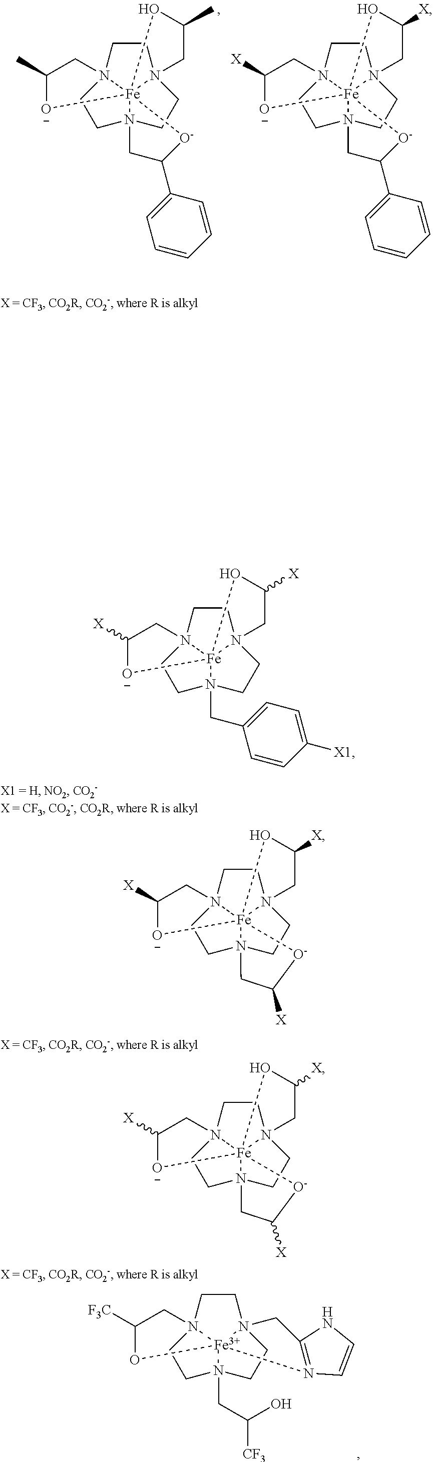

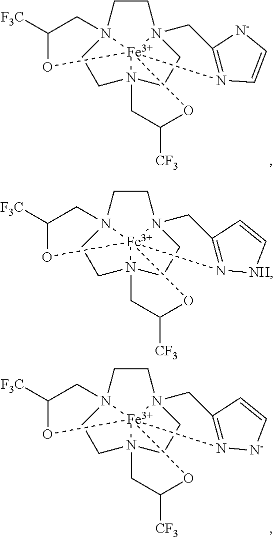

and deprotonated, partially deprotonated, and protonated species thereof (where applicable).

In various embodiments, the macrocyclic compounds, macrocyclic complexes, or compounds of the disclosure are a salt, a partial salt, a hydrate, a polymorph, or a stereoisomer, or a mixture thereof. For example, the macrocyclic compound, macrocyclic complex, or compound is present as a racemic mixture, a single enantiomer, a single diastereomer, or mixture of diastereomers. In certain embodiments, after complexation of the metal ion, the macrocyclic complexes or compounds are present as mixtures of diastereomers and/or conformers, which can be determined by NMR. The diastereomers may arise from the conformation of the macrocyclic core and the directionality of the substituents on the macrocyclic core.

The compounds of the disclosure can have innersphere water or alternatively, a hydroxide ligand. In an embodiment, the compounds have one innersphere ligand (q) which contributes to relaxivity as in Eq. 1.

$${R}_{1} = {R}_{1}^{S S} + {R}_{1}^{I S} \qquad Equation 1$$

$${R}_{1}^{IS} = \frac{q / [ {H}_{2} O ]}{{T}_{1 m} + {τ}_{m}} \qquad Equation 2$$

Eq. 1 shows that relaxivity has contributions from bound water (innersphere, IS) and second-sphere (SS) (outersphere) water. Eq. 2 predicts that greater numbers of bound water molecules and rapid ligand exchange rate constants (short lifetimes for bound water (τm)) are advantageous. Notably r₁, the parameter used to characterize relaxivity, has units of mM⁻¹s⁻¹, and is obtained from a plot of T_{lobs} (s⁻¹) versus contrast agent concentration. There is an analogous relationship for second-sphere waters although the number and residence time is not well defined.

It is desirable that the ratio of the T₁ to T₂ relaxivity (R₁/R₂) of a macrocyclic complex or compound of the present disclosure are close to one (unity). R₂, the transverse relaxivity, is by definition always larger than R₁, the longitudinal relaxivity. In various examples, Fe(III) contrast agents of the present disclosure have desirably low R₂ to give R₁/R₂ ratios close to one. In various examples, a macrocyclic complex or compound of the present disclosure have R₁/R₂ ratios of 0.5 to 0.2 or 0.8 to 0.6.

An Fe(III) complex may have a desirable interaction with water molecules that can enhance relaxation of the protons of the water. Without being bound by any particular theory, it is considered that exchange of innersphere water with bulk water is an important mechanism for proton relaxivity. However, second sphere water interactions may also contribute. Proton exchange of pendants that have OH protons from hydroxyalkyl groups provide an additional mechanism.

This shows that optimization of the interaction of the Fe(III) complex with water molecules to enhance relaxation of the protons of the water is important. Without being bound by any theory, exchange of innersphere water with bulk water is thought to be a dominant mechanism for proton relaxivity in Gd(III) complexes. However, Fe(III) is a much smaller metal ion than Gd(III) (0.78 Å vs. 1.25 Å, respectively). The shorter M−H distance in bound water of Fe(III) compared to Gd(III) suggests that the relative efficiency of the second-sphere, outersphere versus innersphere contributions may differ for the two metal ion complexes.

There are three mechanisms that contribute to paramagnetic relaxation of associated water (1/T_{1 m}): the scalar (contact) contributions, dipole-dipole contributions and Curie spin relaxation. The most important of these for the longitudinal relaxation considered here is the dipole-dipole contribution (1/T₁DD). At field strengths of 1.5 T or greater, 1/T₁DD is defined as shown in Eq. 3 where S is the spin quantum number, ω_{H} is the Larmor frequency of the proton, r_{MH} is the metal ion-proton distance and γ_{H} is the proton gyromagnetic ratio, ge is the electronic g factor, μ_{B} is the Bohr magneton, and μₒ is the permittivity of a vacuum. Notably, the 1/T₁DD term increases (higher relaxivity) with larger total spin (S) which favors Gd(III) over Fe(III). However, the shorter distance of the paramagnetic Fe(III) center to water protons (r_{MH}) favors Fe(III) proton relaxation, especially given the 1/r⁶ dependence.

$$\frac{1}{{T}_{1}^{D D}} = \frac{2}{15} ( \frac{{μ}_{0}}{4 π} ) \frac{{γ}_{H}^{2} {g}_{e}^{2} {μ}_{B}^{2} S ( S + 1 )}{{r}_{M H}^{6}} [ \frac{3 {τ}_{c}}{1 + {ω}_{H}^{2} {τ}_{c}^{2}} ] \qquad Equation 3$$

$$\frac{1}{{τ}_{c}} = \frac{1}{{τ}_{R}} + \frac{1}{{T}_{1 e}} + \frac{1}{{τ}_{m}} \qquad Equation 4$$

The correlation time (τc) for the dipolar relaxation mechanism is influenced by different processes including the lifetime of the bound water (1/τₘ), the rotational motion of the contrast agent (1/τ_{R}) and the longitudinal relaxation of the upaired electrons (1/T₁ₑ). Although any of these three processes can contribute, their importance depends on the strength of the magnetic field. Much of the literature is focused on the importance of these processes at low field strength (<1 T). Under these conditions, the rotational processes or electronic relaxation times may be limiting, and τₘ should be in a narrow range close to 10 ns (kₑₓ=10⁸ s⁻¹). However at higher field strengths (≥1.5 T), simulations show that the optimal τₘ has a larger range (1-100 ns) and rotational motions should have values intermediate between small molecules and proteins. An important parameter is T₁ₑ, the electronic relaxation time. A long T₁ₑ for Fe(III) may result from complexes that have a high degree of symmetry, leading to little zero field splitting and slow relaxation of the electronic state. Also, the coordination sphere needs to favor high spin (S=5/2) and not low spin S=½ Fe(III).

The macrocyclic compounds of the present disclosure are thermodynamically stable and/or kinetically inert towards dissociation. In an embodiment, the macrocyclic compounds are thermodynamically stable and kinetically inert towards dissociation. In an embodiment, the kinetic inertness of the macrocyclic compounds of the present disclosure can be described using a rate constant for dissociation. In an embodiment, the macrocyclic donors and pendant donors do not dissociate appreciably from the metal center (e.g., 1% or less, 0.1% or less, or 0.01% or less dissociation is observed) for up to 24 hours at neutral pH in the presence of 1) 25 mM carbonate, 0.40 mM phosphate, 100 mM NaCl, pH 7.2; 2) pH 4, 100 mM NaCl.

In an embodiment, Fe(III) is high-spin S=5/2. For effective T₁ (longitudinal) relaxation, a paramagnetic spin state is needed. In order to keep Fe(III) in the high-spin state, the ligand (or crystal) field splitting must not be too large. If the crystal field splitting is larger than the pairing energy, a low spin (S=½) state will result. Fe(III) is readily maintained in a high-spin paramagnetic state with a range of ligand donor groups, especially containing anionic oxygen donors.

An Fe(III) complex may have an open coordination site for a water ligand, two alcohol pendants and a third pendant. Ancillary pendant groups such as, for example, aryl groups (e.g., benzyl groups and substituted benzyl groups, such as, for example, methoxy-benzyl groups, and fused ring aryl groups) or alkyl groups (e.g., methyl, ethyl, or branched alkyl groups such as iso-propyl) are particularly effective. The relaxivity of a coordinatively saturated complexes may be enhanced by attachment of larger ancillary pendants to slow rotational correlation times. A third pendant group may be used to close off the coordination sphere around the Fe(III) and an anionic group may enhance the proton exchange of the hydroxylalkyl groups by modulating their pKₐ values.

It is desirable that the electronic relaxation time of the high-spin Fe(III) center is sufficiently long (e.g., greater than 3×10⁻¹¹ s), so that it is not the limiting factor in the correlation time constant as expressed in equation 4 at field strengths of 1.5 Tesla or greater. This can be accomplished by, for example, using macrocyclic ligands that produce high symmetry at the Fe(III) center. It is desirable that the zero field splitting factor (D) is small given that (T₁ₑ)⁻¹ is directly proportional to D² for high-spin Fe(III) complexes in an axially distorted complex.

It is desirable that the Fe(III) complex remain in the trivalent oxidation state and not be reduced by, for example, peroxide, superoxide, ascorbate, or by glutathione at concentrations present in the extracellular medium of cells such as, for example, mammalian cells (e.g., human cells). Normally, a redox potential more negative than 200 mV (<200 mV) versus NHE is sufficient.

For use in methods of the disclsoure, the compounds described herein can be administered as pharmaceutical preparations. Accordingly, they can be provided in a variety of compositions, and can be combined with one or more pharmaceutically acceptable carriers. Some examples of pharmaceutically acceptable carriers can be found in: Remington: The Science and Practice of Pharmacy (2012) 22nd Edition, Philadelphia, PA. Lippincott Williams & Wilkins. The composition can be provided as a liquid, a solution, or a solid, and may be provided in combination with any suitable delivery form or vehicle, examples of which include, but are not limited to, caplets, capsules, tablets, an inhalant, aerosol, or the like.

A composition may comprise one or more standard pharmaceutically acceptable carrier(s). Non-limiting examples of compositions include solutions, suspensions, and emulsions. Non-limiting examples of diluents include distilled water for injection, physiological saline, vegetable oil, alcohol, and the like, and combinations thereof. Further, injections may contain stabilizers, solubilizers, suspending agents, emulsifiers, soothing agents, buffers, preservatives, and the like. The composition may also be formulated into a sterile solid preparation, for example, by freeze-drying, and can be used after sterilized or dissolved in sterile injectable water or other sterile diluent(s) immediately before use. Non-limiting examples of pharmaceutically acceptable carriers can be found in: Remington: The Science and Practice of Pharmacy (2012) 22nd Edition, Philadelphia, PA. Lippincott Williams & Wilkins.

Acceptable carriers, excipients, or stabilizers are nontoxic to recipients at the dosages and concentrations employed, and include, but are not limited to, buffers such as, for example, phosphate, citrate, histidine and other organic acids; antioxidants including, but not limited to, ascorbic acid and methionine; preservatives (such as, for example, octadecyldimethylbenzyl ammonium chloride; hexamethonium chloride; benzalkonium chloride, benzethonium chloride; phenol, butyl or benzyl alcohol; alkyl parabens such as, for example, methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ polyethylene glycol (PEG) and the like. In an embodiment, the pharmaceutical composition may comprise buffer components and stabilizers, including, but not limited to, sucrose, polysorbate 20, NaCl, KCl, sodium acetate, sodium phosphate, arginine, lysine, trehalose, glycerol, and maltose.

Various methods known to those skilled in the art may be used to introduce the compositions of the disclosure to an individual. These methods include but are not limited to intravenous, intramuscular, intracranial, intrathecal, intradermal, subcutaneous, and oral routes. In an embodiment, the composition is administered intravenously.

The necessary solubility of the complexes depends on their effectiveness in producing contrast. For Fe(III) T₁ contrast agents that have good relaxivity, the complexes need 5 mM-100 mM. However, other additives such as human serum albumin (HSA) or meglumine may be used to increase solubility and/or increase relaxivity. Addition of HSA (e.g., 35 mg/mL) to some of the Fe(III) complexes produces higher T₁ relaxivity. Solubility is generally measured in aqueous solution at near neutral pH (e.g., 6.5 to 7.5, including all 0.1 pH values and ranges therebetween) in 100 mM NaCl with 25 mM carbonate and 0.4 mM phosphate. The dose of the composition to be used will necessarily be dependent upon the needs of the individual to whom the composition of the disclosure is to be administered. These factors include, but are not necessarily limited to the weight, age, sex, and medical history of the individual.

In an aspect, the present disclosure provides imaging methods using the macrocyclic complexes and compounds described herein. The imaging methods use magnetic resonance imaging methods. Non-limiting examples of such methods include, Magnetic Resonance Imaging (MRI).

Specifically, the macrocyclic compounds of the present disclosure, which are complexed to Fe(III), can be used as T₁ MRI contrast agents. These complexes may have properties that change with alterations in pH. Such properties make these complexes useful for mapping pH to enable better therapeutic treatment of diseases such as, for example, cancer, stroke and heart disease.

The imaging methods of the present disclosure can be used to image a cell, tissue, organ, vasculature, or a part thereof. The cell, tissue, organ, vasculature can be a part of an individual. By “individual” it is meant a human or non-human animal (e.g., cow, pig, mouse, rat, cat, dog, or other agricultural, pet, or service animal, and the like). In an embodiment, the disclosure provides a method to obtain an image of at least a portion of a cell, tissue, organ, or vasculature comprising the steps of: contacting a cell, tissue, organ, or vasculature with the compounds of the present disclosure, and imaging at least a portion of the cell, tissue, organ, or vasculature to obtain an image of the portion of cell, tissue, organ, or vasculature. At least part of a cell, tissue, or organ can be alive or dead. Likewise, the individual can also be alive or deceased.

Administration may occur by various delivery methods, The compounds or compositions of may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration.

In an embodiment, the macrocyclic complex compound is used as a Fe(III) T₁ MRI contrast agent. This contrast is produced by T₁ weighted imaging to give positive contrast in the region where the iron complexes accumulate. The complexes are high-spin Fe(III) under biologically reducing conditions with either innersphere and/or outersphere water interactions that give a decrease in the T₁ relaxation times of bulk water protons.

The macrocyclic compounds of the present disclosure can be prepared, for example, as described herein.

The following examples are presented to illustrate the present disclosure. They are not intended to be limiting in any manner. Those skilled in the art will recognize that routine modifications to these embodiments can be made which are intended to be within the scope of the disclosure.

### Additional Description of the Present Disclosure

In certain embodiments, the macrocyclic compounds have the following structure:

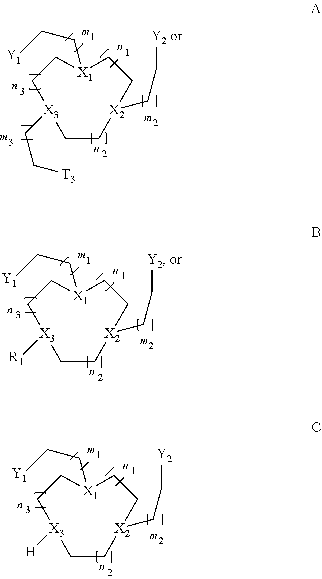

wherein X₁, X₂, X₃, and X₄ are N; Y₁, Y₂, Y₃, and Y₄ are each independently pendant donors comprising N, wherein N has a lone pair of electrons (e.g., amino, benzimidazole, imidazole, aniline, pyrazoyl, triazole, benzotriazole, and the like), or a pendant donor comprising O, wherein O has at least one lone pair of electrons but preferably two or three lone pairs (e.g., ketone, alcohol, alkoxide, carboxylic acid, amide, phenol or phenoxide, or a deprotonated form of the foregoing, such as, for example, a carboxylate ion, an imidazolate ion, a pyrazolate ion or an oxide, including an alkoxide or a phenoxide; m₁, m₂, m₃, and m₄ are each independently 0, 1, or 2; n₁, n₂, n₃, and n₄ are each independently 1 or 2; and R₁, R₂, and R₃ are each independently substituted or unsubstituted aryl, substituted or unsubstituted heteroaryl or substituted or unsubstituted alkyl group, wherein R₁, R₂, and R₃ are not substituted by a pendant donor, wherein the alkyl segment of the alkyl-Y chain (alkyl-Y₁, alkyl-Y₂, alkyl-Y₃ and/or alkyl-Y₄) may each independently be substituted (e.g., Structure a or Structure b) or unsubstituted (Structure c or Structure d). For Structures a-f, the pendent may have either R or S configuration at the chiral carbon:

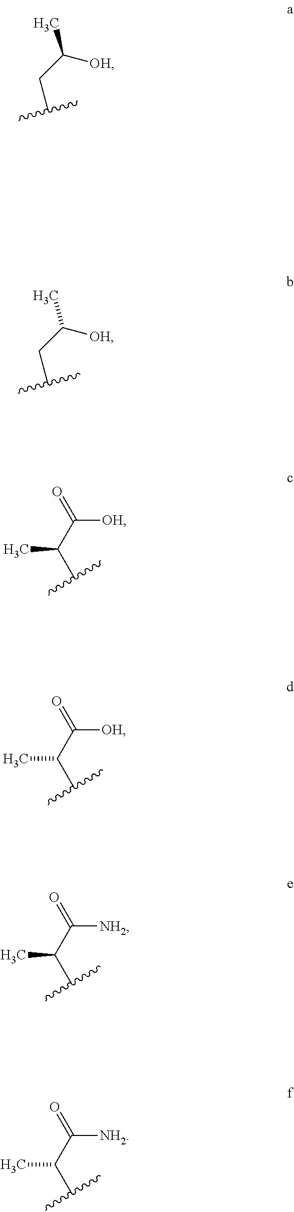

In some embodiments, the macrocycle may have the structure (Scheme IV):

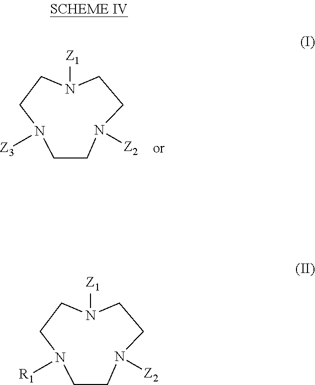

where R₁ is a substituted or unsubstituted aryl, substituted or unsubstituted heteroaryl or a substituted or unsubstituted alkyl and where when the macrocycle has Structure I, Z₁ is H or one of the pendent groups in Scheme V and Z₂ and Z₃ each independently are one of the pendent groups in Scheme V; when the macrocycle has Structure II, Z₁ and Z₂ each independently are one of the pendent groups in Scheme V; and where for Structures I and II, each of Z₁, Z₂, Z₃, as applicable, are selected independently of each other. This paragraph is hereinafter referred to as “Scheme IV.”

The macrocyclic compound has at least one pendant donor on the macrocyclic core. For example, the pendant donor can have the following structure:

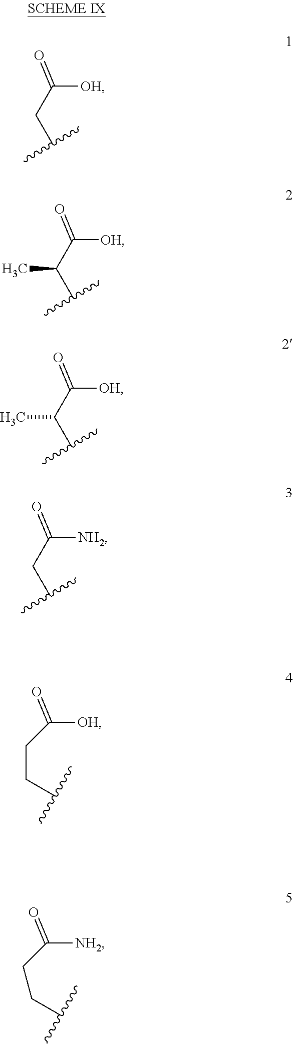

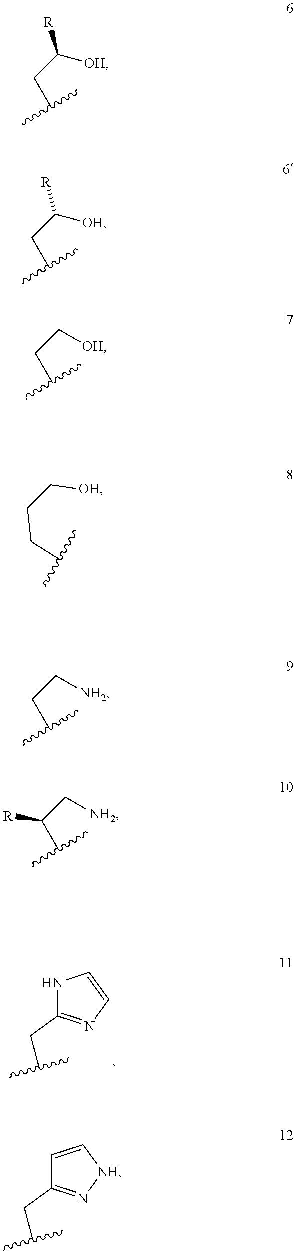

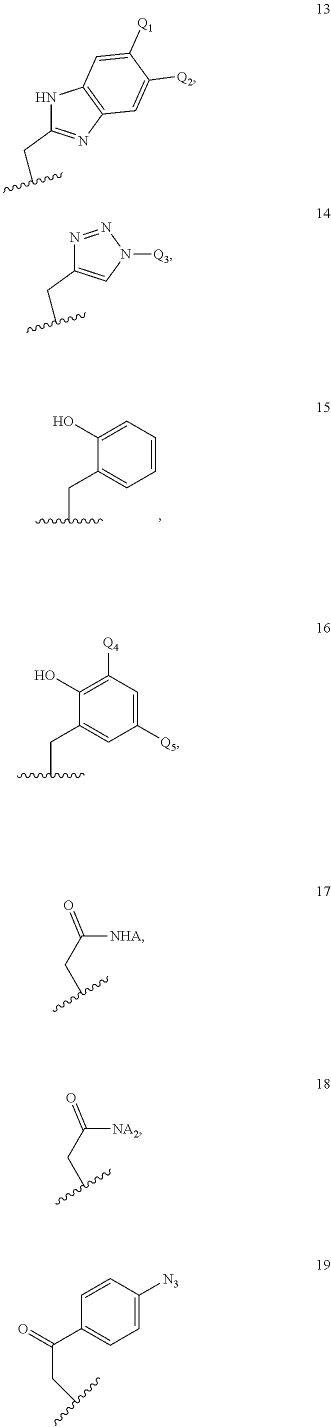

wherein R is methyl, Q₁ and Q₂ are each independently —H, —OCH₃, —CO₂H, or —CH₂CO₂G₄, G₄ is H, C₁ to C₁₂ substituted or unsubstituted alkyl groups of linear or branched structure or PEG group (—CH₂CH₂O—)ₙ (n=1-12, e.g., 1, 2, 3, 4, 5, 6, 7, 8, 9, 10, 11, and 12), Q₃ is H, C₁ to C₁₂ substituted or unsubstituted alkyl groups of linear or branched structure or PEG group (—CH₂CH₂O—)ₙ (n=1-12, e.g., 1, 2, 3, 4, 5, 6, 7, 8, 9, 10, 11, and 12), Q₄ and Q₅ are each independently —H, —OCH₃, —CO₂H, or substituted or unsubstituted alkyl groups of linear or branched structures, A is a substituted or unsubstituted alkyl group of linear or branched structure with C₁ to C₁₂ or is a substituted or unsubstituted aryl group or naturally occurring (e.g., glycine) or synthetic amino acid or analog thereof. Some pendant donors, such as, for example, carboxylic acid, alcohol, imidazole or pyrazole, may deprotonate when complexed with Fe(III) or at certain pH values. Such protonated and deprotonated forms are within the scope of the disclosure. For example, the pendant donor is a carboxylate ion, an imidazolate ion, a pyrazolate ion, or an oxide (e.g., an alkoxide or a phenoxide).

In an embodiment, the subject disclosure provides macrocyclic compounds having the structures and definitions set forth in herein with the following provisos based on the following schemes:

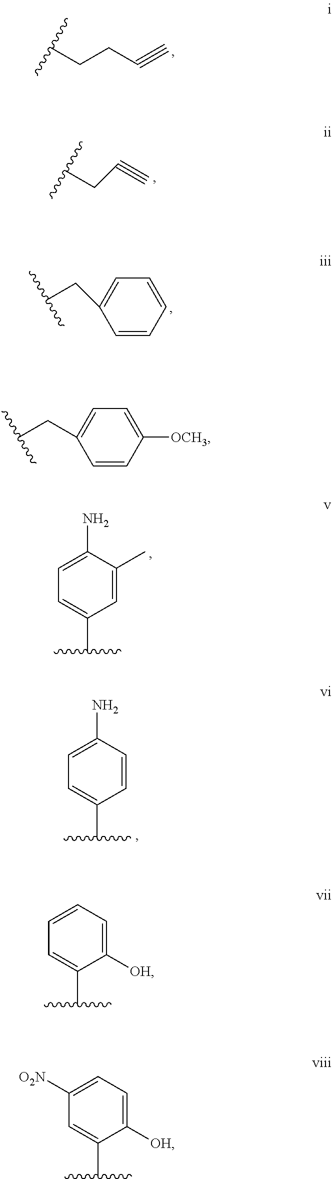

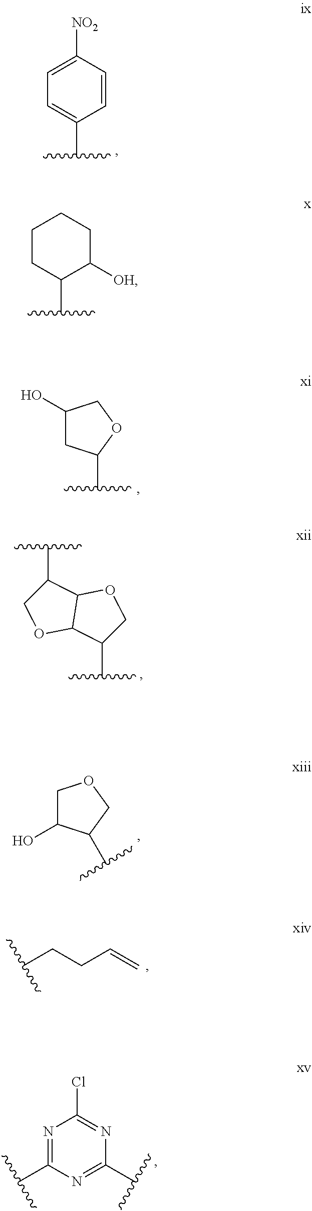

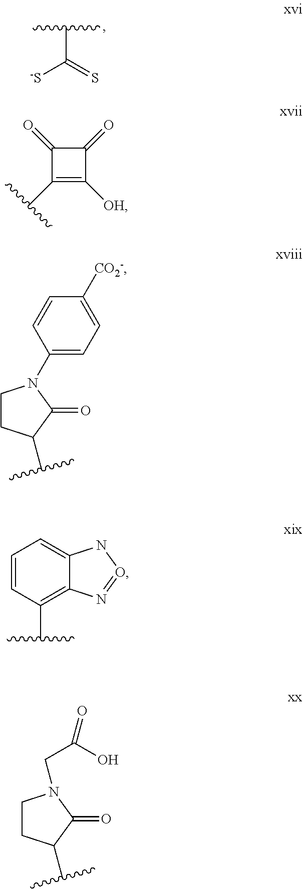

where any or all of the following provisos apply when the macrocycle has structure II (using schemes VIII and IX): when Z₁=Z₂=Structure 1, R₁≠methyl, ethyl, isopropyl, n-hexyl or Structure i, ii, iii, or iv; when Z₁=Z₂=Structure 7, R₁≠Structure v or vi; when Z₁=Z₂=Structure 9, R₁≠ethyl; when Z₁=Z₂=Structure 12, R₁≠ethyl; when Z₁=Z₂=Structure 16 when Q₄=t-butyl and Q₅=OCH₃ or when Q₄=Q₅=t-butyl, R₁≠ethyl or isopropyl; when Z₁=Z₂=Structure 15, R₁≠methyl.

In one embodiment, the subject disclosure provides macrocyclic compounds having the structures and definitions set forth in Schemes IV, VIII, and IX, where any or all of the following provisos apply when the macrocycle has Structure I: when Z₁=Z₂=Structure 1, Z₃≠Structure 1; when Z₁=Z₂=Structure 2, Z₃≠Structure 2; when Z₁=Z₂=Structure 3, Z₃≠Structure 3; when Z₁=Z₂=Structure 6, Z₃≠Structure 6; when Z₁=Z₂=Structure 7, Z₃≠Structure 7; when Z₁=Z₂=Structure 9, Z₃≠Structure 9; when Z₁=Z₂=Structure 11, Z₃≠Structure 11; when Z₁=Z₂=Structure 12, Z₃≠Structure 12; when Z₁=Z₂=Structure 13 when Q₁=Q₂=H, Z₃≠Structure 13 when Q₁=Q₂=H; when Z₁=Z₂=Structure 15, Z₃≠Structure 15; at most two of Z₁, Z₂ or Z₃=Structure 16 when i) Q₄=Q₅=t-butyl, ii) Q₄=Q₅=OCH₃, iii) Q₄=t-butyl and Q₅=OCH₃ or iv) Q₄=OCH₃ and Q₅=t-butyl; when Z₁=H, Z₂≠Structure 1; when Z₁=H, Z₂≠Structure 7; when Z₁=H, Z₂≠Structure 9; when Z₁=H, Z₂≠Structure 13; when Z₁=Z₂=Structure 1, Z₃≠Structure 15; when Z₁=Structure 1 and Z₂=H, Z₃≠Structure 16 when Q=t-butyl; where any or all of the following provisos apply when the macrocycle has Structure III: when Z₁=Structure 1, Z₂≠Structure 1; when Z₁=Structure 17, Z₂≠Structure 17.

In some embodiments, when the macrocycles of Scheme IV are complexed with Fe(III), R₁ does not coordinate to the Fe(III).

In an embodiment, the macrocycles defined according to Schemes IV have at least one pendent donor on the macrocyclic core. For example, said pendent donor can have the following structure (Scheme V)

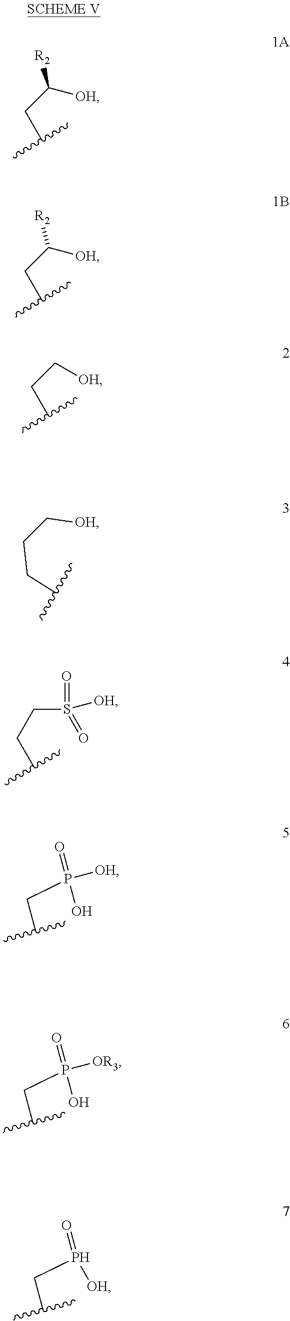

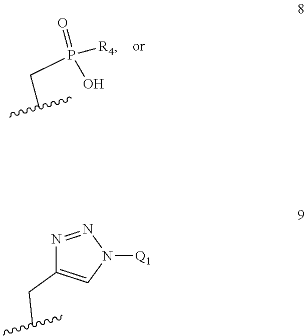

where R₂ is a substituted or unsubstituted alkyl group, a substituted or unsubstituted aromatic group, which may be an aryl group, or a substituted ether; R₃ is a substituted or unsubstituted alkyl or a substituted or unsubstituted aryl group and R₄ is a substituted alkyl (e.g., substituted with a hydroxyl or a carboxylate group, or the like) or unsubstituted alkyl or a substituted or unsubstituted aryl group. Some pendent donors, such as, for example, alcohol, phosphinic acid, phosphonic acid or sulfonic acid may deprotonate when complexed with Fe(III) or at certain pH values. Such protonated and deprotonated forms are within the scope of the present disclosure. For example, the pendent donor may be an alkoxide, phosphinate, phosphonate or sulfonate as shown in Scheme VI.

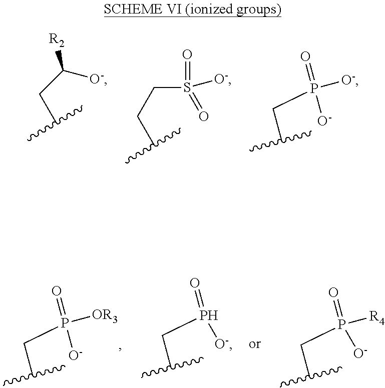

In certain embodiments, the R₁ group (which may be coordinating ancillary groups or non-coordinating ancillary groups) of said macrocycles in Schemes I and II may be a structure according to Scheme VII,

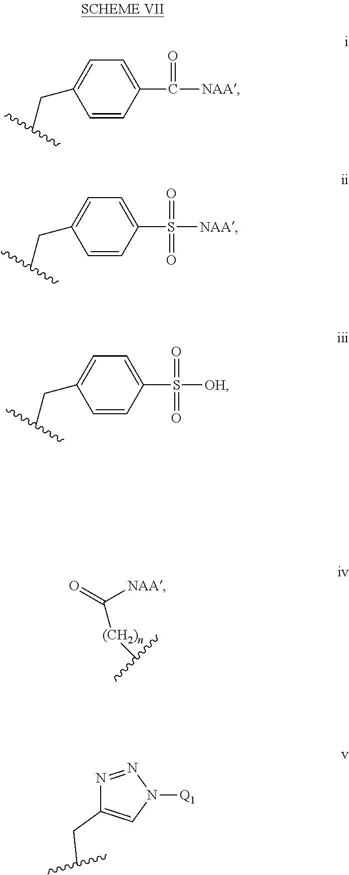

where A and A′ are each independently a substituted or unsubstituted C₁ to C₁₂ alkyl group of linear or branched structure or a proton and Q₁ is aryl substituted with an anionic group (such as, for example, a carboxylate, sulfonate, phosphonate, phosphate ester or phosphinate), an alkyl group substituted with an anionic group (such as, for example, a carboxylate, sulfonate, phosphonate, phosphate ester or phosphinate) or an aralkyl group substituted with an anionic group (such as, for example, a carboxylate, sulfonate, phosphonate, phosphate ester or phosphinate); where at least one of A or A′ is an alkyl group substituted with an anionic group (e.g., an amino acid, especially glycine, serine or aspartic acid).

In some embodiments, when the macrocycle has Structure (I) of Scheme IV and Z₁ and Z₂ are Structure 6 of Scheme V where R₃ is an unsubstituted ethyl, Z₃ is not Structure 6 of Scheme V where R₃ is an unsubstituted ethyl group. In further embodiments, when the macrocycle has Structure (I) of Scheme II and Z₁ and Z₂ are Structure 6 of Scheme V where R₃ is an unsubstituted or substituted ethyl, Z₃ is not Structure 6 of Scheme V where R₃ is an unsubstituted or substituted ethyl group. In added embodiments, when the macrocycle has Structure (I) of Scheme IV and Z₁ and Z₂ are Structure 6 of Scheme V where R₃ is an unsubstituted alkyl, Z₃ is not Structure 6 of Scheme V where R₃ is an unsubstituted alkyl. In still other embodiments, when the macrocycle has Structure (I) of Scheme IV and Z₁ and Z₂ are Structure 6 of Scheme V where R₃ is an unsubstituted or substituted alkyl, Z₃ is not Structure 6 of Scheme V where R₃ is an unsubstituted or substituted alkyl.

In particular embodiments, when the macrocycle has Structure (I) of Scheme IV and Z₁ and Z₂ are Structure 7 of Scheme V, Z₃ is not Structure 7 of Scheme V.

In certain embodiments, when the macrocycle has Structure (I) of Scheme IV and Z₁ and Z₂ are Structure 8 of Scheme V where R₃ is alkyl having a terminal hydroxyl substitution, Z₃ is not Structure 8 of Scheme V where R₃ is not alkyl having a terminal hydroxyl substitution. In further embodiments, when the macrocycle has Structure (I) of Scheme IV and Z₁ and Z₂ are Structure 8 of Scheme V where R₃ is a substituted alkyl, Z₃ is not Structure 8 of Scheme V where R₃ is a substituted alkyl.

In some embodiments, when the macrocycle has Structure (II) of Scheme IV and Z₁ and Z₂ are Structure 8 of Scheme V where R₄ is alkyl having a terminal hydroxyl substitution, R₁ is not alkyl having a terminal aryl group. In additional embodiments, when the macrocycle has Structure (II) of Scheme IV and Z₁ and Z₂ are Structure 8 of Scheme V where R₄ is alkyl having a terminal hydroxyl substitution, R₁ is not substituted alkyl.

In various examples, macrocyclic cores with pendant donors do not have the following structure:

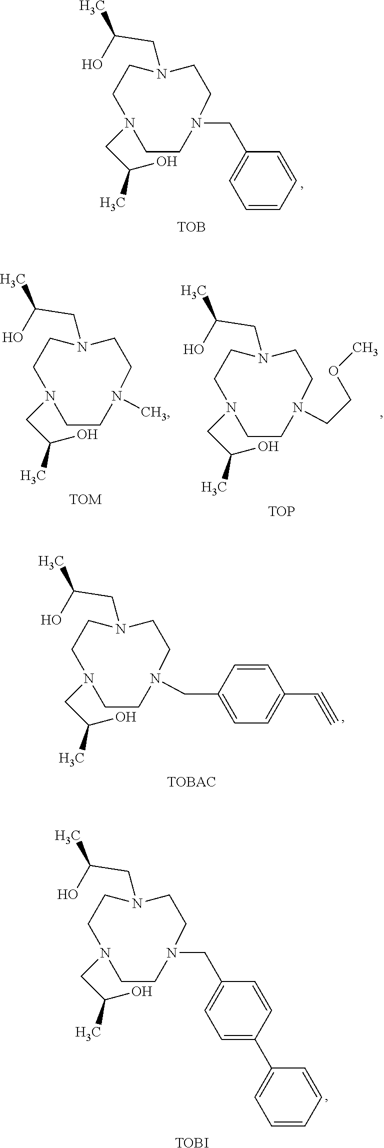

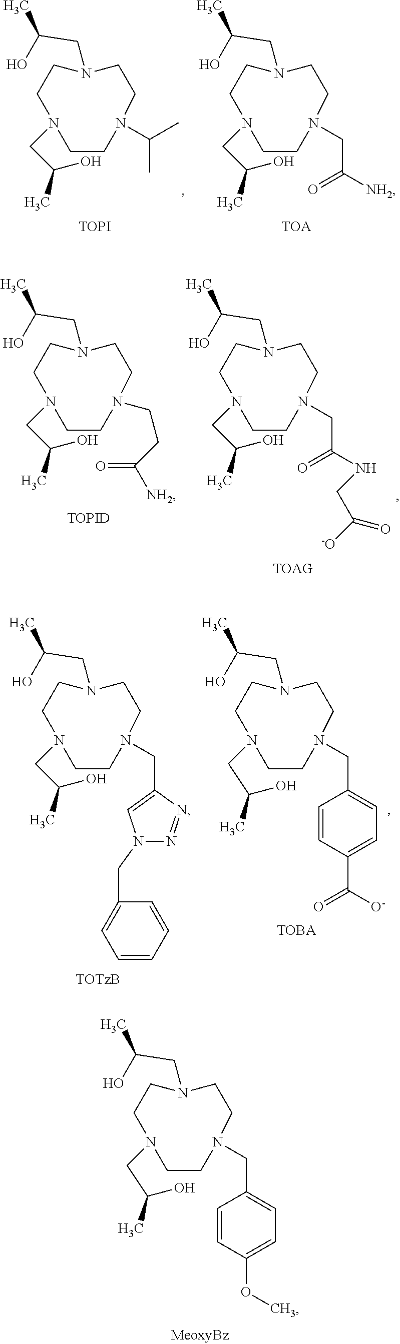

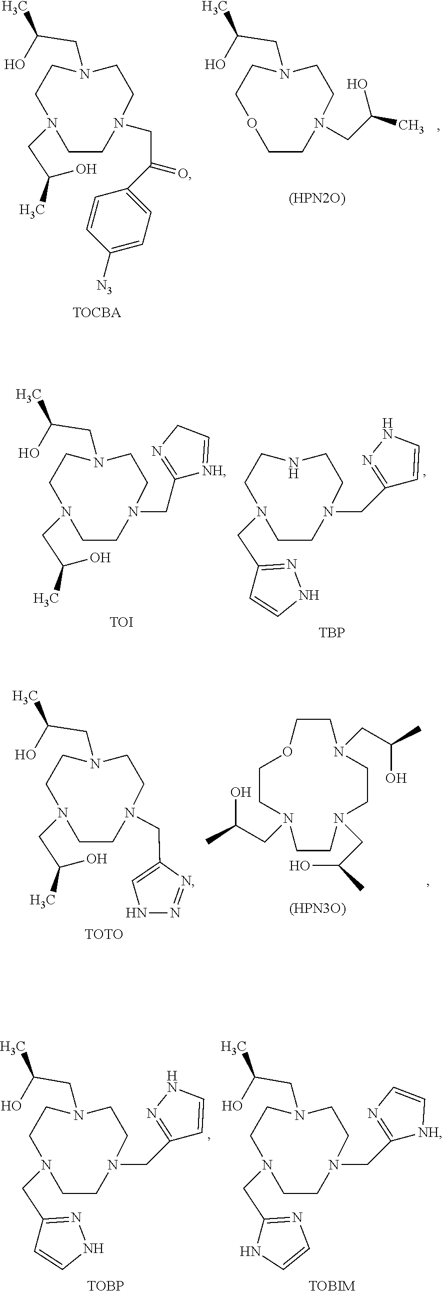

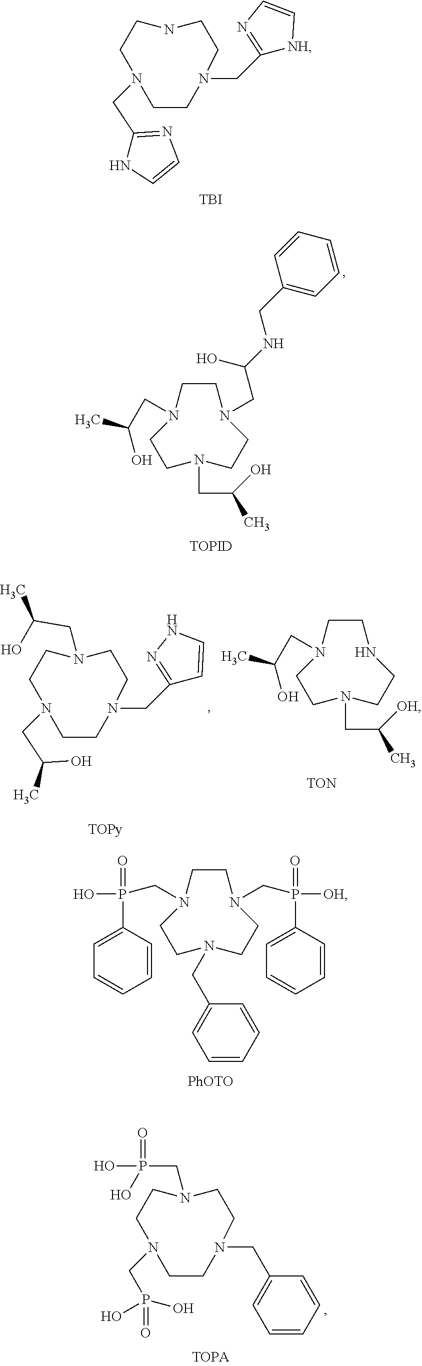

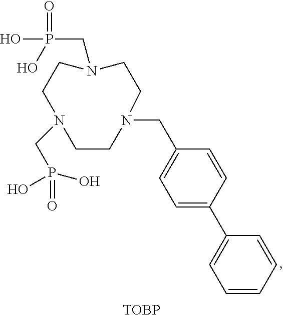 [SMILES: O=P(O)(O)CN1CCN(Cc2ccc(-c3ccccc3)cc2)CCN(CP(=O)(O)O)CC1 ; "TOBP"]

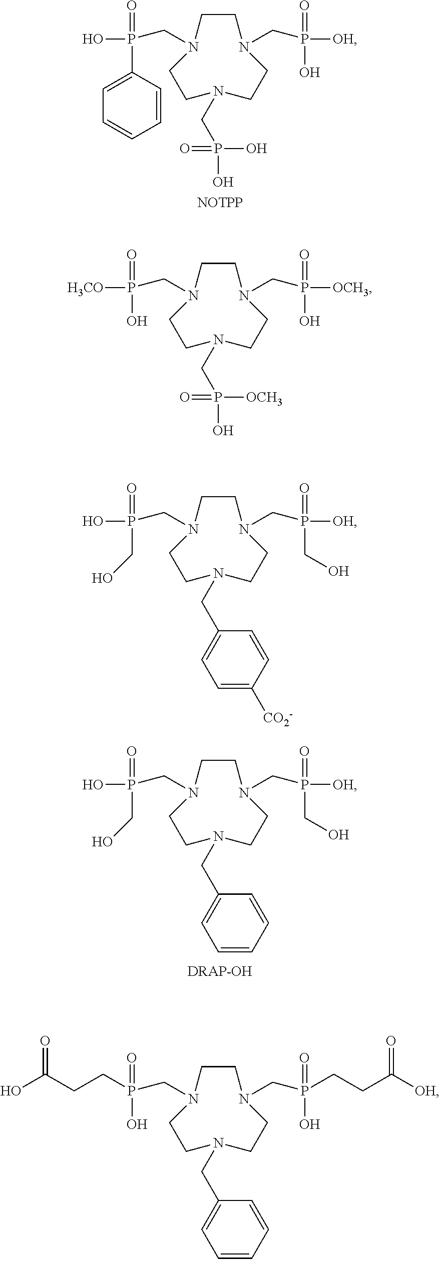

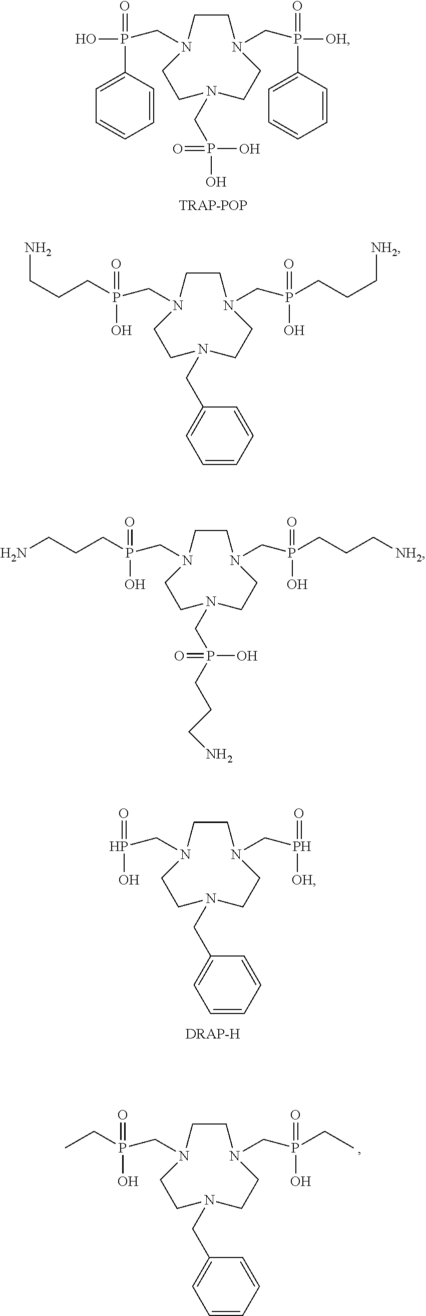

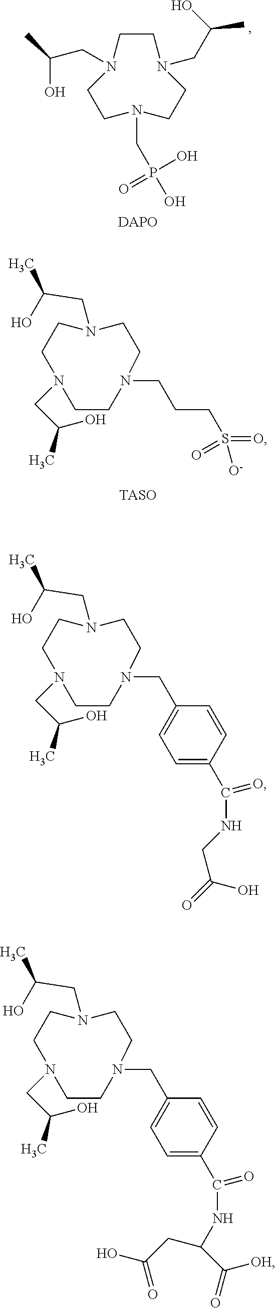 [SMILES: C[C@H](O)CN1CCN(C[C@H](C)O)CCN(CP(=O)(O)O)CC1 ; "DAPO" ; C[C@H](O)CN1CCN(CCCS(=O)(=O)[O-])CCN(C[C@H](C)O)CC1 ; C[C@H](O)CN1CCN(Cc2ccc(C(=O)NCC(=O)O)cc2)CCN(C[C@H](C)O)CC1 ; C[C@H](O)CN1CCN(Cc2ccc(C(=O)NC(CC(=O)O)C(=O)O)cc2)CCN(C[C@H](C)O)CC1]

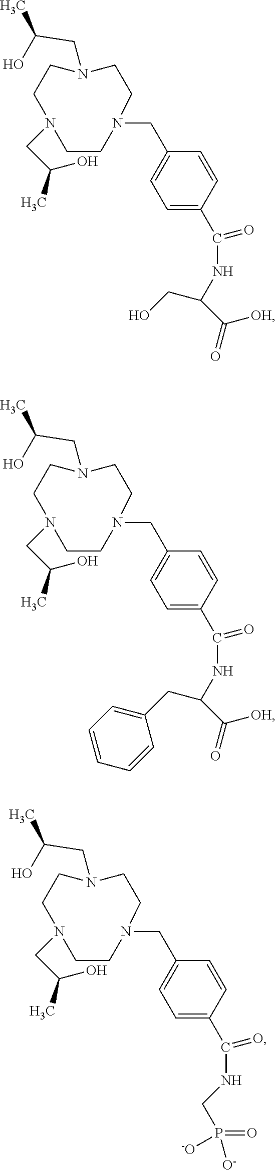

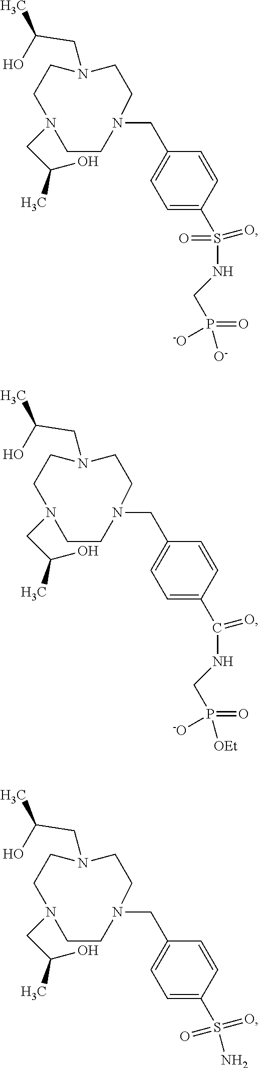

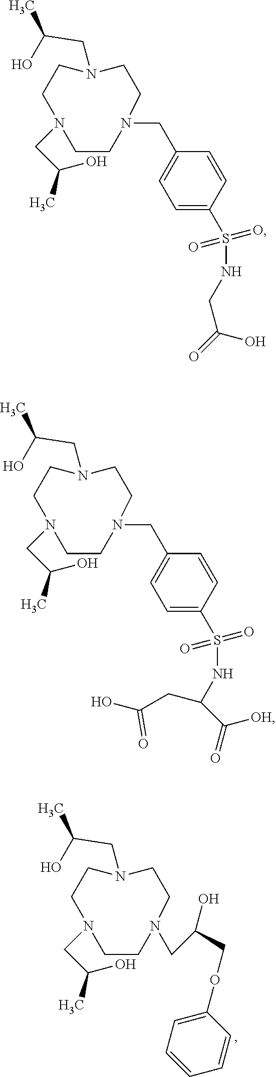 [SMILES: C[C@H](O)CN1CCN(Cc2ccc(S(=O)(=O)NCC(=O)O)cc2)CCN(C[C@H](C)O)CC1 ; C[C@H](O)CN1CCN(Cc2ccc(S(=O)(=O)NC(CC(=O)O)C(=O)O)cc2)CCN(C[C@H](C)O)CC1 ; C[C@H](O)CN1CCN(C[C@@H](O)COc2ccccc2)CCN(C[C@H](C)O)CC1]

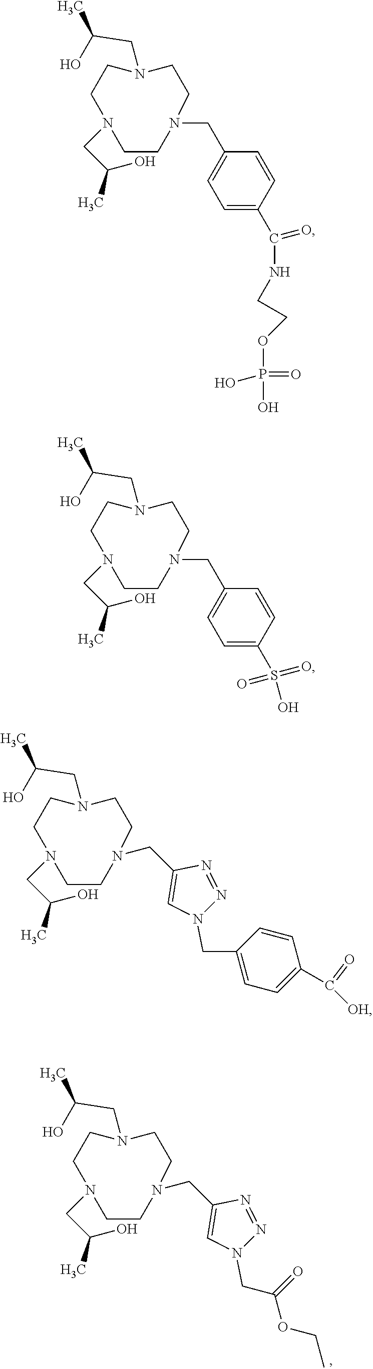 [SMILES: C[C@H](O)CN1CCN(Cc2ccc(C(=O)NCCOP(=O)(O)O)cc2)CCN(C[C@H](C)O)CC1 ; C[C@H](O)CN1CCN(Cc2ccc(S(=O)(=O)O)cc2)CCN(C[C@H](C)O)CC1 ; C[C@H](O)CN1CCN(Cc2cn(Cc3ccc(C(=O)O)cc3)nn2)CCN(C[C@H](C)O)CC1 ; CCOC(=O)Cn1cc(CN2CCN(C[C@H](C)O)CCN(C[C@H](C)O)CC2)nn1]

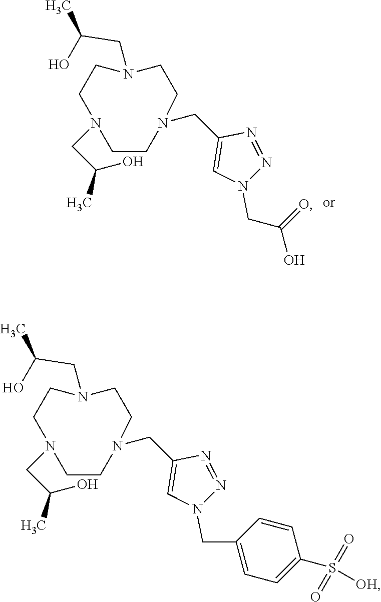 [SMILES: C[C@H](O)CN1CCN(Cc2cn(CC(=O)O)nn2)CCN(C[C@H](C)O)CC1 ; C[C@H](O)CN1CCN(Cc2cn(Cc3ccc(S(=O)(=O)O)cc3)nn2)CCN(C[C@H](C)O)CC1]

where Fe(III) may be complexed to these compounds.

In various examples, complexes of the present disclosure do not have the following structure:

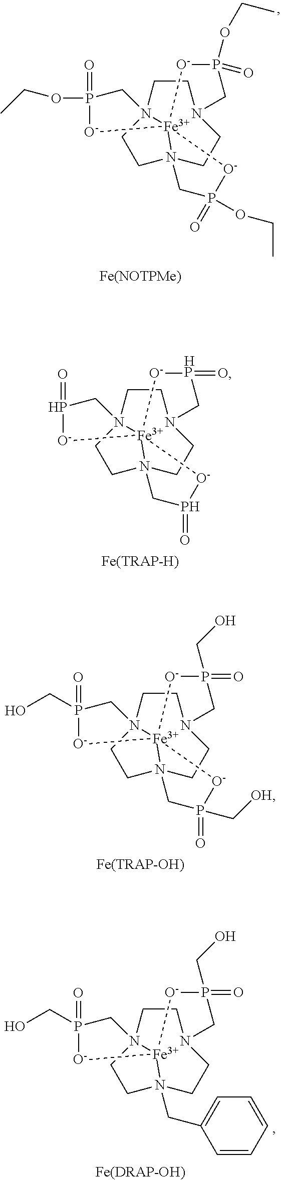

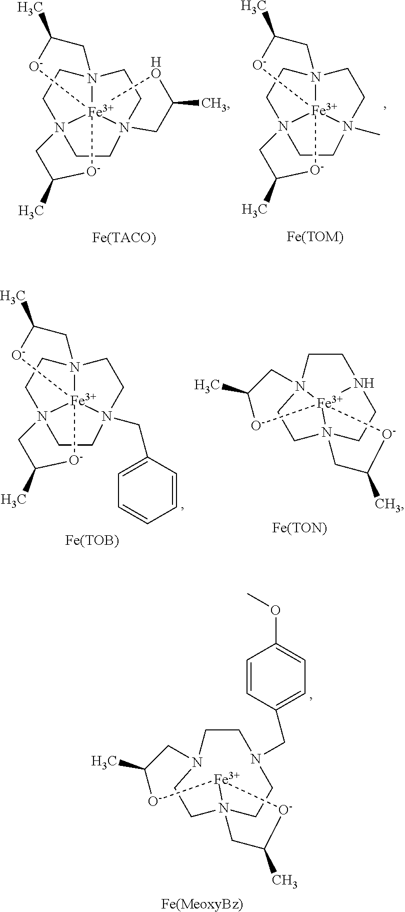

and deprotonated or partially deprotonated species thereof.

In various examples, when the macrocyclic core has the following structure:

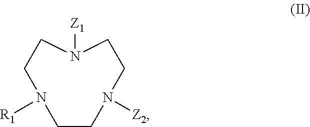

when Z₁ and Z₂ are both

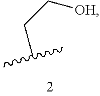 [SMILES: *CCO]

### R₁ is not

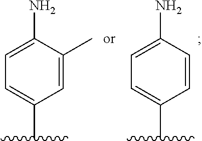

when Z₁ and Z₂ are both

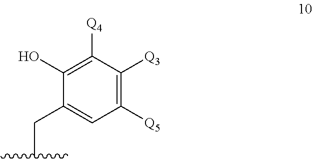

and Q₃ is H, Q₄ is t-butyl, and Q₅ is OCH₃ or Q₃ is H and Q₄ and Q₅ are both t-butyl, R₁ is not ethyl or isopropyl; when Z₁ and Z₂ are both

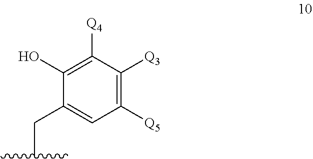

and Q₃, Q₄, and Q₅ are all H, R₁ is not methyl.

In certain embodiments, the complexes used in the methods of the present disclosure may have the following structures:

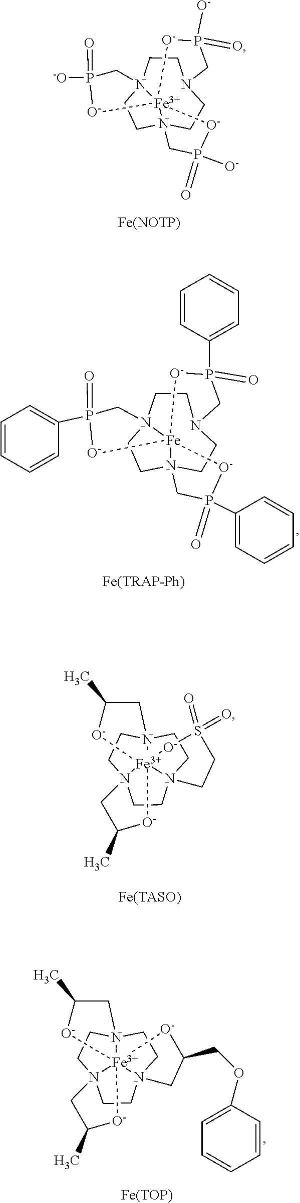

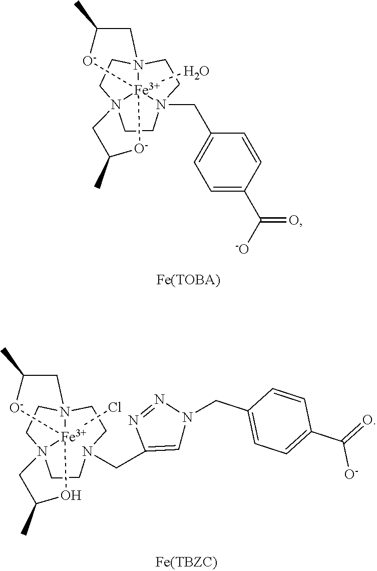

U.S. Pat. No. 11,261,208 and U.S. patent application Ser. No. 16/973,349 are incorporated herein by reference.

The following Statements describe various examples of macrocyclic compounds, macrocyclic complexes, compounds, and compositions of the present disclosure, and uses thereof:

Statement 1. A macrocyclic compound of the present disclosure comprising: a macrocyclic core of the present disclosure (e.g., a macrocyclic core comprising 9 backbone atoms, where at 3 of the atoms in the macrocyclic core is are N atoms, at least two carbon atoms separate a N atom and one or more pendant groups of the present disclosure, where the one or more pendant groups are substituents on (e.g., covalently bound to) the macrocyclic core having the following structure:

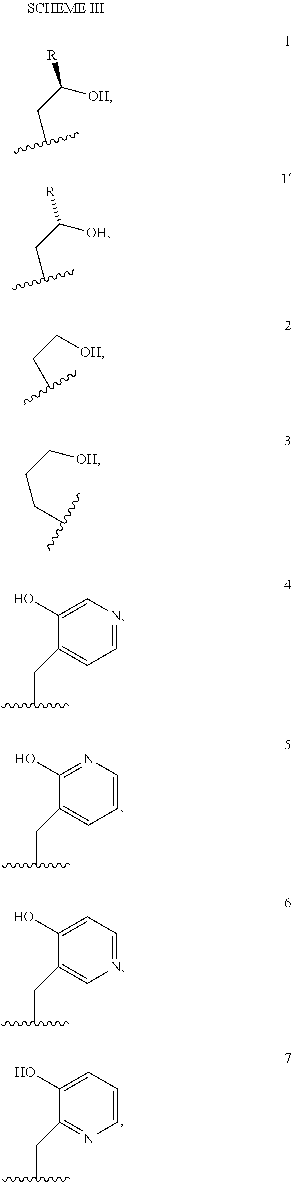

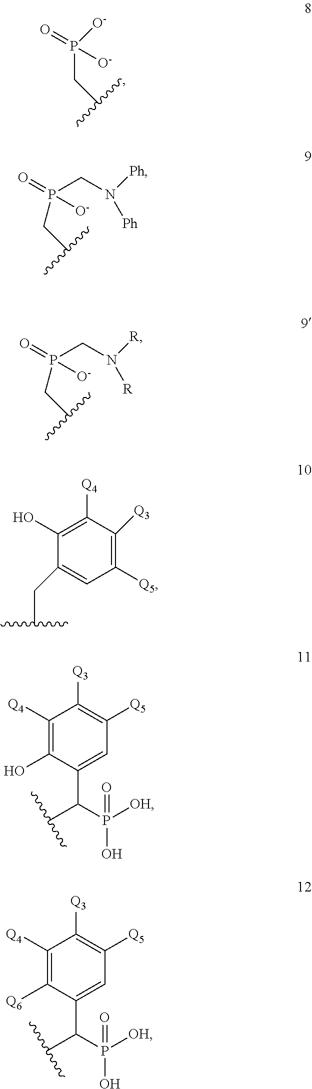

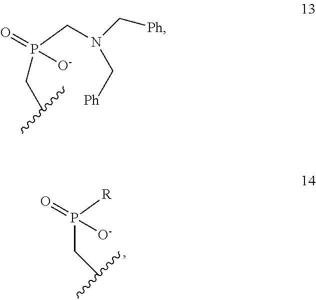

and protonated, partially deprotonated, or completely deprotonated species thereof (where applicable) where Q₃, Q₄ and Q₅ are each independently anionic groups or chosen from —H, —NR₂, —NO₂, —CN, —(CH₂)ₘNR₂, OH, OR, —CH₂P(O)(OH)₂, —(CH₂)ₘP(O)(OH)₂, —SO₃H, and deprotonated species thereof, where m is 1 or 2, where R is H, an alkyl group (e.g., methyl, trifluoromethyl, or the like), an aryl group (e.g., a phenyl group or a phenyl group substituted with a sulfonate), an alkyl carboxylate group, alkyl carboxylic acid group, or the like. The compounds have two of any of 1, 1′, 2, 3, 4, 8, 10, or a combination thereof.
Statement 2. A macrocyclic complex comprising a high-spin Fe(III) cation complexed to a macrocyclic core is a macrocyclic compound of the present disclosure (e.g., a macrocyclic compound according to Statement 1), and/or at least one pendant group substituent of the macrocyclic compound, or a salt, a partial salt, a hydrate, a polymorph, or a stereoisomer thereof, where the macrocyclic compound may exhibit a negative redox potential (e.g., a redox potential of less than 200 vs. normal hydrogen electrode (NHE) in aqueous (e.g., water) solution at a biologically relevant pH (e.g., 6.5-7.5 or 7.2-7.4)).
Statement 3. A macrocyclic compound or complex according to Statements 1 or 2, where at least one or all of the one or more pendant groups is/are covalently bound to a N on the macrocyclic core.
Statement 4. A macrocyclic complex according to Statements 2 or 3, where the macrocyclic complex has at least one open coordination site. In various examples, the macrocyclic complex is coordinatively saturated with no sites for binding water.
Statement 5. A macrocyclic complex according to any one of Statements 2-4, where the macrocyclic complex has at least one water or at least one hydroxide complexed to the high-spin Fe(III) cation.
Statement 6. A macrocyclic compound or macrocyclic complex according to any one of Statements 1-5, where at least one of the pendant groups is substituted at a benzylic position or any carbon the alkyl group leading to the heteroatom of the pendant group.
Statement 7. A macrocyclic compound or macrocyclic complex according to any one of Statements 1-6, where the macrocyclic core is a TACN group.
Statement 8. A macrocyclic complex according to any one of Statements 2-4, where the macrocyclic complex comprises a TACN group and at least one (e.g., one or two) anionic pendant groups.
Statement 9. A macrocyclic complex according to Statement 8, where the anionic pendants are individually chosen from alkoxide pendants, phenoxide pendants, phosphinate pendants, phosphonate pendants and combinations thereof. In various examples, there are two hydroxyalkyl pendant groups or two phenoxide pendant groups.
Statement 10. A macrocyclic complex according to Statement 8 or 9, where the macrocyclic complex further comprises a coordinating pendant group or a non-coordinating pendant.
Statement 11. A macrocyclic compound or macrocyclic complex according to any one of Statements 1-10, where the macrocyclic core has one of the following structures:

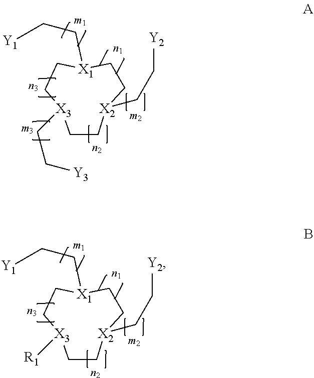

wherein X₁, X₂, and X₃, are N; Y₁, Y₂, or Y₃ are each independently pendant donors comprising O, wherein O has at least one lone pair of electrons but preferably two or three lone pairs (e.g., ketone, alcohol, alkoxide, carboxylic acid, amide, phenol or phenoxide, or a deprotonated form of the foregoing, such as, for example, a carboxylate ion, or an oxide, including an alkoxide or a phenoxide; m₁, m₂, or m₃ are each independently 0, 1, or 2; n₁, n₂, or n₃ are each independently 1 or 2; and R₁ is a substituted or unsubstituted aryl, substituted or unsubstituted heteroaryl or substituted or unsubstituted alkyl group, wherein R₁ is not substituted by a pendant donor, wherein the alkyl segment of the alkyl-Y chain (alkyl-Y₁, alkyl-Y₂, and/or alkyl-Y₃) may each independently be substituted (e.g., Structure a or Structure b) or unsubstituted. For Structures a or b, the pendant may have either R or S configuration at the chiral carbon:

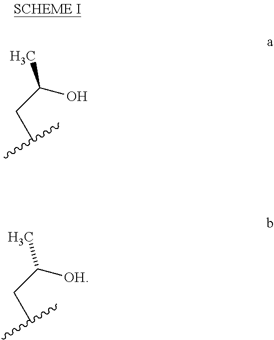

Statement 12. A macrocyclic compound or complex according to any one of Statements 2-11, where the macrocyclic core has the following structure:

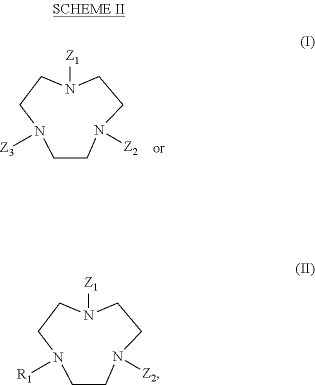

where R₁ is a substituted or unsubstituted aryl group, substituted or unsubstituted heteroaryl group, or substituted or unsubstituted alkyl group, where R₁ is not a substituted by pendant donors; and when the macrocyclic core has Structure I, Z₁ is H or one of the pendant groups in Scheme III and Z₂ and Z₃ each independently is a pendant group (e.g., one of the pendant groups in Scheme III); when the macrocyclic compound has Structure II, Z₁ and Z₂ each independently is a pendant group (e.g., one of the pendant groups in Scheme III).
Statement 13. A macrocyclic complex according to any one of Statements 2-12, where the macrocyclic core with pendant donors has the following structure (to which Fe(III) can be complexed thereto):

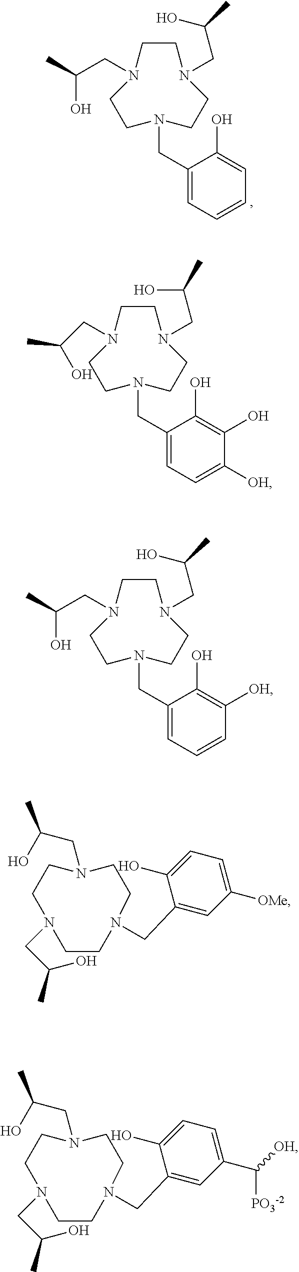

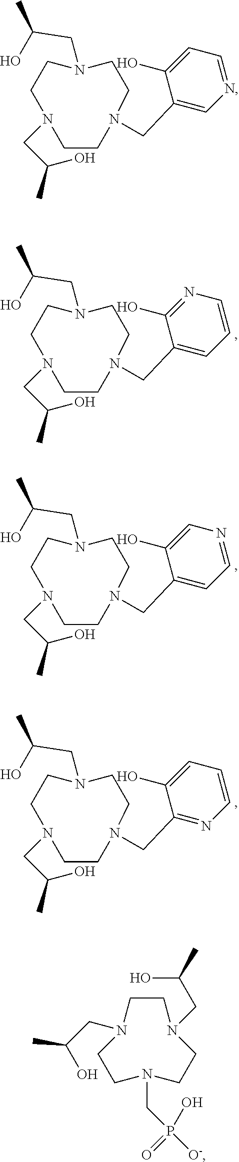

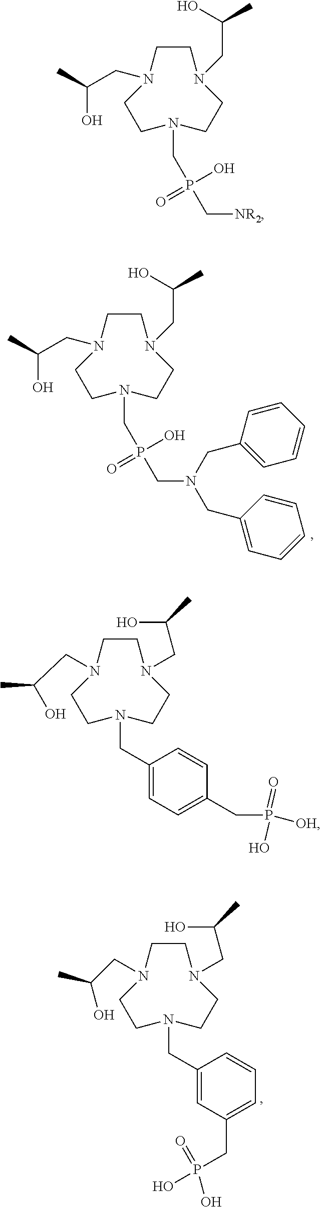

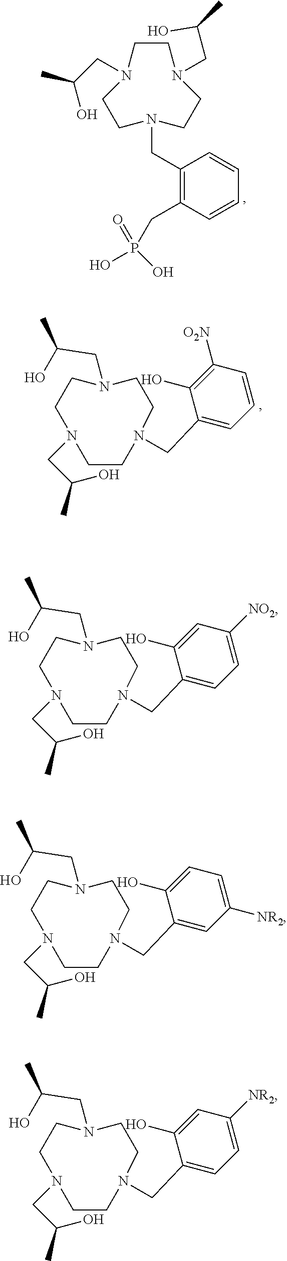

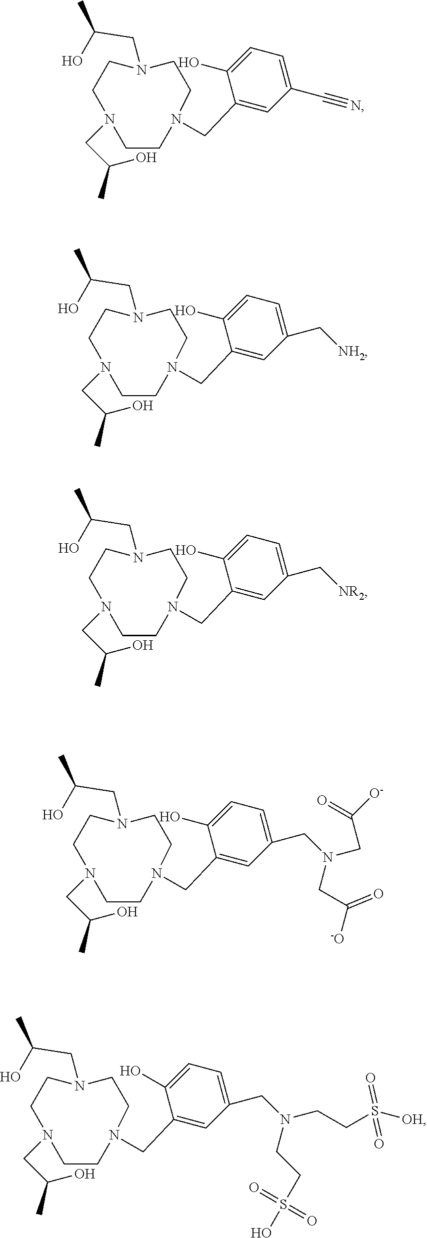

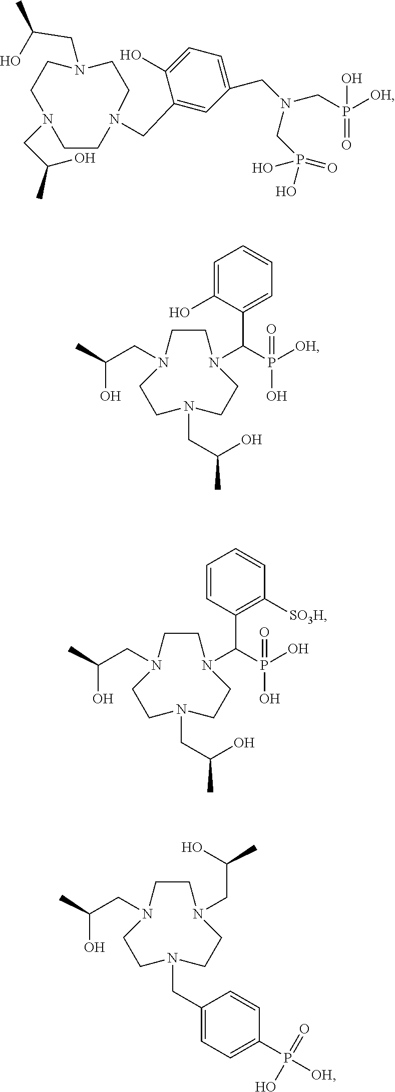 [SMILES: C[C@H](O)CN1CCN(Cc2cc(CN(CP(=O)(O)O)CP(=O)(O)O)ccc2O)CCN(C[C@H](C)O)CC1 ; C[C@H](O)CN1CCN(C[C@H](C)O)CCN(C(c2ccccc2O)P(=O)(O)O)CC1 ; C[C@H](O)CN1CCN(C[C@H](C)O)CCN(C(c2ccccc2S(=O)(=O)O)P(=O)(O)O)CC1 ; C[C@H](O)CN1CCN(Cc2ccc(P(=O)(O)O)cc2)CCN(C[C@H](C)O)CC1]

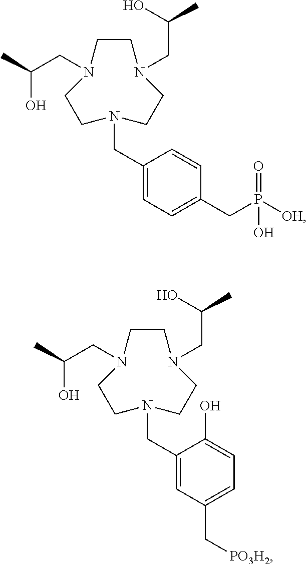

or deprotonated or partially deprotonated species thereof or analogs thereof,
or the macrocyclic complex has the following structure:

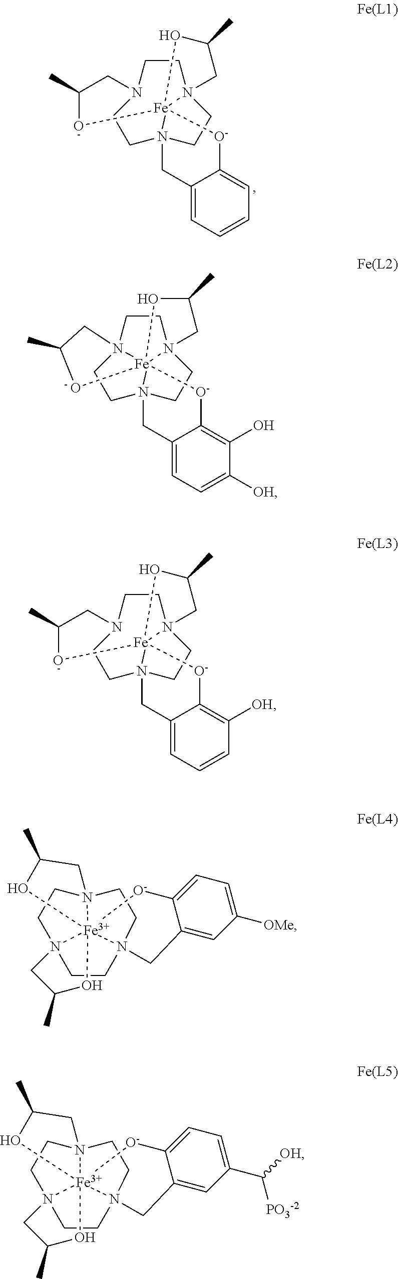

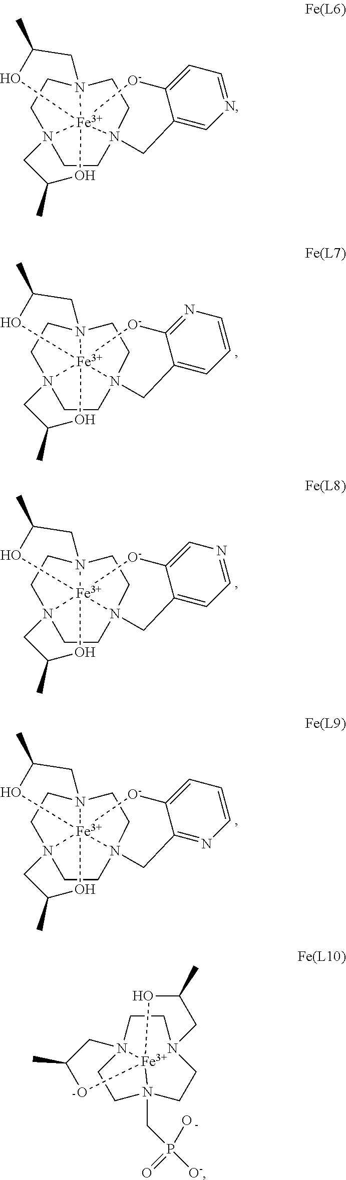

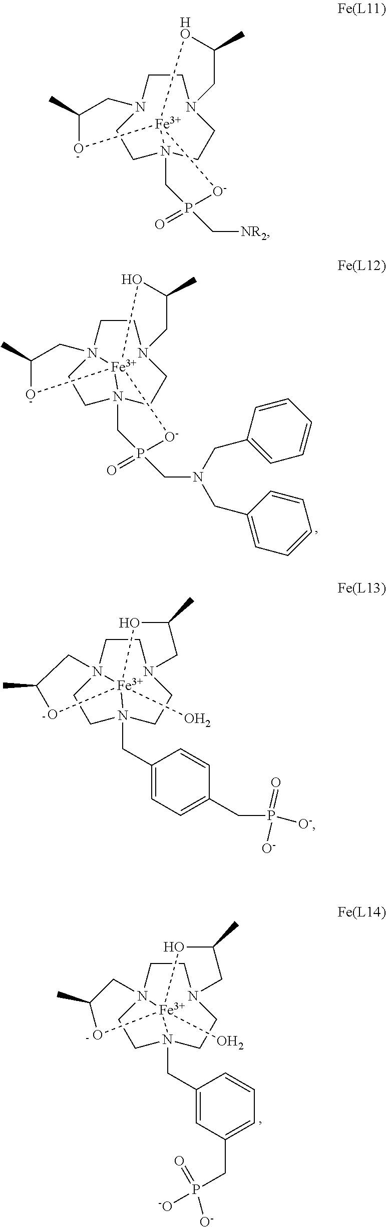

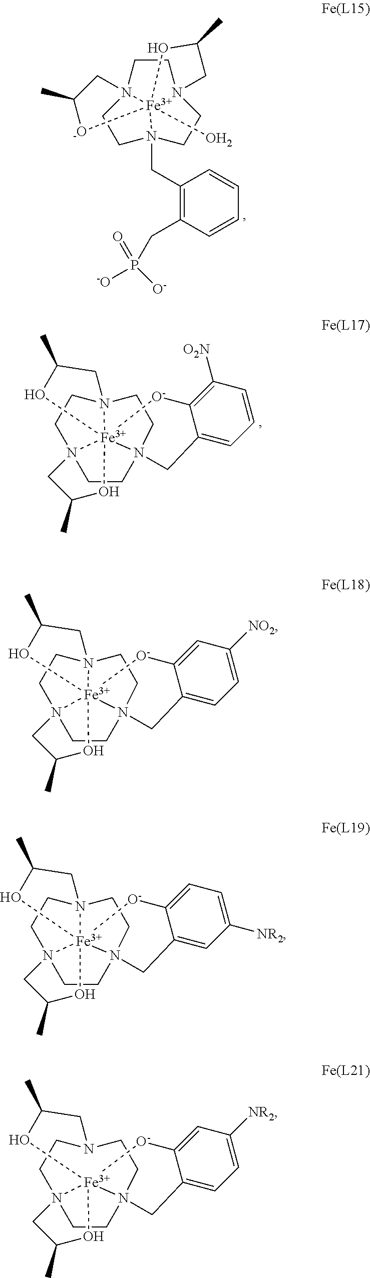

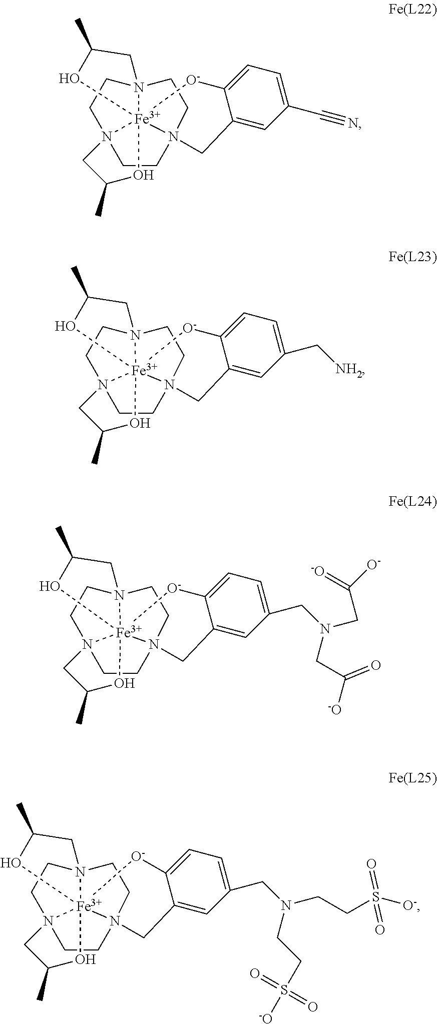

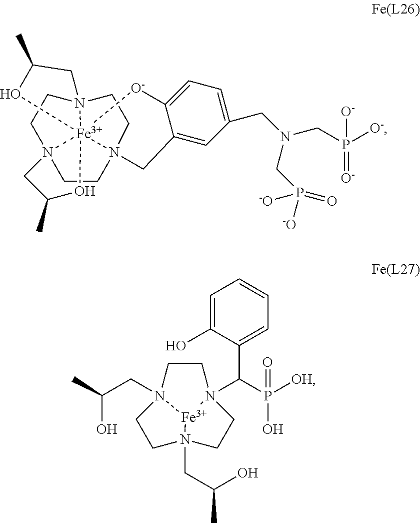

or deprotonated or partially deprotonated species thereof or analogs thereof.
Statement 15. A composition comprising one or more macrocyclic compounds and/or one or more macrocyclic complexes of the present disclosure (e.g., one or more macrocyclic compounds according to Statements 1 and/or one or more macrocyclic complex according to any one of Statements 2-14) and a pharmaceutically acceptable carrier.
Statement 16. A composition according to Statement 15, where the composition further comprises human serum albumin and/or meglumine.
Statement 17. A method to obtain an image of at least a portion of a cell, organ, vasculature or tissue comprising: contacting the cell, organ, vasculature, or tissue with one or more macrocyclic compound and/or one or more macrocyclic complex of the present disclosure (e.g., one or more macrocyclic compound according to Statement 1 and/or one or more macrocyclic complex according to any one of Statements 2-14) and/or one or more composition of the present disclosure (e.g., a composition according to any one of Statements 15-16), and imaging at least a portion of the cell, organ, vasculature, or tissue to obtain an image of the portion of a cell, organ, vasculature, or tissue, where the image is obtained by using magnetic resonance.
Statement 18. A method according to Statement 17, where the cell, organ, vasculature, or tissue is part of an individual.
Statement 19. A method according to Statements 17 or 18, where the image is obtained using magnetic resonance imaging (MRI).
Statement 20. A method according to any one of Statements 17-19, where the macrocyclic compound(s) and/or compound(s) is/are a T₁ agent or T₁ agents.
Statement 21. A macrocyclic core comprising 9 backbone atoms, wherein 3 atoms in the macrocyclic core are N atoms, at least two carbon atoms separate the N atoms, and one or more of the following pendant groups are substituents on the macrocyclic core:

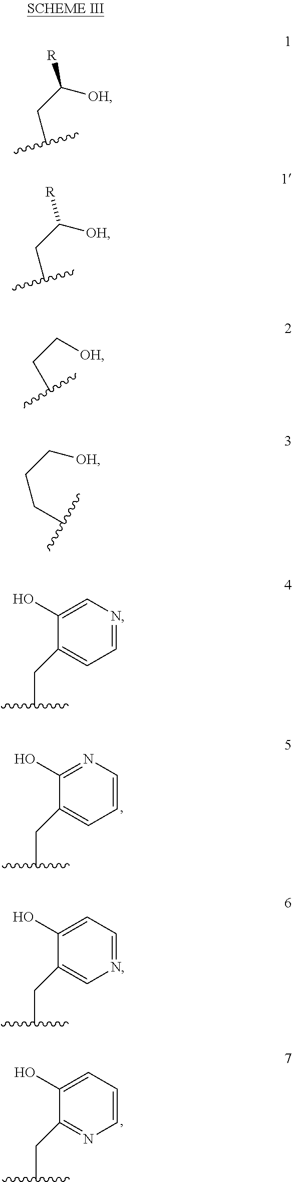

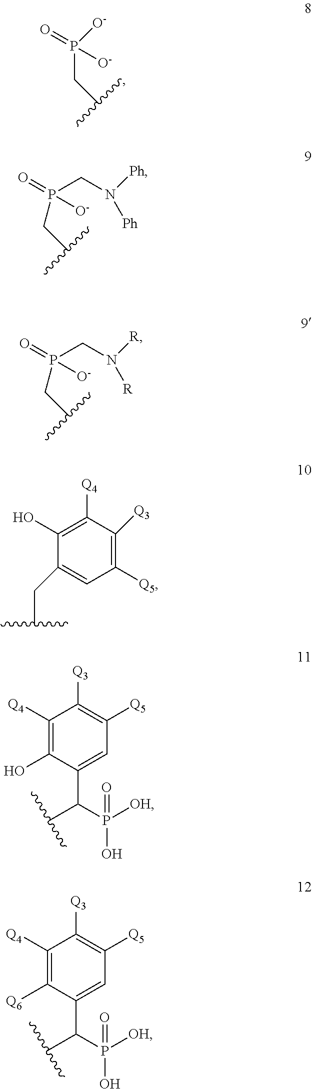

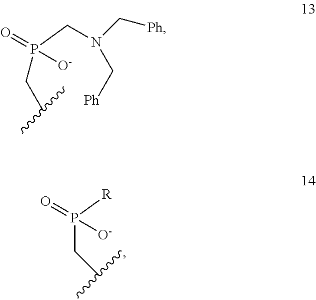

and protonated, partially deprotonated, or completely deprotonated species thereof (where applicable), where Q₃, Q₄ and Q₅ are each independently anionic groups or chosen from —H, —NR₂, —NO₂, —CN, —(CH₂)ₘNR₂, OH, OR, —CH₂P(O)(OH)₂, —(CH₂)ₘP(O)(OH)₂, —SO₃H, and deprotonated species thereof, where m is 1 or 2, where R is H, an alkyl group (e.g., methyl, trifluoromethyl, or the like), an aryl group (e.g., a phenyl group or a phenyl group substituted with a sulfonate), an alkyl carboxylate group, alkyl carboxylic acid group, or the like. The compounds have two of any of 1, 1′, 2, 3, 4, 8, 10, or a combination thereof or a salt, a partial salt, a hydrate, a polymorph, or a stereoisomer thereof.
Statement 22. A macrocyclic core according to Statement 21, where at least one or all of the one or more pendant groups is/are covalently bound to a N on the macrocyclic core.
Statement 23. A macrocyclic core according to Statement 21 or Statement 22, where at least one of the pendant groups is substituted at a benzylic position or any carbon the alkyl group leading to the heteroatom of the pendant group.
Statement 24. A macrocyclic core according to any one of Statements 21-23, where the macrocyclic core is a TACN group.
Statement 25. A macrocyclic core according to any one of Statements 21-23, where the macrocyclic core comprises a TACN moiety and at least one (e.g., one or two) anionic pendant groups.
Statement 26. A macrocyclic core according to Statement 25, where the anionic pendants are individually chosen from alkoxide pendants, phenoxide pendants, and combinations thereof. Statement 27. A macrocyclic core according to Statement 26, further comprises a coordinating pendant group or a non-coordinating pendant group.
Statement 28. A macrocyclic core according to any one of Statements 21-27, where the macrocyclic core has one of the following structures:

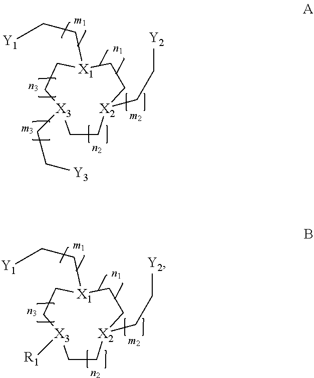

where X₁, X₂, and X₃, are N; Y₁, Y₂, or Y₃ are each independently pendant donors comprising N, wherein N has a lone pair of electrons (e.g., amino, benzimidazole, imidazole, aniline, pyrazoyl, triazole, benzotriazole, and the like) or a pendant donor comprising O, wherein O has at least one lone pair of electrons but preferably two or three lone pairs (e.g., ketone, alcohol, alkoxide, carboxylic acid, amide, phenol or phenoxide, or a deprotonated form of the foregoing, such as, for example, a carboxylate ion, an imidazolate ion, a pyrazolate ion or an oxide, including an alkoxide or a phenoxide; m₁, m₂, or m₃ are each independently 0, 1, or 2; n₁, n₂, or n₃ are each independently 1 or 2; and R₁ is a substituted or unsubstituted aryl, substituted or unsubstituted heteroaryl or substituted or unsubstituted alkyl group, wherein R₁ is not substituted by a pendant donor, wherein the alkyl segment of the alkyl-Y chain (alkyl-Y₁, alkyl-Y₂, and/or alkyl-Y₃) may each independently be substituted (e.g., Structure a or Structure b) or unsubstituted.
Statement 29. A macrocyclic core according to any one of Statements 21-28, having the following structure:

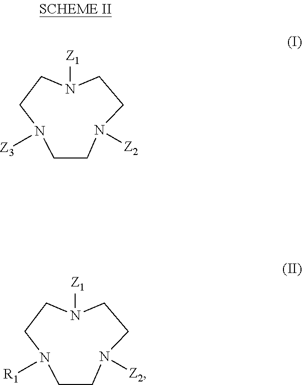

where R₁ is a substituted or unsubstituted aryl group, substituted or unsubstituted heteroaryl group, or substituted or unsubstituted alkyl group, where R₁ is not a substituted by pendant donors; and when the macrocyclic core has Structure I, Z₁ is H or one of the pendant groups in Scheme III and Z₂ and Z₃ each independently is a pendant group (e.g., one of the pendant groups in Scheme III); when the macrocyclic compound has Structure II, Z₁ and Z₂ each independently is a pendant group (e.g., one of the pendant groups in Scheme III).
Statement 30. A macrocyclic core according to any one of Statement 21-29, where the macrocyclic core has the following structure:

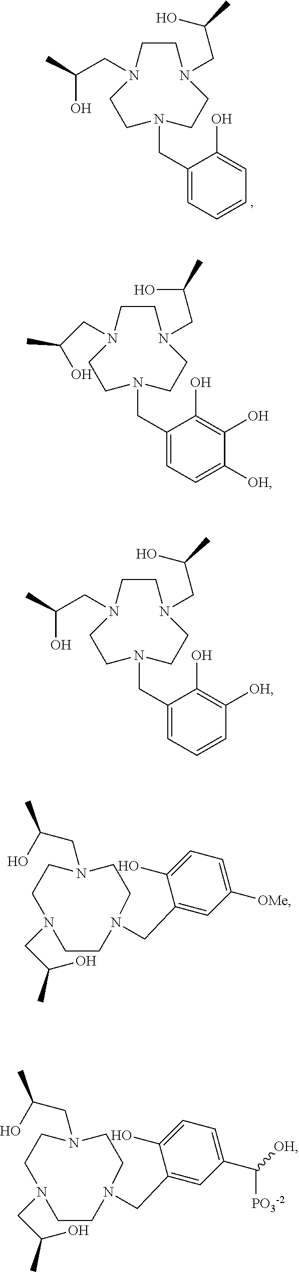

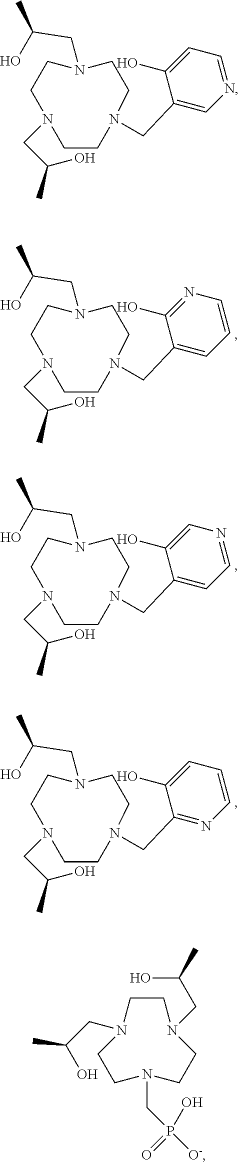

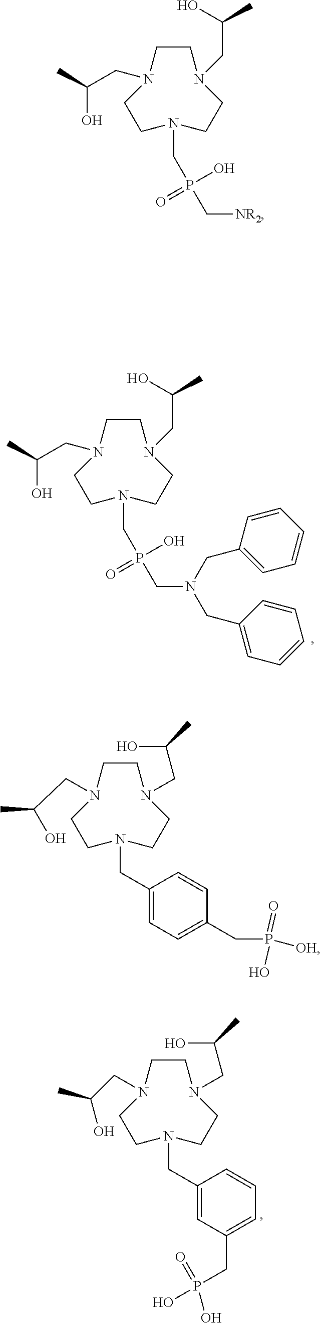

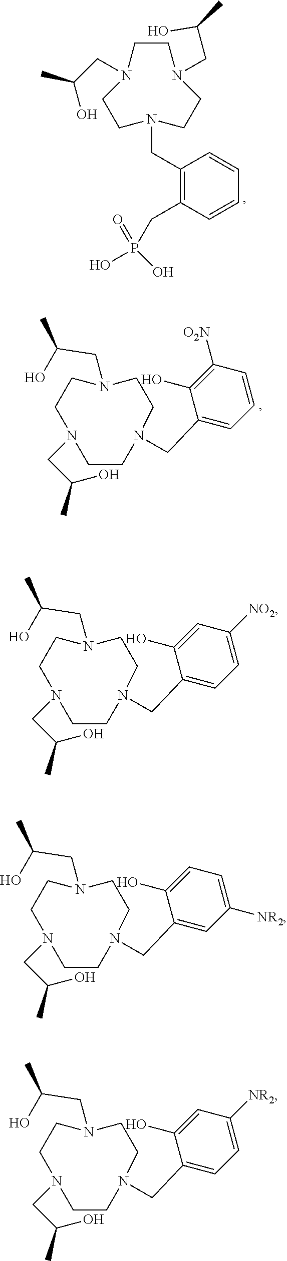

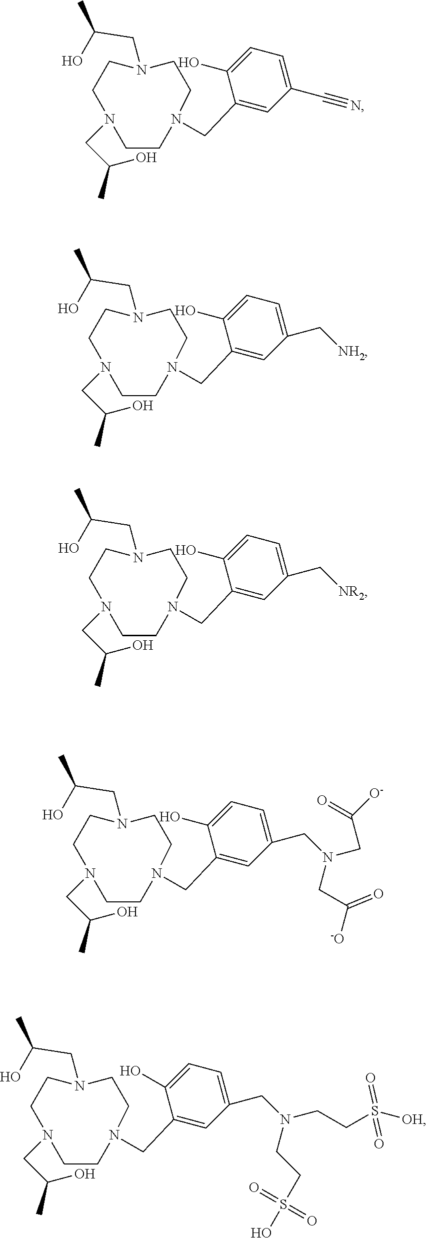

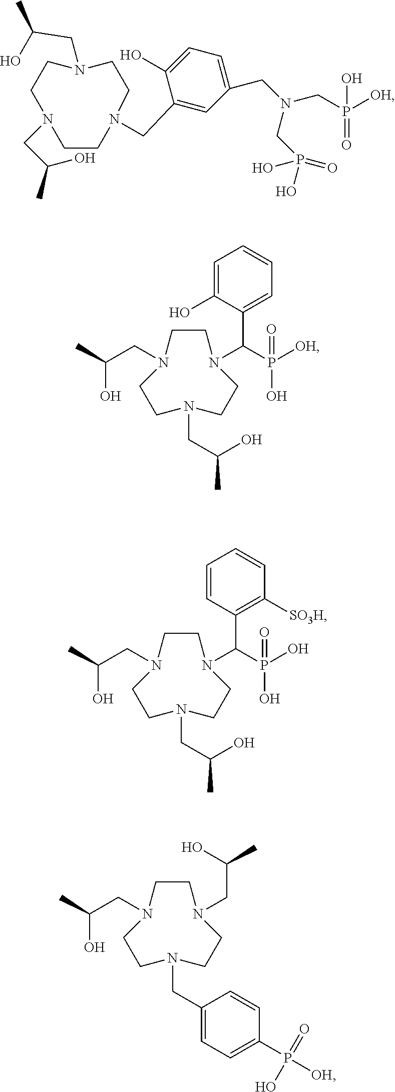 [SMILES: C[C@H](O)CN1CCN(Cc2cc(CN(CP(=O)(O)O)CP(=O)(O)O)ccc2O)CCN(C[C@H](C)O)CC1 ; C[C@H](O)CN1CCN(C[C@H](C)O)CCN(C(c2ccccc2O)P(=O)(O)O)CC1 ; C[C@H](O)CN1CCN(C[C@H](C)O)CCN(C(c2ccccc2S(=O)(=O)O)P(=O)(O)O)CC1 ; C[C@H](O)CN1CCN(Cc2ccc(P(=O)(O)O)cc2)CCN(C[C@H](C)O)CC1]

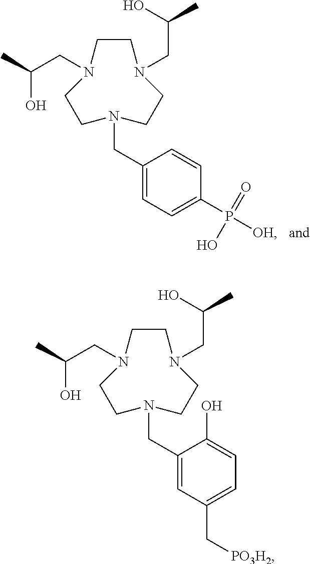

or deprotonated, partially deprotonated, or protonated species thereof (where applicable) or the macrocyclic complex has the following structure:

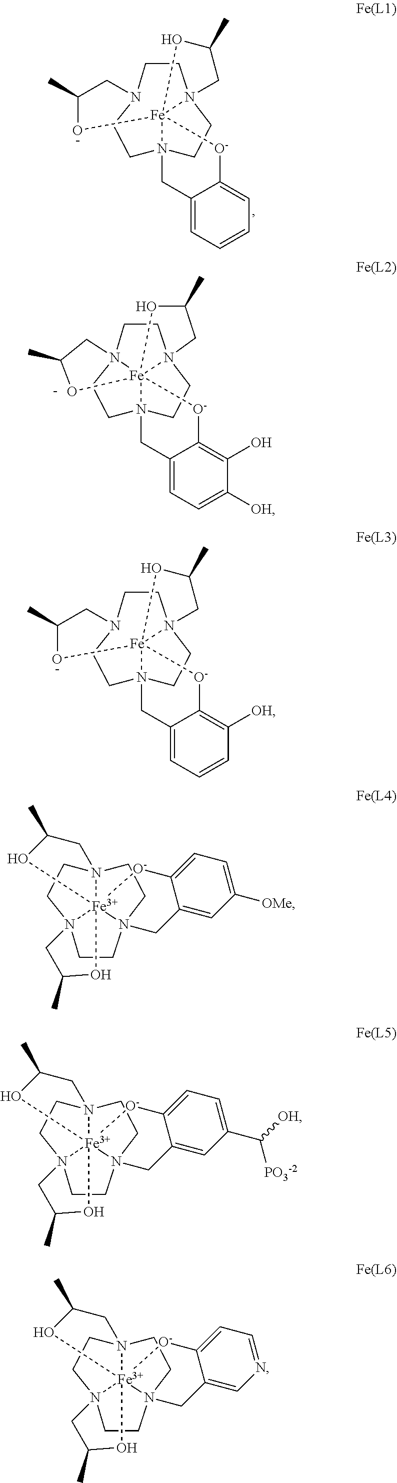

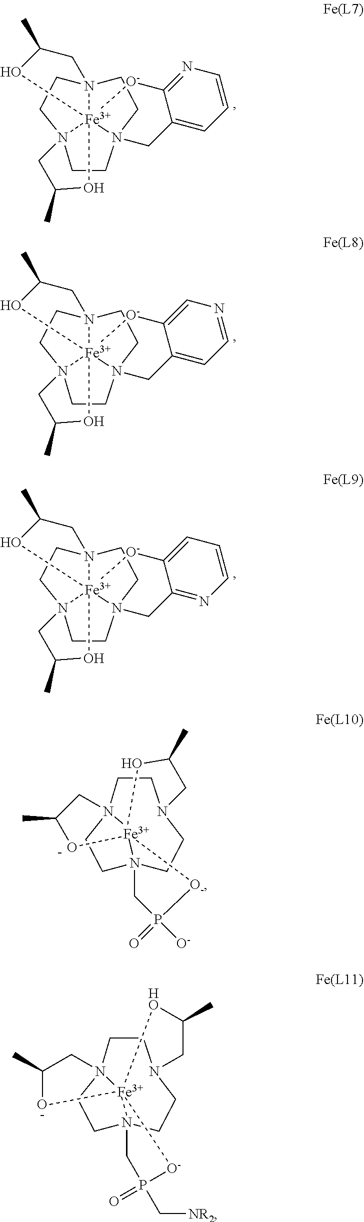

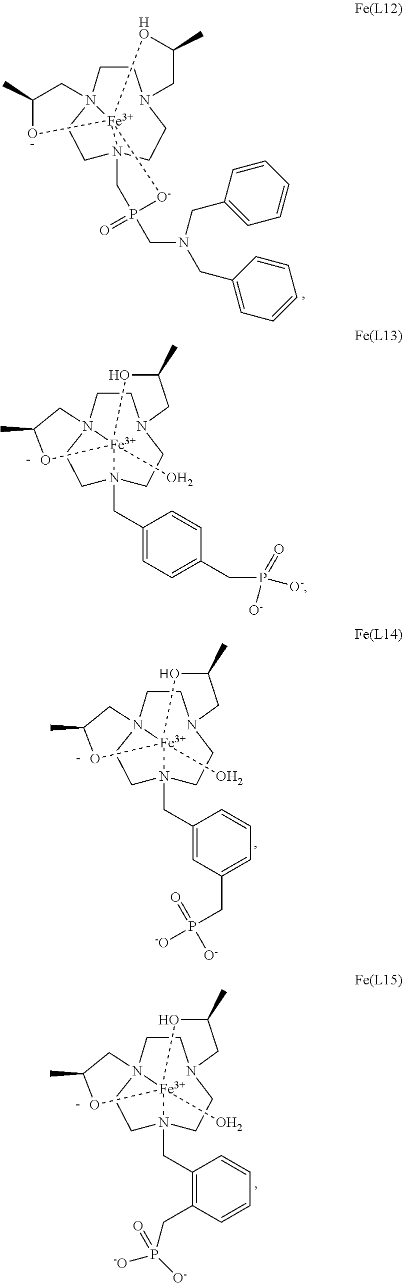

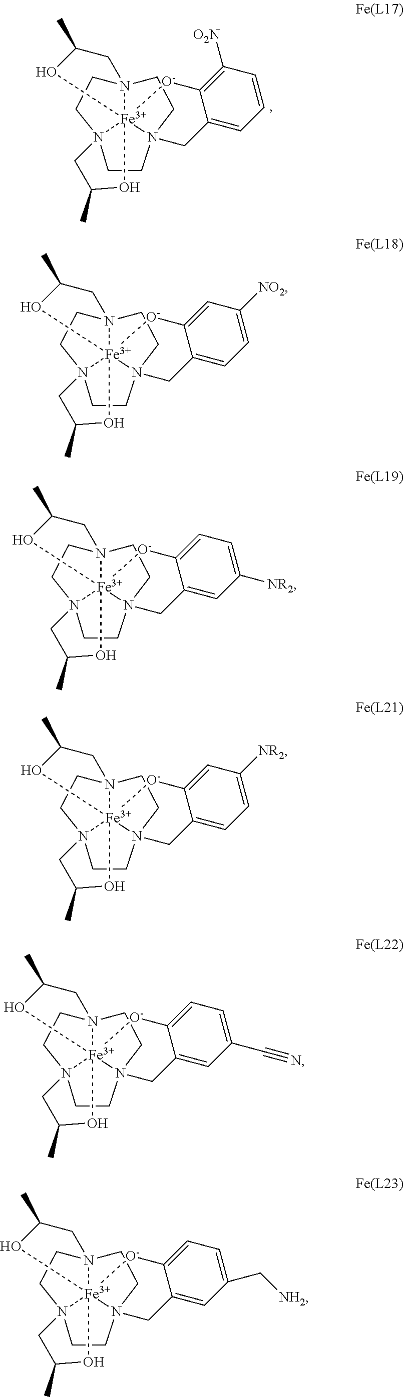

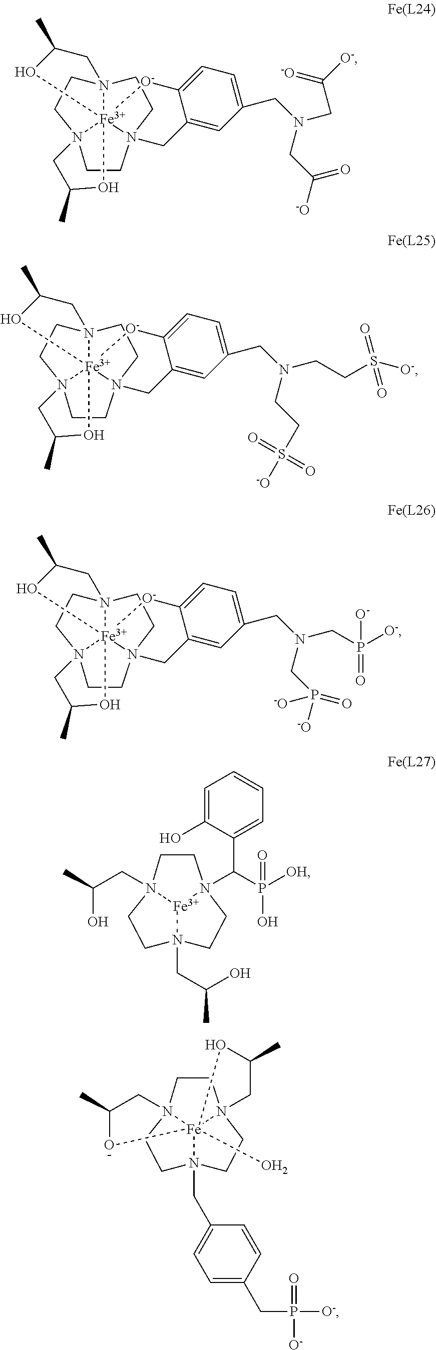

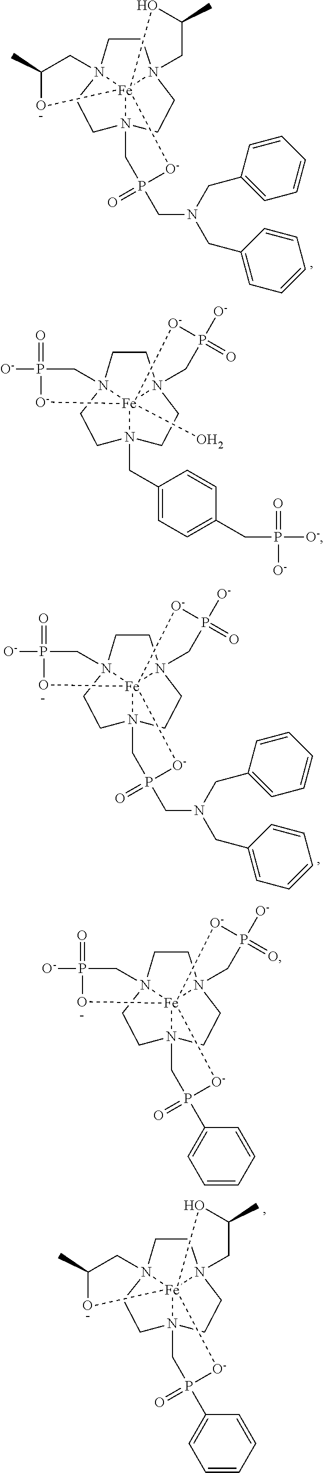

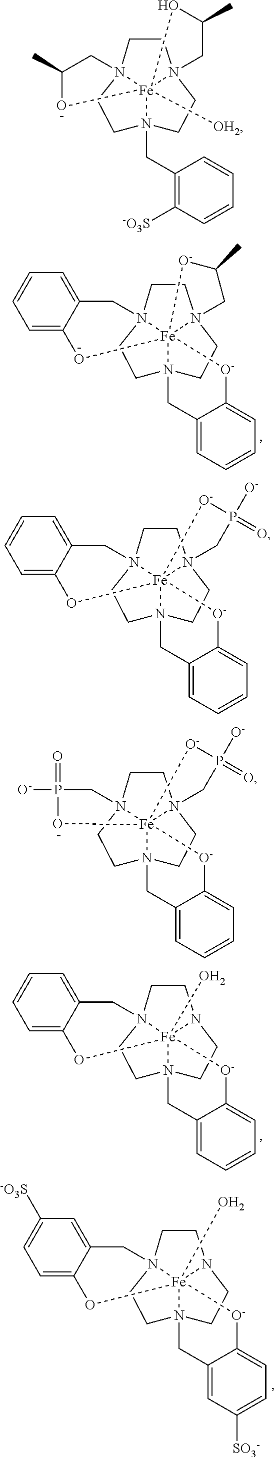

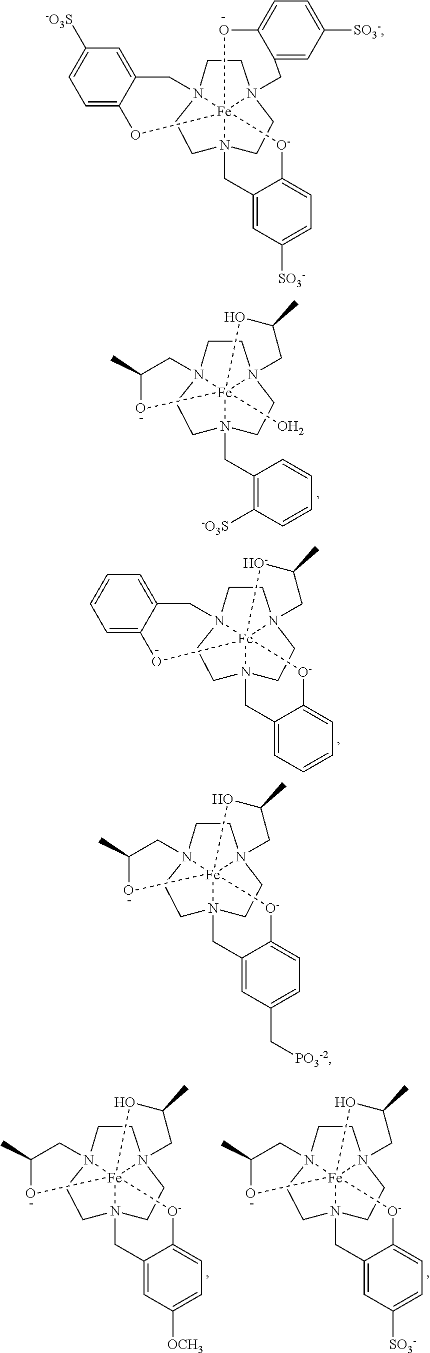

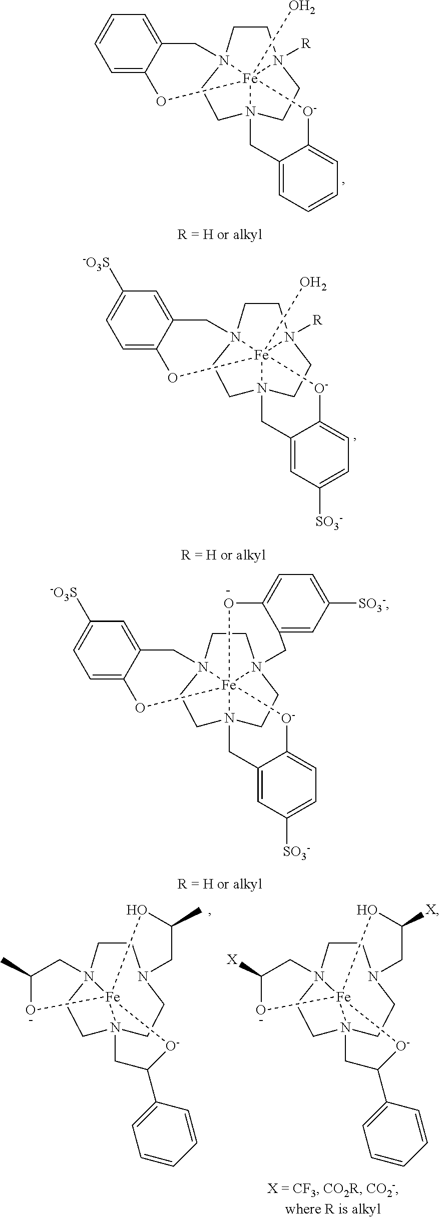

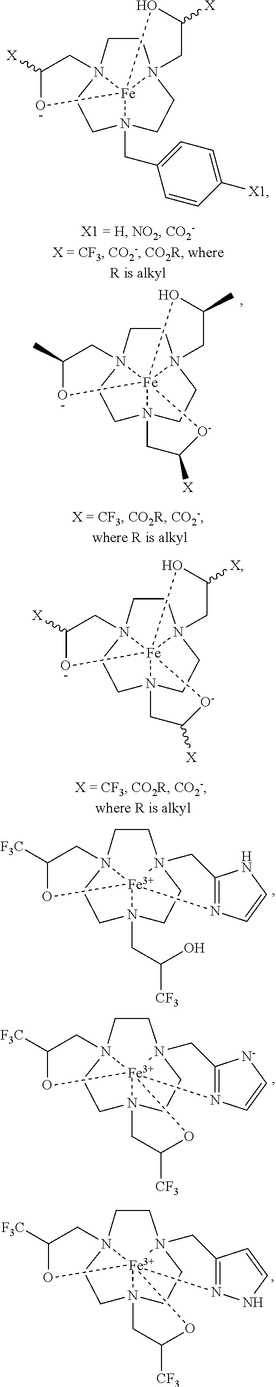

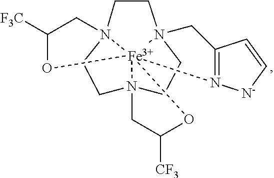

and deprotonated, partially deprotonated, and protonated species thereof (where applicable).
Statement 31. A macrocyclic complex having the following structure:

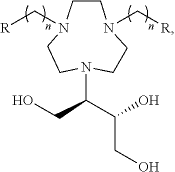

where the tri(hydroxy)butyl group(s) and —(CH₂)ₙR groups are pendant groups and each R is independently selected from alkyl groups; aryl groups; heteroaryl groups; alkyl groups comprising one or more —OH groups, one or more sulfonic acid groups, one or more carboxylic acid groups, one or more phosphonic acid groups, one or more alkyl groups, or the like, or combinations thereof; aryl groups comprising one or more —OH groups, one or more sulfonic acid groups, one or more carboxylic acid groups, one or more phosphonic acid groups, one or more alkyl groups, or the like, or combinations thereof, heteroaryl groups comprising one or more —OH groups, one or more sulfonic acid groups, one or more carboxylic acid groups, one or more phosphonic acid groups, one or more alkyl groups, or the like, or combinations thereof; and H; or a salt, a partial salt, a hydrate, a polymorph, or a stereoisomer thereof; n is 1, 2, or 3, wherein a high-spin Fe(III) cation is complexed to the macrocyclic core and/or at least one pendant group substituent of the macrocyclic compound.
Statement 32. A macrocyclic complex according to Statement 31, wherein the macrocyclic complex further comprises a coordinating pendant group or a non-coordinating pendant group.
Statement 33. A macrocyclic complex according to Statement 31 or Statement 32, wherein at least one of the pendant groups is substituted at a benzylic position or any carbon the alkyl group leading to the heteroatom of the pendant group.
Statement 34. A macrocyclic complex according to Statement 31 or Statement 32, wherein the pendant groups are chosen from:

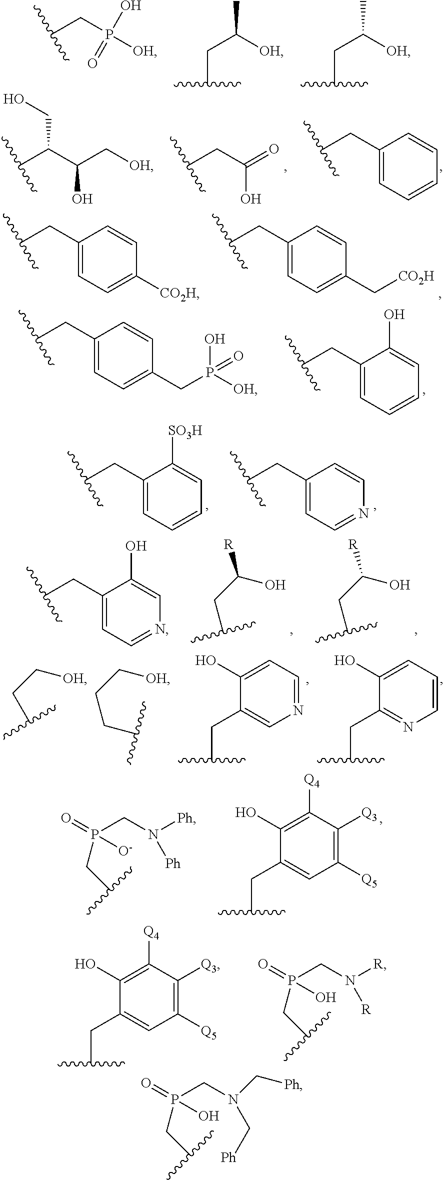

and protonated, partially deprotonated, and deprotonated species thereof (where applicable).
Statement 35. A macrocyclic complex according to any one of Statement 31-34, wherein the macrocyclic complex has at least one open coordination site.
Statement 36. A macrocyclic complex according to any one of Statements 31-35, wherein the macrocyclic complex has at least one water or at least one hydroxide complexed to the high-spin Fe(III) cation.
Statement 37. A macrocyclic complex according to any one of Statements 31-36, wherein the macrocyclic complex has the following structure:

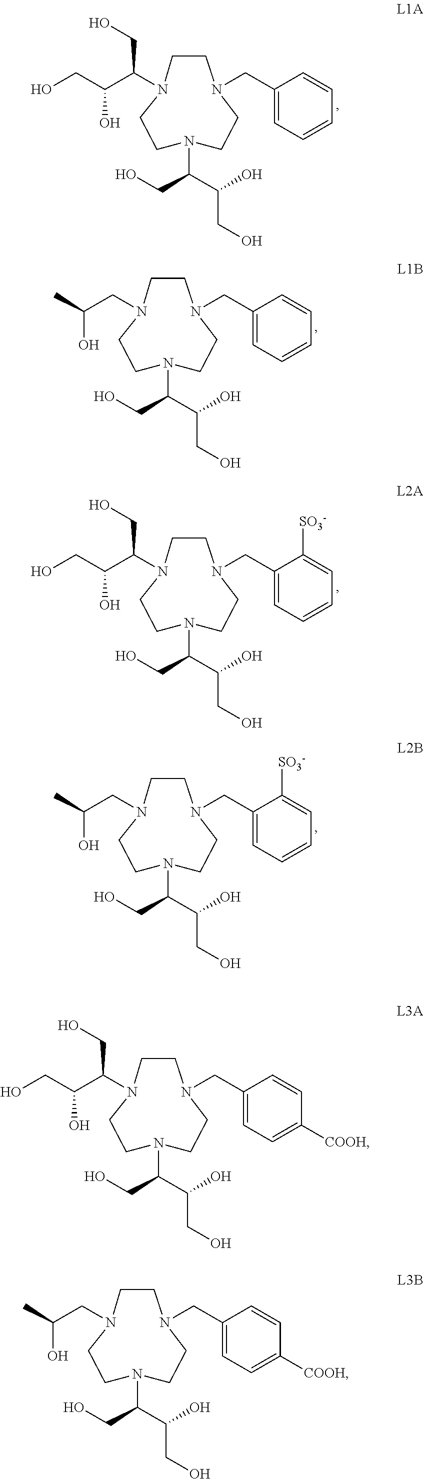

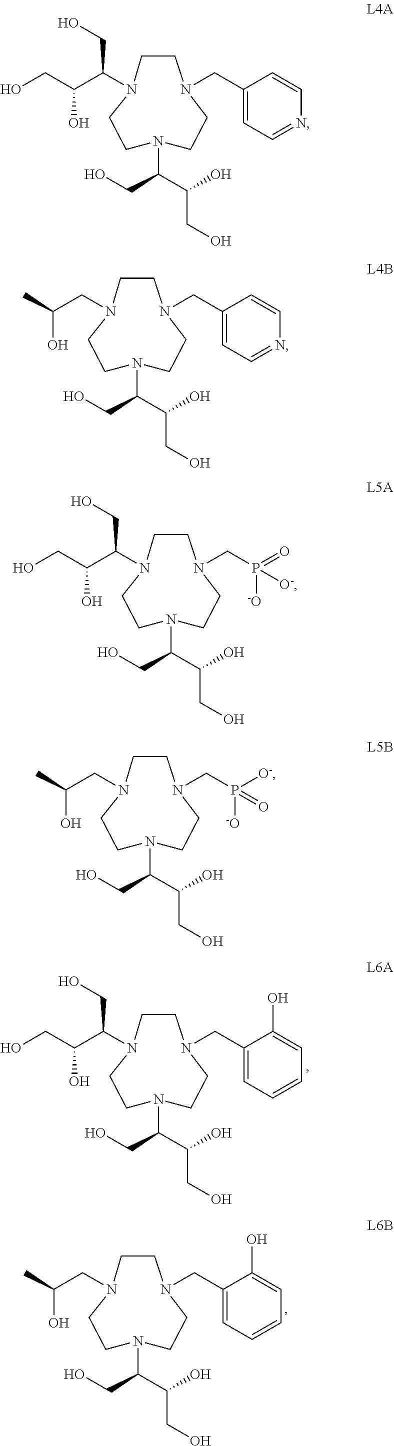

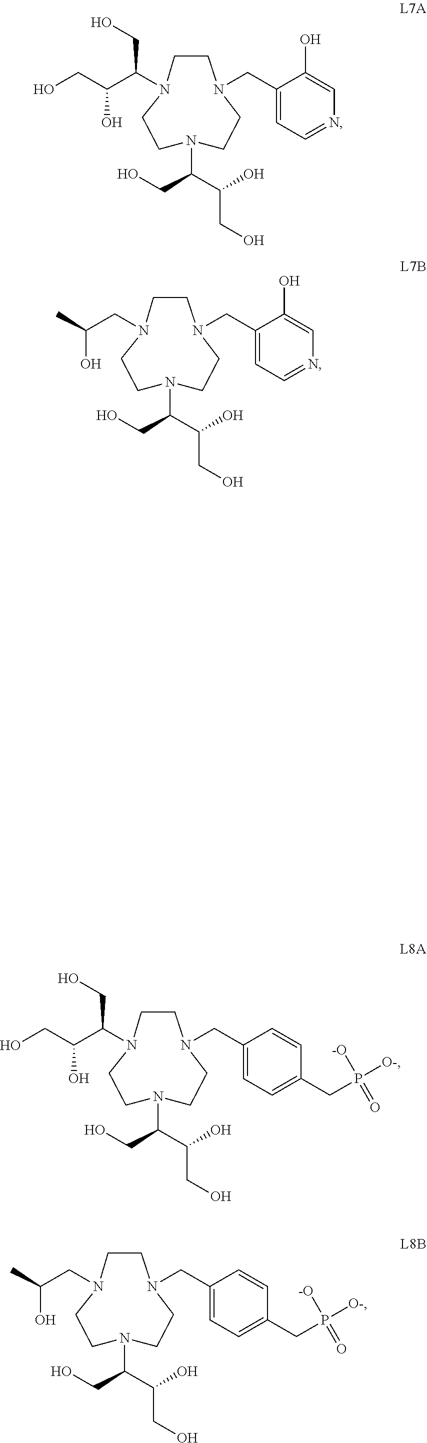

and protonated, partially deprotonated, and deprotonated species thereof (where applicable), wherein Fe(III) is complexed.
Statement 38. A macrocyclic complex according to Statement 31, wherein the macrocyclic complex has the following structure:

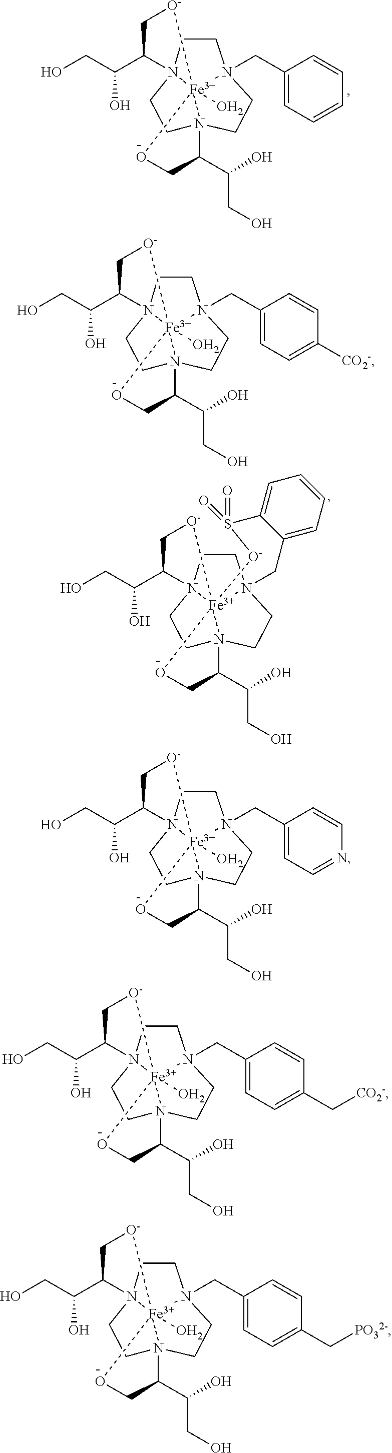

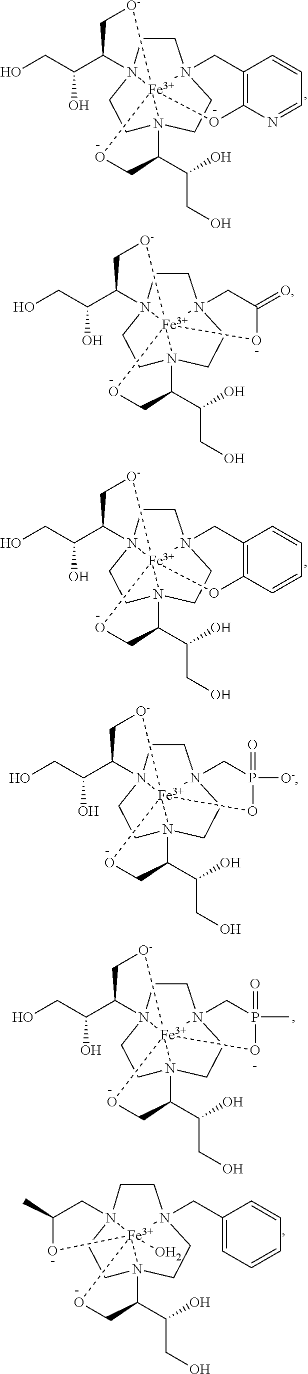

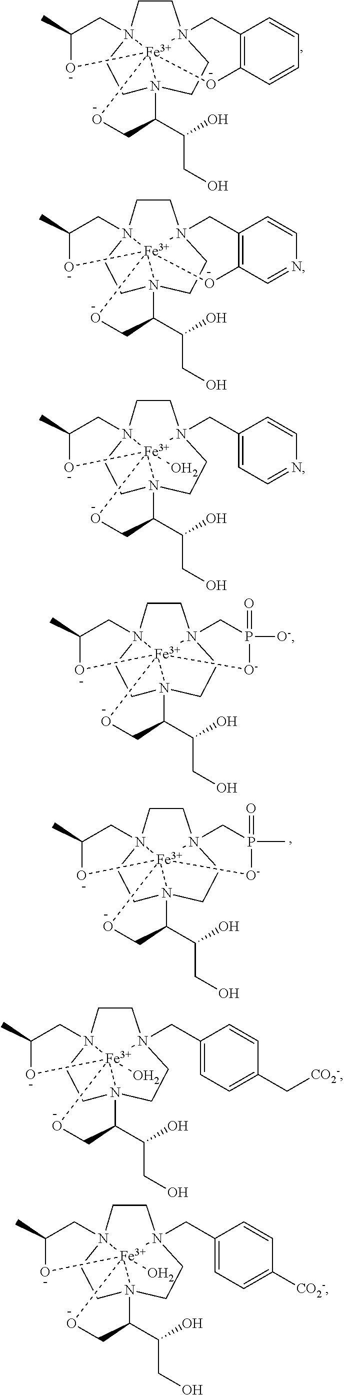

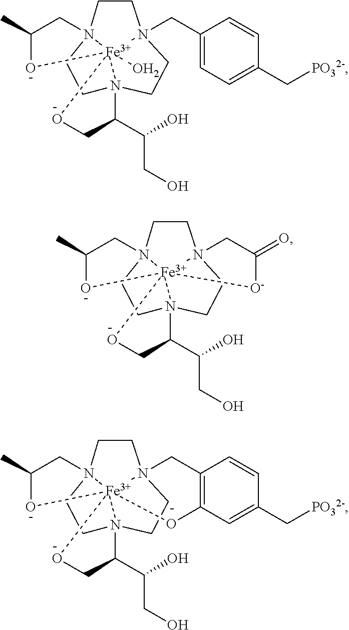

and protonated, partially deprotonated, and deprotonated species thereof (where applicable).
Statement 39. A macrocyclic complex according to Statement 31, wherein the macrocyclic complex has the following structure:

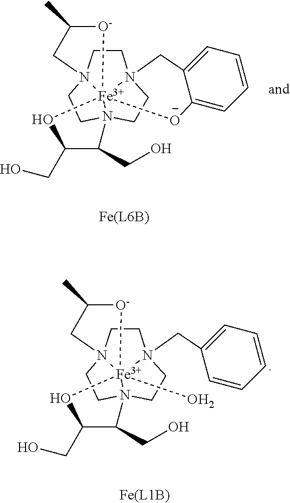

Statement 40. A composition comprising one or more macrocyclic complexes according to any one of Statements 31-39, and a pharmaceutically acceptable carrier.
Statement 41. A composition according to Statement 40, wherein the composition further comprises human serum albumin and/or meglumine.
Statement 42. A method to obtain an image of at least a portion of a cell, organ, vasculature or tissue comprising: contacting the cell, organ, vasculature, or tissue with one or more macrocyclic complex according to any one of Statements 31-39, and imaging at least a portion of the cell, organ, vasculature, or tissue to obtain an image of the portion of a cell, organ, vasculature, or tissue, wherein the image is obtained by using magnetic resonance.
Statement 43. A method according to Statement 42, wherein the cell, organ, vasculature, or tissue is part of an individual.
Statement 44. A method according to Statement 12 or Statement 13, wherein the image is obtained using magnetic resonance imaging (MRI).
Statement 45. A method according to any one of Statements 42-44, wherein the macrocyclic complex(es) is/are a T₁ agent or T₁ agents.
Statement 46. A macrocyclic compound having the following structure:

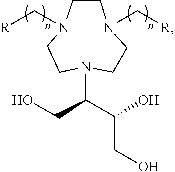

where the tri(hydroxy)butyl group(s) and —(CH₂)ₙR groups are pendant groups and each R is independently selected from alkyl groups; aryl groups; heteroaryl groups; alkyl groups comprising one or more —OH groups, one or more sulfonic acid groups, one or more carboxylic acid groups, one or more phosphonic acid groups, one or more alkyl groups, or the like, or combinations thereof, aryl groups comprising one or more —OH groups, one or more sulfonic acid groups, one or more carboxylic acid groups, one or more phosphonic acid groups, one or more alkyl groups, or the like, or combinations thereof, heteroaryl groups comprising one or more —OH groups, one or more sulfonic acid groups, one or more carboxylic acid groups, one or more phosphonic acid groups, one or more alkyl groups, or the like, or combinations thereof; and H; or a salt, a partial salt, a hydrate, a polymorph, or a stereoisomer thereof; n is 1, 2, or 3.
Statement 47. A macrocyclic compound according to Statement 46, further comprising a coordinating pendant group or a non-coordinating pendant group.
Statement 48. A macrocyclic compound according to Statement 46 or Statement 47, wherein at least one of the pendant groups is substituted at a benzylic position or any carbon the alkyl group leading to the heteroatom of the pendant group.
Statement 49. A macrocyclic compound according to Statement 46 or Statement 47, wherein the pendant groups are chosen from:

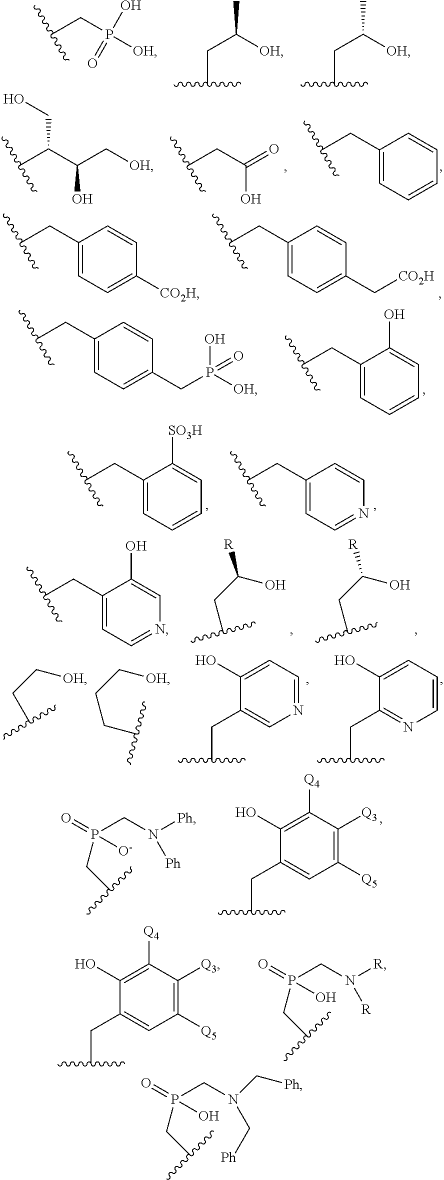

and protonated, partially deprotonated, and deprotonated species thereof (where applicable).
Statement 50. A macrocyclic compound according to any one of Statements 46-49, wherein the macrocyclic compound has at least one open coordination site.
Statement 51. A macrocyclic compound according to any one of the preceding claims, wherein the macrocyclic compound has the following structure:

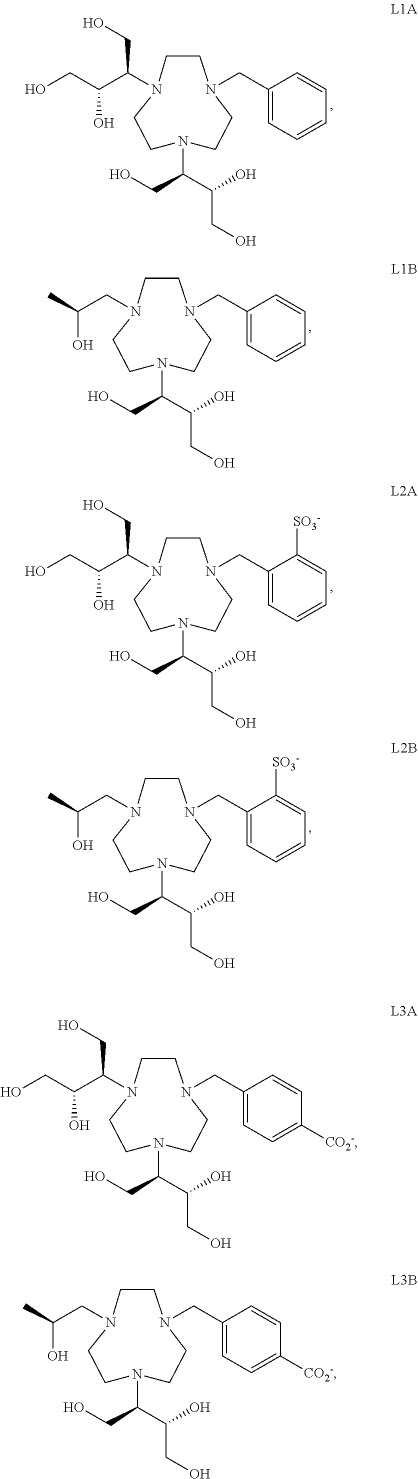

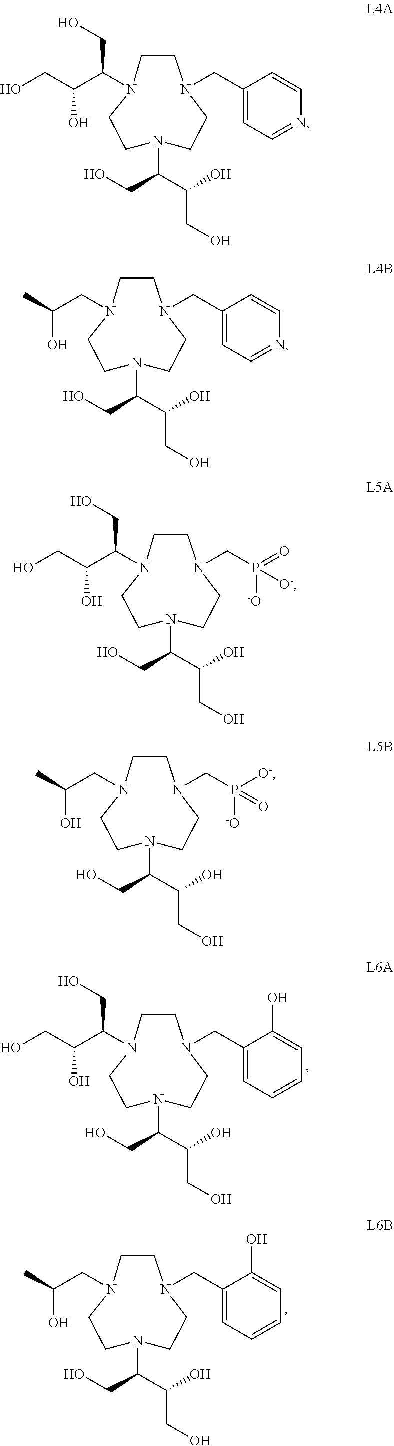

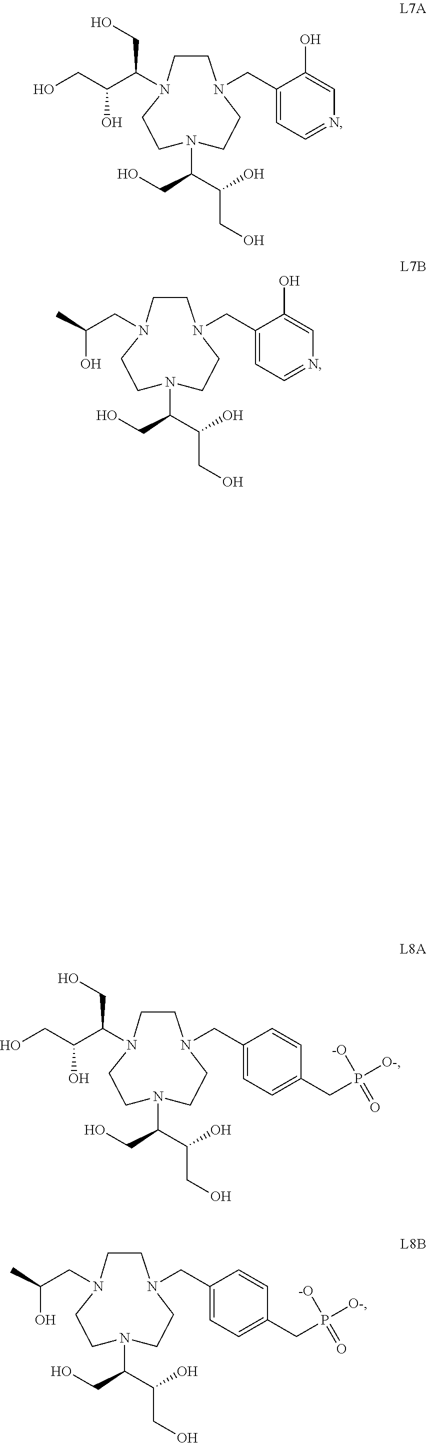

and protonated and deprotonated analogs thereof.
Statement 52. A macrocyclic complex comprising:
a macrocyclic core, wherein the macrocyclic core is a TACN group, an S-substituted TACN, or an O-substituted TACN group having the following structure:

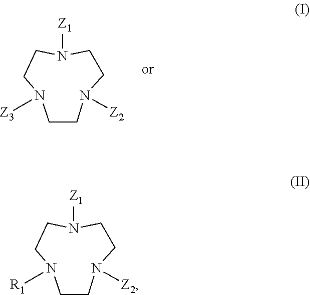

and Z₁, Z₂, and Z₃ are each independently H or one or more of the following pendant groups:

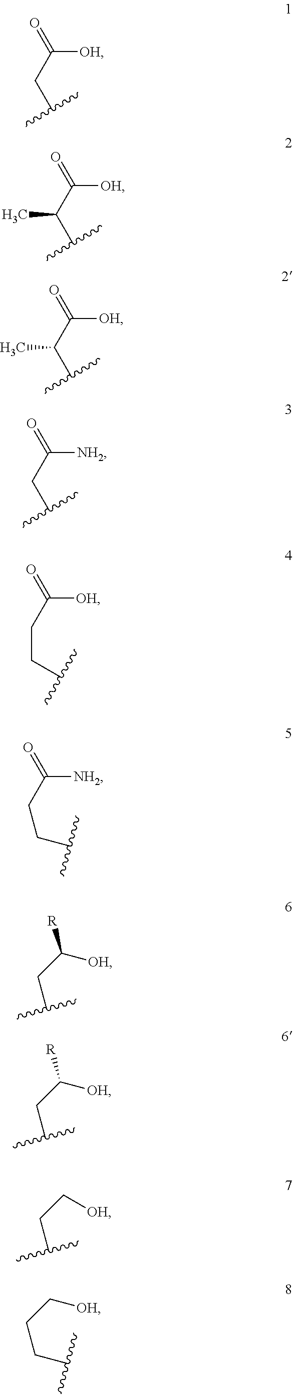

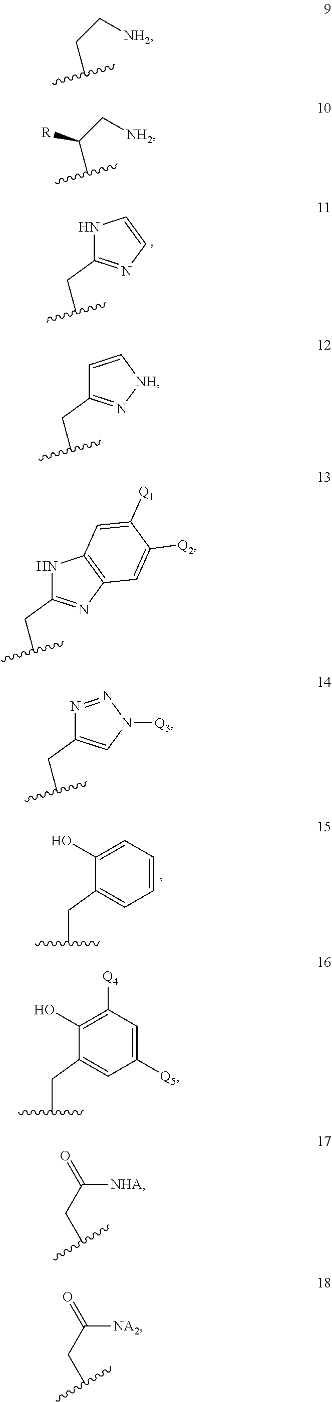

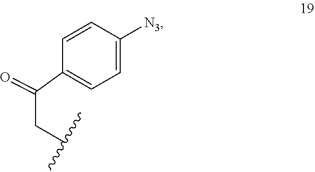 [SMILES: *CC(=O)c1ccc(N=[N+]=[N-])cc1]

or a deprotonated analog thereof or a stereoisomer thereof, wherein R is methyl, R₁, R₂, and R₃ are each independently a substituted or unsubstituted aryl, substituted or unsubstituted heteroaryl, or substituted or unsubstituted alkyl group, and R₁, R₂, and R₃ are not pendant donors; Q₁ and Q₂ are each independently H, OCH₃, CO₂H, or CH₂CO₂G₄, G₄ is H, C₁ to C₁₂ substituted or unsubstituted alkyl groups of linear or branched structure or a (—CH₂CH₂O—)ₙ group, wherein n is 1-2, Q₃ is H, C₁ to C₁₂ substituted or unsubstituted alkyl groups of linear or branched structure or a (—CH₂CH₂O—)ₙ group, wherein n is 1-12, Q₄ and Q₅ are each independently H, OCH₃, CO₂H, or substituted or unsubstituted alkyl groups of linear or branched structures, A is a substituted or unsubstituted alkyl group of linear or branched structure with C₁ to C₁₂ or is a substituted or unsubstituted aryl group or an amino acid, and a high-spin Fe(III) cation complexed to the macrocyclic core and at least one pendant group, or a salt, a partial salt, a hydrate, a polymorph, or a stereoisomer thereof, wherein the macrocyclic compound exhibits a redox potential of less than 0 vs. normal hydrogen electrode (NHE) in an aqueous medium at a pH of 6.5-7.5, wherein the macrocyclic core has Structure I, Z₁ is H and Z₂ and Z₃ are each independently a pendant group; wherein the macrocyclic core has Structure II, Z₁ and Z₂ are each independently a pendant group wherein for all Structures I and II, each of Z₁, Z₂, and Z₃, as applicable, are selected independently of each other.
   Statement 53. The macrocyclic complex of Statement 52, wherein at least one or more pendant groups is covalently bound to a N on the macrocyclic core.
   Statement 54. The macrocyclic complex of Statement 52, wherein the macrocyclic complex has at least one open coordination site.
   Statement 55. The macrocyclic complex of Statement 52, wherein the macrocyclic complex has at least one water or at least one hydroxide complexed to the high-spin Fe(III) cation.
   Statement 56. The macrocyclic complex of Statement 52, wherein at least one of the pendant groups is substituted at a benzylic position or any carbon the alkyl group leading to the heteroatom of the pendant group.
   Statement 57. The macrocyclic complex of Statement 52, wherein the macrocyclic complex comprises a TACN moiety and at least one anionic pendant groups.
   Statement 58. The macrocyclic complex of Statement 57, wherein the anionic pendants are individually chosen from carboxylate pendants, imidazolate pendants, pyrazolate pendants, alkoxide pendants, and phenoxide pendants.
   Statement 59. The macrocyclic complex of Statement 58, wherein the macrocyclic complex further comprises a coordinating pendant group or a non-coordinating pendant group.
   Statement 60. The macrocyclic complex of Statement 52, wherein the macrocyclic core has the following structure:

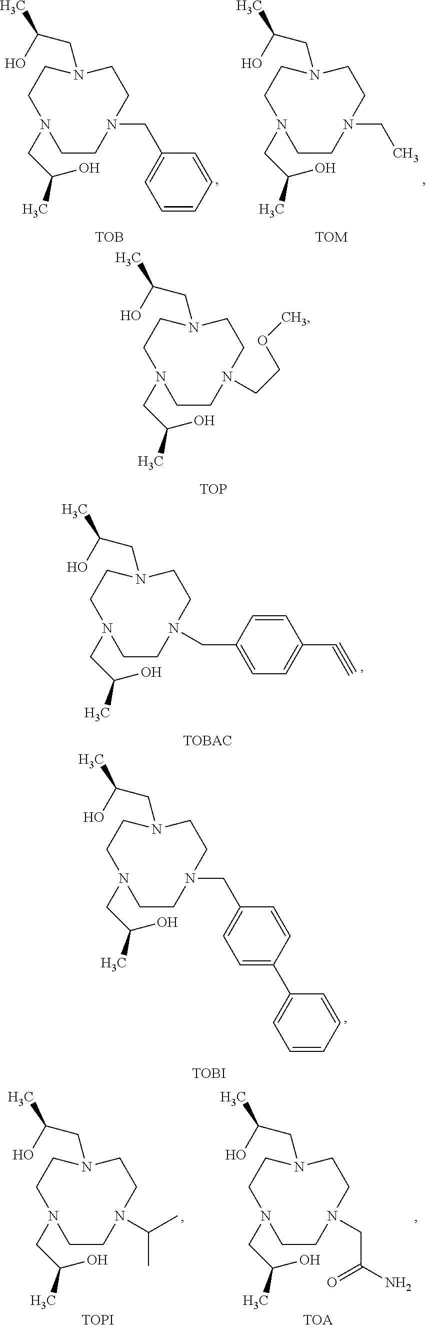

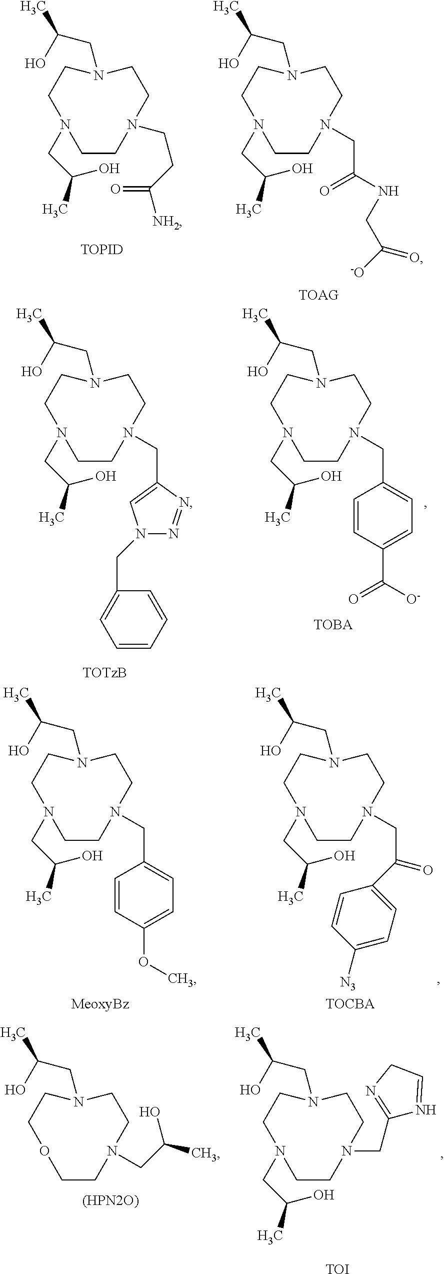

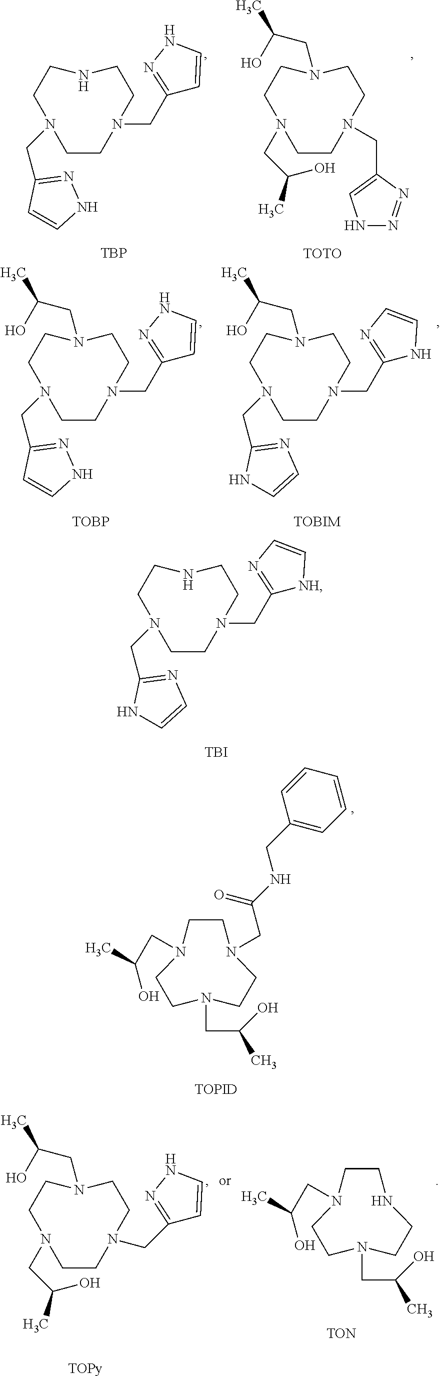

Statement 61. The macrocyclic complex of Statement 52, wherein the macrocyclic complex has one of the following structures:

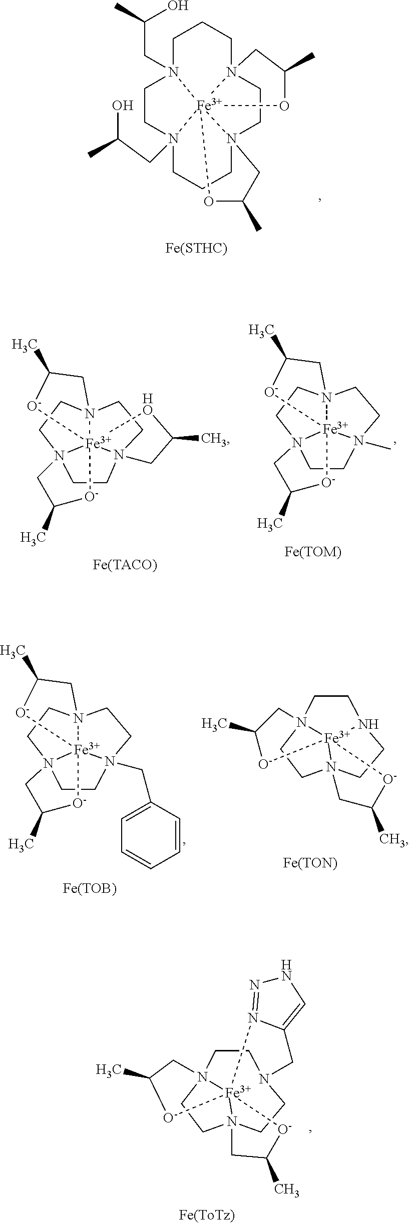

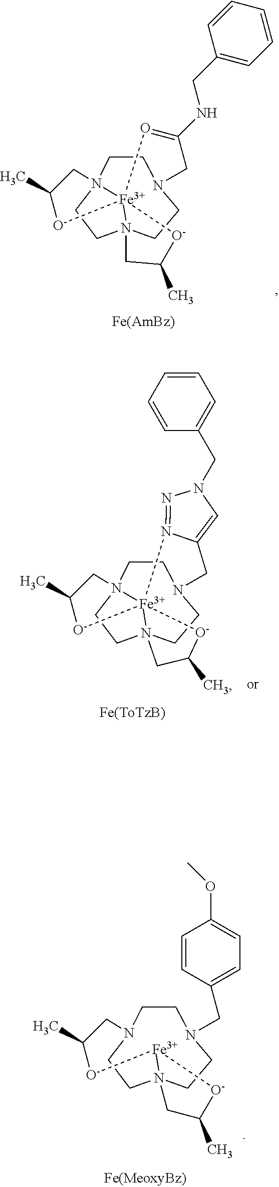

Statement 62. A composition comprising one or more macrocyclic complexes of Statement 52 and a pharmaceutically acceptable carrier.
Statement 63. The composition of Statement 62, wherein the composition further comprises human serum albumin and/or meglumine.
Statement 64. A method to obtain an image of at least a portion of a cell, organ, vasculature, or tissue comprising: contacting the cell, organ, vasculature, or tissue with one or more macrocyclic complexes of Statement 52, and imaging at least a portion of the cell, organ, vasculature, or tissue to obtain an image of the portion of a cell, organ, vasculature, or tissue, wherein the image is obtained by using magnetic resonance.
Statement 65. The method of Statement 64, wherein the cell, organ, vasculature, or tissue is part of an individual.
Statement 66. The method of Statement 65, wherein the image is obtained using magnetic resonance imaging (MRI).
Statement 67. The method of Statement 65, wherein the macrocyclic complexes are T₁ agents.
Statement 68. A macrocyclic complex comprising: a 1,4,7-triazacyclononane (TACN) moiety or an O-substituted TACN moiety having a structure:

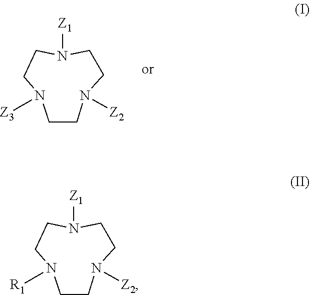

wherein R₁, Z₁, Z₂, and Z₃ are anionic pendent groups independently chosen from:

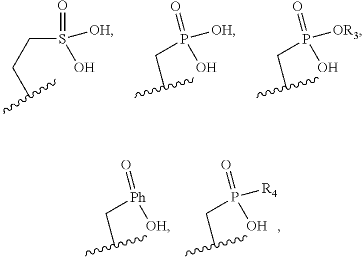

deprotonated analogs thereof, and combinations thereof, wherein R₃ is a substituted or unsubstituted alkyl or a substituted or unsubstituted aryl group and R₄ is a substituted alkyl or unsubstituted alkyl or a substituted or unsubstituted aryl group; and a high-spin Fe(III) cation complexed to the TACN moiety and at least one anionic pendent group substituent of the TACN moiety, or a high-spin Fe(III) cation complexed to the O-substituted TACN moiety and at least one anionic pendent group substituent of the O-substituted TACN moiety, wherein: when the macrocyclic complex is:

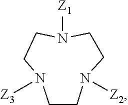

Z₁ and Z₂ are

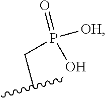 [SMILES: *CP(=O)(O)O]

### and Z₃ is not

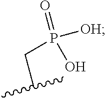 [SMILES: *CP(=O)(O)O]

and
when the macrocyclic complex is:

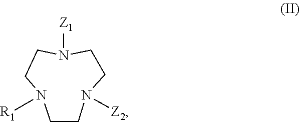

Z₁ and Z₂ are

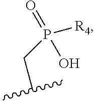

wherein R⁴ is an unsubstituted aryl, and Z₃ is not

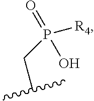

wherein R⁴ is an unsubstituted aryl.
Statement 69. The macrocyclic complex of Statement 68, wherein at least one or all of the one or more pendent groups is covalently bound to a nitrogen atom on the TACN moiety or an O-substituted TACN moiety.
Statement 70. The macrocyclic complex of Statement 68, wherein the macrocyclic complex has at least one open coordination site.
Statement 71. The macrocyclic complex of Statement 68, wherein the macrocyclic complex has at least one water complexed to the high-spin Fe(III) cation.
Statement 72. A macrocyclic complex comprising: a 1,4,7-triazacyclononane (TACN) moiety having a structure:

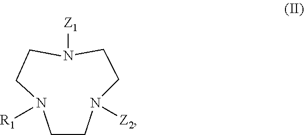

wherein Z₁ and Z₂ are anionic pendent groups independently chosen from:

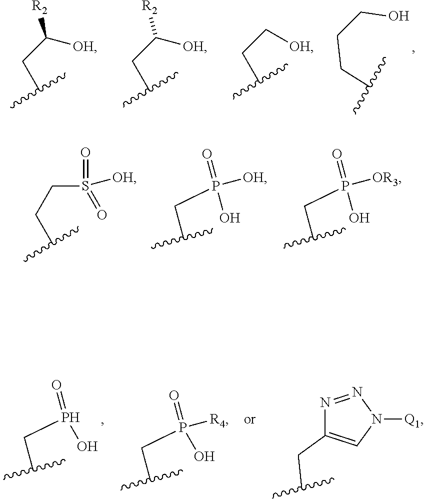

deprotonated analogs thereof, and combinations thereof, R₁ is an anionic pendent groups independently chosen from:

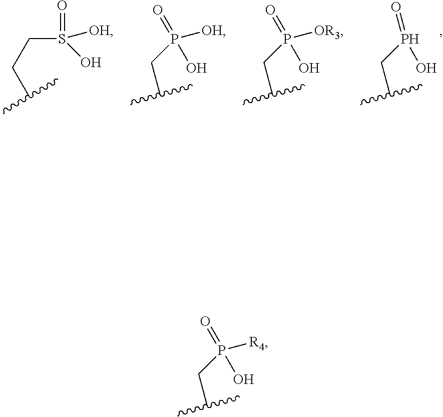

deprotonated analogs thereof, and combinations thereof, wherein R₂ is a substituted or unsubstituted alkyl group, a substituted or unsubstituted aromatic group or a substituted ether; R₃ is a substituted or unsubstituted alkyl or a substituted or unsubstituted aryl group; R₄ is a substituted alkyl or unsubstituted alkyl or a substituted or unsubstituted aryl group; and Q₁ is aryl substituted with an anionic group, an alkyl group substituted with an anionic group or an aralkyl group substituted with an anionic group; and a high-spin Fe(III) cation complexed to the TACN moiety and at least one anionic pendent group substituent of the TACN moiety.
Statement 73. The macrocyclic complex of Statement 72, wherein at least one or all of the one or more pendent groups is covalently bound to a nitrogen atom on the TACN moiety.
Statement 74. The macrocyclic complex of Statement 72, wherein the macrocyclic complex has at least one open coordination site.
Statement 75. The macrocyclic complex of Statement 72, wherein the macrocyclic complex has at least one water and/or at least one hydroxide complexed to the high-spin Fe(III) cation.
Statement 76. A macrocyclic complex comprising a macrocyclic core with the following structure:

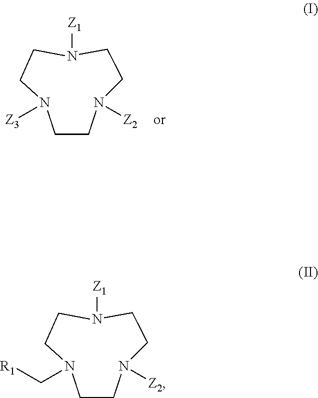

wherein
R₁ is a substituted or unsubstituted phenyl group, wherein the phenyl group optionally does not have an —OH group, a substituted or unsubstituted heteroaryl group, a substituted or unsubstituted alkyl group wherein the substituted or unsubstituted alkyl group is not a methyl group,
Z₁, Z₂, and Z₃ are independently chosen from one or more of the following pendant groups.

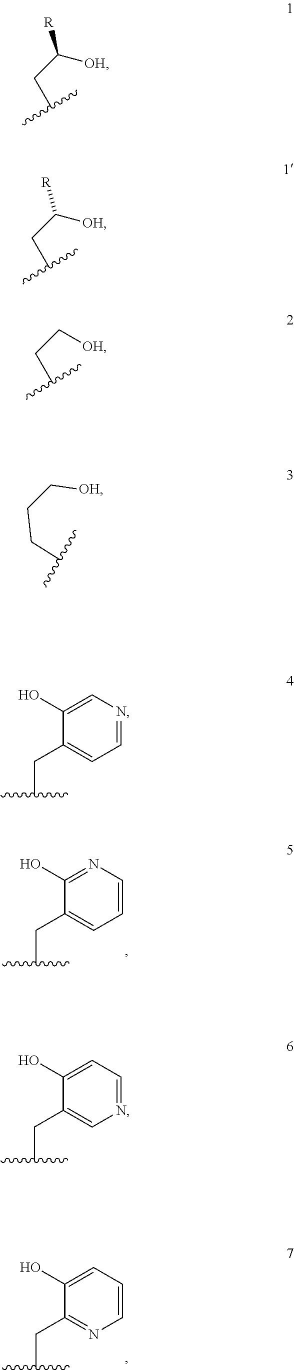

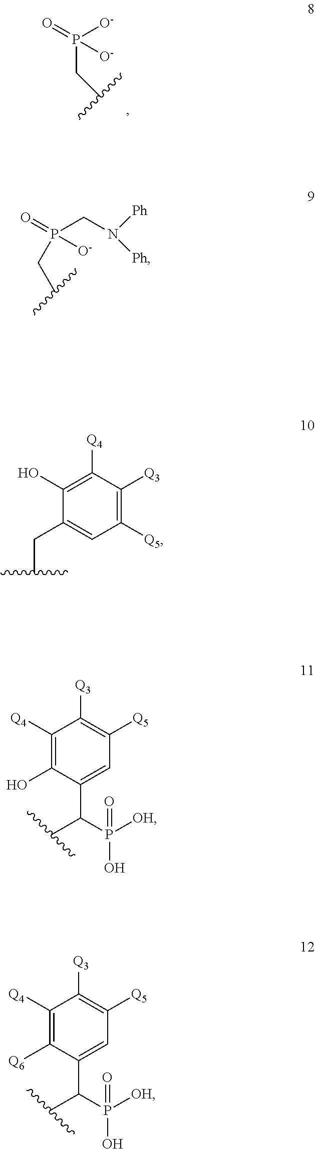

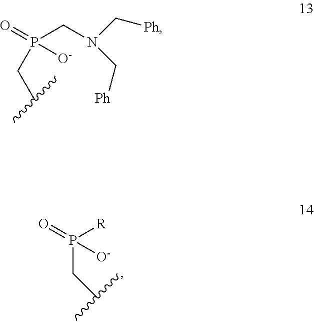

and protonated, partially deprotonated, or completely deprotonated species thereof, wherein Q₃, Q₄ and Q₅ are each independently anionic groups or chosen from —H, —NR₂, —NO₂, —CN, —(CH₂)ₘNR₂, OH, OR, —P(O)OH₂, —(CH₂)ₘPO(OH)₂, —SO₃H, and deprotonated species thereof, wherein m is 1 or 2 and R is an alkyl group, CF₃ group, aryl group, alkyl carboxylate, or alkyl carboxylic acid; and
the macrocyclic complex has two of any of 1, 1′, 2, 3, 4, 8, 10, or a combination thereof and not all the pendant groups are the same, and a high-spin Fe(III) cation complexed to the macrocyclic core and/or at least one pendant group substituent of the macrocyclic compound, with the provisos:
i) when two of the pendant groups are structures 1, 1′, 2, 3, or any combination thereof, then the third pendent group is not 1, 1′, 2, or 3 or optionally, 1, 1′, 2, 3, 10 or any combination thereof, wherein Q₃, Q₄, and Q₅ are H, then then the third pendent group is not 1, 1′, 2, 3 or 10, wherein Q₃, Q₄, and Q₅ are H;
ii) when two of the pendant groups are structures 1, 1′, 2, 3, 8, 14 or any combination thereof, then the third pendent group is not 1, 1′, 2, 3, 8, or 14;
iii) when two of the pendant groups are structures 1, 1′, 2, 3, 10, or any combination thereof and R of structures 1 and/or 1′ is phenyl or alkyl, then the R₁ is not a substituted or unsubstituted heteroaryl, a substituted or unsubstituted alkyl group;
iv) the macrocyclic core does not have the following structure:

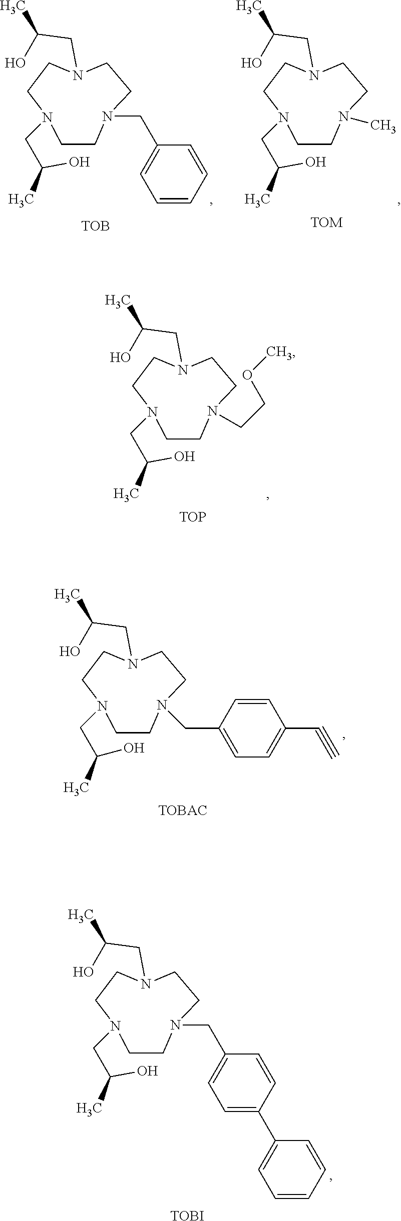

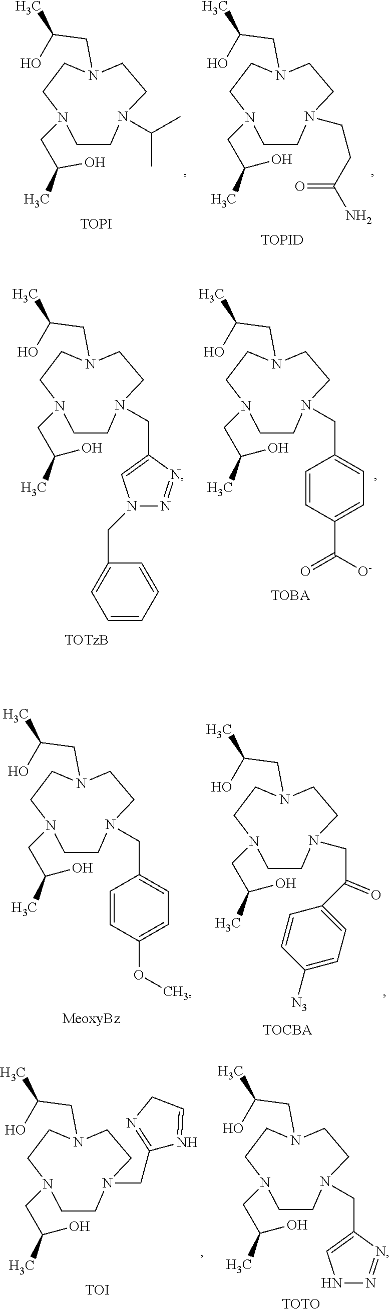

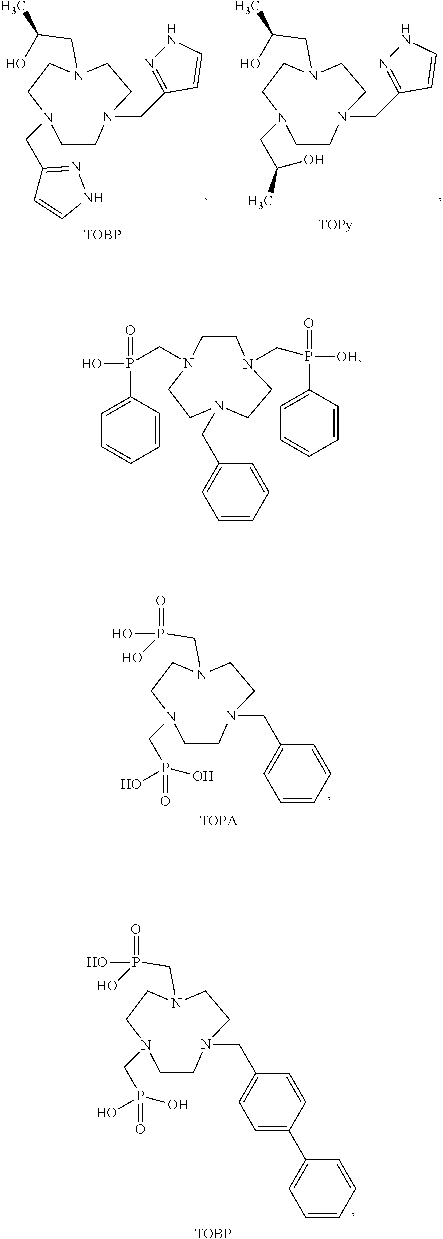

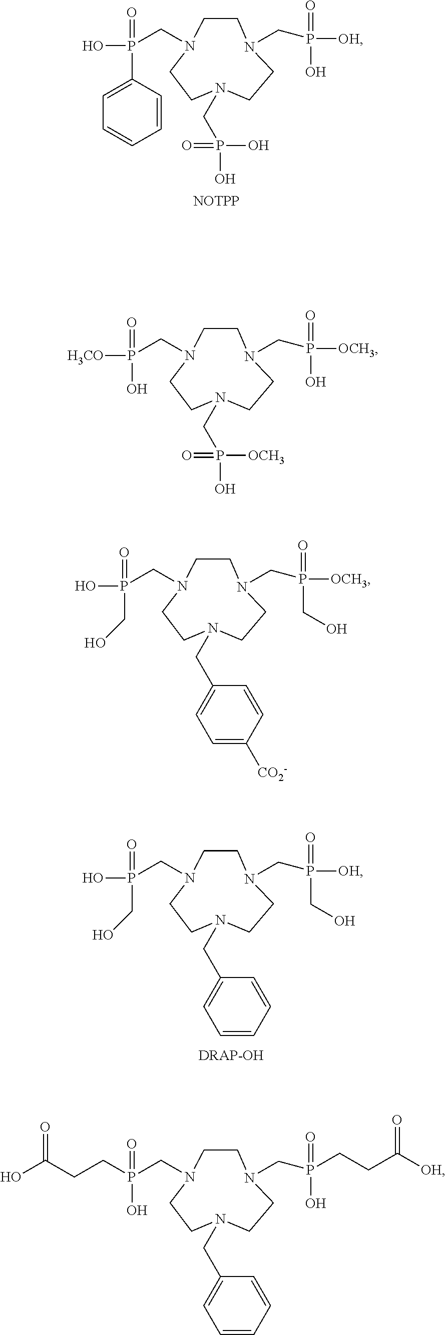

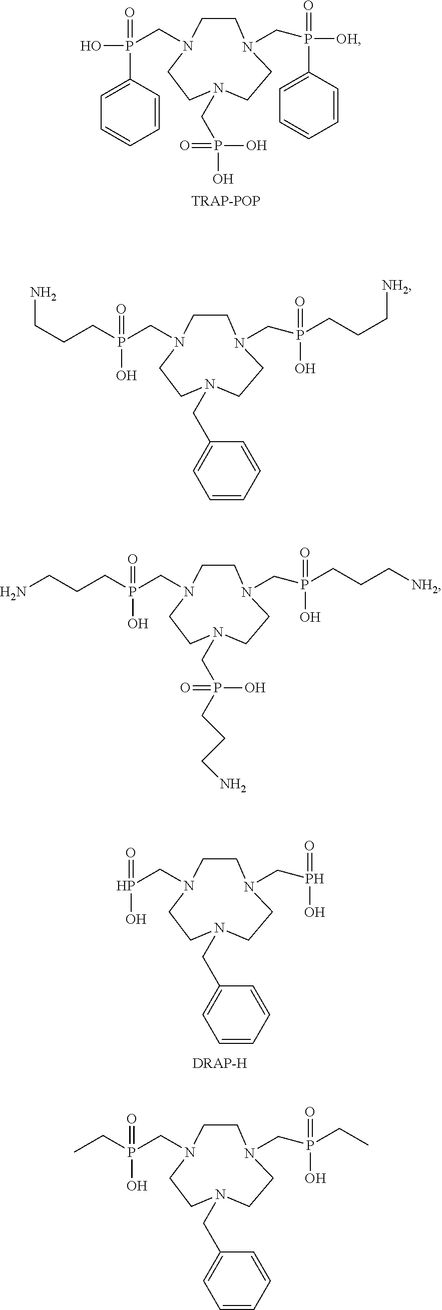

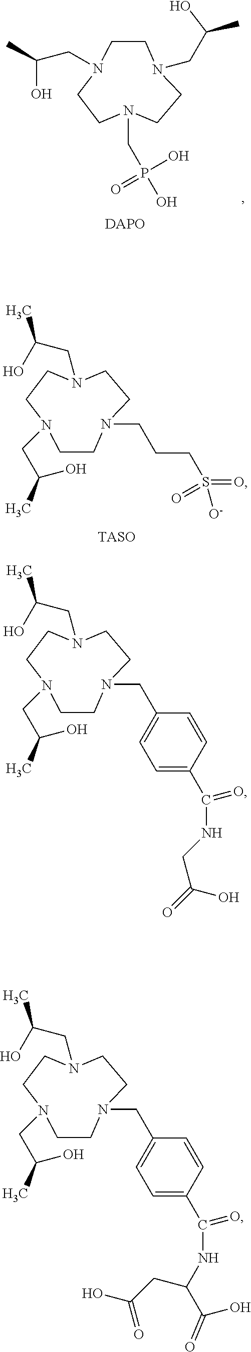

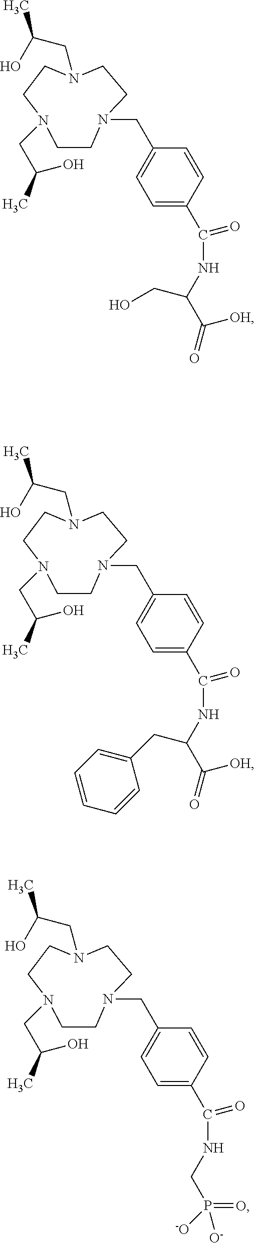

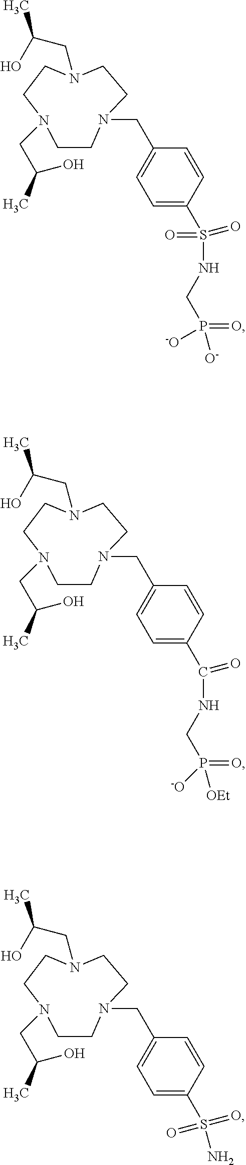

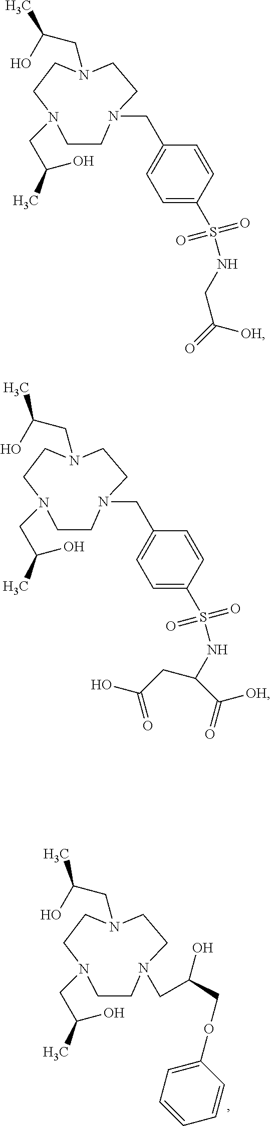 [SMILES: C[C@H](O)CN1CCN(Cc2ccc(S(=O)(=O)NCC(=O)O)cc2)CCN(C[C@H](C)O)CC1 ; C[C@H](O)CN1CCN(Cc2ccc(S(=O)(=O)NC(CC(=O)O)C(=O)O)cc2)CCN(C[C@H](C)O)CC1 ; C[C@H](O)CN1CCN(C[C@@H](O)COc2ccccc2)CCN(C[C@H](C)O)CC1]

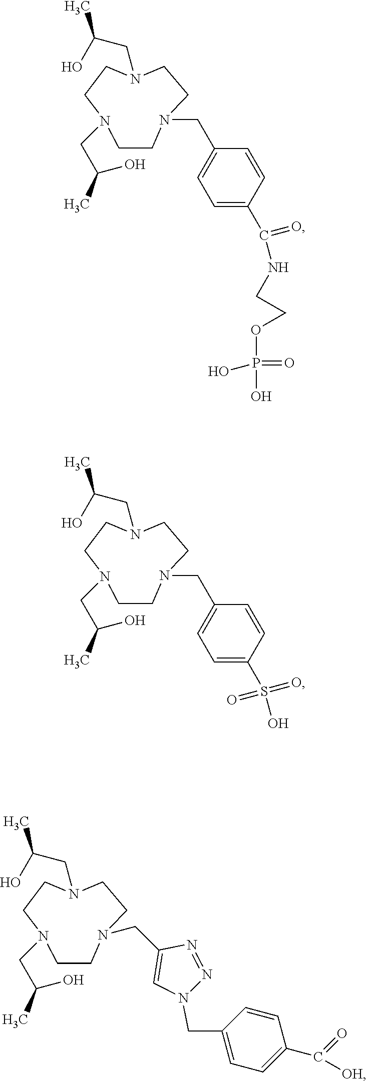 [SMILES: C[C@H](O)CN1CCN(Cc2ccc(C(=O)NCCOP(=O)(O)O)cc2)CCN(C[C@H](C)O)CC1 ; C[C@H](O)CN1CCN(Cc2ccc(S(=O)(=O)O)cc2)CCN(C[C@H](C)O)CC1 ; C[C@H](O)CN1CCN(Cc2cn(Cc3ccc(C(=O)O)cc3)nn2)CCN(C[C@H](C)O)CC1]

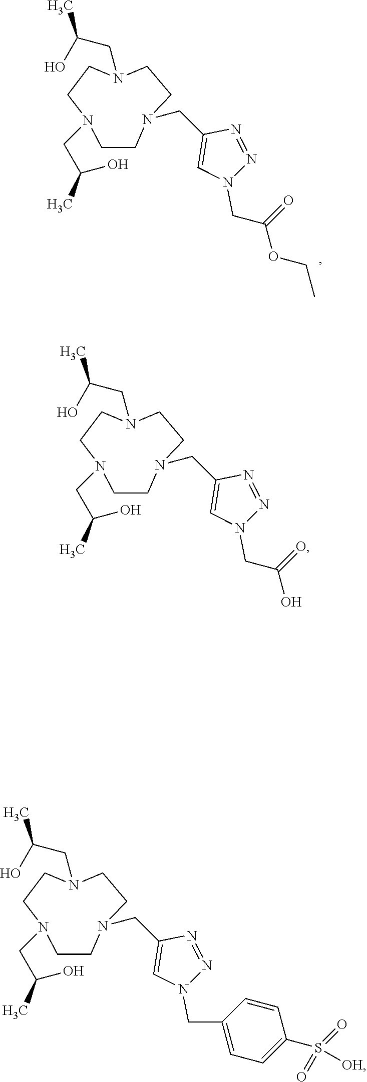 [SMILES: CCOC(=O)Cn1cc(CN2CCN(C[C@H](C)O)CCN(C[C@H](C)O)CC2)nn1 ; C[C@H](O)CN1CCN(Cc2cn(CC(=O)O)nn2)CCN(C[C@H](C)O)CC1 ; C[C@H](O)CN1CCN(Cc2cn(Cc3ccc(S(=O)(=O)O)cc3)nn2)CCN(C[C@H](C)O)CC1]

wherein high-spin Fe(III) is coordinated thereto; and
v) the macrocyclic complex does not have the following structure:

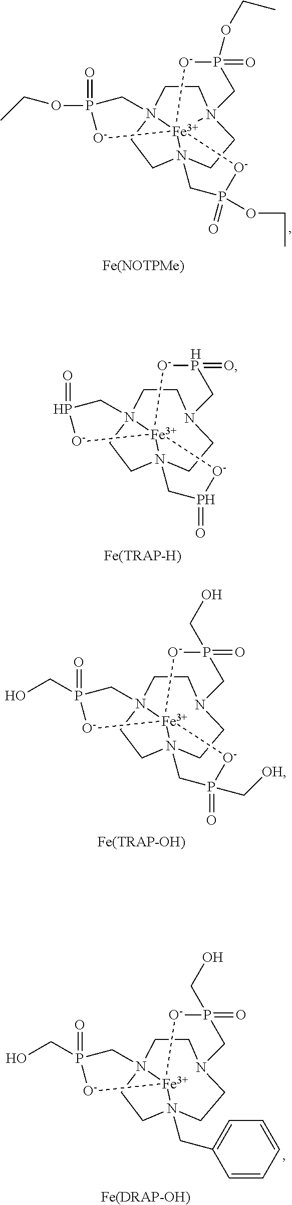

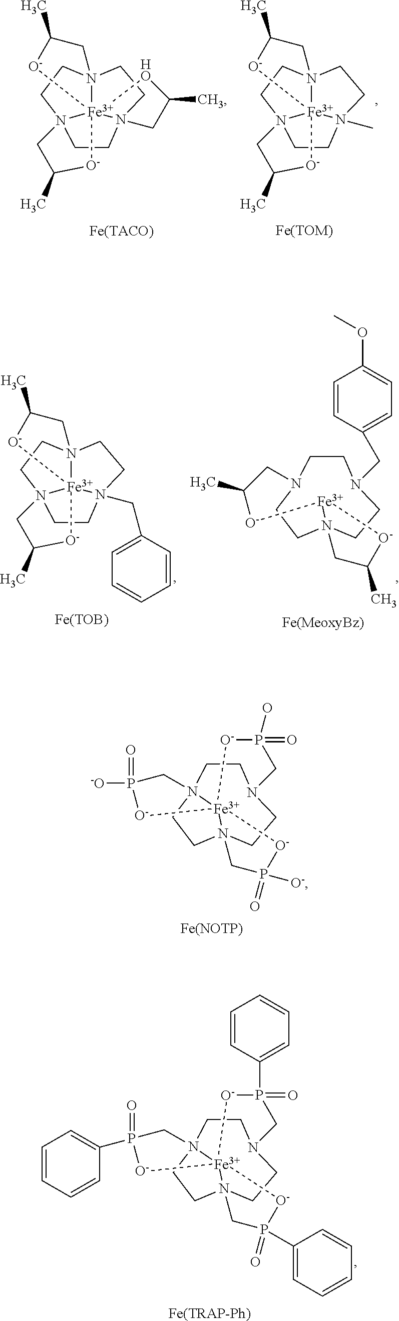

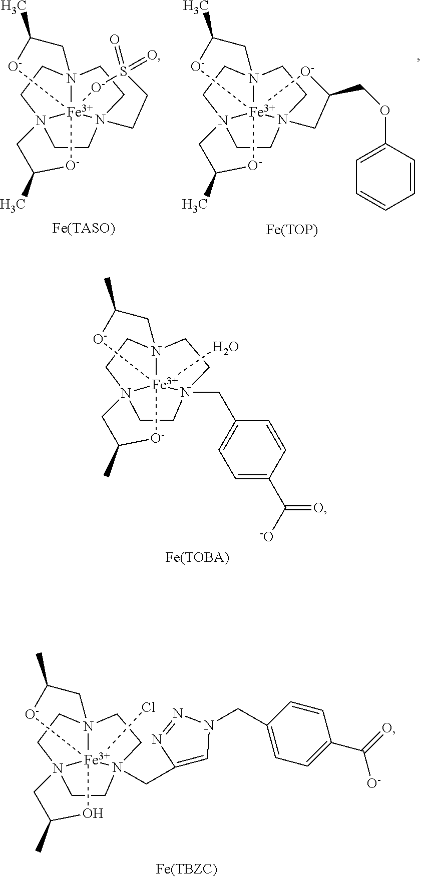

wherein the Fe(III) is high spin Fe(III).
Statement 77. The macrocyclic complex according to Statement 76, wherein at least one or all of the one or more pendant groups is/are covalently bound to a N on the macrocyclic core.
Statement 78. The macrocyclic complex according to Statement 76 or Statement 77, wherein the macrocyclic complex has at least one water or at least one hydroxide complexed to the high-spin Fe(III) cation.
Statement 79. The macrocyclic complex according to any one of Statements 76-78, wherein Z₁, Z₂, and Z₃ are independently chosen from.

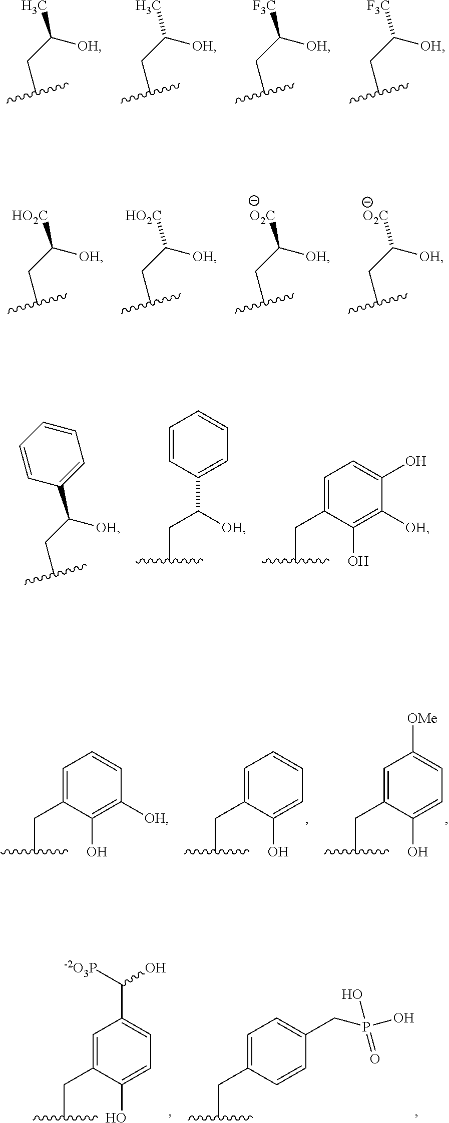

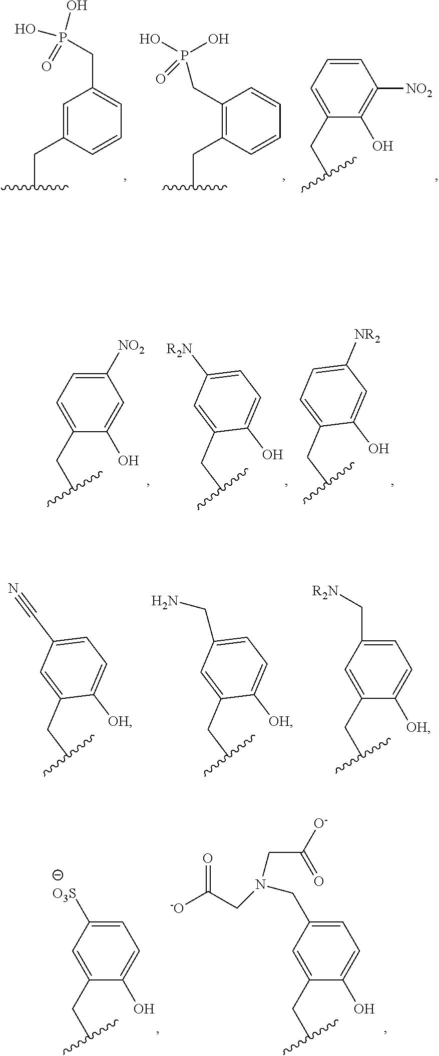

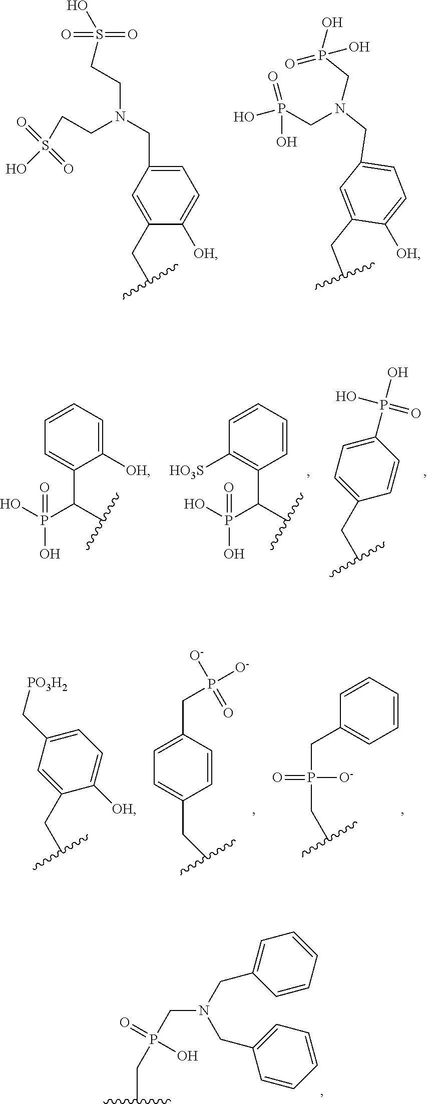

and protonated, deprotonated, and partially deprotonated species thereof (where applicable).
Statement 80. The macrocyclic complex according to any one of Statements 76-79, wherein the macrocyclic core has the following structure to which high-spin Fe(III) is complexed thereto:

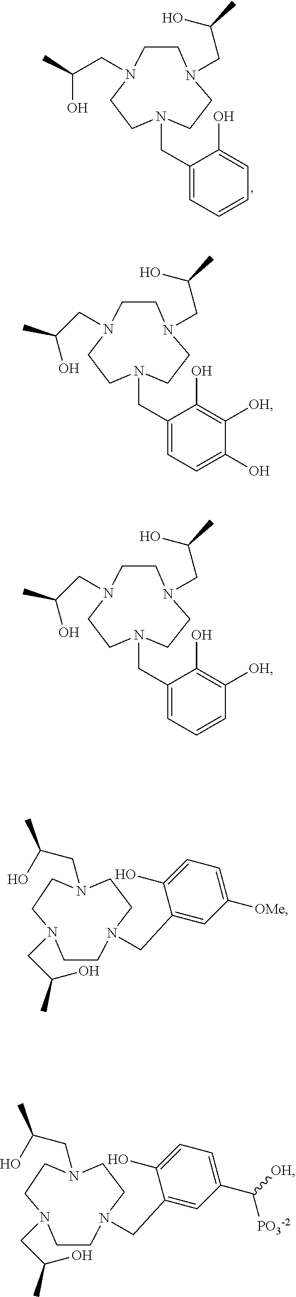

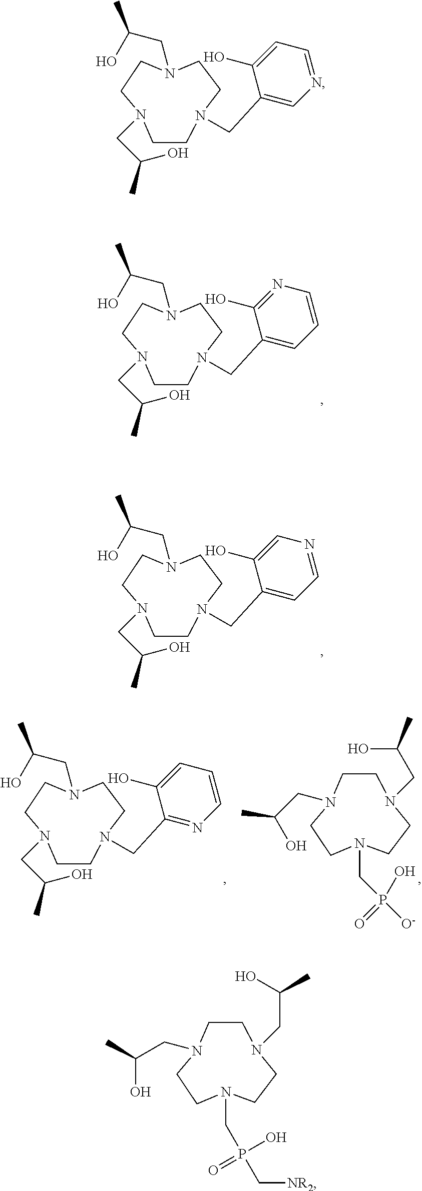

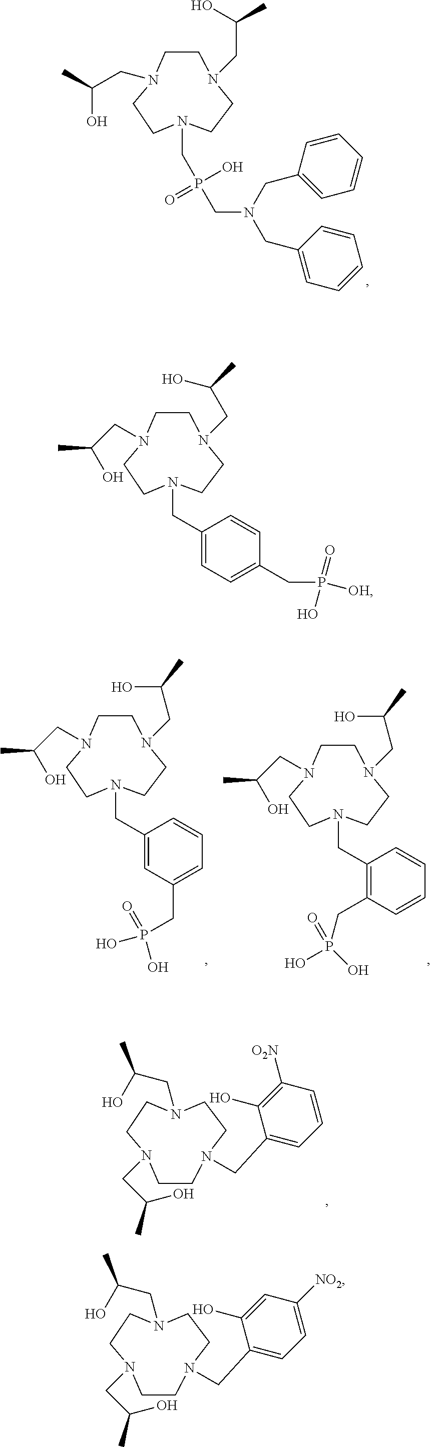

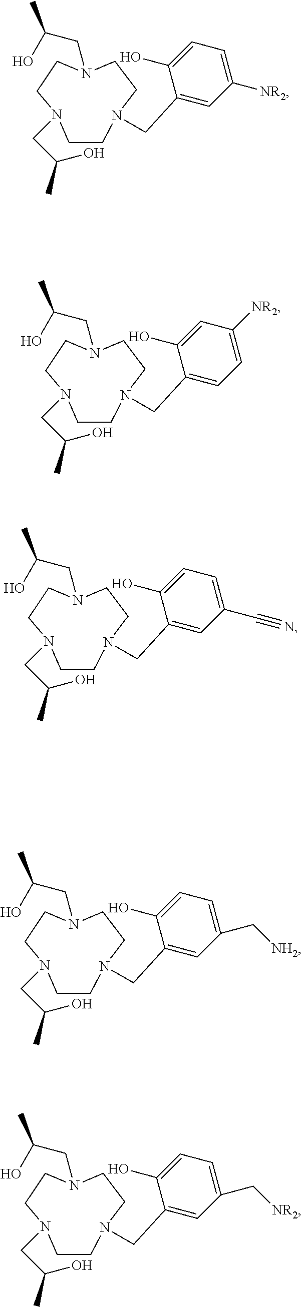

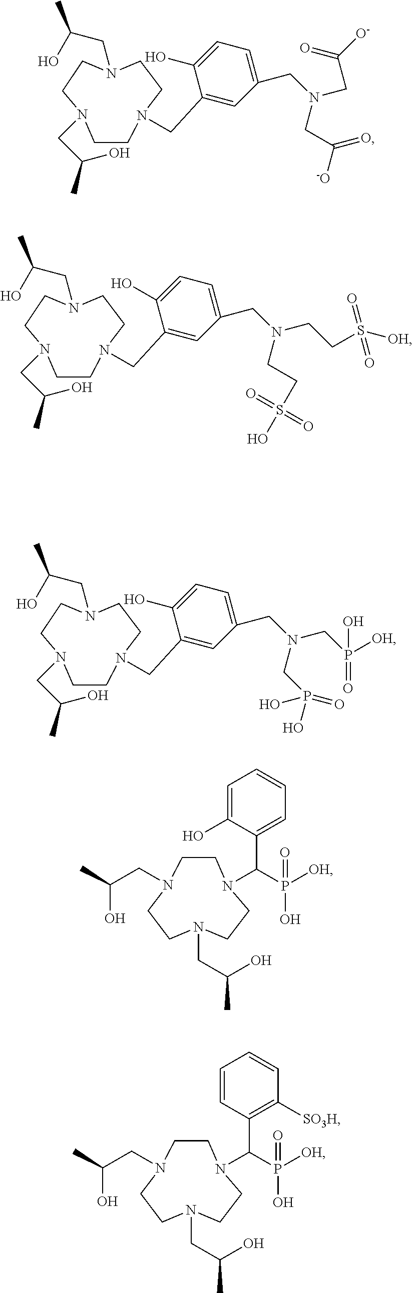

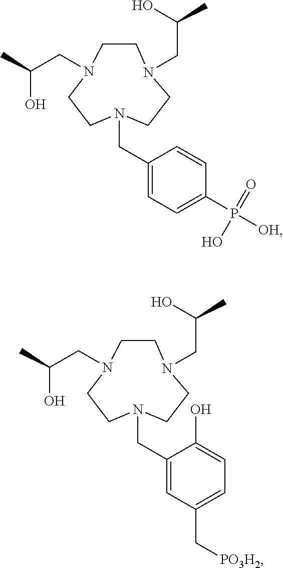

or protonated, deprotonated, or partially deprotonated species thereof (where applicable).
Statement 81. The macrocyclic complex according to any one of Statements 76-79, wherein the macrocyclic complex has the following structure:

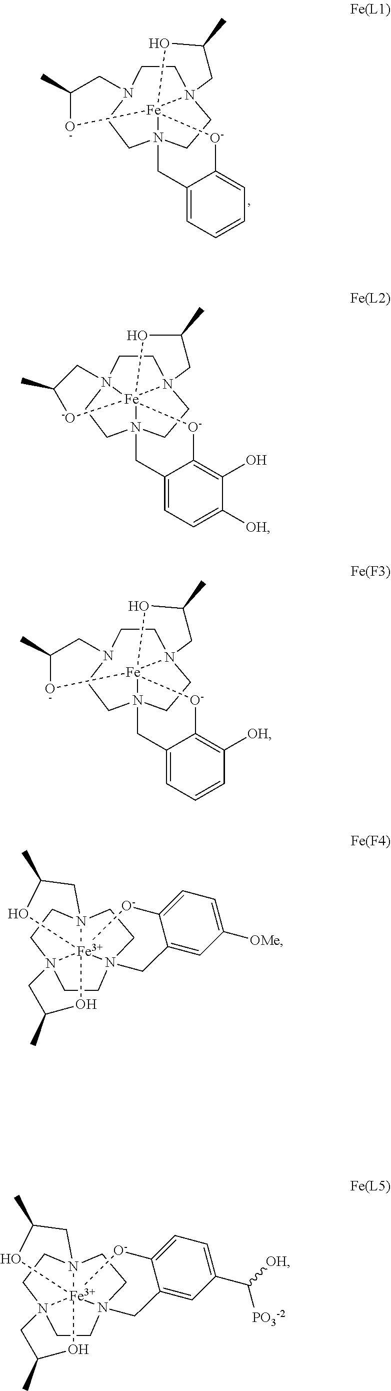

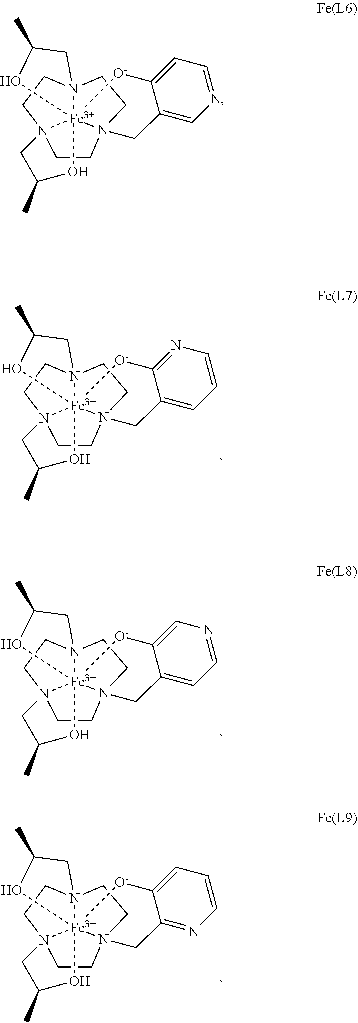

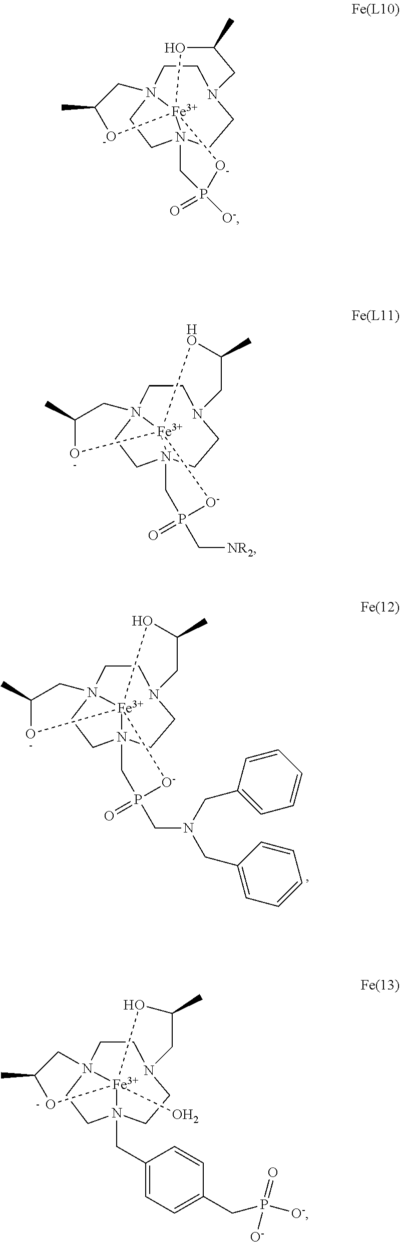

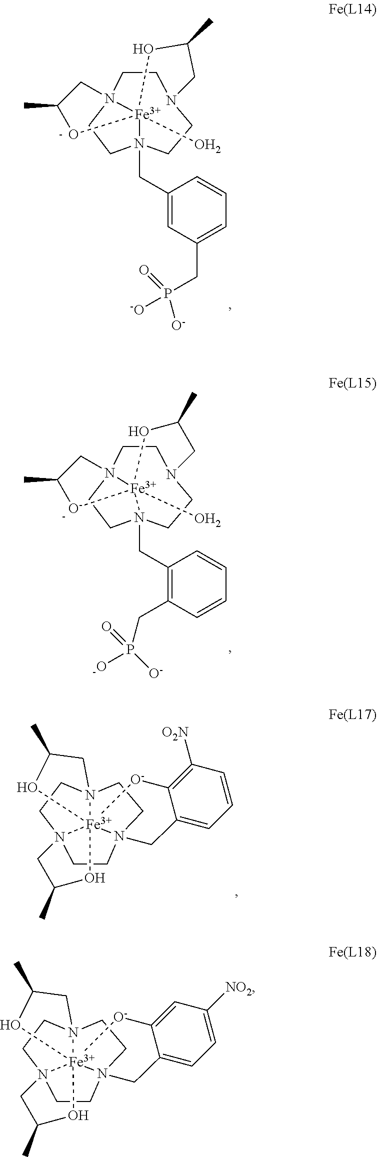

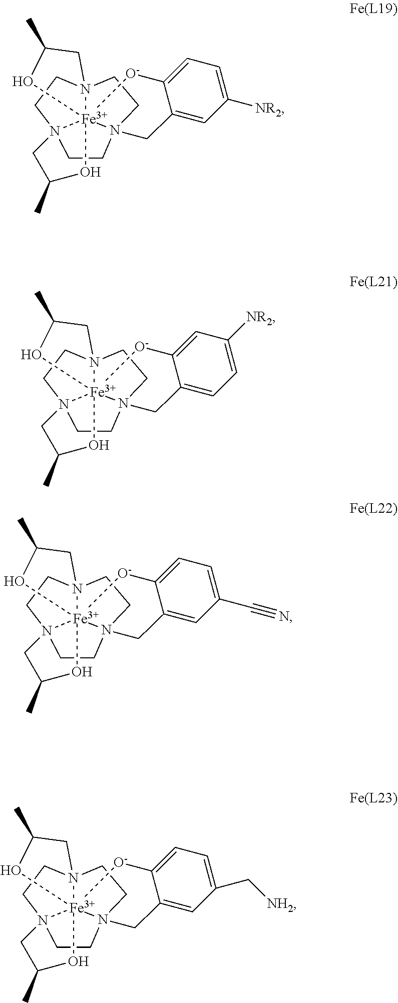

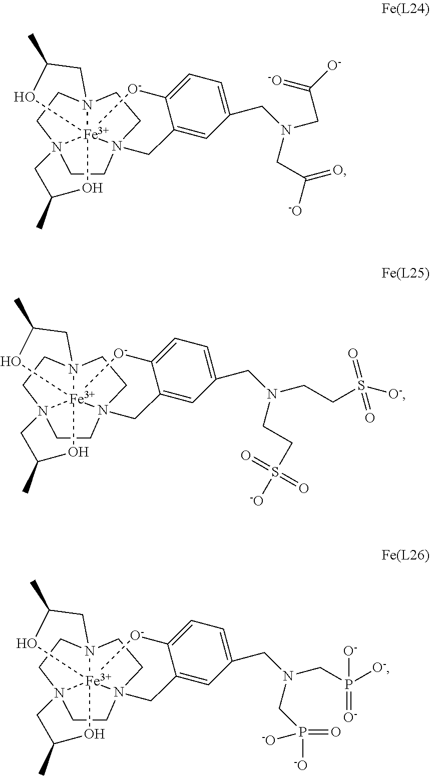

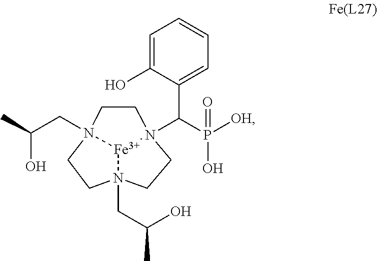

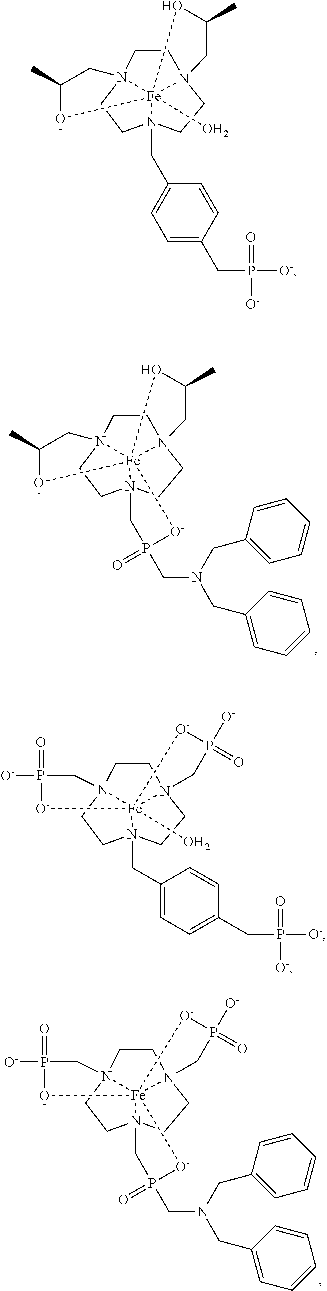

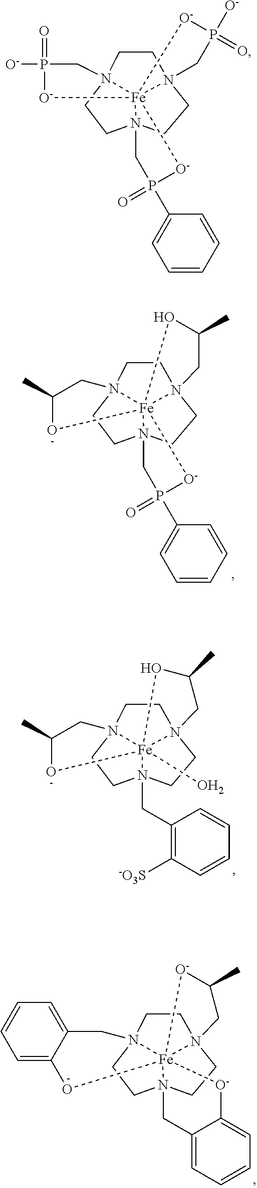

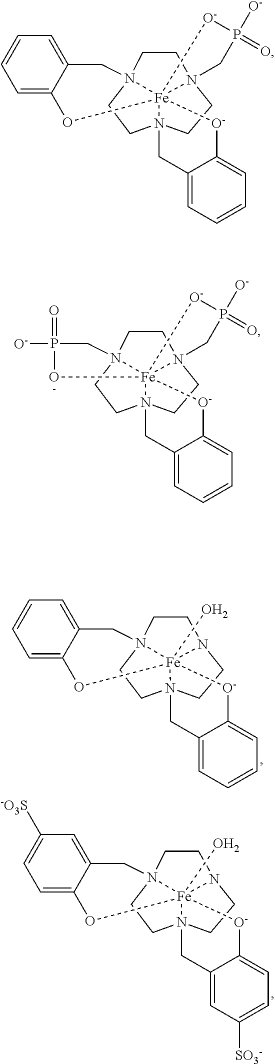

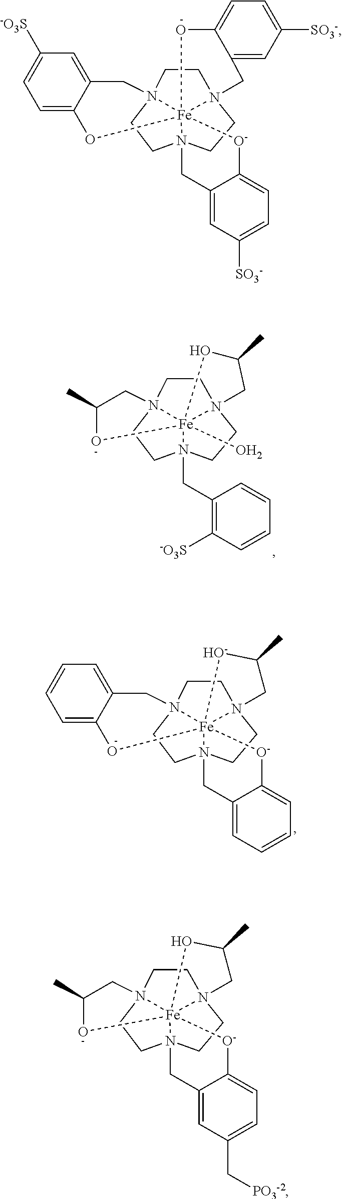

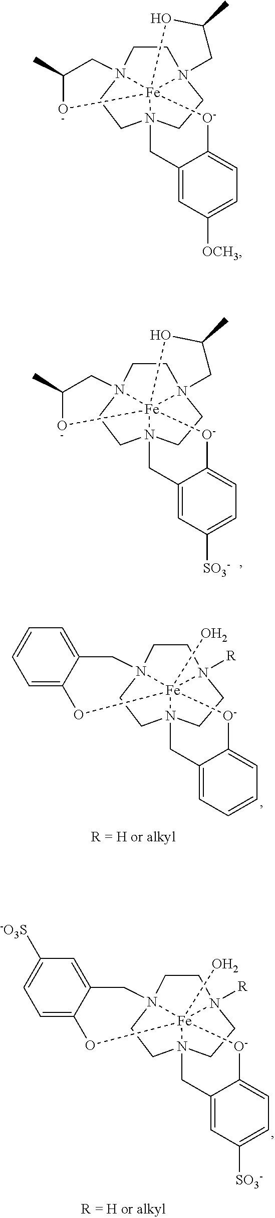

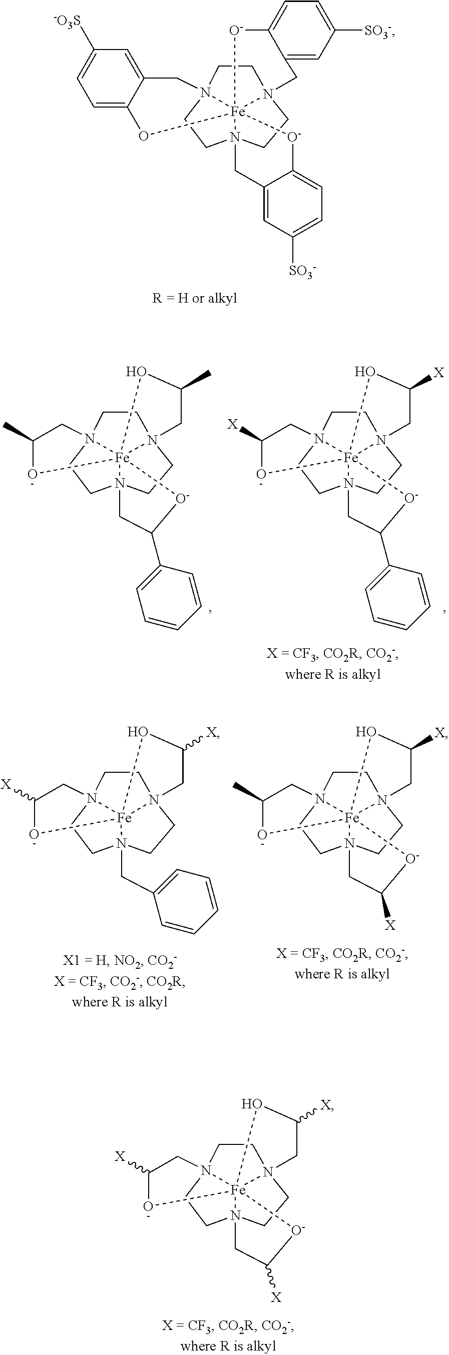

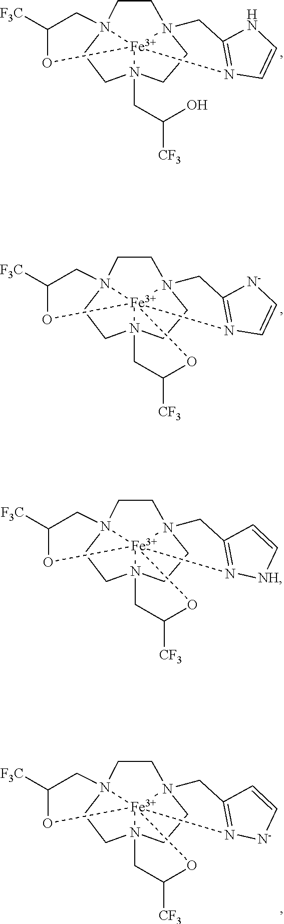

or deprotonated, partially deprotonated, or protonated species thereof (where applicable).
Statement 82. A composition comprising one or more macrocyclic complex according to any one of Statements 76-81.
Statement 83. The composition according to Statement 82, wherein the composition further comprises human serum albumin and/or meglumine.
Statement 84. A method to obtain an image of at least a portion of a cell, organ, vasculature or tissue comprising: contacting the cell, organ, vasculature, or tissue with one or more macrocyclic complex according to any one of Statements 76-81 or a composition according to Statement 82 or Statement 83, and imaging at least a portion of the cell, organ, vasculature, or tissue to obtain an image of the portion of a cell, organ, vasculature, or tissue, wherein the image is obtained by using magnetic resonance.
Statement 85. The method according to Statement 84, wherein the cell, organ, vasculature, or tissue is part of an individual.
Statement 86. The method according to Statement 84 or Statement 85, wherein the image is obtained using magnetic resonance imaging (MRI).
Statement 87. The method according to any one of Statements 84-86, wherein the macrocyclic complex(es) is/are a T₁ agent or T₁ agents.

The following examples are presented to illustrate the present disclosure. The examples are not intended to be limiting in any matter.

### Example 1

The following example describes methods of using a compound of the present disclosure.

The following complex has desirable solubility, a r₁ relaxivity of about 1.5 mM⁻¹ s⁻¹ at 33° C. and neutral pH at 1.4 Tesla and it was tolerated in mice when injected at 50 umol/kg. MRI scans showed desirable T1 weighted enhanced contrast at 4.7 Tesla:

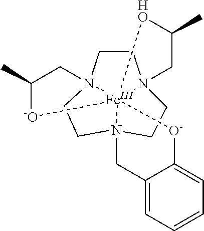

### Example 2

The following example provides synthetic details of macrocyclic complexes and compounds of the present disclosure.

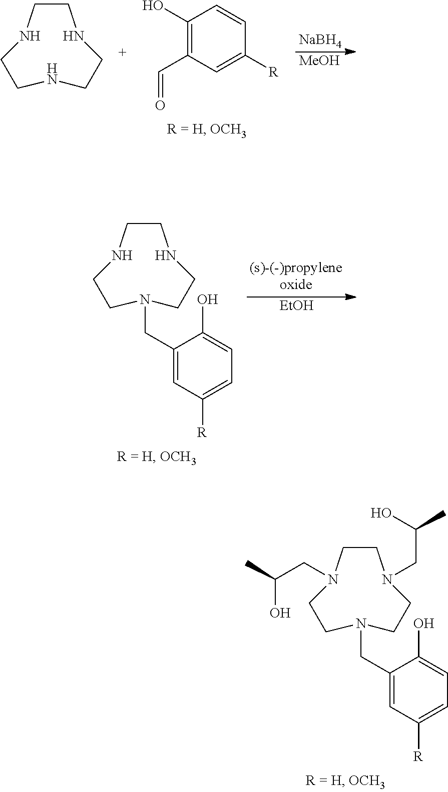

### Synthesis of TOP (R=H) or TOP-Me (R=OCH3)

1,4,7-Triazacyclononane (1.0 g, 7.74 mmol) was dissolved in 40.0 mL MeOH and stirred in a 100 mL round bottom flask under Ar (g). Acetic acid (0.222 mL, 3.87 mmol) was added to the solution, followed by addition of aldehyde (0.421 mL, 3.87 mmol salicylaldehyde or 0.493 mL, 3.87 mmol 5-methoxysalicylaldehyde). Reaction was stirred for 4-6 h until completion of imine formation (monitored by TLC/ESI-MS). Solid sodium borohydride (0.366 g, 9.68 mmol) was then slowly added to the solution. After 1 h the reaction was quenched with 40.0 mL H₂O. MeOH was then removed under vacuum, and the pH of the water solution was raised to 10 using 1M NaOH solution. Crude product was extracted with Chloroform (3×80 mL), combined organic layers dried with anhydrous sodium sulfate and dried under vacuum. The crude product was used without purification. The crude product was dissolved in 20.0 mL EtOH and stirred in a 25 mL round bottom flask. (s)-(−)propylene oxide (0.812 mL, 11.61 mmol) was added to the solution and reaction monitored until completion using ESI-MS. Product was purified on silica resin (100% Ethyl Acetate→8:2:1 Ethyl acetate:methanol: 10% aqueous ammonium hydroxide) and isolated as an oil. [R=H, brown oil, 559 mg, R=OCH₃, 28.4 mg] ESI-MS (m/z): R=H, 352.42 (100), 410.38 (5). R=OCH₃, 382.91 (100), 440.62 (10).

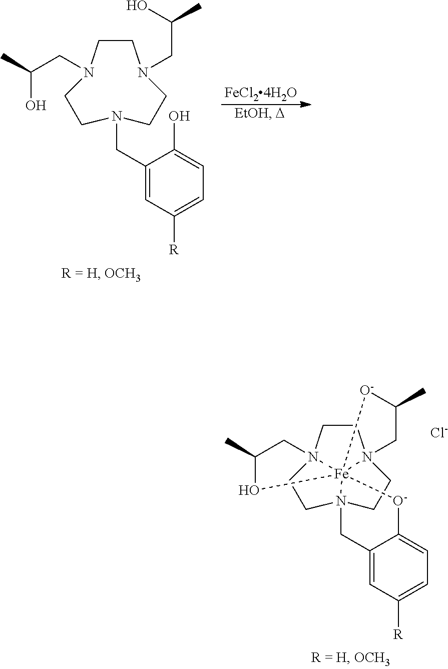

### Synthesis of Fe(TOP) or Fe(TOP-Me) Above

TACN-phenol ligand (0.142 mmol−R=H, 0.05 g, R=OCH₃, 0.054 g) was dissolved in 4.0 mL EtOH and heated to 70° C. Ferrous chloride tetrahydrate (0.142 mmol, 0.029 g), dissolved in 1.0 mL EtOH, was slowly added to the ligand solution. Alternately, FeCl₃ was added to produce the iron complex. After completion (monitored by ESI-MS), solution was cooled to room temperature and volume reduced to 2.0 mL, followed by slow addition of diethyl ether until iron complex precipitated (about 10.0 mL). Complex was filtered and washed with diethyl ether, then dried under vacuum. Iron complexes were isolated as purple solids. Yield ˜50% (˜0.034 g). ESI-MS (m/z): R=H, 478.23 (100), R=OCH₃, 508.26 (100). μ_{eff}=6.16±0.3.

Synthesis of pendant for L12 was prepared according to methods known in the art.

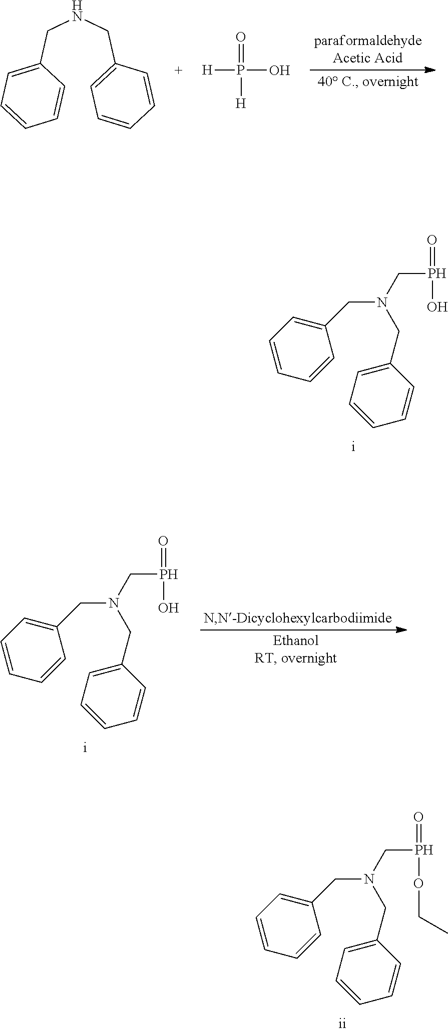

### Synthesis of L12

DACO (1,4-bis(2-hydroxypropyl)-1,4,7-triazacyclononane) is dissolved in 2-3 mL pyridine and heated to 40° C. The pendant precursor (ii) in 2-3 mL pyridine, is added to the heating solution of DACO. The reaction mixture is stirred at 40° C. for 2-3 days. Upon completion of the reaction, the solvent is removed under reduced pressure and dissolved in diethyl ether. The product is purified using a silica gel column with a diethyl ether:methanol gradient. The product elutes at 95% diethyl ether 5% methanol. Mass

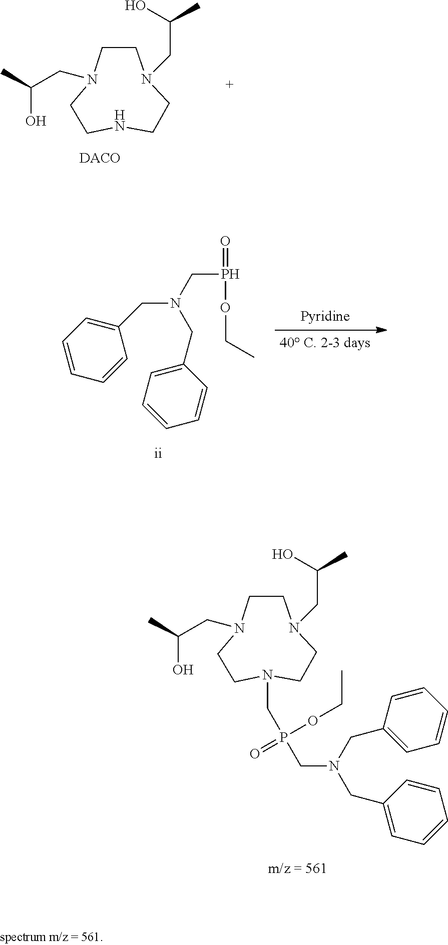

Purified product from the previous step is dissolved in 50% aqueous pyridine and heated to 70° C. The reaction mixture stirred for 1-2 days or until the ethyl protecting group is fully removed. m/z=533.8

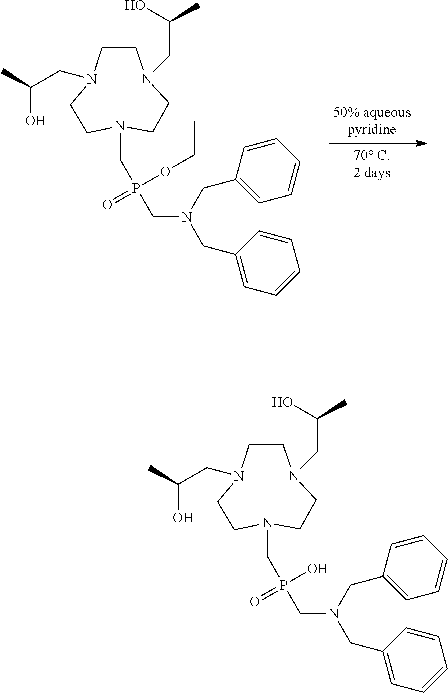 [SMILES: CCOP(=O)(CN1CCN(C[C@H](C)O)CCN(C[C@H](C)O)CC1)CN(Cc1ccccc1)Cc1ccccc1 ; C[C@H](O)CN1CCN(C[C@H](C)O)CCN(CP(=O)(O)CN(Cc2ccccc2)Cc2ccccc2)CC1]

### Synthesis of Fe(L12)

Deprotected ligand is dissolved in 2-3 mL of ethanol and heated to 60° C. One equivalent of iron(II) bromide in 2-3 mL of ethanol, is added slowly. Upon finishing the addition of the iron salt, the solution is left to stir at 60° C. for 8 hours.

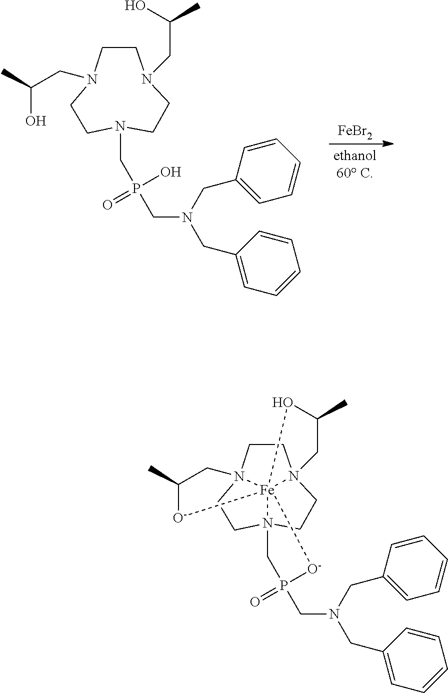

### Synthesis of L13

Taken from literature.

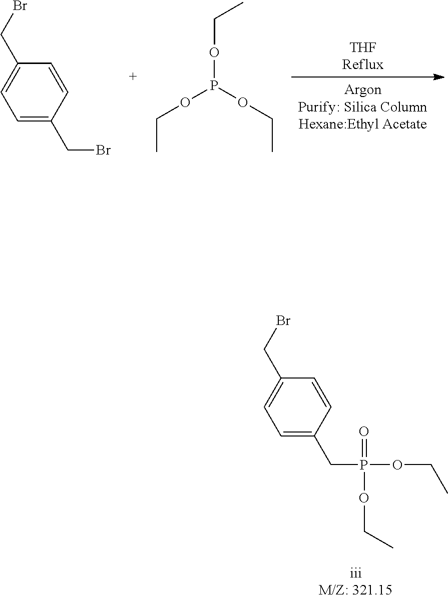

DACO and the precursor (iii) are dissolved in minimal chloroform and added to acetonitrile. The solution is allowed to stir overnight at room temperature. The solvent is removed under reduced pressure and purified using a silica gel column. The protected product eludes with 99% DCM 1% Methanol.

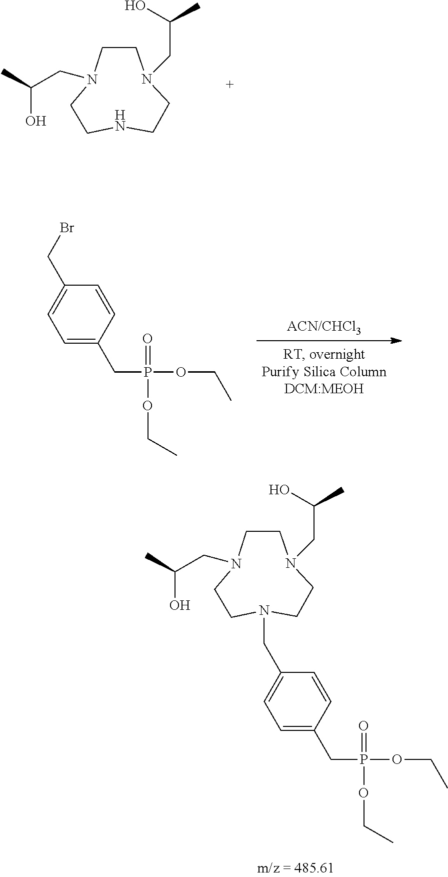 [SMILES: C[C@H](O)CN1CCNCCN(C[C@H](C)O)CC1 ; CCOP(=O)(Cc1ccc(CBr)cc1)OCC ; CCOP(=O)(Cc1ccc(CN2CCN(C[C@H](C)O)CCN(C[C@H](C)O)CC2)cc1)OCC]

The purified product is dissolved in minimal amounts of methylene chloride. Seven equivalents of bromotrimethyl silane is added and the solution is left to reflux for 18 hours.

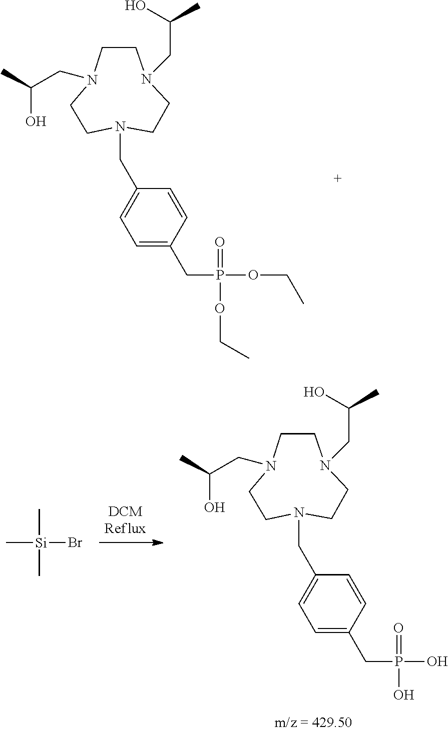

Deprotected ligand (L13) is dissolved in 2-3 mL of ethanol and heated to 60° C. One equivalent of iron(II) bromide in 2-3 mL of ethanol, is added slowly. Upon finishing the addition of the iron salt, the solution is left to stir at 60° C. for 8 hours.

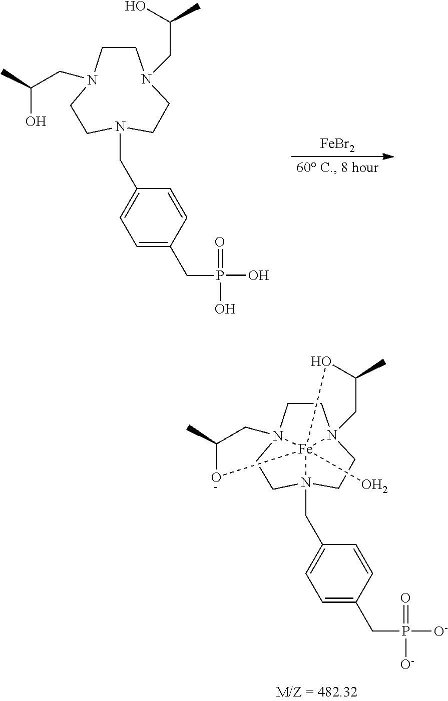

### Example 3

The following example provides synthetic details of macrocyclic complexes and compounds of the present disclosure.

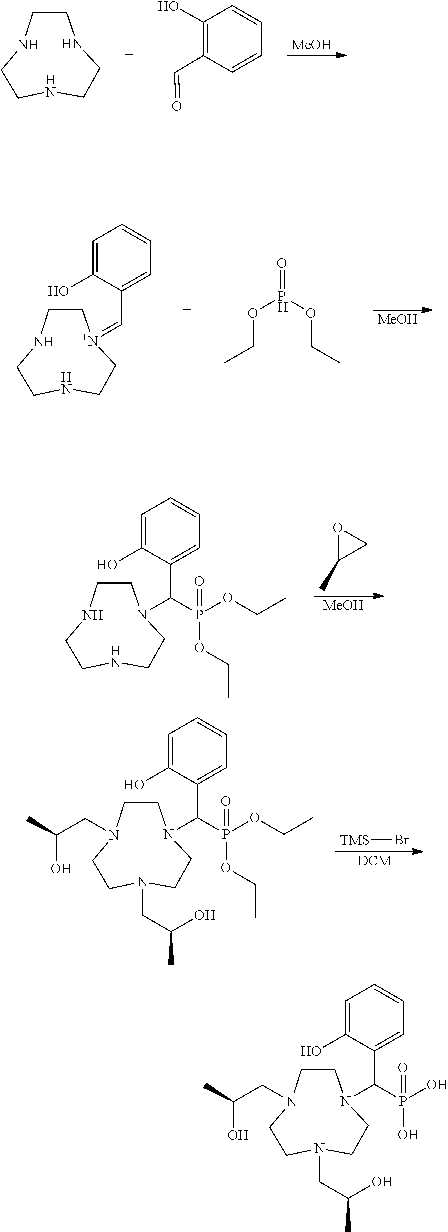

### Example 4

The following example provides synthetic details of macrocyclic complexes and compounds of the present disclosure.

### Synthesis of 2,2′-((1,4,7-triazonane-1,4-diyl)bis(methylene))diphenol with Fe(III) Coordinated Thereto

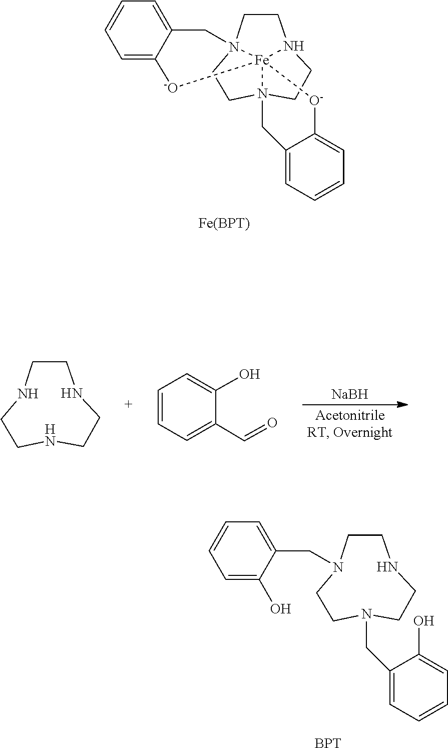

1,4,7-triazacyclononane (7.8 mmol) was dissolved in 35 mL of acetonitrile and was put on a stir plate. Salicylaldehyde (19.2 mmol, 2.45 equiv.) was dissolved in a separate 35 mL of acetonitrile and put in an addition funnel. The salicylaldehyde solution was added dropwise to the 1, 4, 7-triazacyclononane (about 1 drop every 25 seconds). Once the addition was complete, the mixture was stirred for 18 hours at room temperature. After 18 hours, sodium borohydride (39.1 mmol, 5 equiv.) was added slowly. As the sodium borohydride was added, the product formed as a white solid and precipitated out of solution. The solid was collected via vacuum filtration and was recrystallized from a mixture of methanol and acetonitrile with heat yielding a white solid. MS-ESI+342.7 (M+H⁺, 100%). ¹H NMR (300 MHZ, D₂O): 2.94 (4H, CH₂, TACN), 3.27 (8H, CH₂, TACN), 3.92 (CH₂, N—CH₂-Phenol), 6.81 (4H, CH, Phenol), 7.16 (CH, Phenol).

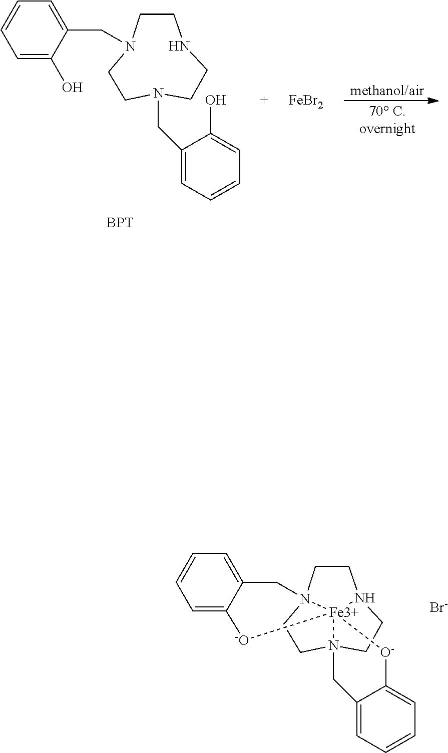

2, 2′-((1, 4, 7-triazacyclononane-1,4-diyl)bis(methylene))diphenol (0.3 mmol) was dissolved in 5 mL methanol and was stirred at 70° C. Iron (II) bromide (0.3 mmol) was dissolved in 5 mL of methanol. Once the ligand solution reached 70° C., the iron solution was added dropwise. The mixture stirred overnight and then the volume was reduced to 2-3 mL using a rotary evaporator. The solution was added dropwise to a solution of stirring diethyl ether where the product formed as a red-purple solid that was collected and washed with diethyl ether. MS-ESI+395.20 (M+H⁺, 100%). μ_{eff}=5.96 by using Evans method.

### Synthesis of (S)-2,2′-((7-(2-hydroxypropyl)-1,4,7-triazonane-1,4-diyl)bis(methylene))diphenol, with Fe(III) Chelated Thereto

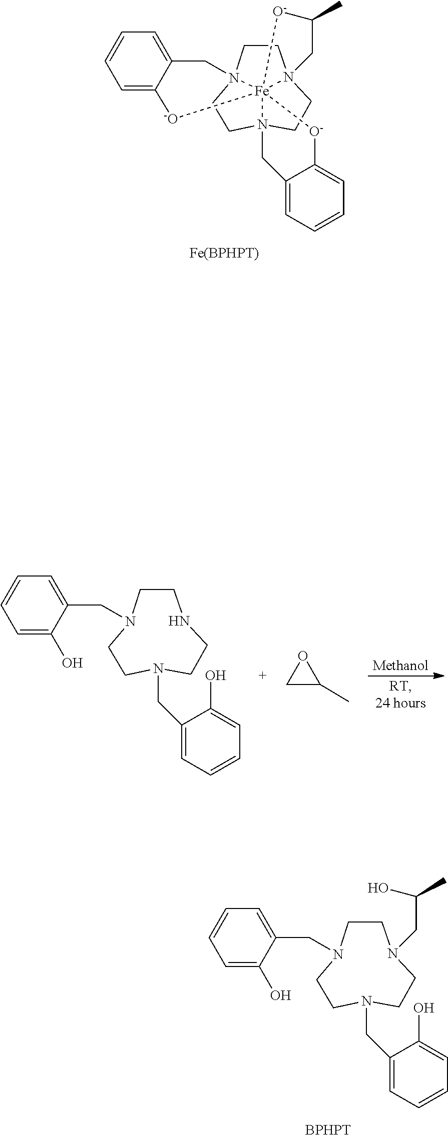

2,2′-(1,4,7-triazacyclononane-1,4-diyl)bis(methylene))diphenol was synthesized as previously stated. The first ligand (0.1 mmol) was dissolved in 10 mL of methanol, and S-(−) propylene oxide (0.21 mmol, 2 equiv.) was added. The mixture was stirred at room temperature for 24 hours, after which solvent and excess propylene oxide was removed on a rotary evaporator. (S)-2, 2′-(7-2-hydroxypropyl)-1, 4, 7-triazacyclononane-1,4-diyl)bis(methylene))diphenol was purified using a silica gel column using a gradient of hexanes and ethyl acetate followed by ethyl acetate and methanol. Impurities were removed during the gradient with hexanes and ethyl acetate, and clean product eluded with 90% ethyl acetate, 10% methanol. MS-ESI+400.67 (M+H⁺, 100%), 422.50 (M+Na⁺, 35%).

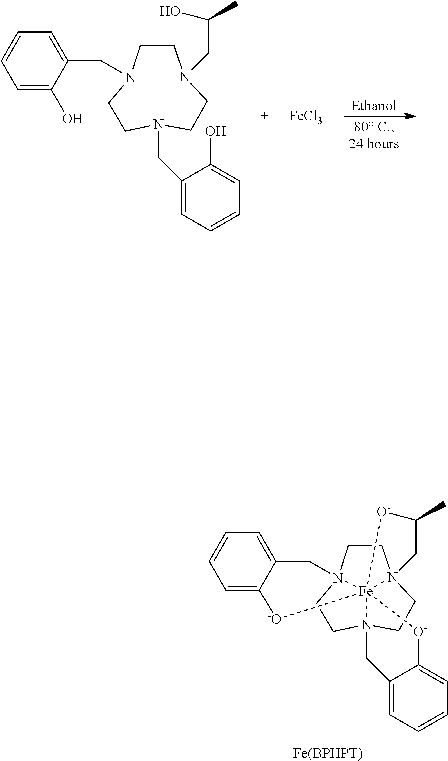

(S)-2, 2′-((7-2-hydroxypropyl)-1,4,7-triazacyclononane-1,4-diyl)bis(methylene))diphenol (0.1 mmol) was dissolved in 5 mL ethanol and stirred at 80° C. Iron (III) chloride (0.15 mmol, 1.5 equiv.) was dissolved in 5 mL ethanol and added dropwise to the ligand solution. The mixture was stirred at 80° C. for 24 hours, after which the volume was reduced to 3 mL. The remaining solution was stirred as diethyl ether was added slowly, causing the iron complex to precipitate out. The complex was collected via centrifuge and washed with ether until the supernatant became clear and then the solid was dried on a vacuum line. MS-ESI+453.83 (M+H⁺, 100%).

### Example 5

The following example provides synthetic details of macrocyclic complexes and compounds of the present disclosure.

### Synthesis of 3-formyl-4-hydroxybenzenesulfonate Sodium Salt

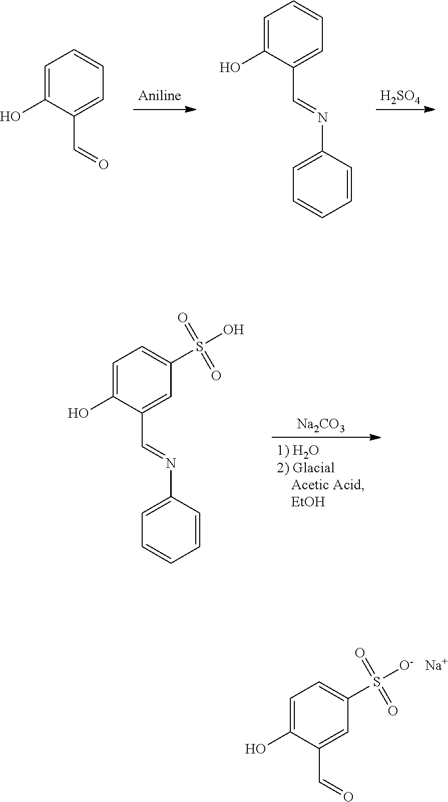

Salicylaldehyde (5.38 mL, 50.0 mmol) and Aniline (4.61 mL, 50.0 mmol) were stirred neat at 50° C. for 4 h. Solution was then placed in a chemical freezer until a yellow solid precipitates. Precipitate was filtered and washed with cold EtOH, then recrystallized in hot EtOH to produce Salicylaniline as yellow needle-like crystals in quantitative yield. ESI-MS (m/z): [M−H]⁺=198.20 (100).

Salicylaniline (3.50 g, 17.8 mmol) was dissolved in 10.0 mL concentrated sulfuric acid and heated to 105-110° C. for about 3 h. Upon completion, solution was carefully poured into 100 mL ice water in which a brown precipitate was formed. The solution was then heated to boil, and filtered by gravity filtration. The filtrate was cooled in an ice bath until a brown solid precipitates. Precipitate was filtered, washed, producing 4-hydroxy-3-[(phenylimino)methyl]-benzenesulfonic acid as a brown solid. Yield 2.53 g (51%). ESI-MS (m/z): [M−H]⁺=278.24 (100).

4-hydroxy-3-[(phenylimino)methyl]-benzenesulfonic acid (2.53 g, 9.12 mmol) was dissolved in 17.0 mL and stirred in a 50 mL Erlenmeyer flask. Sodium carbonate (1.02 g, 9.62 mmol) was then added slowly over 30 min. Solution was then boiled vigorously for 2 h, replenishing any lost water during the process. The solution was cooled to room temperature. 10.2 mL of glacial acetic acid was then added to the solution, followed by an equal volume EtOH (˜30.0 mL). Solution was cooled in a chemical freezer for several hours to produce fine beige crystals. These crystals were filtered and washed with cold EtOH to produce 3-formyl-4-hydroxybenzenesulfonate sodium salt as a yellow-brown crystalline solid. Yield 1.51 g (73%). ESI-MS (m/z) negative mode: [M]⁻¹=201.17 (100).

### Synthesis of 3-((4,7-bis((S)-2-hydroxypropyl)-1,4,7-triazonan-1-yl)methyl)-4-hydroxybenzenesulfonate Sodium Salt

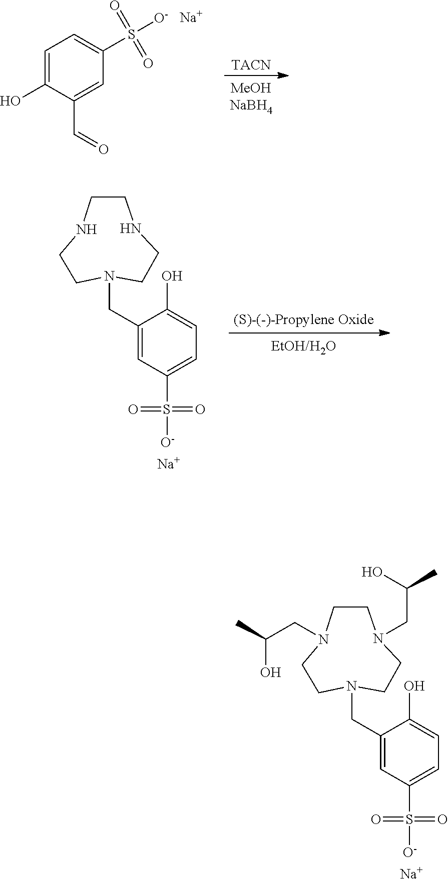

TACN (0.500 g, 3.87 mmol) was dissolved in 25.0 mL MeOH and stirred in a 50 mL round bottom flask. 3-formyl-4-hydroxybenzenesulfonate sodium salt (0.867 g, 3.87 mmol), dissolved in 10.0 mL MeOH, was added slowly over 1 h using a 10.0 mL addition funnel. Solution was stirred overnight for about 12 h. Sodium borohydride (0.366 g, 9.68 mmol) was then added slowly and solution allowed to react for 1 h. Solvent was removed under pressure, and crude was ran through a small plug of basic alumina with a 90:10 DCM/MeOH solution as the eluding solvent. Crude product was isolated as a yellow oil. Crude 3-((1,4,7-triazonan-1-yl)methyl)-4-hydroxybenzenesulfonate sodium salt was used without further purification. ESI-MS (m/z): [M−H]⁺=316.37 (100), [M−Na]⁺=338.31 (30).

Crude 3-((1,4,7-triazonan-1-yl)methyl)-4-hydroxybenzenesulfonate sodium salt (0.328 g, 0.970 mmol) was dissolved in 10 mL 50:50 EtOH/water mixture and stirred in a 20 mL scintillation vial. (S)-(−)-Propylene Oxide was then added (349 μL, 4.84 mmol) and allowed to react overnight about 12 h. Solvent was removed under vacuum, and crude 3-((4,7-bis((S)-2-hydroxypropyl)-1,4,7-triazonan-1-yl)methyl)-4-hydroxybenzenesulfonate sodium salt obtained by column chromatography using basic alumina, DCM/MeOH (100:0 to 80:20, 0:100 flush). ESI-MS (m/z): [M]⁻=430.31 (100).

### Synthesis of (S)-3,3′-((7-(2-hydroxypropyl)-1,4,7-triazonane-1,4-diyl)bis(methylene))bis(4-hydroxybenzenesulfonate) Disodium Salt

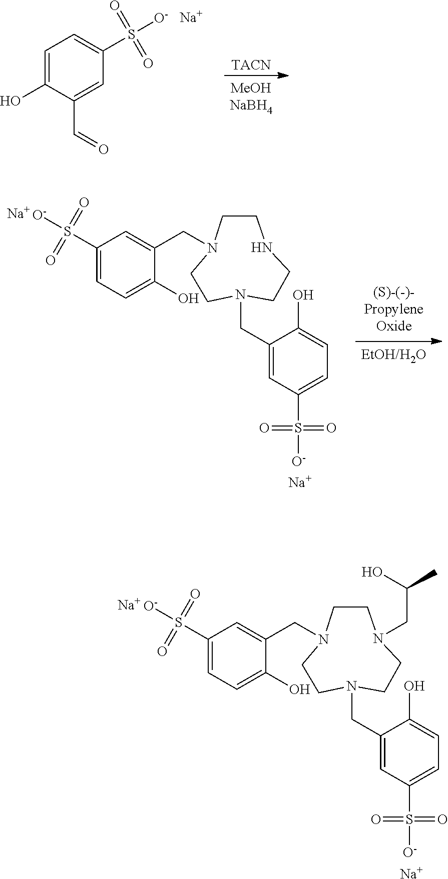

TACN (0.500 g, 3.87 mmol) was dissolved in 25.0 mL MeOH and stirred in a 50 mL round bottom flask. 3-formyl-4-hydroxybenzenesulfonate sodium salt (1.73 g, 7.74 mmol), dissolved in 10.0 mL MeOH, was added slowly over 1 h using a 10.0 mL addition funnel. Solution was stirred overnight for about 12 h. Sodium borohydride (0.732 g, 19.4 mmol) was then added slowly and solution allowed to react for 1 h. Solvent was removed under pressure, and crude was ran through a small plug of basic alumina with a 90:10 DCM/MeOH solution as the eluding solvent. Crude product was isolated as a yellow oil. Crude 3,3′-((1,4,7-triazonane-1,4-diyl)bis(methylene))bis(4-hydroxybenzenesulfonate) disodium salt was used without further purification. ESI-MS (m/z): [M−H]⁻=500.19 (100).

Crude 3,3′-((1,4,7-triazonane-1,4-diyl)bis(methylene))bis(4-hydroxybenzenesulfonate) disodium salt (0.529 g, 0.970 mmol) was dissolved in 10 mL 50:50 EtOH/water mixture and stirred in a 20 mL scintillation vial. (S)-(−)-Propylene Oxide was then added (175 μL, 2.43 mmol) and allowed to react overnight about 12 h. Solvent was removed under vacuum, and crude (S)-3,3′-((7-(2-hydroxypropyl)-1,4,7-triazonane-1,4-diyl)bis(methylene))bis(4-hydroxybenzenesulfonate) disodium salt obtained by column chromatography using basic alumina, DCM/MeOH (100:0 to 80:20, 0:100 flush). ESI-MS (m/z): [M−H]⁻=558.25 (100).

### Synthesis of 3,3′-((7-methyl-1,4,7-triazonane-1,4-diyl)bis(methylene))bis(4-hydroxybenzenesulfonate) Disodium Salt

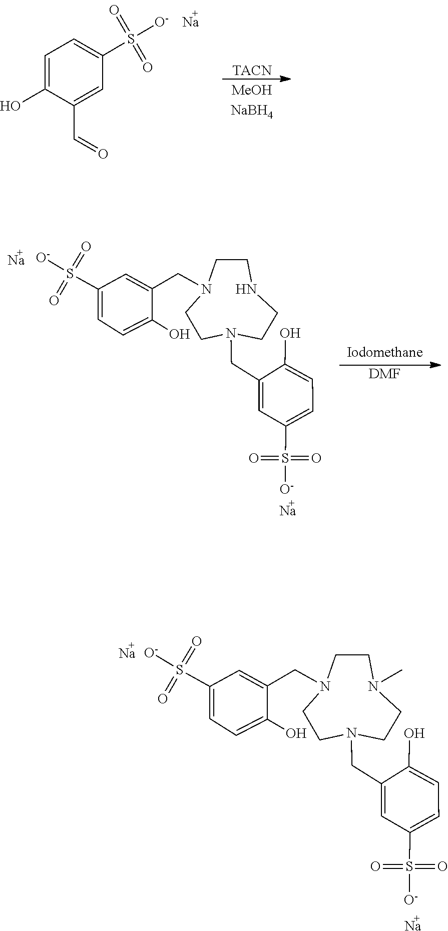

TACN (0.500 g, 3.87 mmol) was dissolved in 25.0 mL MeOH and stirred in a 50 mL round bottom flask. 3-formyl-4-hydroxybenzenesulfonate sodium salt (1.73 g, 7.74 mmol), dissolved in 10.0 mL MeOH, was added slowly over 1 h using a 10.0 mL addition funnel. Solution was stirred overnight for about 12 h. Sodium borohydride (0.732 g, 19.4 mmol) was then added slowly and solution allowed to react for 1 h. Solvent was removed under pressure, and crude was ran through a small plug of basic alumina with a 90:10 DCM/MeOH solution as the eluding solvent. Crude product was isolated as a yellow oil. Crude 3,3′-((1,4,7-triazonane-1,4-diyl)bis(methylene))bis(4-hydroxybenzenesulfonate) disodium salt was used without further purification. ESI-MS (m/z) negative mode: [M−H]⁻=500.19 (100).

Crude 3,3′-((1,4,7-triazonane-1,4-diyl)bis(methylene))bis(4-hydroxybenzenesulfonate) disodium salt (0.529 g, 0.97 mmol) was dissolved in 10 mL DMF and heated to about 60° C. while stirring in a 20 mL scintillation vial. Iodomethane (68.0 μL, 1.07 mmol) was then added and allowed to react overnight for about 12 h. Solvent was removed under vacuum, and crude 3,3′-((7-methyl-1,4,7-triazonane-1,4-diyl)bis(methylene))bis(4-hydroxybenzenesulfonate) disodium salt obtained by column chromatography using basic alumina, DCM/MeOH (100:0 to 80:20, 0:100 flush). ESI-MS (m/z): [M−H]⁻=514.21 (100).

### Synthesis of 3,3′,3″-((1,4,7-triazonane-1,4,7-triyl)tris(methylene))tris(4-hydroxybenzenesulfonate) trisodium salt (L1), [Fe(L1)Na₃]

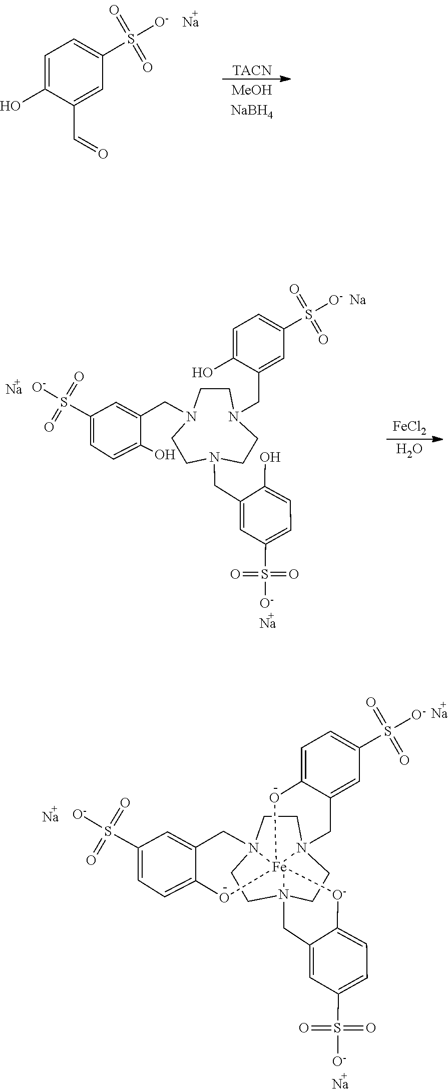

TACN (0.250 g, 1.94 mmol) was dissolved in 25.0 mL MeOH and stirred in a 100 mL round bottom flask. 3-formyl-4-hydroxybenzenesulfonate sodium salt (2.60 g, 11.6 mmol), dissolved in 20.0 mL MeOH, was added slowly over 1 h using a 25 mL addition funnel. Solution was stirred overnight for about 12 h. Sodium borohydride (0.550 g, 14.6 mmol) was then added slowly and solution allowed to react for 1 h. Solvent was removed under vacuum, and crude was dissolved in 10 mL MeOH. Solution was heated to boil, then allowed to cool to room temperature. Solution was placed in chemical freezer overnight, about 12 h. The precipitate that formed was filtered and washed with cold EtOH to produce 3,3′,3″-((1,4,7-triazonane-1,4,7-triyl)tris(methylene))tris(4-hydroxybenzenesulfonate) trisodium salt as a white solid. Yield 0.512 g (˜35%). ESI-MS (m/z): [M−H]²⁻=343.00 (100%).

3,3′,3″-((1,4,7-triazonane-1,4,7-triyl)tris(methylene))tris(4-hydroxybenzenesulfonate) trisodium salt (0.206 g, 0.273 mmol) was dissolved in 5 mL water and stirred in a 20 mL scintillation vial at about 60′° C. Anhydrous Ferrous Chloride (0.035 g, 0.273 mmol), dissolved in 5 mL water, was added gradually to the stirring solution. Reaction was monitored by ESI-MS, and completion occurred after about 2 h. Solvent was removed under vacuum, and crude oil dissolved in 2 mL MeOH. Addition of diethyl ether precipitated a dark red-brown solid which was then filtered and washed with cold EtOH, then dried under vacuum. Subsequent precipitation and washing produced [Fe(L1)Na₃] as a red-brown solid. Yield 0.140 g (˜63%). ESI-MS (m/z): [M]³⁻=245.82 (100).

### Synthesis of diethyl (3-formyl-4-hydroxybenzyl)phosphonate

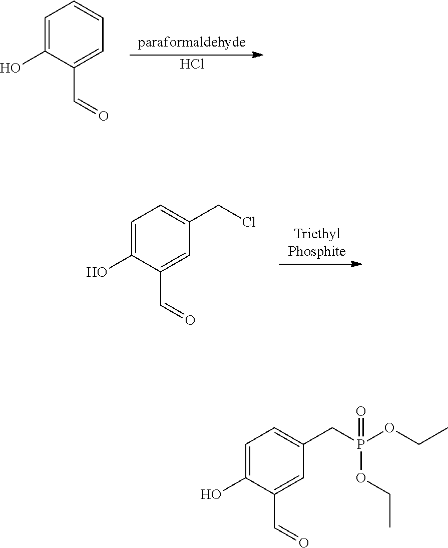 [SMILES: O=Cc1ccccc1O ; Cl ; O=Cc1cc(CCl)ccc1O ; CCOP(=O)(Cc1ccc(O)c(C=O)c1)OCC]

Paraformaldehyde (1.58 g, 50.0 mmol) was dissolved in 60 mL of 12 M HCl and stirred in a 100 mL round bottom flask. Once all of the paraformaldehyde had dissolved, Salicylaldehyde (5.38 mL, 50.0 mmol) was added slowly with a 10 mL addition funnel. The solution was stirred for 3 days and the pink precipitate which formed was filtered and washed with water until the filtrate was clear. The slightly pink solid was dissolved in diethyl ether, dried with anhydrous sodium sulfate and solvent removed under vacuum. Crude product was recrystallized from hot hexanes to produce a white solid. 5-(chloromethyl)-2-hydroxybenzaldehyde was used directly in the next reaction without further purification.

5-(chloromethyl)-2-hydroxybenzaldehyde (2.25 g, 13.2 mmol) was added to a 10 mL round bottom flask with a stir bar. Triethyl Phosphite (2.54 mL, 14.5 mmol) was added very slowly to the round bottom flask with care taken to not allow the reaction to overflow the round bottom flask. Once all of the triethyl phosphite has been added, the flask was fitted with a condenser and reaction was heated to about 90° C. overnight about 12 h. The condenser was then replaced with a vacuum distillation set up and liquid impurities are removed. (3-formyl-4-hydroxybenzyl)phosphonate was then purified by column chromatography, silica Hexane/EtOAc (90:10 to 0:100) as an off-white oil. Yield 3.31 g (˜92%). ESI-MS (m/z): [M−H]⁺=273.65 (100).

### Synthesis of (3-((4,7-bis((S)-2-hydroxypropyl)-1,4,7-triazonan-1-yl)methyl)-4-hydroxybenzyl)phosphonic acid (TPP), [Fe(TPP)Cl]

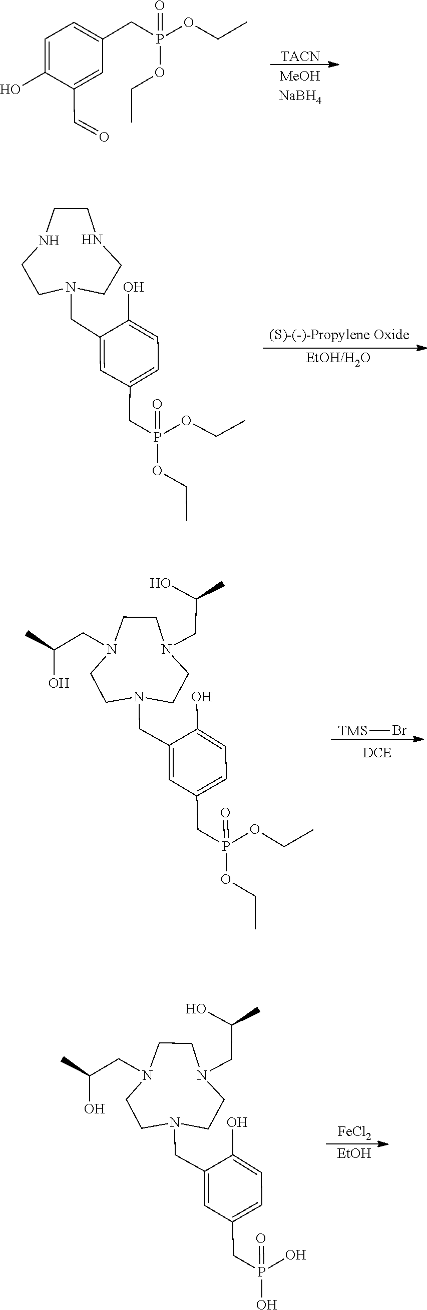

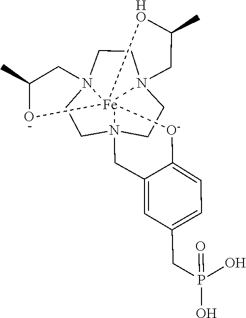

TACN (1.23 g, 9.25 mmol) was dissolved in 25 mL MeOH and stirred in a 50 mL round bottom flask. (3-formyl-4-hydroxybenzyl)phosphonate (2.52 g, 9.25 mmol), in 10 mL MeOH, was added slowly to the round bottom flask using a 10 mL addition funnel. Solution was stirred overnight for about 12 h. Sodium Borohydride (0.875 g, 23.1 mmol) was then added slowly and solution allowed to stir for 1 h. Solvent was removed under pressure, and crude product was purified by column chromatography, basic alumina CHCl₃/MeOH (99:1 to 0:100). Diethyl (3-((1,4,7-triazonan-1-yl)methyl)-4-hydroxybenzyl)phosphonate was isolated as a yellow oil. Yield 2.14 g (˜60%). ESI-MS (m/z): [M−H]⁺=386.64 (100).

Diethyl (3-((1,4,7-triazonan-1-yl)methyl)-4-hydroxybenzyl)phosphonate (1.07 g, 2.78 mmol) was dissolved in 18 mL of a 50:50 EtOH/water mixture and stirred in a 20 mL scintillation vial. (S)-(−)-Propylene Oxide was then added (1.00 mL, 13.9 mmol) and allowed to react overnight about 12 h. Solvent was removed under vacuum, and crude diethyl (3-((4,7-bis((S)-2-hydroxypropyl)-1,4,7-triazonan-1-yl)methyl)-4-hydroxybenzyl)phosphonate obtained by column chromatography using basic alumina, DCM/MeOH (100:0 to 80:20, 0:100 flush). ESI-MS (m/z): [M−H]⁺=502.77 (100), [(M−2H)/2]⁺=252.47 (35).

Diethyl (3-((4,7-bis((S)-2-hydroxypropyl)-1,4,7-triazonan-1-yl)methyl)-4-hydroxybenzyl)phosphonate (0.255 g, 0.508 mmol) was dissolved in 10 mL DCE in a 25 mL round bottom flask. While stirring, TMS-Br (678 μL, 5.08 mmol) was added slowly over about 10 min. A condenser was added and the solution was refluxed overnight for about 12 h. The reaction was then quenched with 10 mL water. The water later is collected, and the DCE was washed with water (3×25 mL). Water layers were combined and solvent removed under vacuum. Crude product was washed with cold EtOH to produce (3-((4,7-bis((S)-2-hydroxypropyl)-1,4,7-triazonan-1-yl)methyl)-4-hydroxybenzyl)phosphonic acid as a white solid. Yield 0.075 g (˜33%). ESI-MS (m/z): [M−H]⁺=446.61 (100).

(3-((4,7-bis((S)-2-hydroxypropyl)-1,4,7-triazonan-1-yl)methyl)-4-hydroxybenzyl)phosphonic acid (L2) (0.031 g, 0.070 mmol) was dissolved in 2 mL EtOH and heated to 60° C. while stirring in a 1 DRAM vial. Anhydrous Ferrous Chloride (0.009 g, 0.070 mmol) was then dissolved in 1 mL EtOH, and slowly added to the solution. Solution was stirred overnight for about 12 h. Solvent was removed under vacuum, and the crude oil was dissolved in 1 mL MeOH. Iron complex [Fe(L2)Cl] was then precipitated from solution using diethyl ether, producing a dark purple solid. Yield 0.024 g (˜65%). ESI-MS (m/z): [M]⁺=499.24 (100).

### Synthesis of (2S,2′S)-1,1′-(7-((3-hydroxypyridin-4-yl)methyl)-1,4,7-triazonane-1,4-diyl)bis(propan-2-ol) (L3), [Fe(L3)Cl]

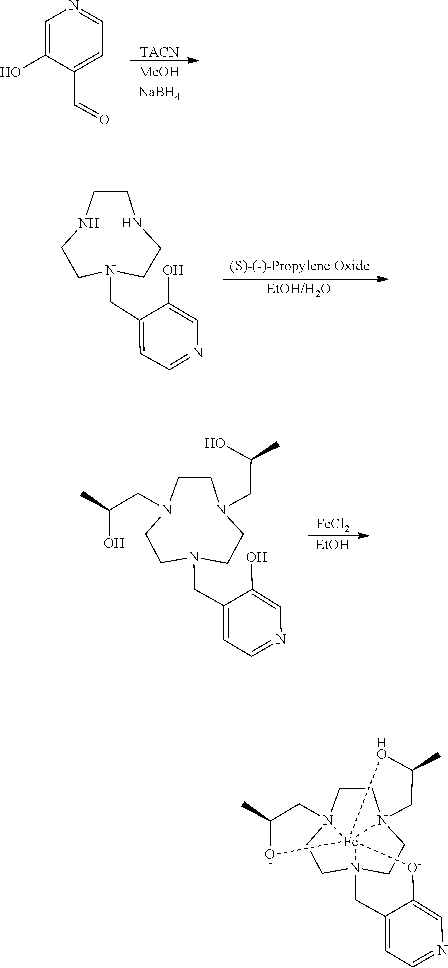

TACN (0.500 g, 3.87 mmol) was dissolved in 25 mL MeOH and stirred in a 50 mL round bottom flask. 3-Hydroxy-4-pyridinecarboxaldehyde (0.491 g, 3.87 mmol), in 10 mL MeOH, was added slowly to the round bottom flask using a 10 mL addition funnel. Solution was stirred overnight for about 12 h. Sodium Borohydride (0.366 g, 9.68 mmol) was then added slowly and solution allowed to stir for 1 h. Solvent was removed under pressure, and crude product was purified by column chromatography, basic alumina DCM/MeOH (100:0 to 80:20, 0:100 flush). Crude 4-((1,4,7-triazonan-1-yl)methyl)pyridin-3-ol was isolated as an orange-yellow oil. Crude used in consequent reaction without further purification. ESI-MS (m/z): [M−H]⁺=237.50 (100).

Crude 4-((1,4,7-triazonan-1-yl)methyl)pyridin-3-ol (0.295 g, 1.25 mmol) was dissolved in 10 mL 80:20 EtOH/water mixture and stirred in a 20 mL scintillation vial. (S)-(−)-Propylene Oxide was then added (451 μL, 6.25 mmol) and allowed to react overnight about 12 h. Solvent was removed under vacuum, and crude (2S,2′S)-1,1′-(7-((3-hydroxypyridin-4-yl)methyl)-1,4,7-triazonane-1,4-diyl)bis(propan-2-ol) (L3) obtained by column chromatography using basic alumina, DCM/MeOH (100:0 to 80:20, 0:100 flush). Crude product isolated as a yellow oil. ESI-MS (m/z): [M−H]⁺=353.34 (100).

(2S,2′S)-1,1′-(7-((3-hydroxypyridin-4-yl)methyl)-1,4,7-triazonane-1,4-diyl)bis(propan-2-ol) (L3) (0.038 g, 0.108 mmol) was dissolved in 2 mL EtOH and heated to 60° C. while stirring in a 1 DRAM vial. Anhydrous Ferrous Chloride (0.014 g, 0.108 mmol) was then dissolved in 1 mL EtOH, and slowly added to the solution. Solution was stirred overnight for about 12 h. Solvent was removed under vacuum, and the crude oil was dissolved in 1 mL MeOH. Iron complex [Fe(L3)Cl] was then precipitated from solution using diethyl ether, producing an orange solid. ESI-MS (m/z): [M]⁺=406.76 (100). Longitudinal relaxivity at 33° C., 1.4 T, 100 mM NaCl, 10 mM HEPES, pH 7 is 1.3±0.1 mM⁻¹s⁻¹.

### Synthesis of (2S,2′S)-1,1′-(7-(2-hydroxy-5-methoxybenzyl)-1,4,7-triazonane-1,4-diyl)bis(propan-2-ol) (L4), [Fe(L4)Cl]

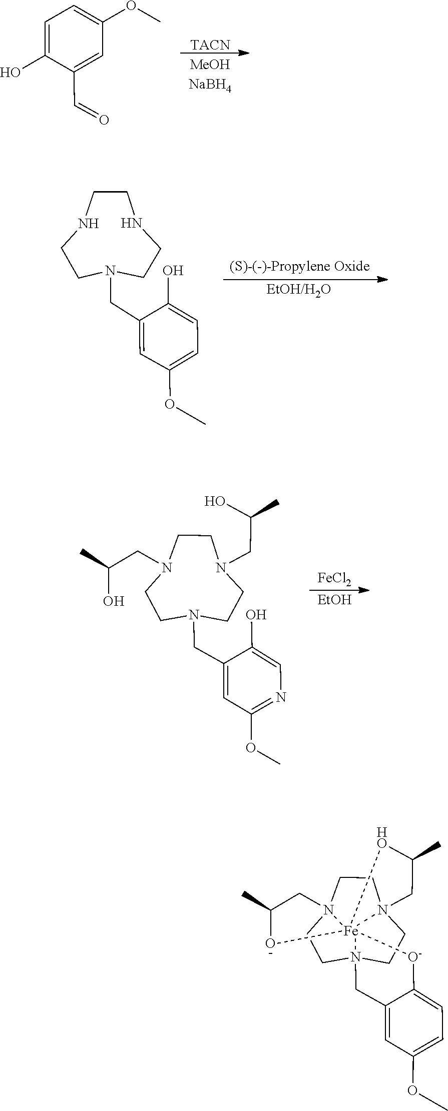

TACN (0.500 g, 3.87 mmol) was dissolved in 25 mL MeOH and stirred in a 50 mL round bottom flask. 2-hydroxy-5-methoxybenzaldehyde (493 μL, 3.87 mmol), in 10 mL MeOH, was added slowly to the round bottom flask using a 10 mL addition funnel. Solution was stirred overnight for about 12 h. Sodium Borohydride (0.366 g, 9.68 mmol) was then added slowly and solution allowed to stir for 1 h. Solvent was removed under pressure, and crude product was purified by column chromatography, basic alumina DCM/MeOH (100:0 to 80:20, 0:100 flush). Crude 2-((1,4,7-triazonan-1-yl)methyl)-4-methoxyphenol was isolated as an yellow oil. Crude used in consequent reaction without further purification. ESI-MS (m/z): [M−H]⁺=266.66 (100).

Crude 2-((1,4,7-triazonan-1-yl)methyl)-4-methoxyphenol (0.371 g, 1.40 mmol) was dissolved in 10 mL 80:20 EtOH/water mixture and stirred in a 20 mL scintillation vial. (S)-(−)-Propylene Oxide was then added (488 μL, 6.98 mmol) and allowed to react overnight about 12 h. Solvent was removed under vacuum, and crude (2S,2′S)-1,1′-(7-(2-hydroxy-5-methoxybenzyl)-1,4,7-triazonane-1,4-diyl)bis(propan-2-ol) (L4) obtained by column chromatography using basic alumina, DCM/MeOH (100:0 to 80:20, 0:100 flush). Crude product isolated as a yellow oil. ESI-MS (m/z): [M−H]⁺=382.93 (100).

(2S,2′S)-1,1′-(7-(2-hydroxy-5-methoxybenzyl)-1,4,7-triazonane-1,4-diyl)bis(propan-2-ol) (L4) (0.028 g, 0.074 mmol) was dissolved in 2 mL EtOH and heated to 60° C. while stirring in a 1 DRAM vial. Ferrous Chloride Tetrahydrate (0.015 g, 0.074 mmol) was then dissolved in 1 mL EtOH, and slowly added to the solution. Solution was stirred overnight for about 12 h. Solvent was removed under vacuum, and the crude oil was dissolved in 1 mL MeOH. Iron complex [Fe(L4)Cl] was then precipitated from solution using diethyl ether, producing an orange solid. ESI-MS (m/z): [M]⁺=436.36 (100).

### Synthesis of (2R,2′R,2″R)-3,3′,3″-(1,4,7-triazonane-1,4,7-triyl)tris(2-hydroxypropanoate) trilithium salt (L5), [Fe(L5)Cl]

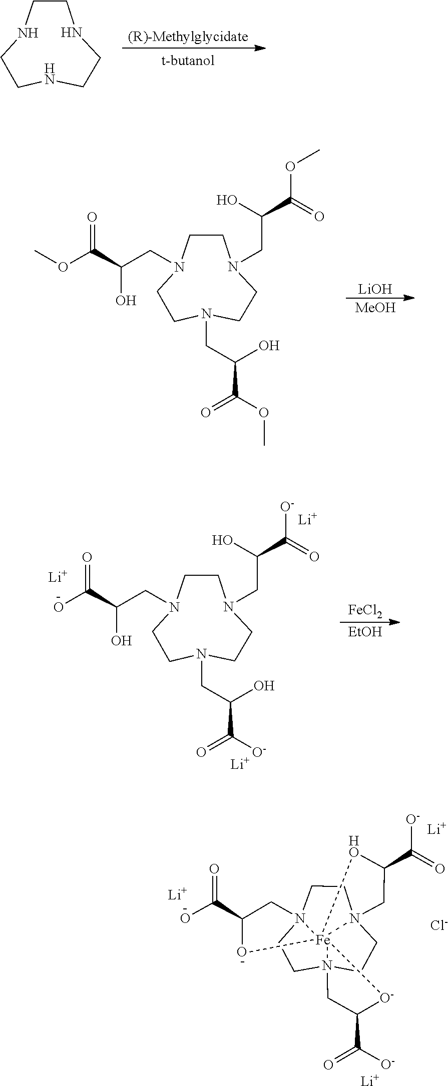

TACN (0.100 g, 0.773 mmol) was dissolved in 10 mL t-butanol and stirred in a 20 mL scintillation vial. (R)-Methylglycidate (332 μL, 7.73 mmol) was added and solution stirred overnight for about 12 h. Solvent was removed under pressure to yield crude trimethyl 3,3′,3″-(1,4,7-triazonane-1,4,7-triyl)(2R,2′R,2″R)-tris(2-hydroxypropanoate) as a red oil. Crude used in consequent reaction without further purification. ESI-MS (m/z): [M−H]⁺=436.82 (100).

3,3′,3″-(1,4,7-triazonane-1,4,7-triyl)(2R,2′R,2″R)-tris(2-hydroxypropanoate) (crude oil from previous step) was stirred in a 4 mL solution of MeOH:2M LiOH overnight for about 12 h. Solvent was removed under pressure and producing crude (2R,2′R,2″R)-3,3′,3″-(1,4,7-triazonane-1,4,7-triyl)tris(2-hydroxypropanoate) (L5) as a red-brown solid. Crude used in consequent reaction without further purification. ESI-MS (m/z): [M−2H]⁻=392.33.

(2R,2′R,2″R)-3,3′,3″-(1,4,7-triazonane-1,4,7-triyl)tris(2-hydroxypropanoate) (L5) (0.195 g, 0.473 mmol) was dissolved in 5 mL water and stirred in a 20 mL scintillation vial. Solution was heated at about 60° C. Anhydrous Ferrous Chloride (0.060 g, 0.473 mmol) was dissolved in 5 mL water and slowly added to the solution. Solution was stirred overnight for about 12 h. Solvent was removed under pressure to yield crude [Fe(L5)Cl] as a red-brown solid. ESI-MS (m/z): [M−Cl]⁻=481.18 (100).

### Example 6

This example provides a description of synthesis of macrocycles of the present disclosure.

### Synthesis of 1-benzyl(2-sulfonate), 4,7-bis(2-hydroxypropyl) 1,4,7-triazacyclononane

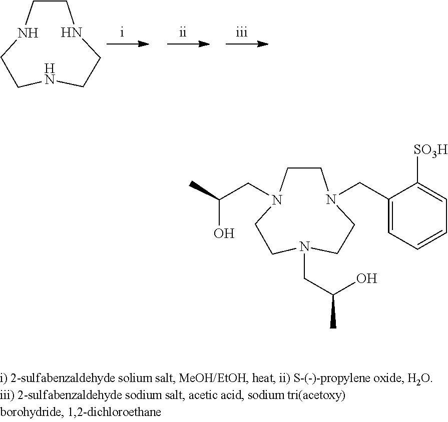 [SMILES: C1CNCCNCCN1 ; C[C@H](O)CN1CCN(Cc2ccccc2S(=O)(=O)O)CCN(C[C@H](C)O)CC1]

198 mg of 1,4,7-triazacyclononane (TACN, 1.55 mmol) is stirred in 10 mL ethanol. At that time, 2-sulfobenzaldehyde sodium salt is dissolved in 14 mL methanol, and added dropwise to the reaction. The reaction is set to reflux for two hours, wherein the reaction changes color to pale yellow. The reaction is taken off heat and allowed to cool with stirring. Crude material is columned and fractions that show a m/z of 321 on negative mode [M−H⁺+Na⁺] were pooled and reacted with 3.6 equivalents of s-(−)-propylene oxide in water. After one week, data showed abundance of bis (2-hydroxypropyl) ligand (m/z=246 [M+H⁺], and so crude was dried and redissolved in 1,2-dichloroethane with 1 equivalent of 2-sulfobenzaldehyde sodium salt (0.197 g) and 1 equivalent of glacial acetic acid (54 μL) and allowed to stir for an hour before adding 1.2 equivalents of sodium tri(acetoxy)borohydride (240 mg) that, within an hour, begins to show formation of product. Two days later, the reaction is quenched with 20 mL 1 M NaOH, and the dichloroethane separated from the aqueous. The aqueous is washed with dichloromethane (30 mL) and the organic is dried down. Organic oil is then columned on silica using ethyl acetate:methanol: 10% aqueous ammonium hydroxide. Column isolated as yellow oil. Mass Spectrometry (ESI): m/z=416 [M+H⁺], 438 [M+Na⁺].

Coordination of iron to 1-benzyl(2-sulfonate), 4,7-bis(2-hydroxypropyl) 1,4,7-triazacyclononane.

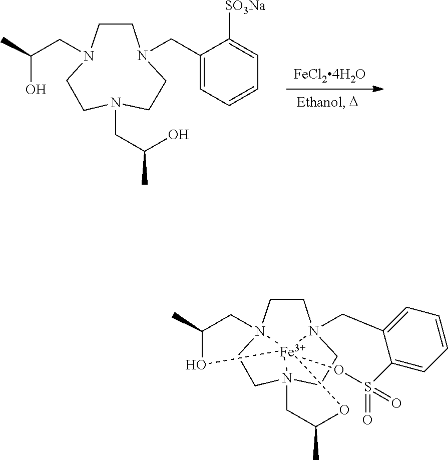

Crude ligand (44.6 mg, 0.102 mmol assuming sulfonic acid) was set to stir in 2 mL ethanol. Then, 31.7 mg of ferrous chloride tetrahydrate (0.159 mmol) was dissolved in 1 mL ethanol. The iron solution was added to the solution of stirring ligand, and allowed to stir under heat. Solid is isolated via precipitation with diethyl ether, and further drying on speedvac. MS, ESI: 469.8 [Fe(L−2H⁺)⁺].

Preparation of 1-formyl, 4,7-bis(2-hydroxypropyl) 1,4,7-triazacyclonane.

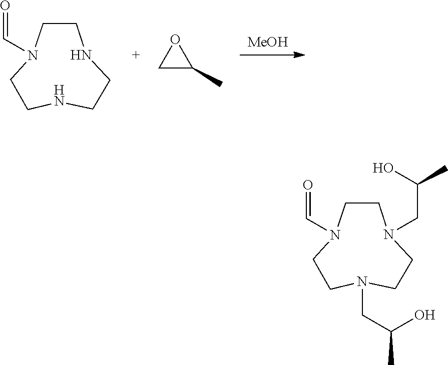

The synthesis of 1-formyl 1,4,7-triazacyclononane is already published previously (Creaser, P. et al. Aust. J. Chem. 2003, 56, 61-64). To 0.9925 g of this material, is added 993 μL of 98% s-(−)-propylene oxide in 10 mL methanol overnight. The following day, crude is dried down and columned on silica using EtOAc:MeOH (0-60% MeOH). Product isolated dried down to 1.433 g oil, and has M/Z=274 [M+H⁺]

Preparation of 1,4-bis(2-hydroxypropyl) 1,4,7-triazacyclononane.

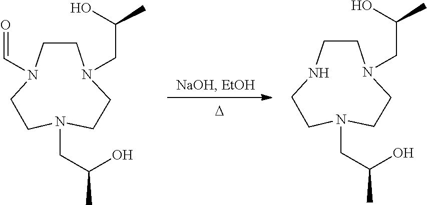

Product of the previous step is dissolved in 30 mL 92% Ethanol, 5 mL water, with 2.5 g NaOH and stirred at 75-80° C. After 4 days the reaction is taken off the heat, and allowed to cool before adding 20 mL water and washing with chloroform (100 mL in 3 washes). Organic washes are dried over sodium sulfate and filtered before being dried down to oil, 1.156 g. Mass spec, +ve mode, m/z=246 [M+H⁺].

Preparation of 1-(benzyl-2-sulfonate)-4,7-bis(hydroxypropyl) 1,4,7-triazacyclononane).

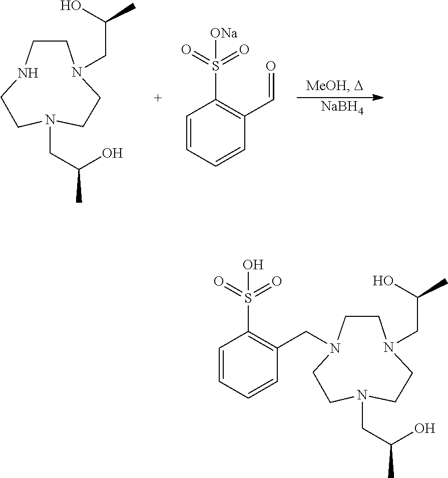

Approximately 50 mg of oil (0.204 mmol) is collected by dissolving the crude in 12 mL methanol, 500 μL drawn up and diluted to 5 mL with methanol. To this solution is added 88 mg 2-sulfobenzaldehyde sodium salt (0.423 mmol), and the reaction is set to near reflux for two hours on a hot plate. After 2 hours, the reaction is allowed to cool before adding 31 mg sodium borohydride (0.819 mmol) and stirred for one hour. Afterward, the reaction is quenched with 3M HCl until bubbling stops, and then the solution is basified with 10M NaOH. The solution is diluted further with 5 mL water. Product is extracted in dichloromethane, and dried down. The organic oil was then purified on silica using 8:2:1→3:2:1 EtOAc:MeOH: 10% aqueous ammonium hydroxide to yield yellow oil, Mass spec M/Z (+ve mode)=416.3 [M⁻+2H+], 438.3 [M⁻+H⁺+Na⁺].

### Example 7

This example provides a description of synthesis of macrocycles of the present disclosure.

Addition of Phenacyl Bromide and reduction to alcohol.

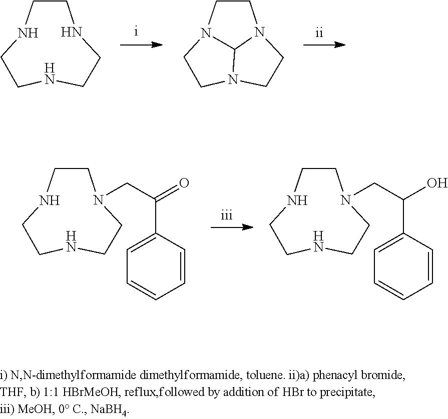 [SMILES: C1CNCCNCCN1 ; C1CN2CCN3CCN1C23 ; O=C(CN1CCNCCNCC1)c1ccccc1 ; OC(CN1CCNCCNCC1)c1ccccc1]

Crude protected TACN ligand (7.05 mmol) is dissolved in 15 mL tetrahydrofuran, filtered to remove insoluble material, and then set to stir in a round bottom flask. Then, phenacyl bromide (1.447 g, 7.27 mmol) is dissolved in 20 mL THE and added via addition funnel over the course of 45 minutes. In that time, a solid began to form and the reaction turned milky. It was stirred overnight, and the following day the solid was filtered to give a beige solid that was washed with 60 mL diethyl ether. The material was then dissolved in 100 mL of a 1:1 MeOH:HBr solution and refluxed for 6 hours. Afterward, the reaction was cooled to room temperature before an additional 50 mL HBr was added. The reaction was allowed to sit in the freezer overnight. The resulting solid was collected via filtration and washed with diethyl ether to give 6.64 g of an HBr salt.

2.36 g of this solid is then dissolved in 25 mL water, neutralized with 0.35 g NaOH, and an additional pellet added to bring the pH of the solution >10. The resulting solution is washed with chloroform (3×30 mL). Organic layers are pooled together, dried over sodium sulfate, and filtered before being brought to dryness over a rotary evaporator, mass of oil 0.5661. Mass spec m/z=248 [M+H⁺]. This oil is then set to stir in 25 mL methanol in a round bottom flask placed in an ice bath (<10° C.). To the cold solution stirring is added 1.88 g sodium borohydride (NaBH₄, 49.7 mmol), slowly. The reaction was stirred in the ice bath for 10 minutes before removing the bath and allowing the reaction to stir at room temperature for 3 hours. When the data by mass spectrometer showed that the reaction had gone to completion (M/Z=250 [M+H⁺]), the solution was neutralized with hydrochloric acid (HCl) to quench remaining borohydride. Then, sodium hydroxide pellets (8 pellets) are added to raise the pH of solution above 10, and then the product is collected via aqueous-organic extraction (chloroform, 3×50 mL). Organic layers are pooled, dried over sodium sulfate, and filtered before being rotovapped to an oil, in a massed round-bottom flask, to 0.3431 g. This oil is used in the next step without further purification.

Addition of Hydroxypropyl Groups.

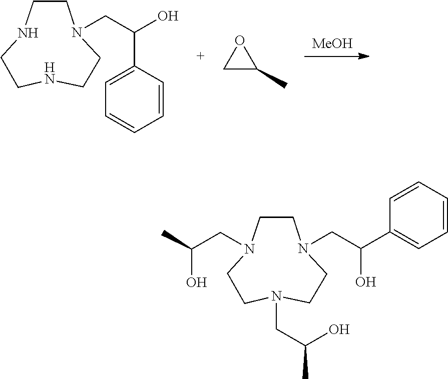

The crude isolated in the previous step (0.3431 g) is dissolved in 15 mL methanol, and 2.4 equivalents of 98% s-(−) propylene oxide (236 μL) are added via pipettor. The following day, an additional 300 μL of s-(−)-propylene oxide are added to complete the reaction. On the third day, the reaction is brought to dryness. Ligand is purified by column chromatography on basic alumina (0-5% MeOH in dichloromethane). Mass Spec: M/Z=366. [M+H⁺].

Metalation of Ligand (L) with Iron.

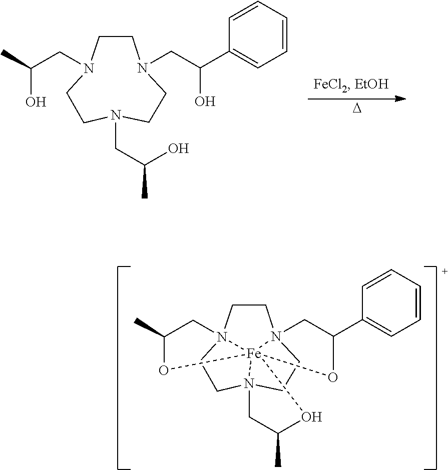

The ligand (0.0433 g, 0.1186 mmol) was dissolved in minimal ethanol. One equivalent of anhydrous ferrous chloride (0.015 g) was dissolved in ethanol and added to the ligand. Solution is heated while stirring, and product is isolated via precipitation with diethyl ether. MS: 419 [Fe(L−2H⁺)⁺]. Complex run on 1.4 T benchtop NMR at 33° C., pH 7.2, to give r₁=1.6 mM⁻¹ s⁻¹.

### Synthesis of bis(glycidate) Phenyl Alcohol TACN

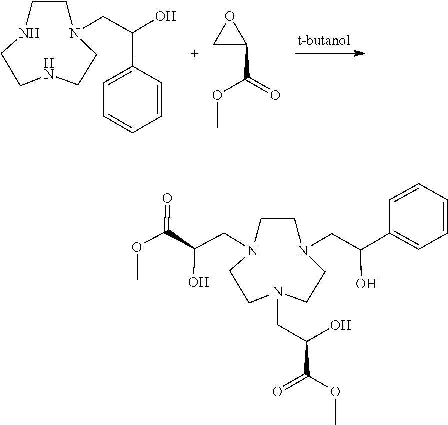

To 0.578 g of phenyl alcohol TACN in 15 mL tert-butanol is added 1.26 mL R-methylglycidate. The reaction is set to stir overnight. The following day, mass spectral analysis shows a major peak of product (M/Z, ESI=454 [M+H⁺]). Major peak remained in material that was insoluble in ethyl acetate (following 6 successive washes with ethyl acetate). 670 mg of the material was isolated. This was used in the next step.

Deprotection of Glycidate.

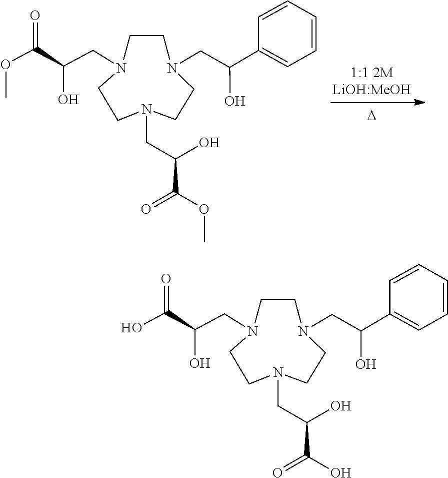

Product from the previous step was dissolved in 18 mL methanol, and then added to 18 mL of 2 M LiOH solution. Reaction was set to 50° C. and stirred overnight. The following day, mass spectroscopy analysis shows conversion to product. (M/Z, ESI⁺ mode: 426.3 [M+H⁺], negative mode: 424.2 [M−H⁺].)

### Example 8

This example provides a description of synthesis of macrocycles of the present disclosure.

TAFO Ligand:

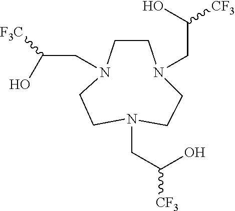 [SMILES: OC(CN1CCN(CC(O)C(F)(F)F)CCN(CC(O)C(F)(F)F)CC1)C(F)(F)F]

To a 50-mL roundbottom flask, TACN (0.2449 g, 1.90 mmol) was added and then dissolved in ethanol. Then 0.6765 g (6.24 mmol) of 1,1,1-Trifluoro-2,3-epoxypropane was added to the flask and allowed to stir overnight at room temperature. Solvent was removed by rotary evaporation to yield a pale yellow oil. ESI-MS m/z: 466.32 (100%) [M+H⁺]⁺. ¹H NMR (400 MHz, CDCl₃, 25° C.): δ 1.22 (t), 2.46-2.99 (m), 3.69 (q), 4.04 (s).

FeTAFO:

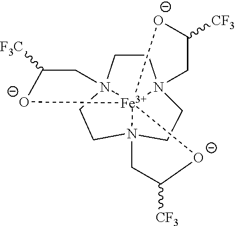

To an ethanolic solution of “TAFO” ligand (0.1335 g, 0.287 mmol), an equimolar ethanolic solution of FeCl₂·4H₂O (0.0582 g) was added and the mixture was allowed to stir at room temperature overnight. After 24 h, 0.0241 g of FeCl₂·4H₂O was added to the reaction mixture. Upon completion, ethyl ether was added to the reaction mixture to precipitate out the complex as a yellow solid, which was then washed twice with ethyl ether. ESI-MS m/z: 519.36 (100%) [M+H⁺]⁺.

NitroBzTAFO Ligand:

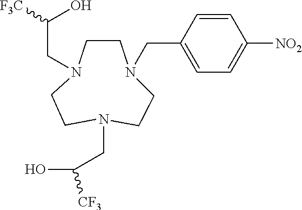 [SMILES: O=[N+]([O-])c1ccc(CN2CCN(CC(O)C(F)(F)F)CCN(CC(O)C(F)(F)F)CC2)cc1]

0.0486 g (0.483 mmol) of “NitroBzTACN” ligand was added to a scintillation vial and dissolved in ethanol. Then 0.1624 g (1.45 mmol) of 1,1,1-Trifluoro-2,3-epoxypropane was added and the reaction was allowed to stir at room temperature overnight. After 24 h, 0.1624 g of 1,1,1-Trifluoro-2,3-epoxypropane was added and the reaction mixture was stirred overnight at room temperature. Solvent was removed by rotary evaporation to yield a pale yellow oil. ESI-MS m/z: 489.40 (100%) [M+H⁺]⁺.

NitroBzTAFO Complex:

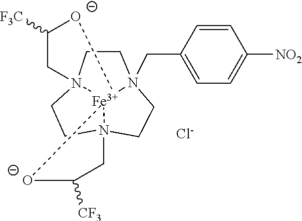

70.1 mg (0.144 mmol) of “NitroBzTAFO” ligand was added to a scintillation vial and dissolved in ethanol. An ethanolic solution of FeCl₂·4H₂O (0.0316 g, 5% mol excess) was added and then allowed to stir overnight at room temperature. After 24 h, 0.0377 g of FeCl₂·4H₂O was added to the reaction mixture and stirred overnight. Upon reaction completion, ethyl ether was added to the reaction mixture to precipitate out the complex as a yellow solid and was then washed twice with ethyl ether. ESI-MS m/z: 542.40 (100%) [M−Cl⁻]⁺.

### Example 9

This example provides a description of synthesis of macrocycles of the present disclosure.

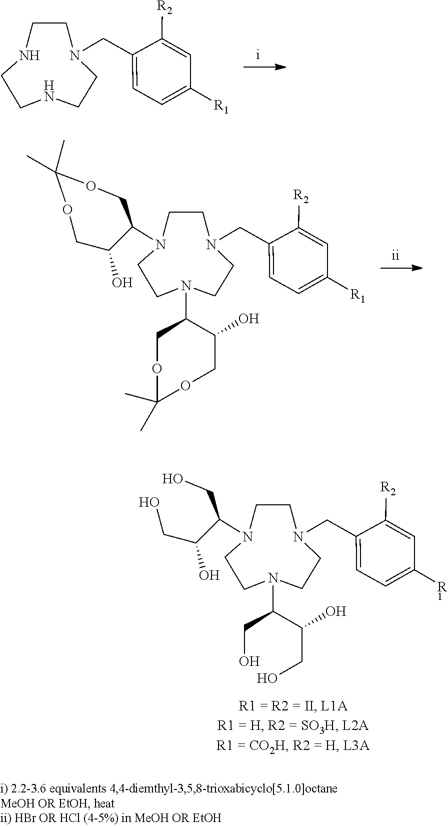

General Procedures.

The ligands are prepared as either their mono- or di-substituted 1,4,7-triazacyclononanes prior to addition of the polyhydroxylated materials. Then, in alcohol, the crude ligand stirs with 1.2-3.6 equivalents of 4,4-dimethyl-3,5,8-trioxabicyclo[5.1.0]octane under heat. Between 2-24 hours, the reaction is complete. Ligand is either purified via column chromatography, or filtered to isolate the solid material. The protected ligand is then deprotected using dilution of concentrated aqueous acid—hydrobromic or hydrochloric—either refluxed in water, or mixed in alcohol (methanol or ethanol) at room temperature. Purification of the liberated polyhydroxylated ligand utilizes neutralization and extraction, or column chromatography. Finally, the iron complexes are prepared by stirring the ligand in ethanol and adding an ethanolic solution of 1) anhydrous ferrous salts, which oxidize to form the ferric complexes for ligands that lack coordinating ancillary groups or 2) ferric salts (FeCl₃) for ligands that have coordinating ancillary groups such as L6A. The material is allowed to stir until complete conversion to metal complex is observed by using mass spectrometry.

Longitudinal Relaxation Time (T₁) and Relaxation Rate (R₁) Determination

Serial dilutions of iron complex are prepared from a stock solution containing 5-10 mM complex (with or without 2 equivalents meglumine) in 100 mM NaCl and 10-20 mM HEPES buffer at pH 7.2. The three concentrations and a blank are tested on a Nanalysis 60Pro NMR set to run with a magnet temperature of 33° C. locked with a deuterated solvent. After calibrating the instrument for the solvent, the proton spectrum of the sample is collected and processed to focus on the water signal. Then the experiment on a single sample are run multiple times focusing on i) the appearance of the water signal on the final scan, ii) the number of data points on the plateau of the curve of peak integration over time, and when iii) increasing delay time does not change the relaxation time, given by the instrument in seconds. The concentrations given are then plotted on a graph of concentration (x-axis) vs the inverse of the relaxation time (1/T₁, s⁻¹). The slope of the regression line is then interpolated as the relaxation rate, R₁, of the complex under these conditions.

Syntheses:

Benzyl TACN and Benzoate TACN were both synthesized previously.

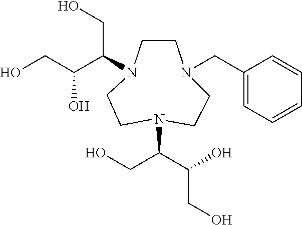 [SMILES: OC[C@@H](O)[C@@H](CO)N1CCN(Cc2ccccc2)CCN([C@H](CO)[C@H](O)CO)CC1]

### Synthesis of the L1A Ligand Shown Above

Step 1. Procedure to make bis(tri(hydroxy)butyl)-protected benzyl TACN with formal name of (5S,6R)-6-(4-benzyl-7-((5R,6R)-6-hydroxy-2,2-dimethyl-1,3-dioxepan-5-yl)-1,4,7-triazonan-1-yl)-2,2-dimethyl-1,3-dioxepan-5-ol.

Benzyl TACN is dissolved in water and basified to pH 10. The ligand is then extracted into chloroform, and the organic solution is dried with sodium sulfate and filtered before being evaporated down to oil. The oil is weighed on a balance to determine moles of starting material. The 675 mg of material is stirred in 6 mL absolute ethanol and under heat (70° C.). To the stirring solution is added 2.2-3.6 equivalents of 4,4-dimethyl-3,5,8-trioxabicyclo[5.1.0]octane via a pipettor (1.44 mL). The reaction is allowed to stir under heat overnight in a flask fitted with a water condenser. The following day, analysis by using mass spectrometry gives the desired peak (508 m/z ratio, M+H⁺), and the reaction is stopped and the solution is dried to oil. The oily material is then dissolved in organic solvent and loaded onto a silica column (˜15-20× silica by mass crude) and passed through the column. First, 4-4.5 column volumes (CV) of 4:1 hexanes:ethyl acetate are passed to remove excess unreacted epoxide, then 1 CV of 1:4 hexanes:ethyl acetate. Then, 3 CV of 8:2:1 ethyl acetate:methanol:aqueous ammonium hydroxide (10%) is passed through the column to elute the product, which has a retention factor of 0.35-0.5 in the 8:2:1 solution.

Step 2. Deprotection of Epoxides to Produce Hydroxyl Groups.

The ligand is stirred in a solution of 3% acid (HBr or HCl) in alcohol (methanol or ethanol) and shaken for about 3-5 minutes. The solution is analyzed by mass spectrometry to ensure deprotection of the group (m/z: 428 M+H⁺). The ligand solution is allowed to stand overnight to form a solid. If no solid precipitates, then the solution is worked up for product purification. The acidic solution is neutralized with sodium hydroxide solution, and then the solution is concentrated on a rotary evaporator. The resulting solid is then extracted with absolute ethanol, and ethanolic solution transferred into a flask, and dried down to oil.

Example of Deprotection Purification Via Neutralization

800 mg of protected ligand was stirred in acidic alcohol solution (4% HCl in EtOH). After analysis by mass spectrometry shows the desired product (m/z 428, M+H⁺) the solution is basified with potassium hydroxide and the solvent is removed under vacuum. Then, the crude salt is washed with absolute ethanol and loaded into centrifuge tubes, where samples are spun down to collect the solution away from unwanted sodium chloride. The liquid is placed in a tared vial and brought to dryness, and then placed under vacuum. The final mass of the material in the vial was 418 mg to be used in metalation.

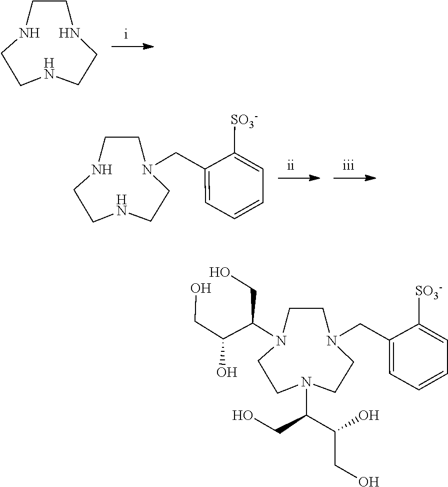

L2A Synthesis.

Step 1. Reductive Amination of methyl(2-sulfonate)TACN

152.8 mg 1,4,7-triazacyclononane is stirred in a 2-neck round bottom flask in 40 mL 1,2-dichloroethane. Then, 1 equivalent of 2-sulfobenzaldehyde sodium salt (208.16 g/mol FW, 246.2 mg white powder) is added to the flask, and then 1 equivalent of glacial acetic acid (0.068 mL, 68 μL) via pipettor. The reaction is stirred overnight. The following day, approximately 3.0 equivalents of sodium tri(acetoxy)borohydride (211.94 g/mol, 760 mg white powder) are added to the flask, to initially produce a clear solution until turbidity returned after 20 minutes. The reaction is allowed to stir for another 2 days, after which the reaction is filtered and the solid collected for analysis by mass spectrometry (ESI-MS=300 m/z, [M⁻+2H⁺], M⁻ represents the sulfonate anion derivative of the product). The crude product is washed with 10 mL 92% ethanol to remove acetate, and the solid is used in the next step of the synthesis without further purification.

Step 2. Addition of 4,4-dimethyl-3,5,8-trioxabicyclo[5.1.0]octane to give 2-((4-((5R,6R)-6-hydroxy-2,2-dimethyl-1,3-dioxepan-5-yl)-7-((5R,6S)-6-hydroxy-2,2-dimethyl-1,3-dioxepan-5-yl)-1,4,7-triazonan-1-yl)methyl)benzenesulfonate.

204.7 mg of the sulfonate TACN material from the previous step (FW=299 g/mol, 0.685 mmol) is set to stir in 1:1 methanol:water (MeOH:H₂O, 10 mL). One pellet of NaOH is added to basify the solution. Then, 2.2 equivalents of 4,4-dimethyl-3,5,8-trioxabicyclo[5.1.0]octane are added (197 μL). The reaction is stirred and heated between 58-64° C. overnight. The following day, the reaction is cooled to RT and mass spectrometry was used to analyze the product. Major product is m/z 610 [M+Na⁺] followed by m/z 588 [M+H⁺] where M is neutral ligand. The reaction is then washed with ethyl acetate (EtOAc, 30 mL, then 20 mL), organic layers are pooled together, dried with sodium sulfate, and filtered. The material is then run on a column (8 g silica, CV determined to be approximately 22 mL) and the crude is eluted first with 100% EtOAc (approximately 2.5 column volume), then 8:2:1 ratio EtOAc:MeOH: 10% aqueous ammonium hydroxide (approximately 3.1 column volume) and finally with 2 column volumes of 3:2:1 EtOAc:MeOH: 10% aqueous ammonium acetate. (Rf product in 8:2:1 EtOAc:MeOH: 10% aqueous ammonium hydroxide, 0.3-0.4).

Step 3. Deprotection to produce 1,2,4-trihydroxybutane pendants to give ligand: 2-((4,7-bis((2R,3 S)-1,3,4-trihydroxybutan-2-yl)-1,4,7-triazonan-1-yl)methyl)benzenesulfonate.

Material purified above (160.8 mg ammonium L3A precursor, 0.266 mmol if correct) is dissolved in 1:2 mixture of hydrobromic acid (HBr) and acetic acid (AcOH) (500 μL HBr, 1 mL AcOH) and shaken for 3 minutes before placing in the freezer. If no precipitate forms, 1-2 mL of 92% ethanol is added and the reaction is allowed to stand in the hood. Crude material is purified by drying down the reaction to oil and was redissolving in water. The reaction is then chromatographed on 3.5 g Dowex 50WX4 cation exchange resin, H⁺ form. Material is loaded on the resin, and 30 mL water is passed through, before eluting product with a 5% aqueous ammonium hydroxide solution. Product elutes after adding ammonium to the column. The crude product is dried in a tared vial and placed on vacuum overnight. Final mass of product is 110.3 mg (0.210 mmol, 79% yield if ammonium salt of product). m/z: 508 [M+H⁺], 530 [M+Na⁺] where M is the neutral ligand.

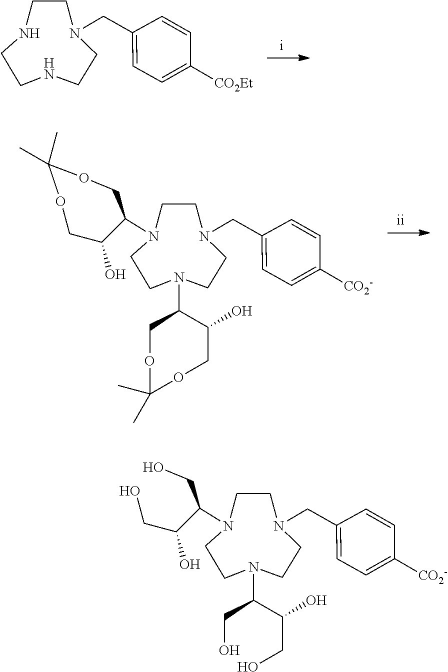

### Synthesis of the L3A Ligand

Step 1. Addition of the protected tri(hydroxy)butyl groups. Protected ethyl 4-((1,4,7-triazacyclononan-1-yl)methyl)benzoate hydrobromide salt product (3.195 g) is deprotected by stirring in 45 mL of 1M NaOH, with minimal methanol added to solubilize the reaction. The reaction is set to stir at 60-70° C. for 2 days. Then, the reaction is dried down and redissolved in minimal ethanol (7 mL). Then, 3.6 equivalents of 4,4-dimethyl-3,5,8-trioxabicyclo[5.1.0] octane are added (3.98 mL), and the reaction is set to stir under heat. Within hours, the reaction solidifies to give an off-white solid. Reaction mixture is filtered to separate solid from liquid. The mass spectrum of the solid was consistent with product (m/z ratio 552=M+H⁺), which is used in the next step of the reaction.

Step 2. Deprotection to Produce Tri(Hydroxy)Butyl Pendants

The solid material from above is dissolved in 30 mL of a 5.6 M HCl solution (14 mL HCl, 16 mL water) and set to stir overnight under a condenser, temperature set at 70° C. The next morning, the solution is analyzed by mass spectrometry and shows the major product (ESI-MS m/z 472 [M+H⁺]). Ligand crude is neutralized and purified. The crude solid is loaded on a silica column, and eluted by using a gradient of methanol in ethyl acetate with 1% formic acid added to the eluent. Product elutes between 50-100% methanol. To remove the unwanted formic acid side materials, the ligand is then run thorough a Dowex 50WX4 ion-exchange resin. The loaded column is first washed with water, and then ligand elutes in 5% aqueous NH₄OH. Material is redissolved in water:ethanol, and left overnight. The following day, compound is dried down under vacuum to produce oil.

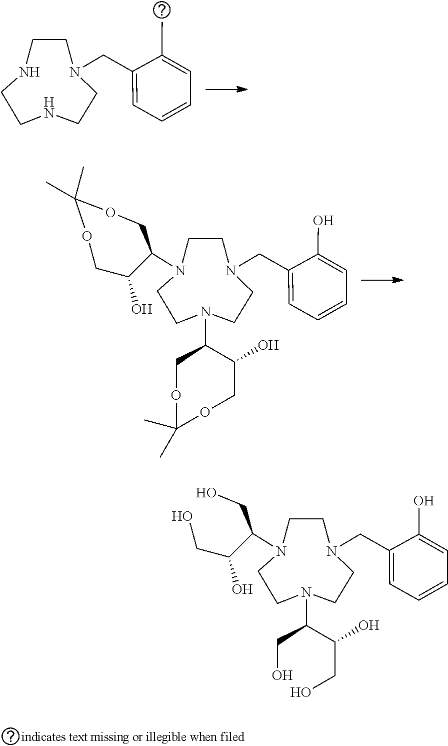

### Synthesis of the L6A Ligand

Step 1. Addition of protected alcohols towards product: (5S,6R)-6-(4-((5R,6R)-6-hydroxy-2,2-dimethyl-1,3-dioxepan-5-yl)-7-(2-hydroxybenzyl)-1,4,7-triazonan-1-yl)-2,2-dimethyl-1,3-dioxepan-5-ol A crude reductive amination of 500 mg 1,4,7-triazacyclononane with salicylaldehyde in methanol is set to stir under heat (55° C. by thermometer) with 2.2 equivalents of 4,4-dimethyl-3,5,8-trioxabicyclo[5.1.0]octane (1.11 mL) for two hours, before adding 1 pellet of sodium hydroxide. The reaction stirs over the weekend, and is checked on mass spectrometry to show 525 (M+H⁺) as major product. A solid precipitate is filtered off and discarded, and the methanolic solution is dried down to half. The solution is then diluted in 10 mL water and washed with dichloromethane (15, 20, 15 mL). Organic layers are pooled, washed with sodium sulfate and dried down to oil. The material can be purified via column chromatography (100% Ethyl Acetate—3:2:1 EtOAc:MeOH: 10% aqueous Ammonium Hydroxide) with desired product coming between 6:3:1→3:2:1 EtOAc:MeOH:NH₄OH (10%_{aq})

Step 2. Deprotection of alcohols. Column fractions of product are combined and washed (10 mL H₂O: 10 mL dichloromethane) and the organic layers are dried with sodium sulfate and filtered before being evaporated in a round bottom flask. The orange oil that comes from the organic is shaken in 1 mL concentrated HCL diluted to 10 mL with methanol for 5 minutes. After 5 minutes, 10 mL water is added, and the reaction is washed with 75 mL chloroform (25 mL×3). The organic layer is discarded, and the aqueous layer neutralized with 10 M NaOH. This material is then washed again with 75 mL CHCl₃, and the organic again discarded. The aqueous is brought to dryness and the salty residue is washed with 30 mL absolute ethanol. The solution is filtered and dried down to yield approximately 400 mg solid, ESI-MS m/z: 444.7 (M+H⁺)

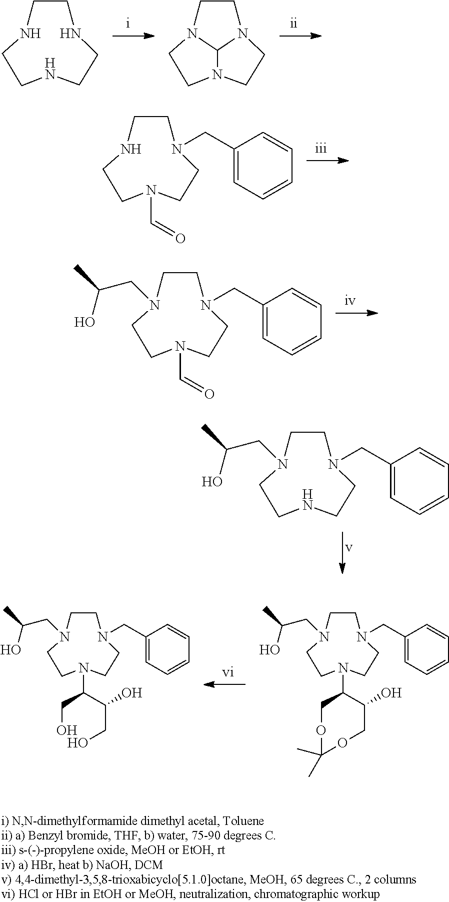

### Synthesis of L1B Ligand

Steps 1 & 2. TACN Preparation (1-benzyl-4-formyl-1,4,7-triazacyclononane)

In a round bottom flask stirs 0.999 g 1,4,7-triazacyclononane and 1.1 equivalent N,N-dimethylformamide dimethyl acetal (1.165 mL) in 15 mL toluene. The reaction stirs for 24 hours at room temperature. Afterward, the solution is decanted into another round bottom flask and dried on a rotary evaporator. After, the crude oil (1.0142 g of material, 94% yield) and is set to stir in 15 mL tetrahydrofuran (THF, dried over sieves). 1 equivalent of Benzyl Bromide (877 μL) is added to another 20 mL THF and added dropwise over the course of 25 minutes. The reaction is sealed to stir, forming a white product quickly. The following day, the solid is collected via vacuum filtration and washed with minimal diethyl ether. The solid is placed in a clean round bottom flask, dissolved in about 12 mL distilled water, and set to stir for 48-72 hours at 76° C. The crude material is then brought to dryness to form oil. Mass Spec (ESI-MS) m/z: 248 (M+H⁺) and assumed 100% conversion from the benzyl addition.

Step 3. Addition of Alcohol 1 to give (S)-4-benzyl-7-(2-hydroxypropyl)-1,4,7-triazonane-1-carbaldehyde. The material from the previous step is dissolved in 20 mL 92% ethanol and 1.2 equivalents of S-(−)-propylene oxide (624 μL) are added to the stirring reaction, along with 5 mL of a 1 M NaOH solution to induce product formation. The reaction stirs overnight, and the following day is analyzed by using mass spectrometry (m/z=306, M+H⁺). The material is used without further purification.

Step 4. Removal of Aldehyde. The material from step 3 is set to stir under heat for 6 hours with 20 mL of 48% HBr added to the reaction to remove the aldehyde and decompose any residual propylene oxide. Afterward, another 20 mL of acid is added to the reaction flask and placed in the freezer overnight. The following day, the solution is made basic with 20 g NaOH, followed by washing the crude twice with 50 mL dichloromethane. The organic layer is collected, dried with sodium sulfate, and dried via rotary evaporator to produce oil (m/z 278, M+H⁺, as well as impurity of 336 m/z) Crude reaction material is used in the next step (1.723 g)

Step 5. Addition of protected tri(hydroxy)butyl. To this crude, 0.85 equivalents of 4,4-dimethyl-3,5,8-trioxabicyclo[5.1.0]octane are added (0.85 equivalents from starting 7.296 mmol of earlier step, 810 μL) to the ligand stirring in hot methanol (65° C.). The following day, analysis by using mass spectrometry shows conversion to product. Ligand is then purified via two columns. The ligand is dissolved in 92% ethanol and run through a column using column volumes (CV) of 100% EtOAc, then 1 CV of 9:1:1 EtOAc:MeOH: 10% aqueous NH₄OH, 2 CV of 8:2:1, and finally 1 CV of 3:2:1 EtOAc:MeOH:NH₄OH_{(aq)}. Fractions that showed product on mass spec were recombined and dried down to run on basic alumina, using a gradient of 0-15% MeOH in DCM. Product is the major product in two fractions collected, and with minimal impurity were collected and dried down to an oil to be used in the next step, resulting in 320.9 mg of oil with m/z 422 [M+H⁺], a 9.8% yield from starting TACN.

Step 6. Deprotection of tri(hydroxy)butyl pendant. The protected ligand from step 5 is set to shake in 3% HCl in EtOH (4 mL HCl in 129 mL 92% EtOH) and set aside. The following day, the solution is neutralized with an aqueous sodium hydroxide solution (5.1 M) dropwise, and monitored by pH meter. When the pH meter read approximately 7.7, the material is dried down, redissolved in 50 mL H₂O, and washed with 80 mL dichloromethane to remove unwanted side product. Aqueous material is kept and dried under vacuum. This material is then redissolved in chloroform:methanol and run on a silica column from 0 to 100% Methanol in CHCl₃. Product eluted from 20% to approximately 50% methanol. Fractions of interest are collected and solvent removed. The resulting solid is washed with ethanol (92%) to give 130.4 mg of product by massing in a tared vial. Material is dried under vacuum and analyzed. (m/z ratio: 382=M+H⁺).

General procedure for complexation: Ligand is stirred in 2 mL absolute ethanol and gently heated. Then, less than one equivalent of iron (ferrous chloride or bromide salt) is added to the reaction via addition of salt dissolved in absolute ethanol. Iron solution is slowly added to the stirred ligand solution via pipette and monitored by mass spec to observe complexation. The rest of the equivalent of iron salt is added after about a day and allowed to keep stirring until <10% free ligand was observed by using mass spectrometry. Ethanolic sodium hydroxide solution can be added to deprotonate the ligand and induce formation of the iron complex, if needed. Formation of the iron complex is identified by ESI-MS. ([M⁺], for L1A=481, L2A=561, L3A=525, L6A=497 ([ML]+H⁺) L1B=435).

### Example 10

This example provides a description of synthesis of macrocycles of the present disclosure. In the following, L denotes the neutral ligand in the mass spectral analysis.

Metalation of TOB Half, Fe(TOB Half):

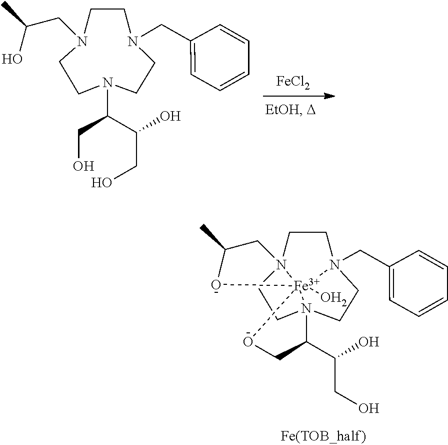

First, 30 mg of ligand shown above (FW=381.5 g/mol, 0.079 mmol) is dissolved in 2 mL ethanol. The ligand solution was spun down via centrifuge to remove any insoluble material, and then set to stir on heat. Then, 8 mg anhydrous ferrous chloride (FW=126.75, 0.063 mmol) was dissolved in an additional 1.5 mL ethanol, and added to the stirring ligand via pipette. The following day, 260 μL from a stock solution of ferrous chloride (0.06 M) was added to the solution, bringing the total amount of iron to 0.085 mmol, or 1.08 equivalents. Product is isolated following ether trituration, and subsequently dried down to a yellow solid. Mass Spec: 435.9 [Fe(L−2H⁺)]⁺. Compound was analyzed on 1.4 T Benchtop NMR at 33° C. for proton relaxivity to give an r₁ value of 1.7 mM⁻¹s⁻¹. Here L is the neutral ligand.

Metalation of TOB2, Fe(TOB2):

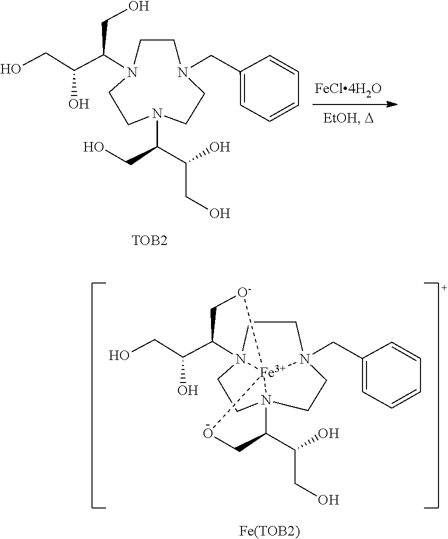

36.3 mg of TOB2 oil (FW=427 g/mol; 0.085 mmol) was dissolved in 3 mL ethanol and set to stir on a hot plate. Then, 17 mg ferrous chloride tetrahydrate (FW: 198.81; 0.085 mmol) was added. The reaction was allowed to stir in heated ethanol overnight. The next day, another 15 mg (0.075 mmol) was added in 1 mL ethanol and stirred overnight. The following day, the solution was removed from heat and a solid product was formed. The solid was triturated with diethyl ether. Yellow solid obtained. ESI-MS: 481.5 [Fe(L−2H⁺)⁺].

Metalation of sulbot2, Fe(sulbot2):

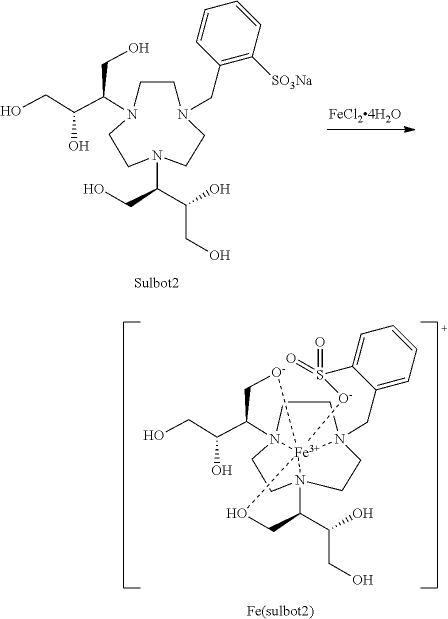

In 2 mL ethanol was stirred 30.2 mg sulbot2 ligand (NH₄⁺ salt, from Dowex resin in previous step, FW of [NH₄(L)]=524.6 g/mol, 0.058 mmol) and set to stir on heat. Then, 10.8 mg of ferrous chloride tetrahydrate (FW 198.81, 0.054 mmol) was dissolved in 2 mL ethanol and added to the stirring ligand dropwise. The following day, the reaction was taken off heat, and allowed to stir an additional 2 days. Afterward, the solution was collected and spun down via centrifuge to remove white precipitate. The solution was then triturated with diethyl ether to isolate a solid that shows desired product on mass spec (20.2 mg). MS: 561.4 [Fe(L−2H⁺)⁺] with ˜25% 583.3 [Fe(L−3H⁺)+Na⁺] and 12% 508.6 [L+H⁺].

Metalation of TOBA2, Fe(TOBA2):

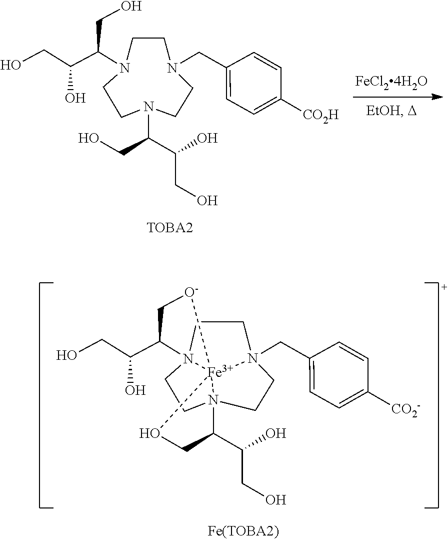

A mass of 24 mg ligand L3A (FW=471.5 g/mol; 0.51 mmol) was dissolved in 2 mL ethanol, with minimal water to solubilize material. Then, 1.5 equivalents of ferrous chloride tetrahydrate were added (FW 198.81 g/mol, 15 mg) and set to stir on heat. Yellow solid that precipitated was collected, washed with diethyl ether, and dried down. Mass spectral analysis shows formation of product, with m/z=525.3 [Fe(L−2H⁺)⁺].

Metalation of phenolTOB2, Fe(phenolTOB2):

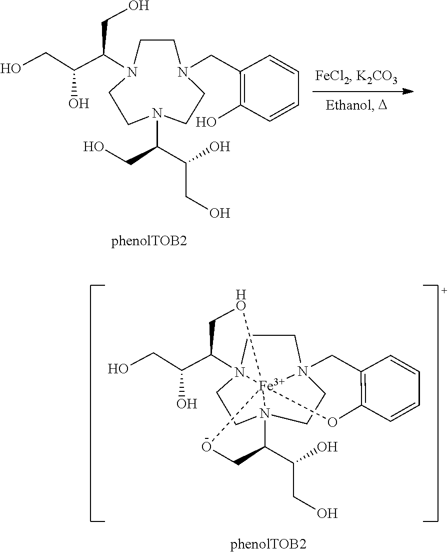

A mass of 35.6 mg ligand (Fw=443 g/mol; 0.0803 mmol) was dissolved and set to stir in 3 mL ethanol. Equimolar anhydrous ferric chloride (FW 162.204 g/mol, 13 mg) was added, and the reaction was allowed to stir under heat. As the reaction stirred, 11 mg potassium carbonate (FW 138.21 g, 0.0803 mmol) was added to basify. When mass spectral analysis showed conversion to product, the reaction was cooled to room temperature, and compound was isolated by precipitation with diethyl ether. MS ESI m/z=497.7 [Fe(L−2H⁺)⁺]. Complex was analyzed for proton water relaxation on 1.4 T benchtop NMR at 33° C., pH 7.0 to give r₁=1.2±0.2 mM⁻¹ s⁻¹.

Although the present disclosure has been described with respect to one or more particular examples, it will be understood that other examples of the present disclosure may be made without departing from the scope of the present disclosure.

1. A macrocyclic complex comprising a macrocyclic core with the following structure: 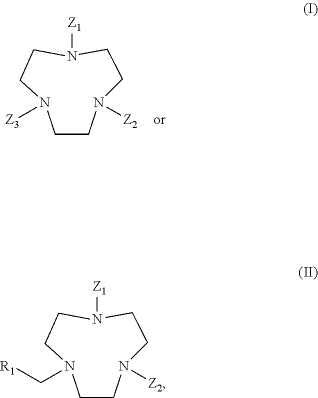
wherein
R₁ is a substituted or unsubstituted phenyl group, a substituted or unsubstituted heteroaryl group, a substituted or unsubstituted alkyl group wherein the substituted or unsubstituted alkyl group is not a methyl group,
Z₁, Z₂, and Z₃ are independently chosen from one or more of the following pendant groups: 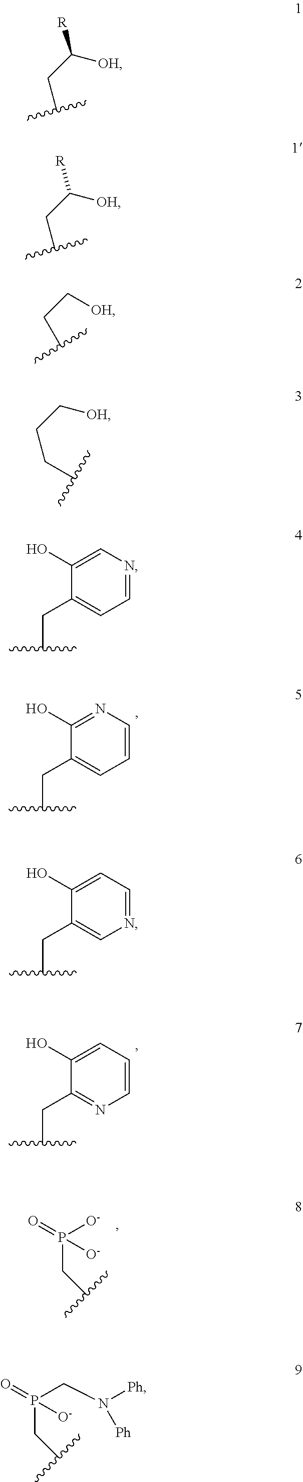 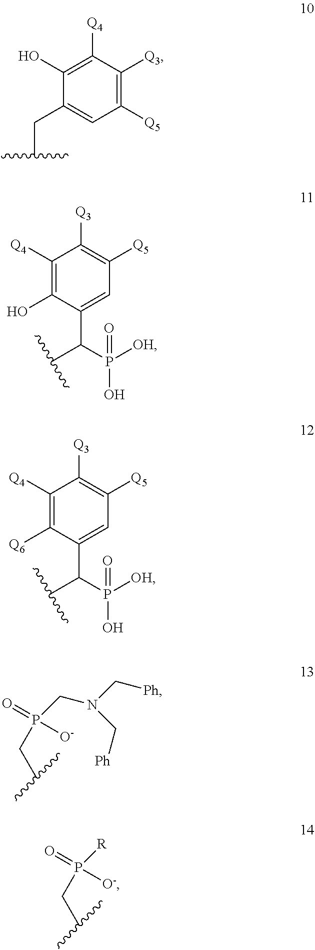
and protonated, partially deprotonated, or deprotonated species thereof,
wherein Q₃, Q₄ and Q₅ are each independently anionic groups or chosen from —H, —NR₂, —NO₂, —CN, —(CH₂)ₘNR₂, OH, OR, —P(O)OH₂, —(CH₂)ₘPO(OH)₂, —SO₃H, and deprotonated species thereof, wherein m is 1 or 2 and R is an alkyl group, CF₃ group, aryl group, alkyl carboxylate, or alkyl carboxylic acid; and
the macrocyclic complex has two of any of 1, 1′, 2, 3, 4, 8, 10, or a combination thereof and not all the pendant groups are the same, and
a high-spin Fe(III) cation complexed to the macrocyclic core and/or at least one pendant group substituent of the macrocyclic compound,
with the provisos:
i) when two of the pendant groups are structures 1, 1′, 2, 3, or any combination thereof, then the third pendent group is not 1, 1′, 2, or 3;
ii) when two of the pendant groups are structures 1, 1′, 2, 3, 8, 14 or any combination thereof, then the third pendent group is not 1, 1′, 2, 3, 8, or 14;
iii) when two of the pendant groups are structures 1, 1′, 2, 3, 10, or any combination thereof and R of structures 1 and/or structure 1′ is phenyl, then the R₁ is not a substituted or unsubstituted heteroaryl, a substituted or unsubstituted alkyl group;
iv) the macrocyclic core does not have the following structure: 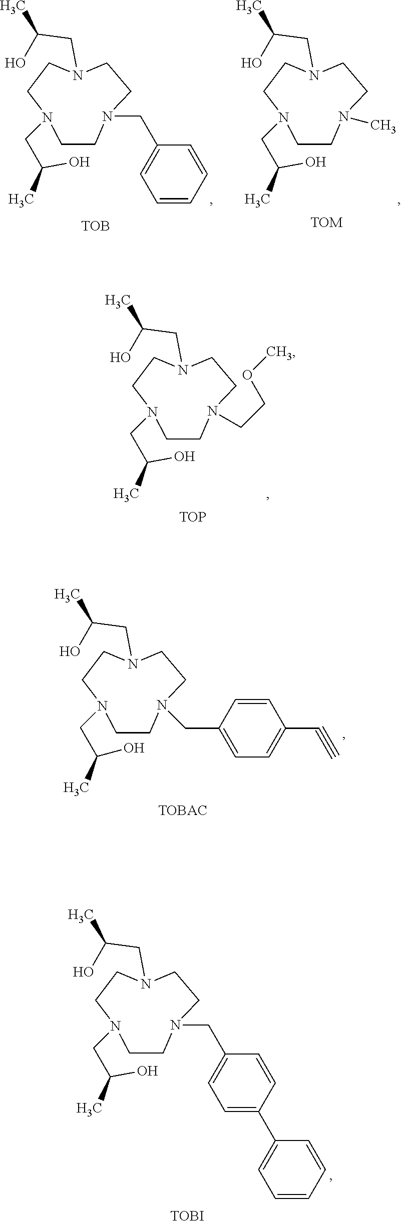 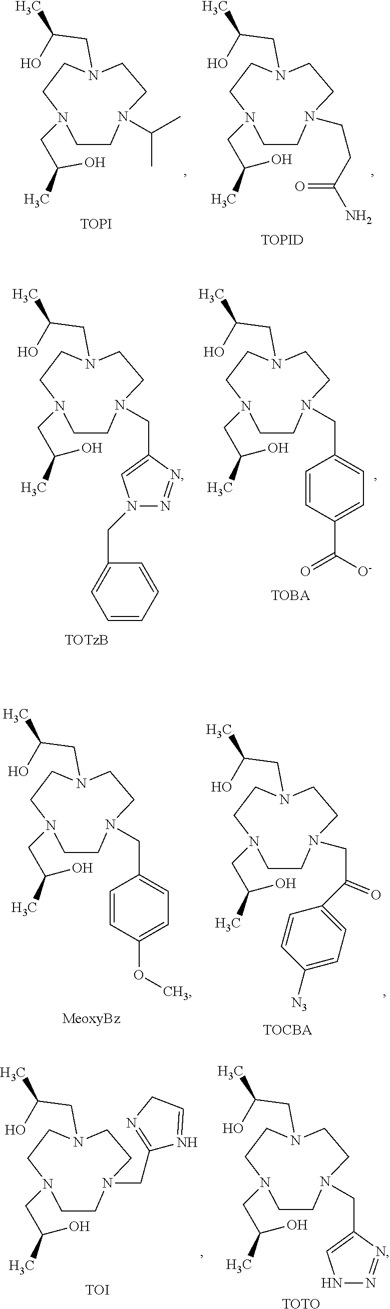 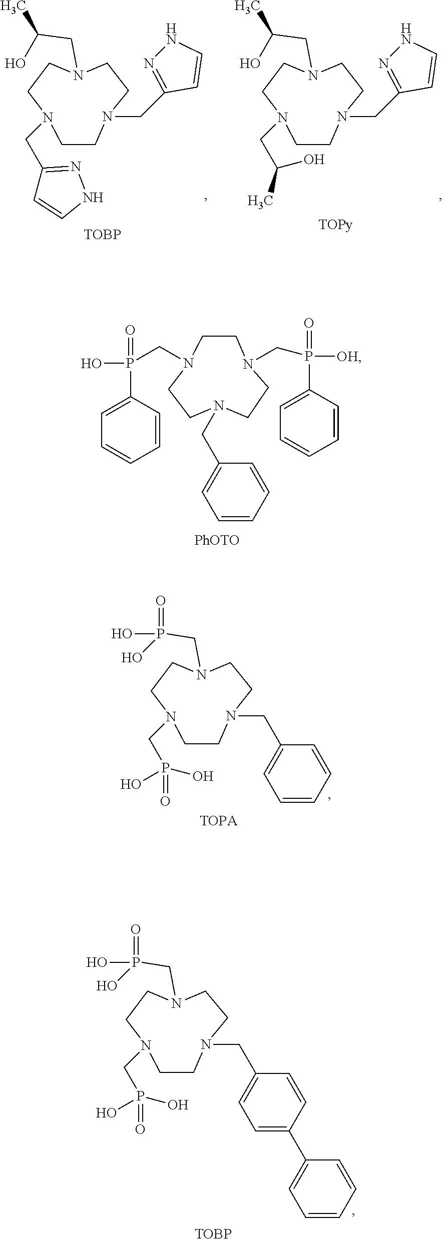 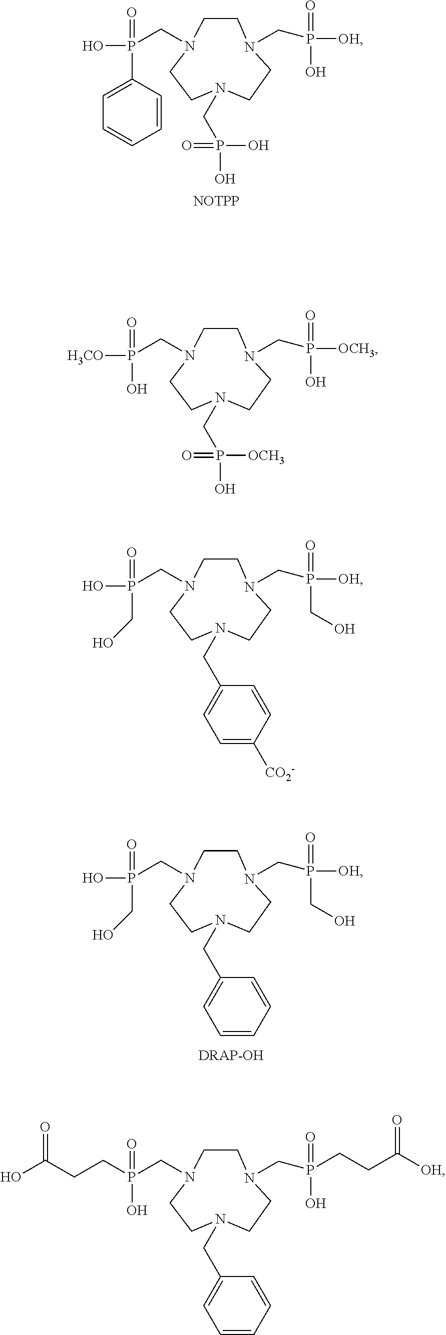 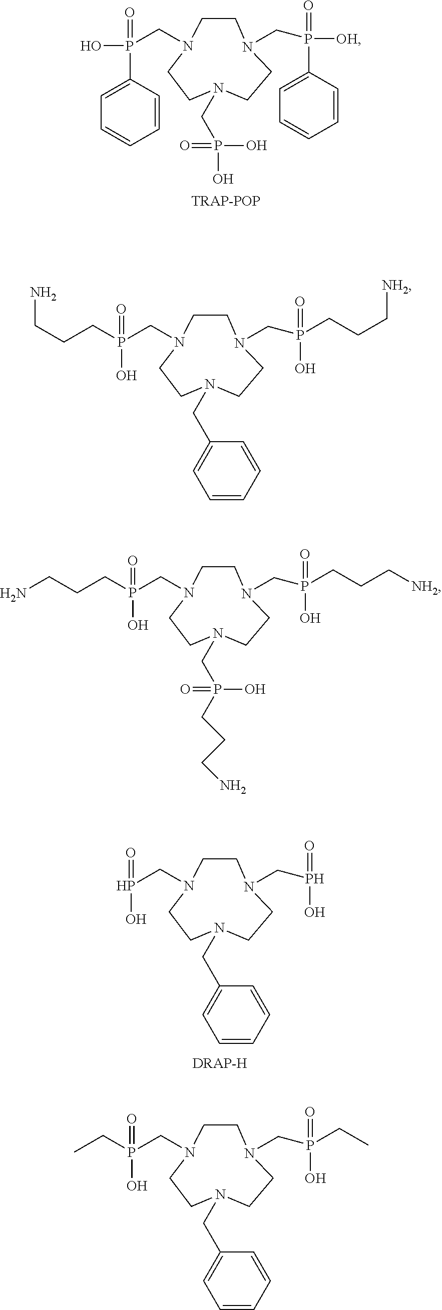 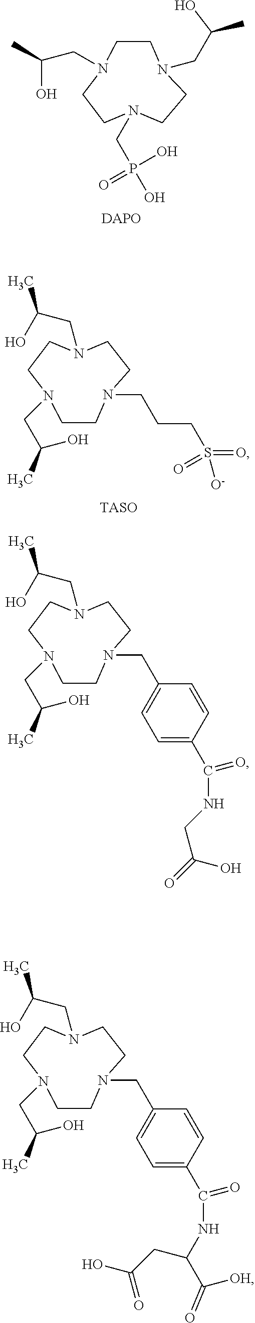 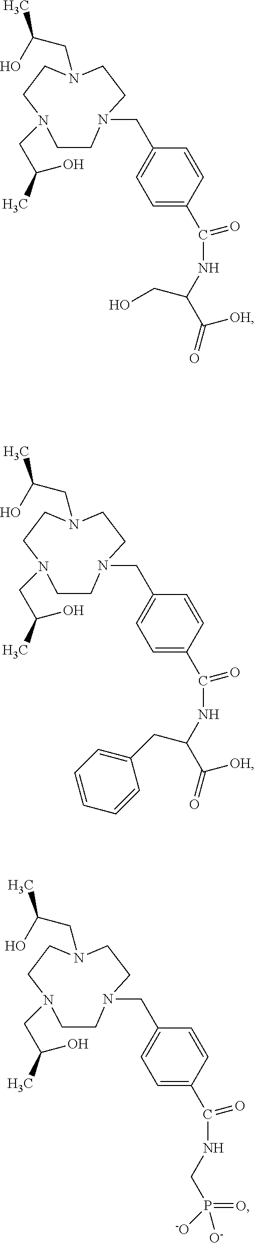 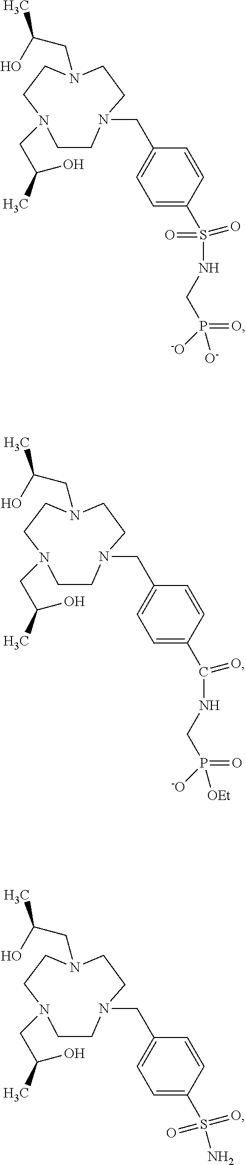 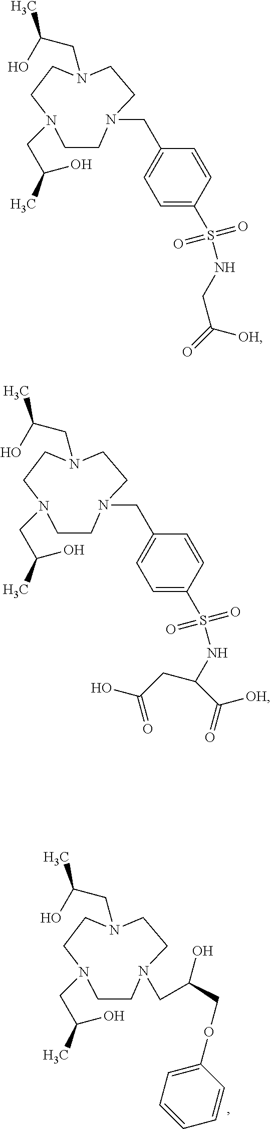 [SMILES: C[C@H](O)CN1CCN(Cc2ccc(S(=O)(=O)NCC(=O)O)cc2)CCN(C[C@H](C)O)CC1 ; C[C@H](O)CN1CCN(Cc2ccc(S(=O)(=O)NC(CC(=O)O)C(=O)O)cc2)CCN(C[C@H](C)O)CC1 ; C[C@H](O)CN1CCN(C[C@@H](O)COc2ccccc2)CCN(C[C@H](C)O)CC1] 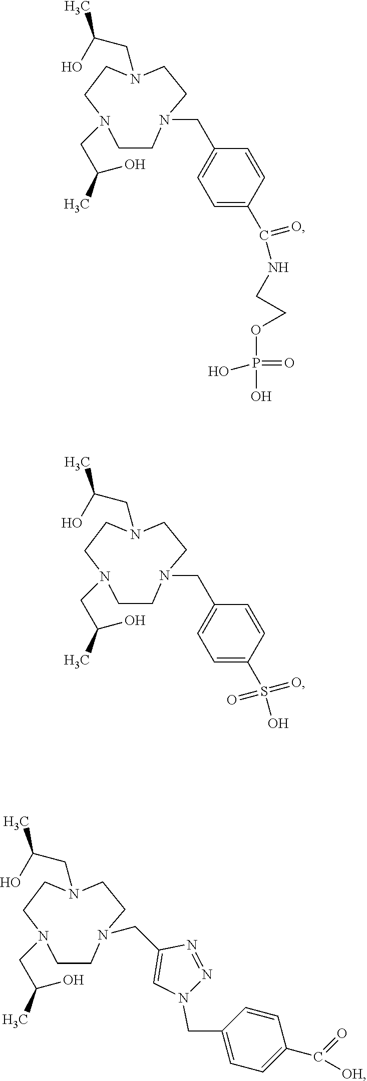 [SMILES: C[C@H](O)CN1CCN(Cc2ccc(C(=O)NCCOP(=O)(O)O)cc2)CCN(C[C@H](C)O)CC1 ; C[C@H](O)CN1CCN(Cc2ccc(S(=O)(=O)O)cc2)CCN(C[C@H](C)O)CC1 ; C[C@H](O)CN1CCN(Cc2cn(Cc3ccc(C(=O)O)cc3)nn2)CCN(C[C@H](C)O)CC1] 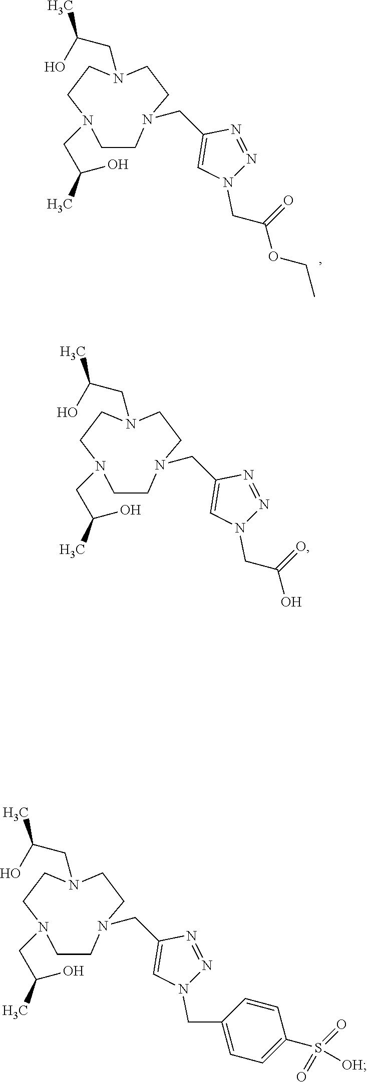 [SMILES: CCOC(=O)Cn1cc(CN2CCN(C[C@H](C)O)CCN(C[C@H](C)O)CC2)nn1 ; C[C@H](O)CN1CCN(Cc2cn(CC(=O)O)nn2)CCN(C[C@H](C)O)CC1 ; C[C@H](O)CN1CCN(Cc2cn(Cc3ccc(S(=O)(=O)O)cc3)nn2)CCN(C[C@H](C)O)CC1]
and
v) the macrocyclic complex does not have the following structure: 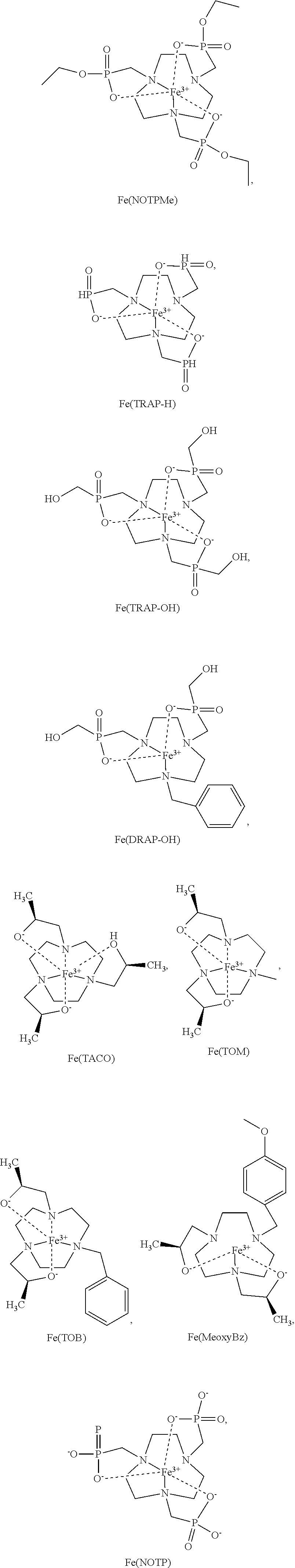 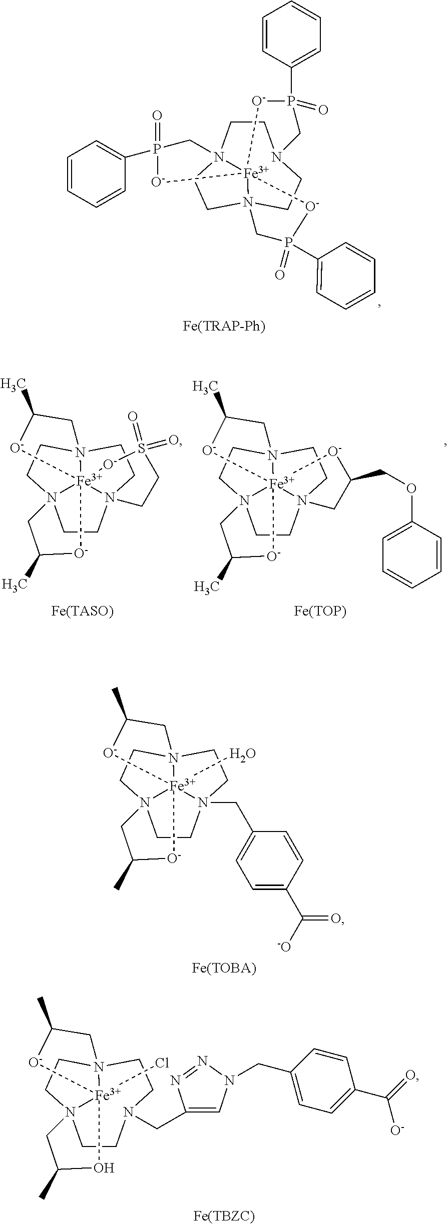
wherein the Fe(III) is high spin Fe(III).

2. The macrocyclic complex of claim 1, wherein at least one or all of the one or more pendant groups is/are covalently bound to a N on the macrocyclic core.

3. The macrocyclic complex of claim 1, wherein the macrocyclic complex has at least one water or at least one hydroxide complexed to the high-spin Fe(III) cation.

4. The macrocyclic complex of claim 1, wherein Z₁, Z₂, and Z₃ are independently chosen from: 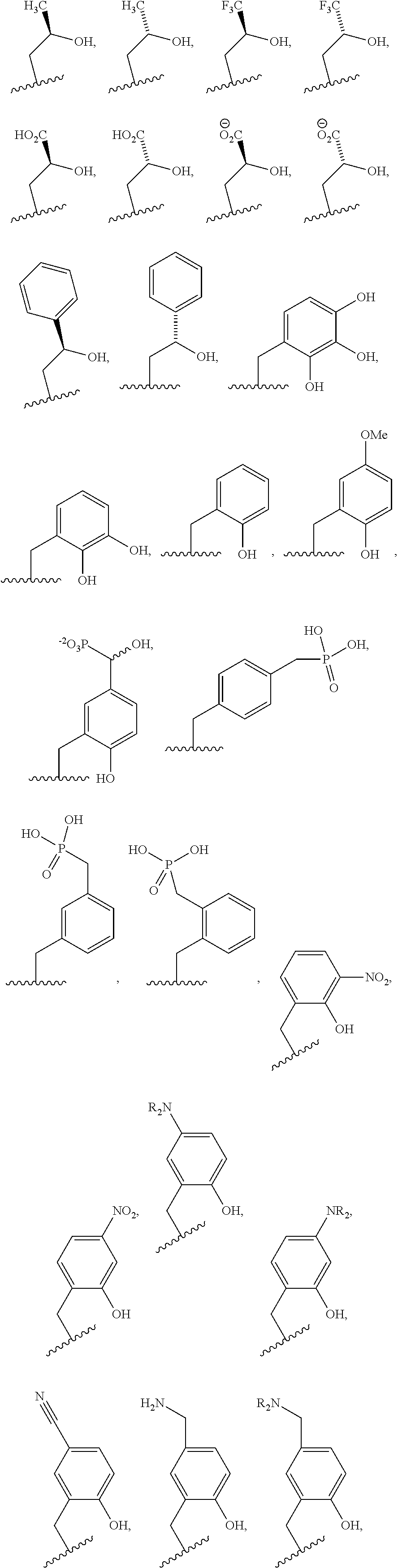 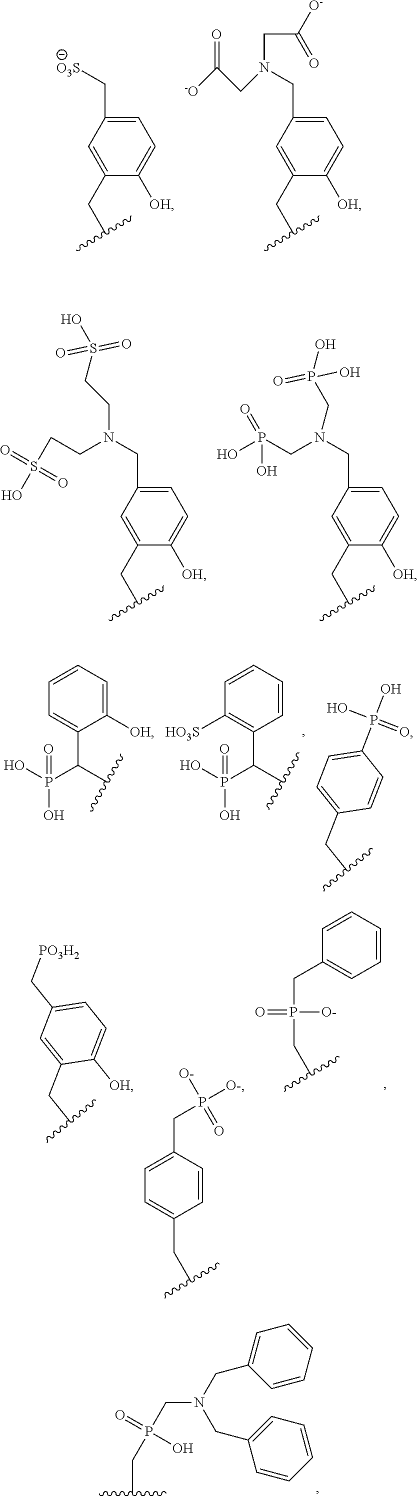
and protonated, deprotonated, and partially deprotonated species thereof (where applicable).

5. The macrocyclic complex of claim 1, wherein the macrocyclic core has the following structure to which high-spin Fe(III) is complexed thereto: 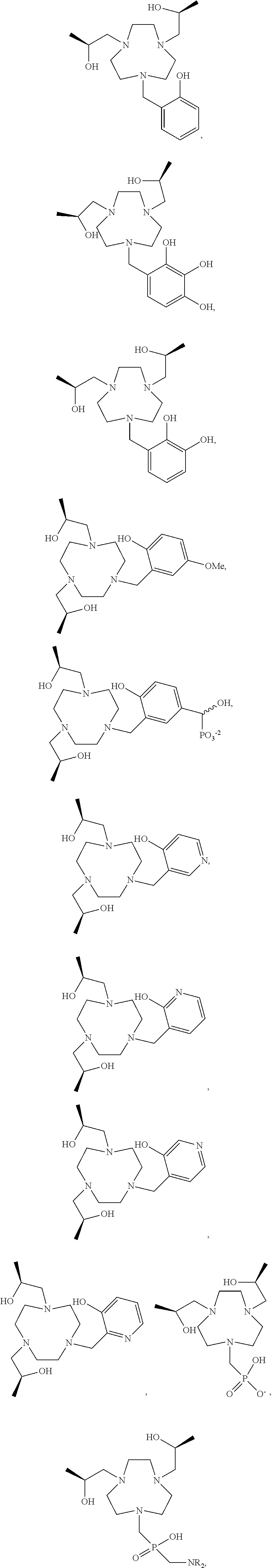 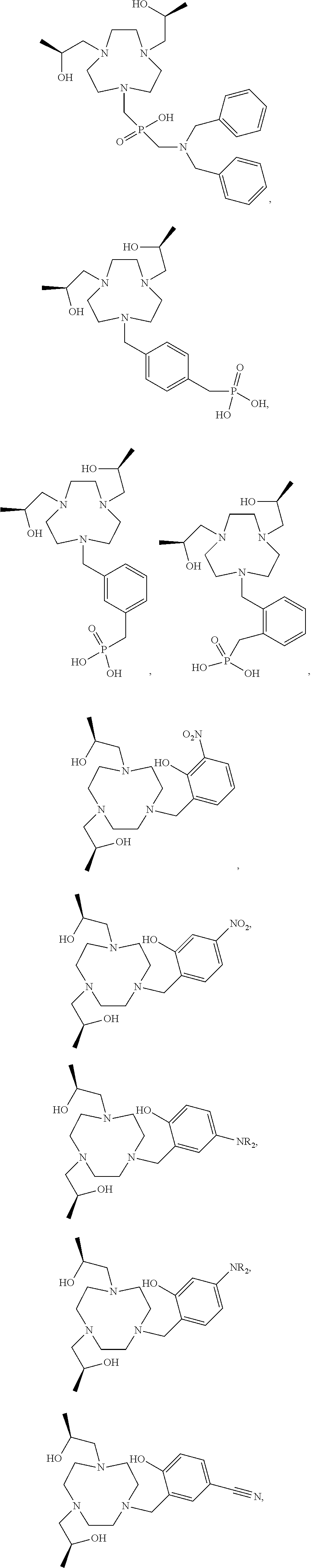 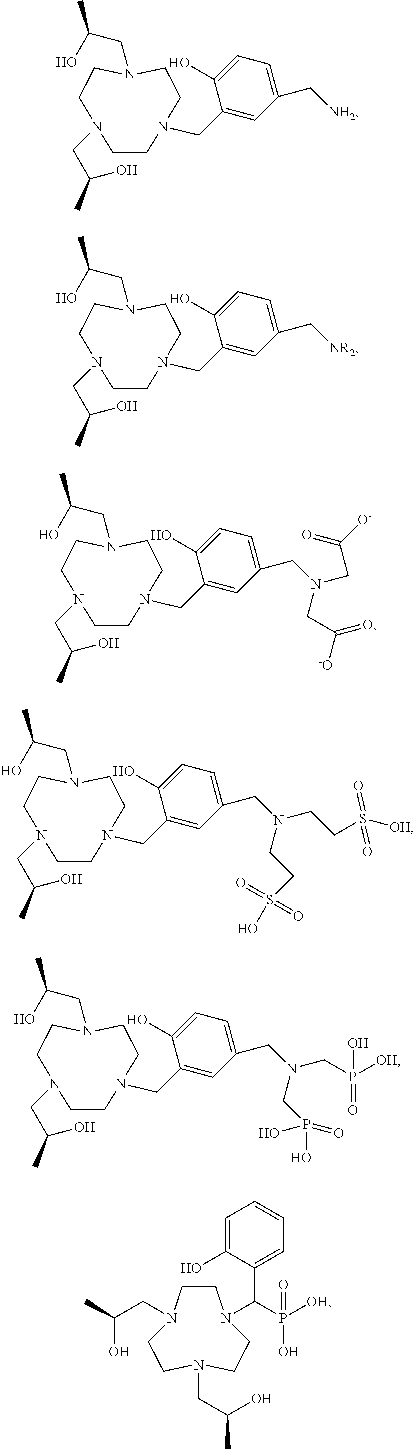 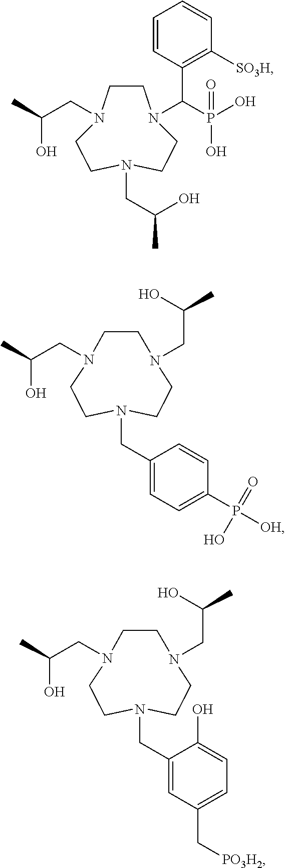
or protonated, deprotonated, or partially deprotonated species thereof (where applicable).

6. The macrocyclic complex of claim 1, wherein the macrocyclic complex has the following structure: 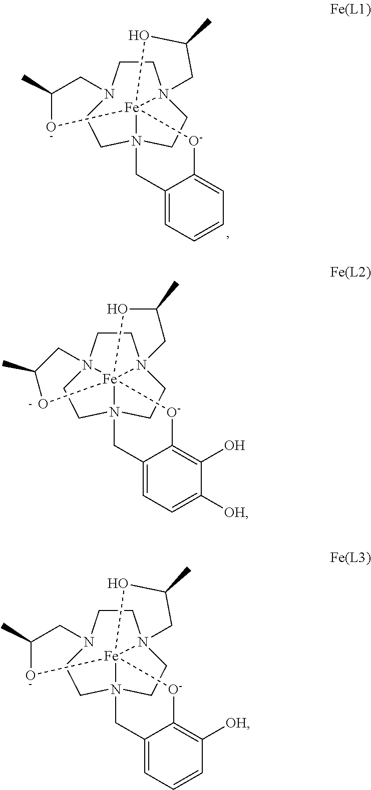 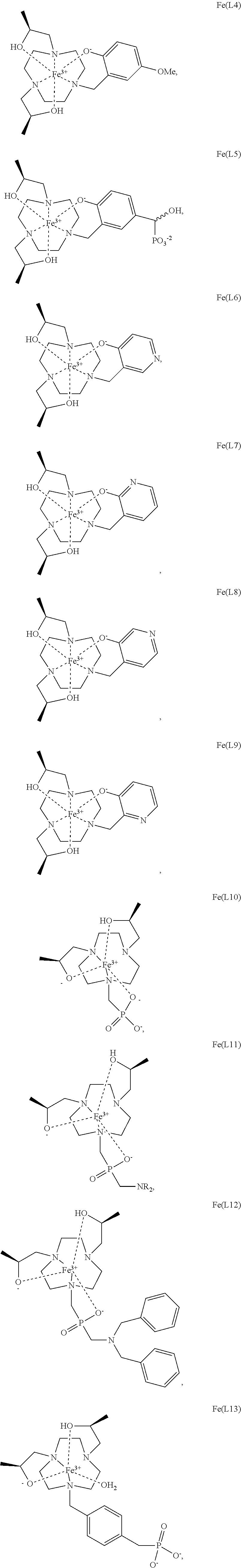 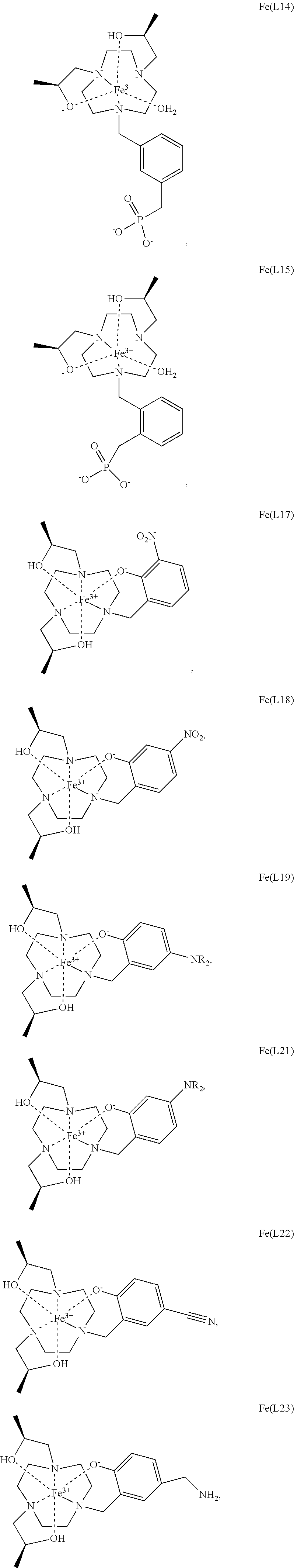 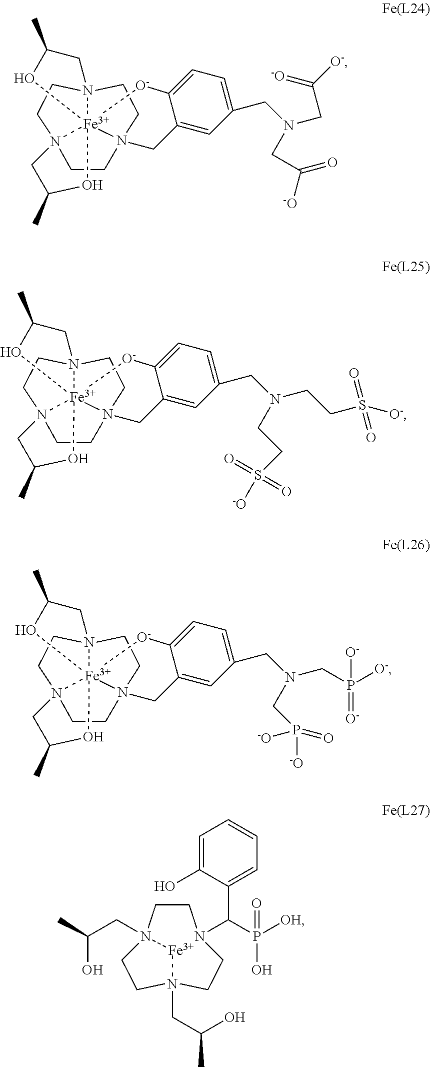 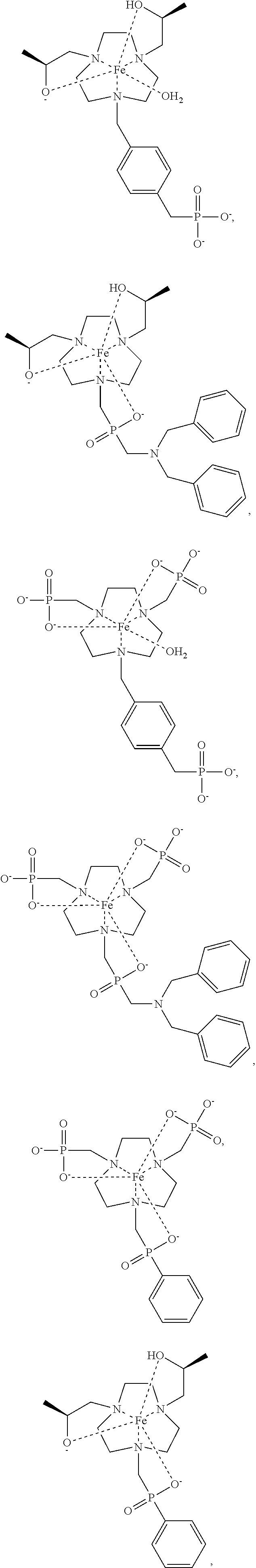 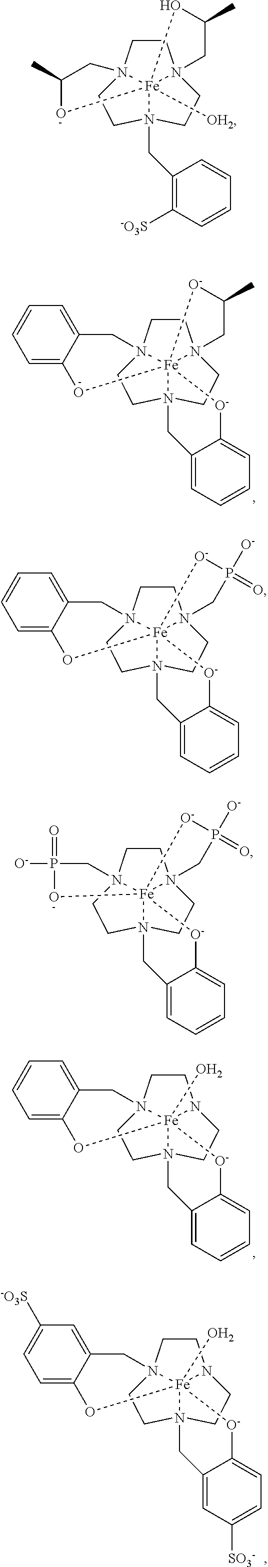 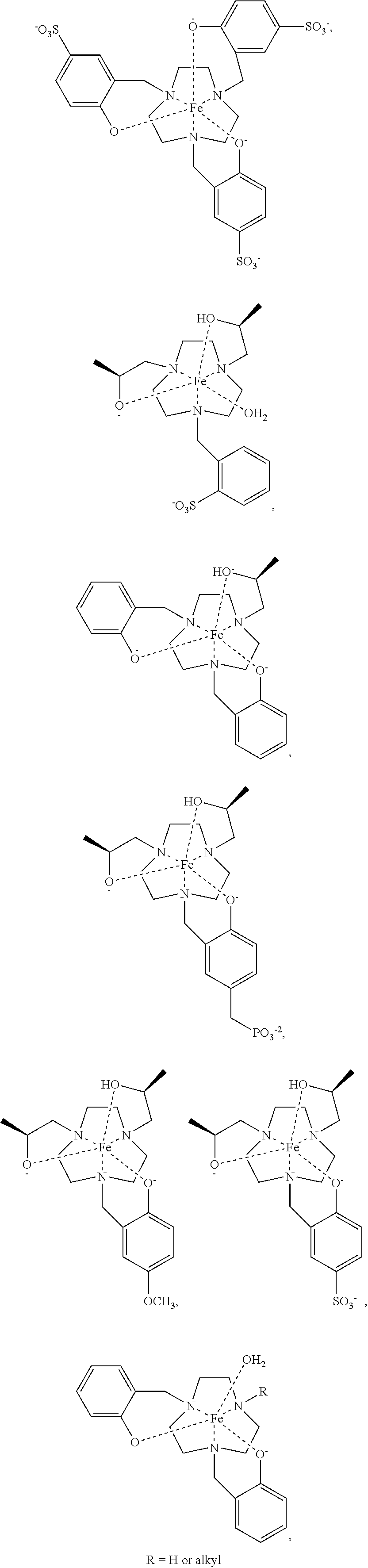 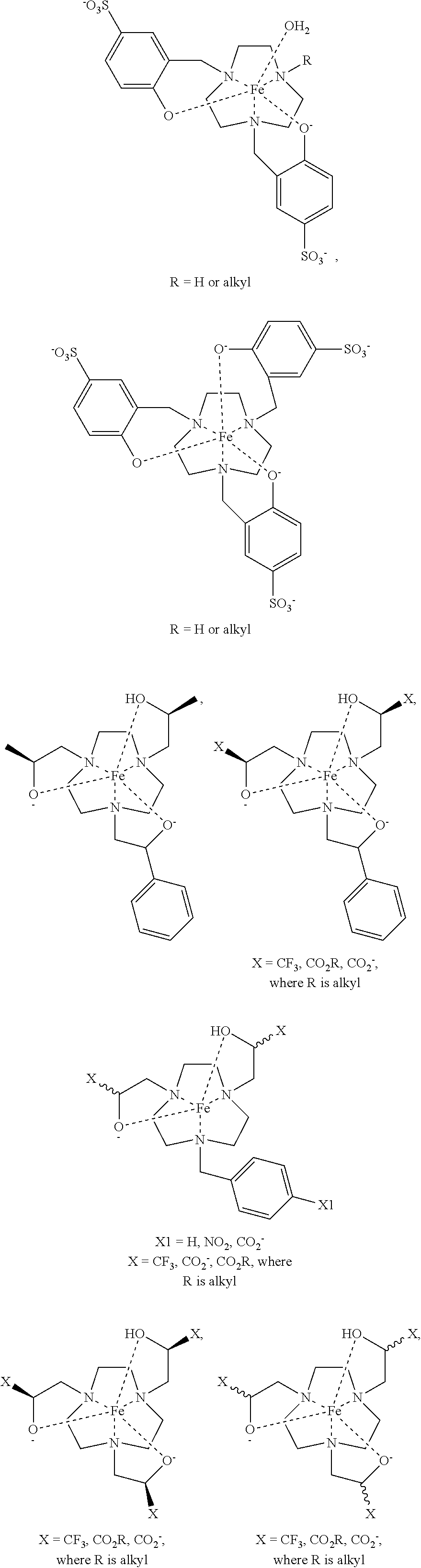 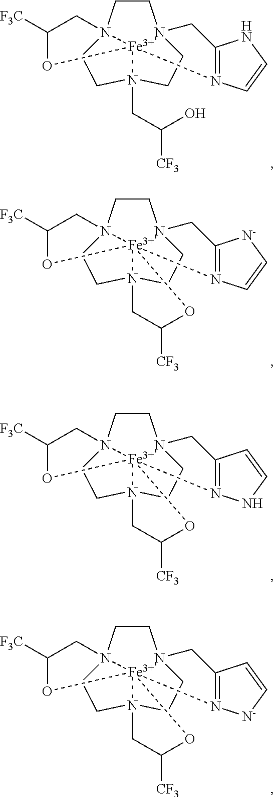
or deprotonated, partially deprotonated, or protonated species thereof (where applicable).

7. A composition comprising one or more macrocyclic complex of claim 1.

8. The composition of claim 7, wherein the composition further comprises human serum albumin and/or meglumine.

9. A method to obtain an image of at least a portion of a cell, organ, vasculature, or tissue comprising:
contacting the cell, organ, vasculature, or tissue with one or more macrocyclic complex of claim 1, and
imaging at least a portion of the cell, organ, vasculature, or tissue to obtain an image of the portion of a cell, organ, vasculature, or tissue,
wherein the image is obtained by using magnetic resonance.

10. The method of claim 9, wherein the cell, organ, vasculature, or tissue is part of an individual.

11. The method of claim 9, wherein the image is obtained using magnetic resonance imaging (MRI).

12. The method of claim 9, wherein the macrocyclic complex is a T₁ agent.

13. A macrocyclic complex having the following structure: 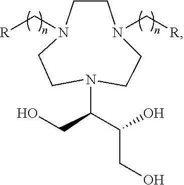
where the tri(hydroxy)butyl group(s) and —(CH₂)ₙR groups are pendant groups and each R is independently selected from alkyl groups; aryl groups; heteroaryl groups; alkyl groups comprising one or more —OH groups, one or more sulfonic acid groups, one or more carboxylic acid groups, one or more phosphonic acid groups, one or more alkyl groups, or combinations thereof, aryl groups comprising one or more —OH groups, one or more sulfonic acid groups, one or more carboxylic acid groups, one or more phosphonic acid groups, one or more alkyl groups, or combinations thereof, heteroaryl groups comprising one or more —OH groups, one or more sulfonic acid groups, one or more carboxylic acid groups, one or more phosphonic acid groups, one or more alkyl groups, or combinations thereof, and H; or a salt, a partial salt, a hydrate, a polymorph, or a stereoisomer thereof;
n is 1, 2, or 3,
wherein a high-spin Fe(III) cation is complexed to the macrocyclic core and/or at least one pendant group substituent of the macrocyclic compound.

14. The macrocyclic complex of claim 13, wherein the macrocyclic complex further comprises a coordinating pendant group or a non-coordinating pendant group.

15. The macrocyclic complex of claim 13, wherein at least one of the pendant groups is substituted at a benzylic position or any carbon the alkyl group leading to the heteroatom of the pendant group.

16. The macrocyclic complex of claim 13, wherein the pendant groups are chosen from: 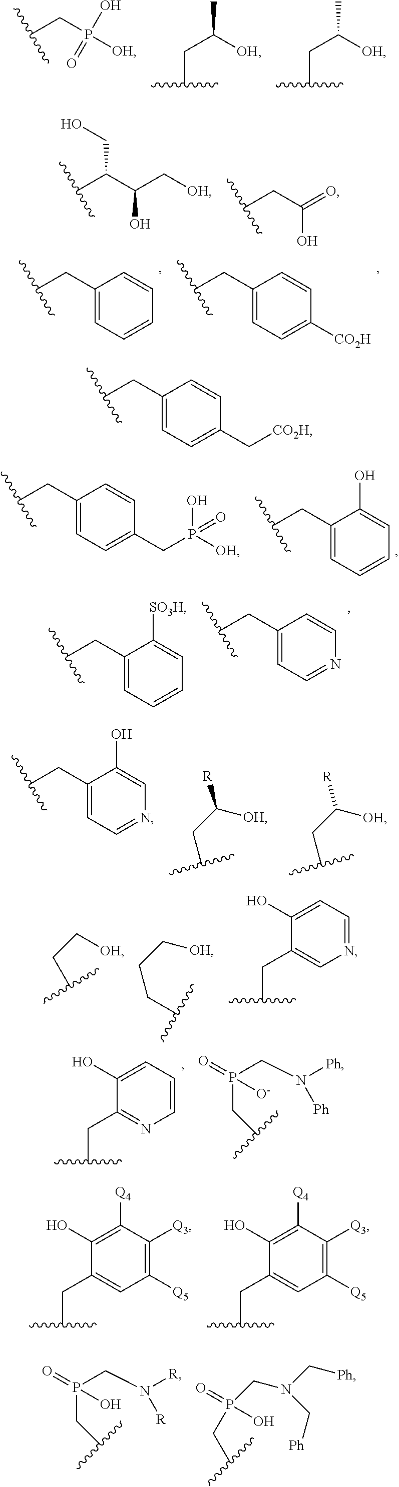
and protonated, partially deprotonated, and deprotonated species thereof (where applicable), Q₃, Q₄ and Q₅ are each independently anionic groups or chosen from —H, —NR₂, —NO₂, —CN, —(CH₂)ₘNR₂, OH, OR, —CH₂PO(OH)₂, —(CH₂)ₘP(O)(OH)₂, —SO₃H, and deprotonated, partially deprotonated, and protonated species thereof (where applicable).

17. The macrocyclic complex of claim 13, wherein the macrocyclic complex has at least one open coordination site.

18. The macrocyclic complex of claim 13, wherein the macrocyclic complex has at least one water or at least one hydroxide complexed to the high-spin Fe(III) cation.

19. The macrocyclic complex of claim 13, wherein the macrocyclic complex has the following structure: 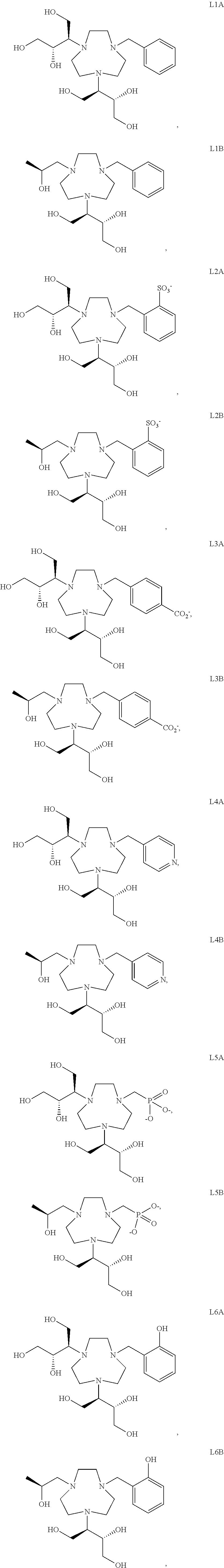 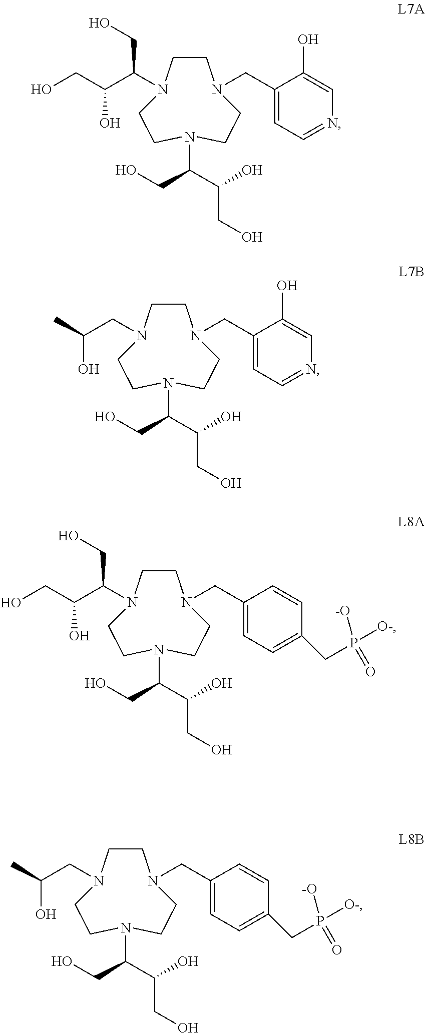
or protonated or deprotonated analogs thereof, wherein high-spin Fe(III) is complexed.

20. A macrocyclic complex of claim 13, wherein the macrocyclic complex has the following structure: 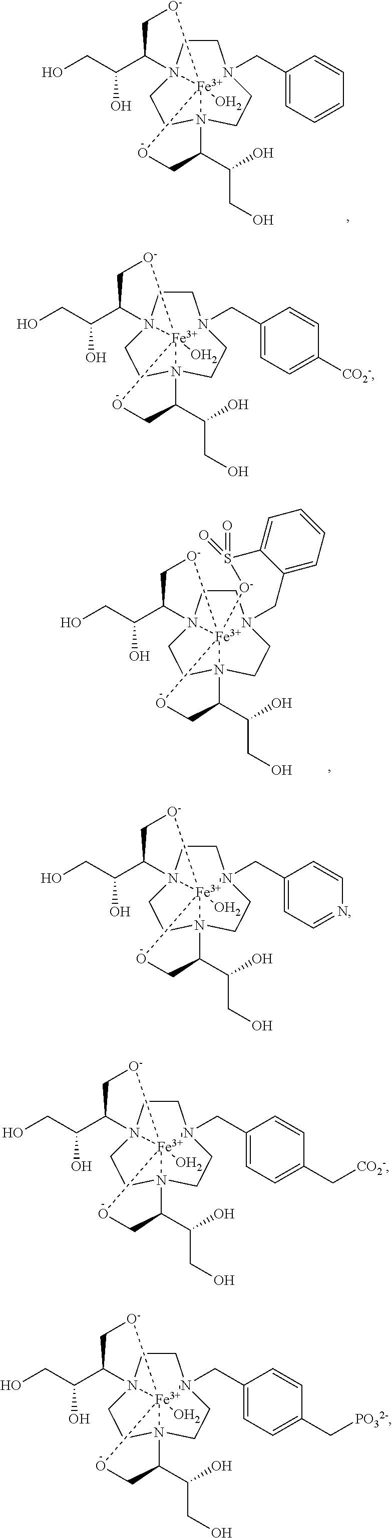 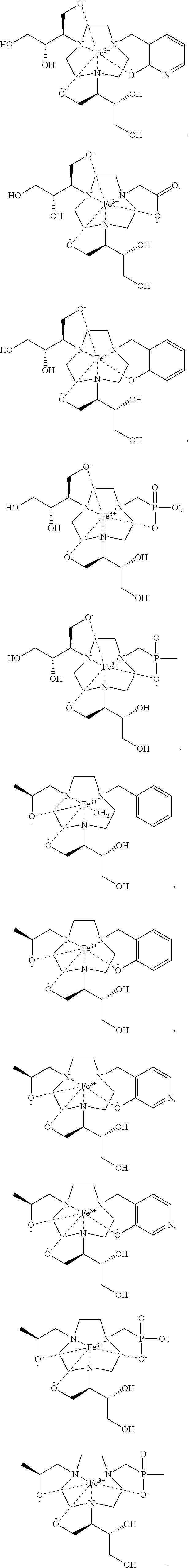 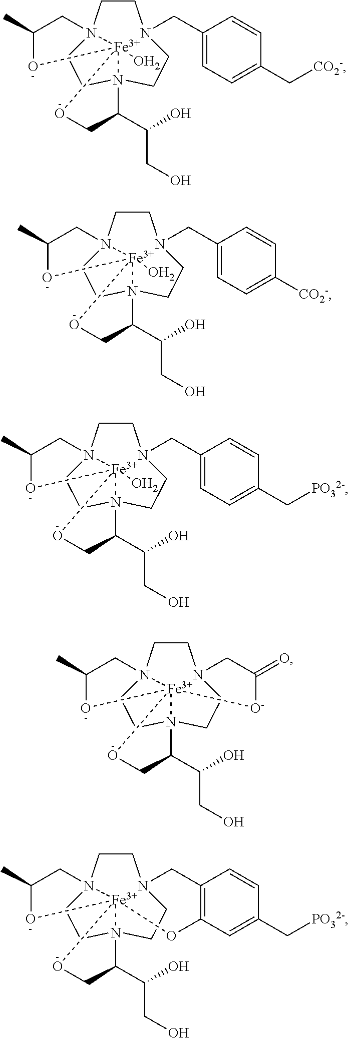
or protonated, partially deprotonated, or deprotonated species thereof.

21. The macrocyclic complex of claim 20, wherein the macrocyclic complex has the following structure:

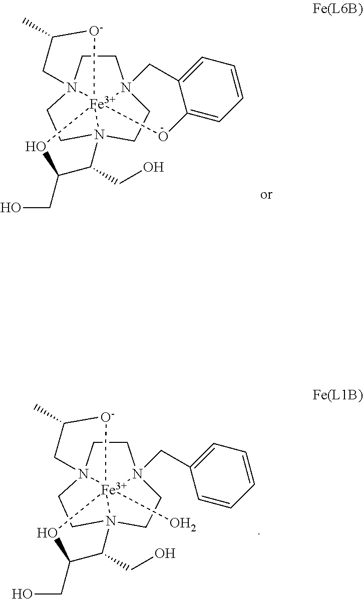

22. A composition comprising one or more macrocyclic complexes of claim 13, and a pharmaceutically acceptable carrier.

23. The composition of claim 22, wherein the composition further comprises human serum albumin and/or meglumine.

24. A method to obtain an image of at least a portion of a cell, organ, vasculature, or tissue comprising:
contacting the cell, organ, vasculature, or tissue with one or more macrocyclic complex of claim 13, and
imaging at least a portion of the cell, organ, vasculature, or tissue to obtain an image of the portion of a cell, organ, vasculature, or tissue,
wherein the image is obtained by using magnetic resonance.

25. The method according to claim 24, wherein the cell, organ, vasculature, or tissue is part of an individual.

26. The method of claim 24, wherein the image is obtained using magnetic resonance imaging (MRI).

27. The method of claim 24, wherein the macrocyclic complex is a T₁ agent.

28. A macrocyclic compound having the following structure: 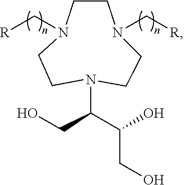
where the tri(hydroxy)butyl group(s) and —(CH₂)ₙR groups are pendant groups and each R is independently selected from alkyl groups; aryl groups; heteroaryl groups; alkyl groups comprising one or more —OH groups, one or more sulfonic acid groups, one or more carboxylic acid groups, one or more phosphonic acid groups, one or more alkyl groups, or combinations thereof, aryl groups comprising one or more —OH groups, one or more sulfonic acid groups, one or more carboxylic acid groups, one or more phosphonic acid groups, one or more alkyl groups, or combinations thereof, heteroaryl groups comprising one or more —OH groups, one or more sulfonic acid groups, one or more carboxylic acid groups, one or more phosphonic acid groups, one or more alkyl groups, or combinations thereof, and H; or a salt, a partial salt, a hydrate, a polymorph, or a stereoisomer thereof;
n is 1, 2, or 3.

29. The macrocyclic compound of claim 28, further comprising a coordinating pendant group or a non-coordinating pendant group.

30. The macrocyclic compound of claim 28, wherein at least one of the pendant groups is substituted at a benzylic position or any carbon the alkyl group leading to the heteroatom of the pendant group.

31. The macrocyclic compound of claim 28, wherein the pendant groups are chosen from: 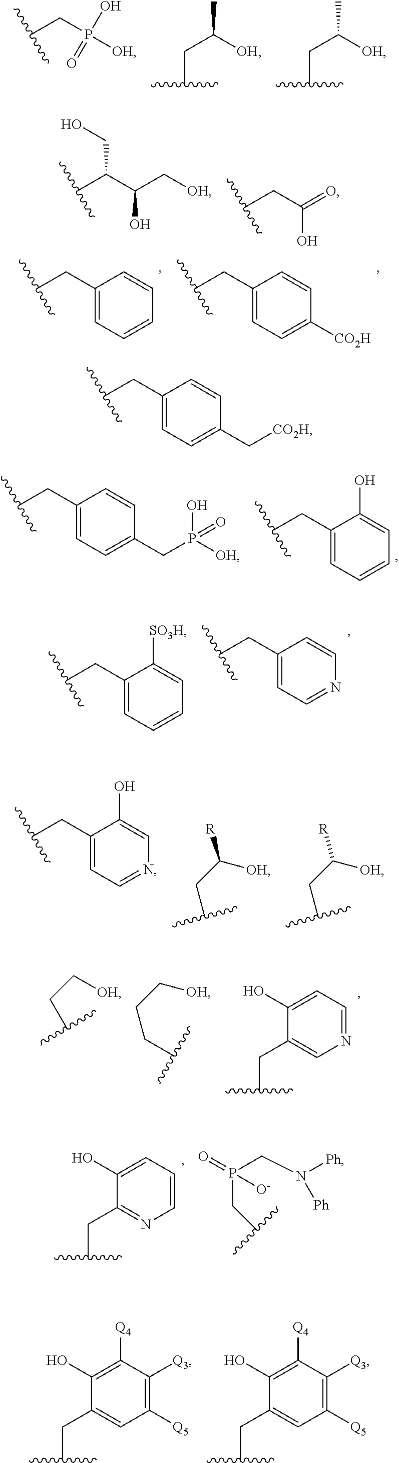 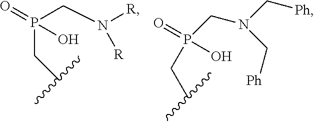
and protonated, partially deprotonated, and deprotonated species thereof (where applicable), Q₃, Q₄ and Q₅ are each independently anionic groups or chosen from —H, —NR₂, —NO₂, —CN, —(CH₂)ₘNR₂, OH, OR, —CH₂PO(OH)₂, —(CH₂)ₘP(O)(OH)₂, —SO₃H, and deprotonated, partially deprotonated, and protonated species thereof (where applicable).

32. The macrocyclic compound of claim 28, wherein the macrocyclic compound has at least one open coordination site.

33. The macrocyclic compound of claim 28, wherein the macrocyclic compound has the following structure: 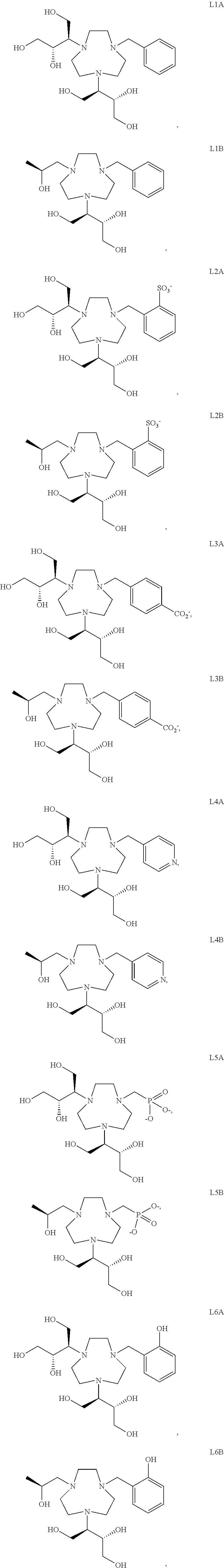 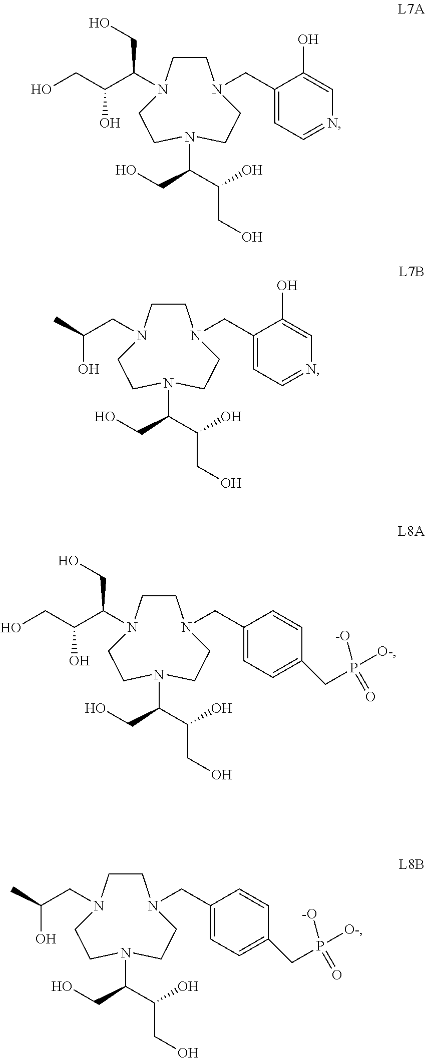
or protonated, partially deprotonated, or deprotonated species thereof.
